SEQUENCE LISTING (187 sequences)

SEQ ID NO: 1 - protein, 21 aa, synthetic construct
  REGION 1..21: note = Description of Artificial Sequence: Synthetic peptide
MALPVTALLLPLALLLHAARP

SEQ ID NO: 2 - protein, 10 aa, synthetic construct
  REGION 1..10: note = Description of Artificial Sequence: Synthetic peptide
GYPYDVPDYA

SEQ ID NO: 3 - protein, 248 aa, synthetic construct
  REGION 1..248: note = Description of Artificial Sequence: Synthetic polypeptide
DIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSS

SEQ ID NO: 4 - protein, 251 aa, synthetic construct
  REGION 1..251: note = Description of Artificial Sequence: Synthetic polypeptide
DIVMTQTPASVSEPVGGTVTIMCQASQSISNWLAWYQQKPGQPPKLLIYQASKLASGVPSRFKGSGSGTEYTLTISDLECADAATYYCQSYYDSGSNVFFAFGGGTKVVVEGGGGGSGGGGSGGGGSGGGGSLSLEESGGDLVKPGASLTLTCTASGFSFSAGYWICWVRQAPGKGLEWIACTYAGRSGSTYYANWVNGRFTIPKTSSTTVTLQMTSLSGADTASYFCARGNAGVAVGALWGPGTLVTVSS

SEQ ID NO: 5 - protein, 264 aa, synthetic construct
  REGION 1..264: note = Description of Artificial Sequence: Synthetic polypeptide
DIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSLSGRSDNHSPLGLAGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSS

SEQ ID NO: 6 - protein, 267 aa, synthetic construct
  REGION 1..267: note = Description of Artificial Sequence: Synthetic polypeptide
DIVMTQTPASVSEPVGGTVTIMCQASQSISNWLAWYQQKPGQPPKLLIYQASKLASGVPSRFKGSGSGTEYTLTISDLECADAATYYCQSYYDSGSNVFFAFGGGTKVVVEGGGGGSGGGGSLSGRSDNHSPLGLAGSGGGGSGGGGSLSLEESGGDLVKPGASLTLTCTASGFSFSAGYWICWVRQAPGKGLEWIACTYAGRSGSTYYANWVNGRFTIPKTSSTTVTLQMTSLSGADTASYFCARGNAGVAVGALWGPGTLVTVSS

SEQ ID NO: 7 - protein, 45 aa, synthetic construct
  REGION 1..45: note = Description of Artificial Sequence: Synthetic polypeptide
GGGGSGGGGSGGGSGSKPIPNPLLGLDSTGGGSGSGGGGSGGGGS

SEQ ID NO: 8 - protein, 45 aa, synthetic construct
  REGION 1..45: note = Description of Artificial Sequence: Synthetic polypeptide
GGGGSGGGGSLVPRGSKPIPNPLLGLDSTLVPRGSGGGGSGGGGS

SEQ ID NO: 9 - protein, 45 aa, synthetic construct
  REGION 1..45: note = Description of Artificial Sequence: Synthetic polypeptide
GGGGSGGGGSGGGSGLSGRSDNHSPLGLAGSGGGSGGGGSGGGGS

SEQ ID NO: 10 - protein, 55 aa, synthetic construct
  REGION 1..55: note = Description of Artificial Sequence: Synthetic polypeptide
GGGGSLSGRSDNHSPLGLAGSKPIPNPLLGLDSTLSGRSDNHSPLGLAGSGGGGS

SEQ ID NO: 11 - protein, 469 aa, synthetic construct
  REGION 1..469: note = Description of Artificial Sequence: Synthetic polypeptide
QVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 363 aa, synthetic construct
  REGION 1..363: note = Description of Artificial Sequence: Synthetic polypeptide
GFHTINATWWANITLVGPPDTPVTWYDTQGLWFCNGSRVKNPQIRHTCNDQNLTLIHVNKTYERTYMGYNRQGTKKEDYKVVVIPPPPATVKPQPEPEYVFVYMGENKTLEGPPGTPVTWFNQDGKKFCEGEKVLHPEFNHTCDKQNLILLFVNFTHDGAYLGYNHQGTQRTHYEVTVLDLFPDSGQMKIENHSEETEQKNDEHHNWQKQGGQKQGGQKTNQTKVNDRRKTAQKRPSKLKPATIEAMLVTVTAGSNLTLVGPKAEGKVTWFDGDLKRPCEPNYRLRHECNNQNLTLINVTKDYEGTYYGTNDKDEGKRYRVKVNTTNSQSVKIQPYTRQTTPDQEHKFELQFETNGNYDSKIP

SEQ ID NO: 13 - protein, 200 aa, synthetic construct
  REGION 1..200: note = Description of Artificial Sequence: Synthetic polypeptide
HDACIPVVGKIGTNVTLNAVDFHPGDHVRWSYGPGGAGYMLCVYTGSWTEYKKPDIIFKCLSNNSLLLINVTVNYTNTYRTLTSLNNWVHNQHHHKFPGWNLDTCYSLTVNENGTFPTTTTKKPTTTTRTTTTTTTKKTTTTRTTTAAKKTTISTTHHKHSSPKKSSTPNSHVEHHVGFEATAAETPLQPSPQHQHVATH

SEQ ID NO: 14 - protein, 115 aa, synthetic construct
  REGION 1..115: note = Description of Artificial Sequence: Synthetic polypeptide
QFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVA

SEQ ID NO: 15 - protein, 111 aa, synthetic construct
  REGION 1..111: note = Description of Artificial Sequence: Synthetic polypeptide
ALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAGT

SEQ ID NO: 16 - protein, 793 aa, synthetic construct
  REGION 1..793: note = Description of Artificial Sequence: Synthetic polypeptide
MALPVTALLLPLALLLHAARPGYPYDVPDYADIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSLVPRGSKPIPNPLLGLDSTLVPRGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - protein, 793 aa, synthetic construct
  REGION 1..793: note = Description of Artificial Sequence: Synthetic polypeptide
MALPVTALLLPLALLLHAARPGYPYDVPDYADIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSGGGSGSKPIPNPLLGLDSTGGGSGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 18 - protein, 796 aa, synthetic construct
  REGION 1..796: note = Description of Artificial Sequence: Synthetic polypeptide
MALPVTALLLPLALLLHAARPGYPYDVPDYADIVMTQTPASVSEPVGGTVTIMCQASQSISNWLAWYQQKPGQPPKLLIYQASKLASGVPSRFKGSGSGTEYTLTISDLECADAATYYCQSYYDSGSNVFFAFGGGTKVVVEGGGGGSGGGGSGGGGSGGGGSLSLEESGGDLVKPGASLTLTCTASGFSFSAGYWICWVRQAPGKGLEWIACTYAGRSGSTYYANWVNGRFTIPKTSSTTVTLQMTSLSGADTASYFCARGNAGVAVGALWGPGTLVTVSSGGGGSGGGGSLVPRGSKPIPNPLLGLDSTLVPRGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 796 aa, synthetic construct
  REGION 1..796: note = Description of Artificial Sequence: Synthetic polypeptide
MALPVTALLLPLALLLHAARPGYPYDVPDYADIVMTQTPASVSEPVGGTVTIMCQASQSISNWLAWYQQKPGQPPKLLIYQASKLASGVPSRFKGSGSGTEYTLTISDLECADAATYYCQSYYDSGSNVFFAFGGGTKVVVEGGGGGSGGGGSGGGGSGGGGSLSLEESGGDLVKPGASLTLTCTASGFSFSAGYWICWVRQAPGKGLEWIACTYAGRSGSTYYANWVNGRFTIPKTSSTTVTLQMTSLSGADTASYFCARGNAGVAVGALWGPGTLVTVSSGGGGSGGGGSGGGSGSKPIPNPLLGLDSTGGGSGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 20 - protein, 793 aa, synthetic construct
  REGION 1..793: note = Description of Artificial Sequence: Synthetic polypeptide
MALPVTALLLPLALLLHAARPGYPYDVPDYADIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSGGGSGLSGRSDNHSPLGLAGSGGGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - protein, 796 aa, synthetic construct
  REGION 1..796: note = Description of Artificial Sequence: Synthetic polypeptide
MALPVTALLLPLALLLHAARPGYPYDVPDYADIVMTQTPASVSEPVGGTVTIMCQASQSISNWLAWYQQKPGQPPKLLIYQASKLASGVPSRFKGSGSGTEYTLTISDLECADAATYYCQSYYDSGSNVFFAFGGGTKVVVEGGGGGSGGGGSGGGGSGGGGSLSLEESGGDLVKPGASLTLTCTASGFSFSAGYWICWVRQAPGKGLEWIACTYAGRSGSTYYANWVNGRFTIPKTSSTTVTLQMTSLSGADTASYFCARGNAGVAVGALWGPGTLVTVSSGGGGSGGGGSGGGSGLSGRSDNHSPLGLAGSGGGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 22 - protein, 803 aa, synthetic construct
  REGION 1..803: note = Description of Artificial Sequence: Synthetic polypeptide
MALPVTALLLPLALLLHAARPGYPYDVPDYADIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSLSGRSDNHSPLGLAGSKPIPNPLLGLDSTLSGRSDNHSPLGLAGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - protein, 806 aa, synthetic construct
  REGION 1..806: note = Description of Artificial Sequence: Synthetic polypeptide
MALPVTALLLPLALLLHAARPGYPYDVPDYADIVMTQTPASVSEPVGGTVTIMCQASQSISNWLAWYQQKPGQPPKLLIYQASKLASGVPSRFKGSGSGTEYTLTISDLECADAATYYCQSYYDSGSNVFFAFGGGTKVVVEGGGGGSGGGGSGGGGSGGGGSLSLEESGGDLVKPGASLTLTCTASGFSFSAGYWICWVRQAPGKGLEWIACTYAGRSGSTYYANWVNGRFTIPKTSSTTVTLQMTSLSGADTASYFCARGNAGVAVGALWGPGTLVTVSSGGGGSLSGRSDNHSPLGLAGSKPIPNPLLGLDSTLSGRSDNHSPLGLAGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - protein, 809 aa, synthetic construct
  REGION 1..809: note = Description of Artificial Sequence: Synthetic polypeptide
MALPVTALLLPLALLLHAARPGYPYDVPDYADIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSLSGRSDNHSPLGLAGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSGGGSGLSGRSDNHSPLGLAGSGGGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 812 aa, synthetic construct
  REGION 1..812: note = Description of Artificial Sequence: Synthetic polypeptide
MALPVTALLLPLALLLHAARPGYPYDVPDYADIVMTQTPASVSEPVGGTVTIMCQASQSISNWLAWYQQKPGQPPKLLIYQASKLASGVPSRFKGSGSGTEYTLTISDLECADAATYYCQSYYDSGSNVFFAFGGGTKVVVEGGGGGSGGGGSLSGRSDNHSPLGLAGSGGGGSGGGGSLSLEESGGDLVKPGASLTLTCTASGFSFSAGYWICWVRQAPGKGLEWIACTYAGRSGSTYYANWVNGRFTIPKTSSTTVTLQMTSLSGADTASYFCARGNAGVAVGALWGPGTLVTVSSGGGGSGGGGSGGGSGLSGRSDNHSPLGLAGSGGGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 26 - protein, 799 aa, synthetic construct
  REGION 1..799: note = Description of Artificial Sequence: Synthetic polypeptide
MALPVTALLLPLALLLHAARPGYPYDVPDYADIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSLVPRGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSLVPRGSKPIPNPLLGLDSTLVPRGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 27 - protein, 802 aa, synthetic construct
  REGION 1..802: note = Description of Artificial Sequence: Synthetic polypeptide
MALPVTALLLPLALLLHAARPGYPYDVPDYADIVMTQTPASVSEPVGGTVTIMCQASQSISNWLAWYQQKPGQPPKLLIYQASKLASGVPSRFKGSGSGTEYTLTISDLECADAATYYCQSYYDSGSNVFFAFGGGTKVVVEGGGGGSGGGGSLVPRGSGGGGSGGGGSLSLEESGGDLVKPGASLTLTCTASGFSFSAGYWICWVRQAPGKGLEWIACTYAGRSGSTYYANWVNGRFTIPKTSSTTVTLQMTSLSGADTASYFCARGNAGVAVGALWGPGTLVTVSSGGGGSGGGGSLVPRGSKPIPNPLLGLDSTLVPRGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 28 - protein, 908 aa, synthetic construct
  REGION 1..908: note = Description of Artificial Sequence: Synthetic polypeptide
MALPVTALLLPLALLLHAARPGYPYDVPDYAGFHTINATWWANITLVGPPDTPVTWYDTQGLWFCNGSRVKNPQIRHTCNDQNLTLIHVNKTYERTYMGYNRQGTKKEDYKVVVIPPPPATVKPQPEPEYVFVYMGENKTLEGPPGTPVTWFNQDGKKFCEGEKVLHPEFNHTCDKQNLILLFVNFTHDGAYLGYNHQGTQRTHYEVTVLDLFPDSGQMKIENHSEETEQKNDEHHNWQKQGGQKQGGQKTNQTKVNDRRKTAQKRPSKLKPATIEAMLVTVTAGSNLTLVGPKAEGKVTWFDGDLKRPCEPNYRLRHECNNQNLTLINVTKDYEGTYYGTNDKDEGKRYRVKVNTTNSQSVKIQPYTRQTTPDQEHKFELQFETNGNYDSKIPGGGGSGGGGSLVPRGSKPIPNPLLGLDSTLVPRGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 29 - protein, 745 aa, synthetic construct
  REGION 1..745: note = Description of Artificial Sequence: Synthetic polypeptide
MALPVTALLLPLALLLHAARPGYPYDVPDYAHDACIPVVGKIGTNVTLNAVDFHPGDHVRWSYGPGGAGYMLCVYTGSWTEYKKPDIIFKCLSNNSLLLINVTVNYTNTYRTLTSLNNWVHNQHHHKFPGWNLDTCYSLTVNENGTFPTTTTKKPTTTTRTTTTTTTKKTTTTRTTTAAKKTTISTTHHKHSSPKKSSTPNSHVEHHVGFEATAAETPLQPSPQHQHVATHGGGGSGGGGSLVPRGSKPIPNPLLGLDSTLVPRGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 30 - protein, 660 aa, synthetic construct
  REGION 1..660: note = Description of Artificial Sequence: Synthetic polypeptide
MALPVTALLLPLALLLHAARPGYPYDVPDYAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAGGGGSGGGGSLVPRGSKPIPNPLLGLDSTLVPRGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 656 aa, synthetic construct
  REGION 1..656: note = Description of Artificial Sequence: Synthetic polypeptide
MALPVTALLLPLALLLHAARPGYPYDVPDYAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAGTGGGGSGGGGSLVPRGSKPIPNPLLGLDSTLVPRGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - protein, 6 aa, synthetic construct
  REGION 1..6: note = Description of Artificial Sequence: Synthetic peptide
LVPRGS

SEQ ID NO: 33 - protein, 42 aa, synthetic construct
  REGION 1..42: note = Description of Artificial Sequence: Synthetic polypeptide
KRGRKKLLYIFKQPFMRPVQTTQEEDGCSCRFPEEEEGGCEL

SEQ ID NO: 34 - protein, 112 aa, synthetic construct
  REGION 1..112: note = Description of Artificial Sequence: Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 41 aa, synthetic construct
  REGION 1..41: note = Description of Artificial Sequence: Synthetic polypeptide
RSKRSRGGHSDYMNMTPRRPGPTRKHYQPYAPPRDFAAYRS

SEQ ID NO: 36 - protein, 16 aa, synthetic construct
  REGION 1..16: note = Description of Artificial Sequence: Synthetic peptide
GLAVSTISSFFPPGYQ

SEQ ID NO: 37 - protein, 45 aa, synthetic construct
  REGION 1..45: note = Description of Artificial Sequence: Synthetic polypeptide
TTTPAPRPPTPAPTIASQPLSLRPEACRPAAGGAVHTRGLDFACD

SEQ ID NO: 38 - protein, 231 aa, synthetic construct
  REGION 1..231: note = Description of Artificial Sequence: Synthetic polypeptide
EPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 24 aa, synthetic construct
  REGION 1..24: note = Description of Artificial Sequence: Synthetic peptide
IYIWAPLAGTCGVLLLSLVITLYC

SEQ ID NO: 40 - protein, 112 aa, synthetic construct; identical to SEQ ID NO: 34
  REGION 1..112: note = Description of Artificial Sequence: Synthetic polypeptide

SEQ ID NO: 41 - protein, 52 aa, synthetic construct
  REGION 1..52: note = Description of Artificial Sequence: Synthetic polypeptide
FFIPLLVVILFAVDTGLFISTQQQVTFLLKIKRTRKGFRLLNPHPKPNPKNN

SEQ ID NO: 42 - protein, 215 aa, synthetic construct
  REGION 1..215: note = Description of Artificial Sequence: Synthetic polypeptide
MDTESNRRANLALPQEPSSVPAFEVLEISPQEVSSGRLLKSASSPPLHTWLTVLKKEQEFLGVTQILTAMICLCFGTVVCSVLDISHIEGDIFSSFKAGYPFWGAIFFSISGMLSIISERRNATYLVRGSLGANTASSIAGGTGITILIINLKKSLAYIHIHSCQKFFETKCFMASFSTEIVVMMLFLTILGLGSAVSLTICGAGEELKGNKVPE

SEQ ID NO: 43 - protein, 41 aa, synthetic construct; identical to SEQ ID NO: 35
  REGION 1..41: note = Description of Artificial Sequence: Synthetic polypeptide

SEQ ID NO: 44 - protein, 18 aa, synthetic construct
  REGION 1..18: note = Description of Artificial Sequence: Synthetic peptide
MIPAVVLLLLLLVEQAAA

SEQ ID NO: 45 - protein, 43 aa, synthetic construct
  REGION 1..43: note = Description of Artificial Sequence: Synthetic polypeptide
LGEPQLCYILDAILFLYGIVLTLLYCRLKIQVRKAAITSYEKS

SEQ ID NO: 46 - protein, 22 aa, synthetic construct
  REGION 1..22: note = Description of Artificial Sequence: Synthetic peptide
GSGATNFSLLKQAGDVEENPGP

SEQ ID NO: 47 - protein, 21 aa, synthetic construct
  REGION 1..21: note = Description of Artificial Sequence: Synthetic peptide
GSGEGRGSLLTCGDVEENPGP

SEQ ID NO: 48 - protein, 136 aa, synthetic construct
  REGION 1..136: note = Description of Artificial Sequence: Synthetic polypeptide
CPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 49 - protein, 157 aa, synthetic construct
  REGION 1..157: note = Description of Artificial Sequence: Synthetic polypeptide
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 50 - protein, 9 aa, synthetic construct
  REGION 1..9: note = Description of Artificial Sequence: Synthetic peptide
CPYSNPSLC

SEQ ID NO: 51 - protein, 37 aa, synthetic construct
  REGION 1..37: note = Description of Artificial Sequence: Synthetic polypeptide
GSGGGGSCPYSNPSLCSGGGGSCPYSNPSLCSGGGGS

SEQ ID NO: 52 - protein, 15 aa, synthetic construct
  REGION 1..15: note = Description of Artificial Sequence: Synthetic peptide
GGGGSGGGGSGGGSG

SEQ ID NO: 53 - protein, 16 aa, synthetic construct
  REGION 1..16: note = Description of Artificial Sequence: Synthetic peptide
LSGRSDNHSPLGLAGS

SEQ ID NO: 54 - protein, 14 aa, synthetic construct
  REGION 1..14: note = Description of Artificial Sequence: Synthetic peptide
GGGSGGGGSGGGGS

SEQ ID NO: 55 - protein, 13 aa, synthetic construct
  REGION 1..13: note = Description of Artificial Sequence: Synthetic peptide
KPIPNPLLGLDST

SEQ ID NO: 56 - protein, 267 aa, synthetic construct
  REGION 1..267: note = Description of Artificial Sequence: Synthetic polypeptide
MALPVTALLLPLALLLHAARPQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIK

SEQ ID NO: 57 - protein, 15 aa, synthetic construct
  REGION 1..15: note = Description of Artificial Sequence: Synthetic peptide
GGGGSGGGGSGGGGS

SEQ ID NO: 58 - protein, 118 aa, synthetic construct
  REGION 1..118: note = Description of Artificial Sequence: Synthetic polypeptide
QVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSS

SEQ ID NO: 59 - protein, 108 aa, synthetic construct
  REGION 1..108: note = Description of Artificial Sequence: Synthetic polypeptide
EIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIK

SEQ ID NO: 60 - protein, 5 aa, synthetic construct
  REGION 1..5: note = Description of Artificial Sequence: Synthetic peptide
YYSWT

SEQ ID NO: 61 - protein, 16 aa, synthetic construct
  REGION 1..16: note = Description of Artificial Sequence: Synthetic peptide
RIHISGVTNHNPSLKS

SEQ ID NO: 62 - protein, 10 aa, synthetic construct
  REGION 1..10: note = Description of Artificial Sequence: Synthetic peptide
SGGTYSAFDI

SEQ ID NO: 63 - protein, 8 aa, synthetic construct
  REGION 1..8: note = Description of Artificial Sequence: Synthetic peptide
SGGSISYY

SEQ ID NO: 64 - protein, 5 aa, synthetic construct
  REGION 1..5: note = Description of Artificial Sequence: Synthetic peptide
HISGV

SEQ ID NO: 65 - protein, 10 aa, synthetic construct
  REGION 1..10: note = Description of Artificial Sequence: Synthetic peptide
SGGTYSAFDI

SEQ ID NO: 66 - protein, 12 aa, synthetic construct
  REGION 1..12: note = Description of Artificial Sequence: Synthetic peptide
RASQSFTSNYLA

SEQ ID NO: 67 - protein, 7 aa, synthetic construct
  REGION 1..7: note = Description of Artificial Sequence: Synthetic peptide
GASTRAI

SEQ ID NO: 68 - protein, 9 aa, synthetic construct
  REGION 1..9: note = Description of Artificial Sequence: Synthetic peptide
QQYGSSPWT

SEQ ID NO: 69 - protein, 12 aa, synthetic construct
  REGION 1..12: note = Description of Artificial Sequence: Synthetic peptide
RASQSFTSNYLA

SEQ ID NO: 70 - protein, 7 aa, synthetic construct
  REGION 1..7: note = Description of Artificial Sequence: Synthetic peptide
GASTRAI

SEQ ID NO: 71 - protein, 9 aa, synthetic construct
  REGION 1..9: note = Description of Artificial Sequence: Synthetic peptide
QQYGSSPWT

SEQ ID NO: 72 - protein, 20 aa, synthetic construct
  REGION 1..20: note = Description of Artificial Sequence: Synthetic peptide
GGGGSGGGGSGGGGSGGGGS

SEQ ID NO: 73 - protein, 66 aa, synthetic construct
  REGION 1..66: note = Description of Artificial Sequence: Synthetic polypeptide
TTTPAPRPPTPAPTIASQPLSLRPEACRPAAGGAVHTRGLDFACDIYIWAPLAGTCGVLLLSLVIT

SEQ ID NO: 74 - protein, 24 aa, synthetic construct
  REGION 1..24: note = Description of Artificial Sequence: Synthetic peptide
ELPTQGTFSNVSTNVSPAKPTTTA

SEQ ID NO: 75 - protein, 490 aa, synthetic construct
  REGION 1..490: note = Description of Artificial Sequence: Synthetic polypeptide
MALPVTALLLPLALLLHAARPQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 76 - protein, 527 aa, synthetic construct
  REGION 1..527: note = Description of Artificial Sequence: Synthetic polypeptide
MALPVTALLLPLALLLHAARPQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKGSGGGGSCPYSNPSLCSGGGGSCPYSNPSLCSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 77 - protein, 523 aa, synthetic construct
  REGION 1..523: note = Description of Artificial Sequence: Synthetic polypeptide
MALPVTALLLPLALLLHAARPGGGGSCPYSNPSLCGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKGGGGSCPYSNPSLCTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 78 - protein, 523 aa, synthetic construct
  REGION 1..523: note = Description of Artificial Sequence: Synthetic polypeptide
MALPVTALLLPLALLLHAARPGGGGSCPYSNPSLCGGGGSCPYSNPSLCGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 79 - protein, 571 aa, synthetic construct
  REGION 1..571: note = Description of Artificial Sequence: Synthetic polypeptide
MALPVTALLLPLALLLHAARPGGGGSCPYSNPSLCSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKGSGGGGSCPYSNPSLCSGGGGSELPTQGTFSNVSTNVSPAKPTTTACPYSNPSLCTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 80 - protein, 24 aa, synthetic construct
  REGION 1..24: note = Description of Artificial Sequence: Synthetic peptide
NSELLSLINDMPITNDQKKLMSNN

SEQ ID NO: 81 - protein, 12 aa, synthetic construct
  REGION 1..12: note = Description of Artificial Sequence: Synthetic peptide
CQFDLSTRRLKC

SEQ ID NO: 82 - protein, 12 aa, synthetic construct
  REGION 1..12: note = Description of Artificial Sequence: Synthetic peptide
CQYNLSSRALKC

SEQ ID NO: 83 - protein, 12 aa, synthetic construct
  REGION 1..12: note = Description of Artificial Sequence: Synthetic peptide
CVWQRWQKSYVC

SEQ ID NO: 84 - protein, 12 aa, synthetic construct
  REGION 1..12: note = Description of Artificial Sequence: Synthetic peptide
CMWDRFSRWYKC

SEQ ID NO: 85 - protein, 25 aa, synthetic construct
  REGION 1..25: note = Description of Artificial Sequence: Synthetic peptide
SFVLNWYRMSPSNQTDKLAAFPEDR

SEQ ID NO: 86 - protein, 19 aa, synthetic construct
  REGION 1..19: note = Description of Artificial Sequence: Synthetic peptide
SGTYLCGAISLAPKAQIKE

SEQ ID NO: 87 - protein, 24 aa, synthetic construct
  REGION 1..24: note = Description of Artificial Sequence: Synthetic peptide
ELPTQGTFSNVSTNVSPAKPTTTA

SEQ ID NO: 88 - protein, 12 aa, synthetic construct
  REGION 1..12: note = Description of Artificial Sequence: Synthetic peptide
GQNDTSQTSSPS

SEQ ID NO: 89 - protein, 18 aa, synthetic construct
  REGION 1..18: note = Description of Artificial Sequence: Synthetic peptide
VHMPLGFLGPRQARVVNG

SEQ ID NO: 90 - protein, 18 aa, synthetic construct
  REGION 1..18: note = Description of Artificial Sequence: Synthetic peptide
VHMPLGFLGPGSARVVNG

SEQ ID NO: 91 - protein, 8 aa, synthetic construct
  REGION 1..8: note = Description of Artificial Sequence: Synthetic peptide
RQARVVNG

SEQ ID NO: 92 - protein, 8 aa, synthetic construct
  REGION 1..8: note = Description of Artificial Sequence: Synthetic peptide
RQARVGSG

SEQ ID NO: 93 - protein, 10 aa, synthetic construct
  REGION 1..10: note = Description of Artificial Sequence: Synthetic peptide
VHMPLGFLGP

SEQ ID NO: 94 - protein, 10 aa, synthetic construct
  REGION 1..10: note = Description of Artificial Sequence: Synthetic peptide
VHMPLSFLGP

SEQ ID NO: 95 - protein, 5 aa, synthetic construct
  REGION 1..5: note = Description of Artificial Sequence: Synthetic peptide
PMAKK

SEQ ID NO: 96 - protein, 6 aa, synthetic construct
  REGION 1..6: note = Description of Artificial Sequence: Synthetic peptide
PMAKGS

SEQ ID NO: 97 - protein, 8 aa, synthetic construct
  REGION 1..8: note = Description of Artificial Sequence: Synthetic peptide
LSGRSDNH

SEQ ID NO: 98 - protein, 8 aa, synthetic construct
  REGION 1..8: note = Description of Artificial Sequence: Synthetic peptide
LSGRSDSH

SEQ ID NO: 99 - protein, 25 aa, synthetic construct
  REGION 1..25: note = Description of Artificial Sequence: Synthetic peptide
GGGGSVHMPLGFLGPRQARVVNGGS

SEQ ID NO: 100 - protein, 25 aa, synthetic construct
  REGION 1..25: note = Description of Artificial Sequence: Synthetic peptide
GGGGSVHMPLGFLGPGSARVVNGGS

SEQ ID NO: 101 - protein, 25 aa, synthetic construct
  REGION 1..25: note = Description of Artificial Sequence: Synthetic peptide
GGGGSGGGGSPMAKKGGGGSGGGGS

SEQ ID NO: 102 - protein, 25 aa, synthetic construct
  REGION 1..25: note = Description of Artificial Sequence: Synthetic peptide
GGGGSGGGGSPMAKGSGGGSGGGGS

SEQ ID NO: 103 - protein, 8 aa, synthetic construct
  REGION 1..8: note = Description of Artificial Sequence: Synthetic peptide
SPLGLAGS

SEQ ID NO: 104 - protein, 7 aa, synthetic construct
  REGION 1..7: note = Description of Artificial Sequence: Synthetic peptide
PLGVRGK

SEQ ID NO: 105 - protein, 5 aa, synthetic construct
  REGION 1..5: note = Description of Artificial Sequence: Synthetic peptide
PMAKG

SEQ ID NO: 106 - protein, 8 aa, synthetic construct
  REGION 1..8: note = Description of Artificial Sequence: Synthetic peptide
GSARVVNG

SEQ ID NO: 107 - protein, 45 aa, synthetic construct
  REGION 1..45: note = Description of Artificial Sequence: Synthetic polypeptide
GGGGSGGGGSGGGGSVHMPLGFLGPRQARVVNGGSGGGGSGGGGS

SEQ ID NO: 108 - protein, 45 aa, synthetic construct
  REGION 1..45: note = Description of Artificial Sequence: Synthetic polypeptide
GGGGSGGGGSGGGGSVHMPLGFLGPGSARVVNGGSGGGGSGGGGS

SEQ ID NO: 109 - protein, 20 aa, synthetic construct
  REGION 1..20: note = Description of Artificial Sequence: Synthetic peptide
GGGGSPMAKKGGGGSGGGGS

SEQ ID NO: 110 - protein, 25 aa, synthetic construct
  REGION 1..25: note = Description of Artificial Sequence: Synthetic peptide
GGGGSGGGGSPMAKKGGGGSGGGGS

SEQ ID NO: 111 - protein, 30 aa, synthetic construct
  REGION 1..30: note = Description of Artificial Sequence: Synthetic polypeptide
GGGGSGGGGSPMAKKGGGGSGGGGSGGGGS

SEQ ID NO: 112 - protein, 35 aa, synthetic construct
  REGION 1..35: note = Description of Artificial Sequence: Synthetic polypeptide
GGGGSGGGGSGGGGSPMAKKGGGGSGGGGSGGGGS

SEQ ID NO: 113 - protein, 45 aa, synthetic construct
  REGION 1..45: note = Description of Artificial Sequence: Synthetic polypeptide
GGGGSGGGGSGGGGSGGGGSPMAKKGGGGSGGGGSGGGGSGGGGS

SEQ ID NO: 114 - protein, 45 aa, synthetic construct
  REGION 1..45: note = Description of Artificial Sequence: Synthetic polypeptide
GGGGSGGGGSGGGGSGGGGSPMAKGSGGGSGGGGSGGGGSGGGGS

SEQ ID NO: 115 - protein, 45 aa, synthetic construct
  REGION 1..45: note = Description of Artificial Sequence: Synthetic polypeptide
GGGGSGGGGSGSSPLGLAGSGGGGSGGGGSPMAKKGGGGSGGGGS

SEQ ID NO: 116 - protein, 45 aa, synthetic construct
  REGION 1..45: note = Description of Artificial Sequence: Synthetic polypeptide
GGGGSGGGGSGGSVHMPLGFLGPGGSGGGSPMAKKGGGGSGGGGS

SEQ ID NO: 117 - protein, 45 aa, synthetic construct
  REGION 1..45: note = Description of Artificial Sequence: Synthetic polypeptide
GGGGSGGGGSGGSPLGVRGKGGGGSGGGGSPMAKKGGGGSGGGGS

SEQ ID NO: 118 - protein, 45 aa, synthetic construct
  REGION 1..45: note = Description of Artificial Sequence: Synthetic polypeptide
GGGGSGGGGSGGSSPLGLAGSGSGGGSRQARVVNGGGGGSGGGGS

SEQ ID NO: 119 - protein, 45 aa, synthetic construct
  REGION 1..45: note = Description of Artificial Sequence: Synthetic polypeptide
GGGGSGGGGSGGSVHMPLGFLGPGGGSRQARVVNGGGGGSGGGGS

SEQ ID NO: 120 - protein, 45 aa, synthetic construct
  REGION 1..45: note = Description of Artificial Sequence: Synthetic polypeptide
GGGGSGGGGSGGSPLGVRGKGGSGGGSRQARVVNGGGGGSGGGGS

SEQ ID NO: 121 - protein, 793 aa, synthetic construct
  REGION 1..793: note = Description of Artificial Sequence: Synthetic polypeptide
MALPVTALLLPLALLLHAARPGYPYDVPDYADIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSGGGGSVHMPLGFLGPRQARVVNGGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 122 - protein, 793 aa, synthetic construct
  REGION 1..793: note = Description of Artificial Sequence: Synthetic polypeptide
MALPVTALLLPLALLLHAARPGYPYDVPDYADIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSGGGGSVHMPLGFLGPGSARVVNGGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 123 - protein, 793 aa, synthetic construct
  REGION 1..793: note = Description of Artificial Sequence: Synthetic polypeptide
MALPVTALLLPLALLLHAARPGYPYDVPDYADIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSGGGGSGGGGSPMAKKGGGGSGGGGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 124 - protein, 793 aa, synthetic construct
  REGION 1..793: note = Description of Artificial Sequence: Synthetic polypeptide
MALPVTALLLPLALLLHAARPGYPYDVPDYADIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSGGGGSGGGGSPMAKGSGGGSGGGGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 125 - protein, 808 aa, synthetic construct
  REGION 1..808: note = Description of Artificial Sequence: Synthetic polypeptide
MALPVTALLLPLALLLHAARPGYPYDVPDYADIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSVHMPLGFLGPRQARVVNGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSGGGGSVHMPLGFLGPRQARVVNGGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 126 - protein, 808 aa, synthetic construct
  REGION 1..808: note = Description of Artificial Sequence: Synthetic polypeptide
MALPVTALLLPLALLLHAARPGYPYDVPDYADIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSVHMPLGFLGPGSARVVNGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSGGGGSVHMPLGFLGPGSARVVNGGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 127 - protein, 808 aa, synthetic construct
  REGION 1..808: note = Description of Artificial Sequence: Synthetic polypeptide
MALPVTALLLPLALLLHAARPGYPYDVPDYADIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSGGGGSPMAKKGGGGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSGGGGSGGGGSPMAKKGGGGSGGGGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 128 - protein, 808 aa, synthetic construct
  REGION 1..808: note = Description of Artificial Sequence: Synthetic polypeptide
MALPVTALLLPLALLLHAARPGYPYDVPDYADIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSGGGGSPMAKGSGGGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSGGGGSGGGGSPMAKGSGGGSGGGGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 129 - protein, 771 aa, synthetic construct
  REGION 1..771: note = Description of Artificial Sequence: Synthetic polypeptide
MALPVTALLLPLALLLHAARPGYPYDVPDYAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAGTGGGGSGGGGSLVPRGSKPIPNPLLGLDSTLVPRGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 130 - protein, 790 aa, synthetic construct
  REGION 1..790: note = Description of Artificial Sequence: Synthetic polypeptide
MALPVTALLLPLALLLHAARPGYPYDVPDYADILLTQSPATLSLSPGERATLSCRASQNIGTSIQWYQQKPGQAPRLLIRSSSESISGIPSRFSGSGSGTDFTLTISSLEPEDFAVYYCQQSNTWPFTFGQGTKLEIKGGGGSGGGGSGGGGSGGGGSEVQLVESGAEVKKPGASVKVSCKASGYTFTNYIIHWVKQEPGQGLEWIGYFNPYNHGTKYNEKFKGRATLTADKSISTAYMELSSLRSEDTAVYYCARSGPYAWFDTWGQGTTVTVSSGGGGSGGGGSLVPRGSKPIPNPLLGLDSTLVPRGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 131 - protein, 790 aa, synthetic construct
  REGION 1..790: note = Description of Artificial Sequence: Synthetic polypeptide
MALPVTALLLPLALLLHAARPGYPYDVPDYADILLTQSPATLSLSPGERATLSCRASQNIGTSIQWYQQKPGQAPRLLIRSSSESISGIPSRFSGSGSGTDFTLTISSLEPEDFAVYYCQQSNTWPFTFGQGTKLEIKGGGGSGGGGSGGGGSGGGGSEVQLVESGAEVKKPGASVKVSCKASGYTFTNYIIHWVKQEPGQGLEWIGYFNPYNHGTKYNEKFKGRATLTADKSISTAYMELSSLRSEDTAVYYCARSGPYAWFDTWGQGTTVTVSSGGGGSGGGGSGGGSGSKPIPNPLLGLDSTGGGSGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 132 - protein, 790 aa, synthetic construct
  REGION 1..790: note = Description of Artificial Sequence: Synthetic polypeptide
MALPVTALLLPLALLLHAARPGYPYDVPDYADILLTQSPATLSLSPGERATLSCRASQNIGTSIQWYQQKPGQAPRLLIRSSSESISGIPSRFSGSGSGTDFTLTISSLEPEDFAVYYCQQSNTWPFTFGQGTKLEIKGGGGSGGGGSGGGGSGGGGSEVQLVESGAEVKKPGASVKVSCKASGYTFTNYIIHWVKQEPGQGLEWIGYFNPYNHGTKYNEKFKGRATLTADKSISTAYMELSSLRSEDTAVYYCARSGPYAWFDTWGQGTTVTVSSGGGGSGGGGSGGGGSVHMPLGFLGPRQARVVNGGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 133 - protein, 790 aa, synthetic construct
  REGION 1..790: note = Description of Artificial Sequence: Synthetic polypeptide
MALPVTALLLPLALLLHAARPGYPYDVPDYADILLTQSPATLSLSPGERATLSCRASQNIGTSIQWYQQKPGQAPRLLIRSSSESISGIPSRFSGSGSGTDFTLTISSLEPEDFAVYYCQQSNTWPFTFGQGTKLEIKGGGGSGGGGSGGGGSGGGGSEVQLVESGAEVKKPGASVKVSCKASGYTFTNYIIHWVKQEPGQGLEWIGYFNPYNHGTKYNEKFKGRATLTADKSISTAYMELSSLRSEDTAVYYCARSGPYAWFDTWGQGTTVTVSSGGGGSGGGGSGGGGSGGGGSPMAKKGGGGSGGGGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 134 - protein, 805 aa, synthetic construct
  REGION 1..805: note = Description of Artificial Sequence: Synthetic polypeptide
MALPVTALLLPLALLLHAARPGYPYDVPDYADILLTQSPATLSLSPGERATLSCRASQNIGTSIQWYQQKPGQAPRLLIRSSSESISGIPSRFSGSGSGTDFTLTISSLEPEDFAVYYCQQSNTWPFTFGQGTKLEIKGGGGSGGGGSVHMPLGFLGPRQARVVNGGSGGGGSEVQLVESGAEVKKPGASVKVSCKASGYTFTNYIIHWVKQEPGQGLEWIGYFNPYNHGTKYNEKFKGRATLTADKSISTAYMELSSLRSEDTAVYYCARSGPYAWFDTWGQGTTVTVSSGGGGSGGGGSGGGGSVHMPLGFLGPRQARVVNGGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 135 - protein, 805 aa, synthetic construct
  REGION 1..805: note = Description of Artificial Sequence: Synthetic polypeptide
MALPVTALLLPLALLLHAARPGYPYDVPDYADILLTQSPATLSLSPGERATLSCRASQNIGTSIQWYQQKPGQAPRLLIRSSSESISGIPSRFSGSGSGTDFTLTISSLEPEDFAVYYCQQSNTWPFTFGQGTKLEIKGGGGSGGGGSGGGGSPMAKKGGGGSGGGGSGGGGSEVQLVESGAEVKKPGASVKVSCKASGYTFTNYIIHWVKQEPGQGLEWIGYFNPYNHGTKYNEKFKGRATLTADKSISTAYMELSSLRSEDTAVYYCARSGPYAWFDTWGQGTTVTVSSGGGGSGGGGSGGGGSGGGGSPMAKKGGGGSGGGGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 136 - protein, 817 aa, synthetic construct
  REGION 1..817: note = Description of Artificial Sequence: Synthetic polypeptide
MALPVTALLLPLALLLHAARPGKPIPNPLLGLDSTGGGGSGGGGSGGGGSGGGGSDIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSGGGSGLSGRSDNHSPLGLAGSGGGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 137 - protein, 817 aa, synthetic construct
  REGION 1..817: note = Description of Artificial Sequence: Synthetic polypeptide
MALPVTALLLPLALLLHAARPGKPIPNPLLGLDSTGGGGSGGGGSGGGGSGGGGSDIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSGGGGSVHMPLGFLGPRQARVVNGGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 138 - protein, 817 aa, synthetic construct
  REGION 1..817: note = Description of Artificial Sequence: Synthetic polypeptide
MALPVTALLLPLALLLHAARPGKPIPNPLLGLDSTGGGGSGGGGSGGGGSGGGGSDIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSGGGGSGGGGSPMAKKGGGGSGGGGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 139 - protein, 832 aa, synthetic construct
  REGION 1..832: note = Description of Artificial Sequence: Synthetic polypeptide
MALPVTALLLPLALLLHAARPGKPIPNPLLGLDSTGGGGSGGGGSGGGGSGGGGSDIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSGGGGSPMAKKGGGGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSGGGGSGGGGSPMAKKGGGGSGGGGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 140 - protein, 817 aa, synthetic construct
  REGION 1..817: note = Description of Artificial Sequence: Synthetic polypeptide
MALPVTALLLPLALLLHAARPGKPIPNPLLGLDSTGGGGSGGGGSGGGGSGGGGSDIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSGGGSGSGGGGSGGGSGGGSGGGSGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 141 - protein, 822 aa, synthetic construct
  REGION 1..822: note = Description of Artificial Sequence: Synthetic polypeptide
MALPVTALLLPLALLLHAARPGYPYDVPDYADIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSLSGRSDNHSPLGLAGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSKPIPNPLLGLDSTGGGSGLSGRSDNHSPLGLAGSGGGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 142 - protein, 821 aa, synthetic construct
  REGION 1..821: note = Description of Artificial Sequence: Synthetic polypeptide
MALPVTALLLPLALLLHAARPGYPYDVPDYADIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSVHMPLGFLGPRQARVVNGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSKPIPNPLLGLDSTGGGGSVHMPLGFLGPRQARVVNGGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 143 - protein, 821 aa, synthetic construct
  REGION 1..821: note = Description of Artificial Sequence: Synthetic polypeptide
MALPVTALLLPLALLLHAARPGYPYDVPDYADIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSVHMPLGFLGPGSARVVNGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSKPIPNPLLGLDSTGGGGSVHMPLGFLGPGSARVVNGGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 144 - protein, 816 aa, synthetic construct
  REGION 1..816: note = Description of Artificial Sequence: Synthetic polypeptide
MALPVTALLLPLALLLHAARPGYPYDVPDYADIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSGGGGSPMAKKGGGGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSKPIPNPLLGLDSTGGGGSPMAKKGGGGSGGGGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 145 - protein, 816 aa, synthetic construct
  REGION 1..816: note = Description of Artificial Sequence: Synthetic polypeptide
MALPVTALLLPLALLLHAARPGYPYDVPDYADIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSGGGGSPMAKGSGGGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSKPIPNPLLGLDSTGGGGSPMAKGSGGGSGGGGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 146 - protein, 778 aa, synthetic construct
  REGION 1..778: note = Description of Artificial Sequence: Synthetic polypeptide
MALPVTALLLPLALLLHAARPGYPYDVPDYADIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSGGGSGGGSGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 147 - protein, 768 aa, synthetic construct
  REGION 1..768: note = Description of Artificial Sequence: Synthetic polypeptide
MALPVTALLLPLALLLHAARPGYPYDVPDYADIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSGGGSGGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 148 - protein, 758 aa, synthetic construct
  REGION 1..758: note = Description of Artificial Sequence: Synthetic polypeptide
MALPVTALLLPLALLLHAARPGYPYDVPDYADIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 149 - protein, 807 aa, synthetic construct
  REGION 1..807: note = Description of Artificial Sequence: Synthetic polypeptide
MALPVTALLLPLALLLHAARPGKPIPNPLLGLDSTGGGGSGGGGSGGGGSGGGGSDIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSGGGGSPMAKKGGGGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 150 - protein, 802 aa, synthetic construct
  REGION 1..802: note = Description of Artificial Sequence: Synthetic polypeptide
MALPVTALLLPLALLLHAARPGKPIPNPLLGLDSTGGGGSGGGGSGGGGSGGGGSDIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSPMAKKGGGGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 151 - protein, 797 aa, synthetic construct
  REGION 1..797: note = Description of Artificial Sequence: Synthetic polypeptide
MALPVTALLLPLALLLHAARPGKPIPNPLLGLDSTGGGGSGGGGSGGGGSGGGGSDIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSPMAKKGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 152 - protein, 792 aa, synthetic construct
  REGION 1..792: note = Description of Artificial Sequence: Synthetic polypeptide
MALPVTALLLPLALLLHAARPGKPIPNPLLGLDSTGGGGSGGGGSGGGGSGGGGSDIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSPMAKKGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 153 - protein, 822 aa, synthetic construct
  REGION 1..822: note = Description of Artificial Sequence: Synthetic polypeptide
MALPVTALLLPLALLLHAARPGKPIPNPLLGLDSTGGGGSGGGGSGGGGSGGGGSDIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSGGGGSPMAKKGGGGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSGGGGSPMAKKGGGGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 154 - protein, 817 aa, synthetic construct
  REGION 1..817: note = Description of Artificial Sequence: Synthetic polypeptide
MALPVTALLLPLALLLHAARPGKPIPNPLLGLDSTGGGGSGGGGSGGGGSGGGGSDIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSGGGGSPMAKKGGGGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSPMAKKGGGGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 155 - protein, 812 aa, synthetic construct
  REGION 1..812: note = Description of Artificial Sequence: Synthetic polypeptide
MALPVTALLLPLALLLHAARPGKPIPNPLLGLDSTGGGGSGGGGSGGGGSGGGGSDIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSGGGGSPMAKKGGGGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSPMAKKGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 156 - protein, 807 aa, synthetic construct
  REGION 1..807: note = Description of Artificial Sequence: Synthetic polypeptide
MALPVTALLLPLALLLHAARPGKPIPNPLLGLDSTGGGGSGGGGSGGGGSGGGGSDIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSGGGGSPMAKKGGGGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSPMAKKGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 157 - protein, 817 aa, synthetic construct
  REGION 1..817: note = Description of Artificial Sequence: Synthetic polypeptide
MALPVTALLLPLALLLHAARPGKPIPNPLLGLDSTGGGGSGGGGSGGGGSGGGGSDIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSGSSPLGLAGSGGGGSGGGGSPMAKKGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 158 - protein, 817 aa, synthetic construct
  REGION 1..817: note = Description of Artificial Sequence: Synthetic polypeptide
MALPVTALLLPLALLLHAARPGKPIPNPLLGLDSTGGGGSGGGGSGGGGSGGGGSDIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSGGSVHMPLGFLGPGGSGGGSPMAKKGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 159 - protein, 817 aa, synthetic construct
  REGION 1..817: note = Description of Artificial Sequence: Synthetic polypeptide
MALPVTALLLPLALLLHAARPGKPIPNPLLGLDSTGGGGSGGGGSGGGGSGGGGSDIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSGGSPLGVRGKGGGGSGGGGSPMAKKGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 160 - protein, 817 aa, synthetic construct
  REGION 1..817: note = Description of Artificial Sequence: Synthetic polypeptide
MALPVTALLLPLALLLHAARPGKPIPNPLLGLDSTGGGGSGGGGSGGGGSGGGGSDIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSGGSSPLGLAGSGSGGGSRQARVVNGGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 161 - protein, 817 aa, synthetic construct
  REGION 1..817: note = Description of Artificial Sequence: Synthetic polypeptide
MALPVTALLLPLALLLHAARPGKPIPNPLLGLDSTGGGGSGGGGSGGGGSGGGGSDIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSGGSVHMPLGFLGPGGGSRQARVVNGGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 162 - protein, 817 aa, synthetic construct
  REGION 1..817: note = Description of Artificial Sequence: Synthetic polypeptide
MALPVTALLLPLALLLHAARPGKPIPNPLLGLDSTGGGGSGGGGSGGGGSGGGGSDIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSGGSPLGVRGKGGSGGGSRQARVVNGGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 163 - protein, 817 aa, synthetic construct
  REGION 1..817: note = Description of Artificial Sequence: Synthetic polypeptide
MALPVTALLLPLALLLHAARPGKPIPNPLLGLDSTGGGGSGGGGSGGGGSGGGGSDIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSGGGSGGGSGSGGGGSGGGGSGGGGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 164 - protein, 807 aa, synthetic construct
  REGION 1..807: note = Description of Artificial Sequence: Synthetic polypeptide
MALPVTALLLPLALLLHAARPGKPIPNPLLGLDSTGGGGSGGGGSGGGGSGGGGSDIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGSGGGSGSGGGGSGGGGSGGGGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 165 - protein, 797 aa, synthetic construct
  REGION 1..797: note = Description of Artificial Sequence: Synthetic polypeptide
MALPVTALLLPLALLLHAARPGKPIPNPLLGLDSTGGGGSGGGGSGGGGSGGGGSDIVMTQTPASVSEPVGGSVTIKCQASQSFYNLLAWYQQKPGQPPKLLIYDASDLASGVPSRFKGSGSGTDFTLTISDLECADAAAYYCQSADGSSYAFGGGTEVVVKGGGGGSGGGGSGGGGSGGGGSQEQLEESGGGLVKPEGSLTLTCTASGVSFSSSYWIYWVRQAPGKGLEWIACIYTGSSGSTYYASWAKGRFTVSETSSTTVTLQMTSLTAADTATYFCARASAWTYGMDLWGPGTLVTVSSGGGGSGGGGSGGGGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 166 - protein, 660 aa, synthetic construct
  REGION 1..660: note = Description of Artificial Sequence: Synthetic polypeptide
MALPVTALLLPLALLLHAARPGYPYDVPDYAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAGGGGSGGGGSGGGSGGGSGSGGGGSGGGGSGGGGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 167 - protein, 656 aa, synthetic construct
  REGION 1..656: note = Description of Artificial Sequence: Synthetic polypeptide
MALPVTALLLPLALLLHAARPGYPYDVPDYAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAGTGGGGSGGGGSGGGSGGGSGSGGGGSGGGGSGGGGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 168 - protein, 771 aa, synthetic construct
  REGION 1..771: note = Description of Artificial Sequence: Synthetic polypeptide
MALPVTALLLPLALLLHAARPGYPYDVPDYAQFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAGTGGGGSGGGGSGGGSGGGSGSGGGGSGGGGSGGGGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 169 - protein, 908 aa, synthetic construct
  REGION 1..908: note = Description of Artificial Sequence: Synthetic polypeptide
MALPVTALLLPLALLLHAARPGYPYDVPDYAGFHTINATWWANITLVGPPDTPVTWYDTQGLWFCNGSRVKNPQIRHTCNDQNLTLIHVNKTYERTYMGYNRQGTKKEDYKVVVIPPPPATVKPQPEPEYVFVYMGENKTLEGPPGTPVTWFNQDGKKFCEGEKVLHPEFNHTCDKQNLILLFVNFTHDGAYLGYNHQGTQRTHYEVTVLDLFPDSGQMKIENHSEETEQKNDEHHNWQKQGGQKQGGQKTNQTKVNDRRKTAQKRPSKLKPATIEAMLVTVTAGSNLTLVGPKAEGKVTWFDGDLKRPCEPNYRLRHECNNQNLTLINVTKDYEGTYYGTNDKDEGKRYRVKVNTTNSQSVKIQPYTRQTTPDQEHKFELQFETNGNYDSKIPGGGGSGGGGSGGGSGGGSGSGGGGSGGGGSGGGGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 170 - protein, 745 aa, synthetic construct
  REGION 1..745: note = Description of Artificial Sequence: Synthetic polypeptide
MALPVTALLLPLALLLHAARPGYPYDVPDYAHDACIPVVGKIGTNVTLNAVDFHPGDHVRWSYGPGGAGYMLCVYTGSWTEYKKPDIIFKCLSNNSLLLINVTVNYTNTYRTLTSLNNWVHNQHHHKFPGWNLDTCYSLTVNENGTFPTTTTKKPTTTTRTTTTTTTKKTTTTRTTTAAKKTTISTTHHKHSSPKKSSTPNSHVEHHVGFEATAAETPLQPSPQHQHVATHGGGGSGGGGSGGGSGGGSGSGGGGSGGGGSGGGGSGGGGSGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 171 - protein, 245 aa, synthetic construct
  REGION 1..245: note = Description of Artificial Sequence: Synthetic polypeptide
DILLTQSPATLSLSPGERATLSCRASQNIGTSIQWYQQKPGQAPRLLIRSSSESISGIPSRFSGSGSGTDFTLTISSLEPEDFAVYYCQQSNTWPFTFGQGTKLEIKGGGGSGGGGSGGGGSGGGGSEVQLVESGAEVKKPGASVKVSCKASGYTFTNYIIHWVKQEPGQGLEWIGYFNPYNHGTKYNEKFKGRATLTADKSISTAYMELSSLRSEDTAVYYCARSGPYAWFDTWGQGTTVTVSS

SEQ ID NO: 172 - protein, 25 aa, synthetic construct
  REGION 1..25: note = Description of Artificial Sequence: Synthetic peptide
GSSPLGLAGSGGGGSGGGGSPMAKK

SEQ ID NO: 173 - protein, 25 aa, synthetic construct
  REGION 1..25: note = Description of Artificial Sequence: Synthetic peptide
GGSVHMPLGFLGPGGSGGGSPMAKK

SEQ ID NO: 174 - protein, 25 aa, synthetic construct
  REGION 1..25: note = Description of Artificial Sequence: Synthetic peptide
GGSPLGVRGKGGGGSGGGGSPMAKK

SEQ ID NO: 175 - protein, 25 aa, synthetic construct
  REGION 1..25: note = Description of Artificial Sequence: Synthetic peptide
GGSSPLGLAGSGSGGGSRQARVVNG

SEQ ID NO: 176 - protein, 25 aa, synthetic construct
  REGION 1..25: note = Description of Artificial Sequence: Synthetic peptide
GGSVHMPLGFLGPGGGSRQARVVNG

SEQ ID NO: 177 - protein, 25 aa, synthetic construct
  REGION 1..25: note = Description of Artificial Sequence: Synthetic peptide
GGSPLGVRGKGGSGGGSRQARVVNG

SEQ ID NO: 178 - protein, 5 aa, synthetic construct
  REGION 1..5: note = Description of Artificial Sequence: Synthetic peptide
GGGGS

SEQ ID NO: 179 - protein, 16 aa, synthetic construct
  REGION 1..16: note = Description of Artificial Sequence: Synthetic peptide
ELPTQGTFSNVSTNVS

SEQ ID NO: 180 - protein, 528 aa, synthetic construct
  REGION 1..528: note = Description of Artificial Sequence: Synthetic polypeptide
MALPVTALLLPLALLLHAARPGGGGSCPYSNPSLCGGGGSQVQLQESGPGLVKPSETLSLTCTVSGGSISYYSWTWVRQPAGQGLEWIGRIHISGVTNHNPSLKSRVSMSIDTSRTQFSLRLTSVTAADTAVYFCARSGGTYSAFDIWGQGTMVTVSSGGGGSGGGGSGGGGSGGGGSEIVLTQSPGTLSLSPGERSTLSCRASQSFTSNYLAWYQQRPGQAPRLLIYGASTRAIGIPDRFSGSGSGTDFTLTISRLEPEDFAVYYCQQYGSSPWTFGQGTKVEIKGGGGSCPYSNPSLCGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 181 - protein, 45 aa, synthetic construct
  REGION 1..45: note = Description of Artificial Sequence: Synthetic polypeptide
GGGGSGGGGSGGGSGSGGGGSGGGSGGGSGGGSGSGGGGSGGGGS

SEQ ID NO: 182 - protein, 45 aa, synthetic construct
  REGION 1..45: note = Description of Artificial Sequence: Synthetic polypeptide
GGGGSGGGGSGGGSGGGSGSGGGGSGGGGSGGGGSGGGGSGGGGS

SEQ ID NO: 183 - protein, 226 aa, synthetic construct
  REGION 1..226: note = Description of Artificial Sequence: Synthetic polypeptide
QFSVLGPSGPILAMVGEDADLPCHLFPTMSAETMELKWVSSSLRQVVNVYADGKEVEDRQSAPYRGRTSILRDGITAGKAALRIHNVTASDSGKYLCYFQDGDFYEKALVELKVAALGSDLHVDVKGYKDGGIHLECRSTGWYPQPQIQWSNNKGENIPTVEAPVVADGVGLYAVAASVIMRGSSGEGVSCTIRSSLLGLEKTASISIADPFFRSAQRWIAALAGT

SEQ ID NO: 184 - protein, 4 aa, synthetic construct
  REGION 1..4: note = Description of Artificial Sequence: Synthetic peptide
GGGS

SEQ ID NO: 185 - protein, 5 aa, synthetic construct
  REGION 1..5: note = Description of Artificial Sequence: Synthetic peptide
SGGGG

SEQ ID NO: 186 - protein, 6 aa, synthetic construct
  REGION 1..6: note = Description of Artificial Sequence: Synthetic peptide
SGGGGS

SEQ ID NO: 187 - protein, 9 aa, synthetic construct
  REGION 1..9: note = Description of Artificial Sequence: Synthetic peptide
YPYDVPDYA

(19) United States
(12) Patent Application Publication
(10) Pub. No.: US 2026/0193310 A1
(43) Pub. Date: Jul. 9, 2026

(54) PROTEASE-ACTIVATING CD45-GATE CAR

(71) Applicants: ALLOGENE THERAPEUTICS, INC., South San Francisco, CA (US); PFIZER INC., New York, NY (US)

(72) Inventors: Shanshan LANG, San Mateo, CA (US); Thomas John VAN BLARCOM, Oakland, CA (US); Michael Thomas BETHUNE, Castro Valley, CA (US); Siler PANOWSKI, Berkeley, CA (US); Nguyen TAN, Berkeley, CA (US); Yi ZHANG, Foster City, CA (US); Barbra Johnson SASU, San Francisco, CA (US); Zhe LI, Burlingame, CA (US)

(73) Assignees: ALLOGENE THERAPEUTICS, INC., South San Francisco, CA (US); PFIZER INC., New York, NY (US)

(21) Appl. No.: 19/288,824

(22) Filed: Aug. 1, 2025

Related U.S. Application Data
(62) Division of application No. 17/557,654, filed on Dec. 21, 2021, now Pat. No. 12,404,315.
(60) Provisional application No. 63/128,667, filed on Dec. 21, 2020.
(60) Provisional application No. 63/289,984, filed on Dec. 15, 2021.

(51) Int. Cl. C07K 14/725; A61K 40/11; A61K 40/15; A61K 40/31; A61K 40/42; C07K 14/705; C07K 16/28; C07K 19/00; C12N 5/078; C12N 5/0783; C12N 5/10; C12N 15/79

(52) U.S. Cl. CPC C07K 14/7051; A61K 40/11; A61K 40/15; A61K 40/31; A61K 40/4224; A61K 40/4252; A61K 40/4257; C07K 14/70521; C07K 14/70596; C07K 16/28; C07K 16/289; C07K 19/00; C12N 5/0634; C12N 5/0636; C12N 5/10; C12N 15/79; A61K 2239/31; A61K 2239/38; A61K 2239/49; A61K 2239/55; A61K 2239/59; C07K 2317/22; C07K 2317/24; C07K 2317/31; C07K 2317/569; C07K 2319/01; C07K 2319/31; C07K 2319/50

(57) ABSTRACT

A reversibly gated effector polypeptide e.g. a chimeric antigen receptor (protease-activating CD45-gate CAR) comprising an extracellular CD45 recruiting domain, a protease-cleavable linker, and a polypeptide comprising an extracellular ligand binding domain, a transmembrane domain, and an intracellular domain. Nucleic acids including vectors and expression vectors that encode the protease-activating CD45-gate CAR and cells including immune cells such as T cells that comprise and express the nucleic acids. Methods of treatment of various conditions including various forms of cancer comprising administering the cells including CAR T cell therapy. In some embodiments, the CD45 gate at least partially inhibits activation of the protease-activating CD45-gate CAR when the protease-activating CD45-gate CAR binds antigen. The inhibition is at least partially diminished, relieved and/or eliminated when the protease-activating CD45-gate CAR is exposed to a protease that can cleave the linker.

### CROSS REFERENCE TO RELATED APPLICATIONS

This application is a divisional of U.S. Application No. 17,557,654, filed Dec. 21, 2021, now U.S. Pat. No. 12,404,315 granted on Sep. 2, 2025, which claims the benefit of priority to U.S. Provisional Application No. 63/128,667, filed on Dec. 21, 2020; and U.S. Provisional Application No. 63/289,984, filed on Dec. 15, 2021, the contents of which are hereby incorporated by reference in their entireties.

### SEQUENCE LISTING

The instant application contains a Sequence Listing which has been submitted electronically in XML file format and is hereby incorporated by reference in its entirety. Said XML copy, created on Feb. 16, 2026, is named AT-043_04US_SL.xml and is 301,234 bytes in size.

### TECHNICAL FIELD

The present disclosure relates generally to the use of immune cells (e.g., T cells) engineered to express a chimeric antigen receptor (CAR) to treat a disease.

### BACKGROUND

Tumor specificity is one of the major hurdles for the development of cancer therapy, such as CAR T cell therapy, in solid tumor indications. This differentiates solid tumor targets from many hematologic targets (e.g. CD19, CD20, BCMA), in that expression of these hematologic targets is heavily restricted to the hematologic lineage. Although their expression can be found on healthy hematologic cells and this can lead to the destruction of both healthy and cancerous cells, repopulation of normal hematologic cells from their progenitor stem cells makes these targets acceptable. However, many solid tumor targets exhibit an appreciable level of expression in some healthy organs or tissues, and the collateral destruction of these healthy tissues by CAR T cells may lead to severe adverse side effects and even death in patients (“on-target off-tumor toxicity”). Hence, it is useful to develop effective strategies and improve the anti-tumor specificity of CAR T cells to address potential on-target off-tumor toxicities. There exists a need for a cell-based cancer therapy, such as CAR T cells, with reduced on-target off-tumor toxicity.

### SUMMARY

The present disclosure provides, among other things, chimeric antigen receptors (CARs) modified to improve target specificity in CAR T cell therapy, and related nucleic acids, expression vectors, cells, compositions and methods of treatment of cancer and other conditions amenable to CAR T cell therapy. The present disclosure provides a modification to CARs, the CD45 gate, that at least partially suppresses the modified CAR's activation in the presence of the CAR's antigen, thereby reducing the CAR's activity relative to the CAR not so modified or relative to the CAR not modified. This suppression and reduction in activity is partially or fully relieved when the modified CAR is in the presence of certain proteases, as occurs at the site of a tumor that is the CAR T cell therapy target. Disclosed herein are at least the following aspects and embodiments of the disclosure. Also provided is an anti-MUC16 CAR, including an anti-MUC16 CAR for use as a component of the protease-activating CD45-gate CAR disclosed herein.

CD45 is a phosphatase that plays a dynamic role in regulating T cell activation (1). It was found to directly activate TCR signaling by dephosphorylating other kinases, e.g. LCK, which activates these kinases and the downstream TCR signaling (2). CD45 is also able to directly dephosphorylate CD3zeta, which leads to the deactivation of TCR signaling (3).

Accordingly, in one aspect, provided herein is a protease-activating CD45-gate effector polypeptide comprising a CD45 recruiting domain connected to an effector entity optionally by a linker. In some embodiments, the effector entity can be reversibly modulated by CD45. In some embodiments, the effector entity is activated when the CD45 recruiting domain is released from the protease-activating CD45-gate effector by protease cleavage. In some embodiments, the CD45 recruiter is released by protease cleavage at the linker. In some embodiments, the effector entity comprises a chimeric antigen receptor (CAR). In some embodiments, the effector entity comprises a T cell receptor (TCR). In some embodiments, the CD45 recruiting domain (e.g. an anti-CD45 scFv) is prepared, using no more than ordinary skill in the art, from the CD45 antibodies disclosed in, for example, WO2005/026210, WO20131048804, WO2017009473, WO2020092654, EP1664122B1, and U.S. Pat. No. 6,106,834.

In one aspect, provided herein is a protease-activating CD45-gate chimeric antigen receptor (CD45-gate CAR) comprising an extracellular domain, a transmembrane domain, and an intracellular domain, wherein the extracellular domain comprises: a CD45 recruiting domain, a linker comprising one or more protease cleavage sites that can be cleaved by at least one protease, and an antigen binding domain. In some embodiments of the protease-activating CD45-gate CAR, the intracellular domain comprises at least one signaling domain that can be reversibly inactivated by CD45. In an embodiment, the linker is between the CD45 recruiting domain and the antigen binding domain. In an embodiment, the CD45 recruiting domain comprises one or more linkers. In an embodiment, the CD45 recruiting domain comprises an anti-CD45 antibody comprising a heavy chain variable domain (VH) and a light chain variable domain (VL). In some embodiments, the linker is between the VH and VL of the anti-CD45 antibody. In some embodiments, the protease-activating CD45-gate CAR comprises additional flexible regions e.g. placed between domains.

In a further aspect, provided herein is a protease-activating CD45-gate chimeric antigen receptor (CD45-gate CAR) comprising an extracellular domain, a transmembrane domain, and an intracellular domain, wherein the extracellular domain comprises:
a CD45 recruiting domain,
an antigen binding domain, and
a linker connecting the carboxy terminus of the CD45 recruiting domain to the amino terminus of the antigen binding domain, wherein the linker comprises at least one protease cleavage site that is recognized by a protease,
and further wherein the intracellular domain comprises at least one (e.g. one or more than one) signaling domain that can be reversibly inactivated by CD45.

In some embodiments, the CD45 recruiting domain comprises one or more of an anti-CD45 antibody antigen binding domain, an anti-CD45 NANOBODY®, an anti-CD45 scFv, a viral protein binder of CD45, a truncated viral protein binder of CD45, an anti-CD45 Fab, an anti-CD45 camelid VHH, a CD45-binding protein, an endogenous CD45 binder, or a truncated endogenous CD45 binder. In some embodiments, the CD45 recruiting domain comprises one or more of a peptide or protein that interacts with CD45, for example, full-length or truncated UL11, full-length or truncated sec49K, and full-length or truncated BTN3A1. In some embodiments, the CD45 recruiting domain comprises a truncated BTN3A1, e.g. a truncated BTN3A1 having the amino acid sequence of SEQ ID NO: 14 or of SEQ ID NO: 15.

In some embodiments, the CD45 recruiting domain comprises an anti-CD45 scFv. In some embodiments, the CD45 recruiting domain comprises an anti-CD45 antibody antigen binding domain. In some embodiments, the CD45 recruiting domain comprises one or more of truncated UL11, truncated sec49K, and truncated BTN3A1. In some embodiments, the CD45 recruiting domain comprises the amino acid sequence of SEQ ID NO: 3, SEQ ID NO: 4, SEQ ID NO: 5, SEQ ID NO: 6, SEQ ID NO: 12, SEQ ID NO: 13, SEQ ID NO: 14 or SEQ ID NO: 15.

In some embodiments, the antigen binding domain specifically binds BCMA, MUC16 (also known as CA125), EGFR, EGFRVIII, MUC1, Flt-3, WT-1, CD20, CD23, CD30, CD38, CD70, CD33, CD133, MHC-WT1, TSPAN10, MHC-PRAME, MHC-NY-ESO1, HER2 (ERBB2), CAIX (Carbonic anhydrase IX), LIV1, ADAM10, CHRNA2, LeY, NKG2D, CS1, CD44v6, ROR1, CD19, Claudin-18.2 (Claudin-18A2, or Claudin18 isoform 2), PSCA, DLL3 (Delta-like protein 3, Drosophila Delta homolog 3, Delta3), Mud 7 (Mucin17, Muc3, Muc3), FAP alpha (Fibroblast Activation Protein alpha), Ly6G6D (Lymphocyte antigen 6 complex locus protein G6d, c6orf23, G6D, MEGT1, NG25), PSMA, MSLN, or RNF43 (E3 ubiquitin-protein ligase RNF43, RING finger protein 43).

In some embodiments, the antigen binding domain specifically binds MUC16. In some embodiments, the antigen binding domain specifically binds MUC16 and the protease-activating CD45-gate CAR comprises the amino acid sequence of any of SEQ ID NOs: 11, 56, 75-79, and 121-170, each of which can have or not have the signal sequence of SEQ ID NO:1. In some embodiments, the antigen binding domain specifically binds MUC16 and the protease-activating CD45-gate CAR comprises the amino acid sequence of SEQ ID NO: 75, with or without the signal sequence of SEQ ID NO:1. In some embodiments, the antigen binding domain specifically binds MUC16 and the protease-activating CD45-gate CAR comprises the amino acid sequence of SEQ ID NO: 76, with or without the signal sequence of SEQ ID NO:1. In some embodiments, the extracellular domain comprises the amino acid sequence of any one of SEQ ID NOs: 3, 4, 5, 6, 12, 13, 14, 15, 56, and 56 without the signal sequence of SEQ ID NO:1.

In some embodiments, the antigen binding domain specifically binds to an antigen expressed by a certain type of tumor and the protease cleavage sites can be recognized and cleaved by a protease that the same type of tumor expresses. In some embodiments, the protease-activating CD45-gate CAR both (1) can specifically recognize and bind to an antigen expressed by a tumor and (2) comprises a CD45 recruiting domain-CAR linker that can be cleaved by a protease that the tumor secretes and/or that is present and/or active in the tumor microenvironment.

In some embodiments, the antigen binding domain specifically binds to a breast cancer tumor antigen or a colorectal cancer tumor antigen and one or more of the at least one protease cleavage sites can be recognized and cleaved by uPA. In some embodiments, the protease-activating CD45-gate CAR's antigen binding domain specifically recognizes and binds to an antigen characteristic of any of cervical, breast, ovarian and colorectal cancers and at least one of its protease cleavage sites can be recognized and cleaved by MMP-2 and/or MMP-9. In some embodiments, the protease-activating CD45-gate CAR's antigen binding domain specifically recognizes and binds to an antigen characteristic of any of breast and ovarian cancers and at least one of its protease cleavage sites can be recognized and cleaved by matriptase.

In some embodiments, the linker comprises a GS sequence on either or both ends of each of the one or more protease cleavage sites. In some embodiments, the GS sequence is a polypeptide that consist of only glycine and serine residues. In some embodiments, each GS sequence comprises a series of linked GS peptides, a GS peptide being, for example, a peptide having an amino acid sequence comprising only glycine and serine residues, e.g. a peptide having an amino acid sequence comprising one or more glycine residues followed by one or more serine residues, for example, a peptide having an amino acid sequence of GS, GGS, GGGS (SEQ ID NO: 184), or GGGGS (SEQ ID NO:178), wherein the linked GS peptides are linked in any order and in any combination. In some embodiments, the GS sequence comprises any combination of 1, 2, 3, 4, 5, 6, 7, 8, 9 or 10 GS peptides or comprises between 1 and 15 GS peptides in any order and combination.

In some embodiments, the linker of the protease-activating CD45-gate CAR disclosed herein has a length of 15-100 amino acids, 30-100 amino acids, 15-75 amino acids, 20-50 amino acids, 20 amino acids, 25 amino acids, 30 amino acids, 35 amino acids, 40 amino acids, 45 amino acids, or 50 amino acids, or about 20 amino acids, about 25 amino acids, about 30 amino acids, about 35 amino acids, about 40 amino acids, about 45 amino acids, or about 50 amino acids.

In some embodiments of the protease-activating CD45-gate CAR disclosed herein, the linker comprises one or more protease cleavage sites that comprise the amino acid sequence of SEQ ID NO: 32, 91-98, 103-105 or SEQ ID NO: 106. In some embodiments, the linker comprises one or more amino acid sequences of SEQ ID NO: 53, 89, or 90. In some embodiments, the linker comprises one or more amino acid sequences of SEQ ID NO: 8-10, 99-102, 107-120, 172-176 or SEQ ID NO: 177. In some embodiments, the linker comprises two or more protease cleavage sites and each cleavage site is the same as or different from any of the other cleavage sites. In some embodiments, the number of protease cleavage sites is 1, 2, 3, 4, 5, 6, 7, 8, 9, 10, or is between 1 and 15. In some embodiments, when the linker comprises more than one protease cleavage site, each protease cleavage site is independently the same as or different from one or more of the other protease cleavage sites.

In some embodiments, the linker comprises one protease cleavage site (e.g. any of the protease cleavage sites disclosed herein, e.g. a cleavage site having the amino acid sequence of, for example, any of SEQ ID NOs: 32, 89-98 and 103-106) (SEQ ID NOs: 89 and 90 each have two protease cleavage sites, a MMP2/9 site and a MTSP site.). In some embodiments, the protease cleavage site comprises the amino acid sequence of SEQ ID NO: 32 [LVPRGS]. In some embodiments, the protease cleavage site comprises the amino acid sequence of SEQ ID NO: 91. In some embodiments, the protease cleavage site comprises the amino acid sequence of SEQ ID NO: 92. In some embodiments, the protease cleavage site comprises the amino acid sequence of SEQ ID NO:93. In some embodiments, the protease cleavage site comprises the amino acid sequence of SEQ ID NO:94. In some embodiments, the protease cleavage site comprises the amino acid sequence of SEQ ID NO:95. In some embodiments, the protease cleavage site comprises the amino acid sequence of SEQ ID NO:96. In some embodiments, the protease cleavage site comprises the amino acid sequence of SEQ ID NO:97. In some embodiments, the protease cleavage site comprises the amino acid sequence of SEQ ID NO:98. In some embodiments, the protease cleavage site comprises the amino acid sequence of SEQ ID NO: 103. In some embodiments, the protease cleavage site comprises the amino acid sequence of SEQ ID NO:104. In some embodiments, the protease cleavage site comprises the amino acid of any one of SEQ ID NOs: 89, 90, 91, 92, 93, 94, 95, 96, 97, 98, 103, 104, 105, and 106. In some embodiments, the linker comprises multiple protease cleavage sites (for example, any of the protease cleavage sites known and/or disclosed herein, e.g. a cleavage site having the amino acid sequence of any of SEQ ID NOs: 32, 89-98 and 103-104), such as two, three, four, five, six or more protease cleavage sites, e.g. up to ten or up to fifteen protease cleavage sites, and each cleavage site is the same as or different from any of the other cleavage sites. In some embodiments, one or more than one of the multiple protease cleavage sites comprises the amino acid sequence of SEQ ID NO: 32 (amino acid sequence LVPRGS). In some embodiments, the CD45 recruiting domain comprises one or more than one protease cleavage sites. In some embodiments, the linker amino acid sequence is or comprises the amino acid sequence of any one or more of (e.g. 1, 2, 3, 4, 5, 6 or more) of SEQ ID NOs: 99-102, 172-177, and 108-120. In some embodiments, the linker comprises one copy or more than one copy (e.g. 1, 2, 3, 4, 5, 6 or more) of the amino acid sequence of any of SEQ ID NOs: 89-98 and 103-106.

In some embodiments, the protease cleavage site, or one or more of the multiple protease cleavage sites, is recognized by one or more (e.g. 1, 2, 3, 4, 5, 6, 7, 8, 9, 10, or 15, or any range between any two of these values) of thrombin, trypsin, plasmin, prostate-specific antigen (PSA), urokinase plasminogen activator (uPA), urokinase plasminogen activator receptor (uPAR), matrix metalloproteinase (MMP), matriptase (MT-SP1), legumain, a disintegrin and metalloproteinase (ADAM), and transmembrane Serine Protease (TMPRSS). Alternatively stated, in some embodiments, the protease that recognizes the protease cleavage site, or that recognizes one or more of the multiple protease cleavage sites, is one or more of thrombin, trypsin, plasmin, prostate-specific antigen (PSA), urokinase plasminogen activator (uPA), urokinase plasminogen activator receptor (uPAR), matrix metalloproteinase (MMP), matriptase (MT-SP1), legumain, a disintegrin and metalloproteinase (ADAM), transmembrane Serine Protease (TMPRSS), Granzyme B, activated protein C, Caspase, Cathepsin, Chymase, Elastase, Guanidinobenzoatase, HtrAl, Human Neutrophil Elastase, Lactoferrin, Marapsin, NS3/4A, PACE4, tissue plasminogen activator (tPA), DESC1, DPP-4, FAP, Hepsin, Matriptase-2, secretase, kallikrein-related peptidase (KLK), and tryptase, or a serine protease, a cysteine-type lysosomal protease, a metalloproteinase, a coagulation factor protease, or an aspartyl-type lysosomal protease. In some embodiments, the protease cleavage site comprises an amino acid sequence that is cleaved by a protease or type of protease listed herein. In some embodiments, the protease cleavage site or one or more of the multiple protease cleavage sites is recognized by an endogenous protease or by a protease which is present in a tumor microenvironment. In some embodiments, the protease that recognizes the protease cleavage site, or that recognizes one or more of the multiple protease cleavage sites, is a protease which is present in a tumor microenvironment.

In some embodiments, the linker comprises, in any order, including e.g. from the amino to the carboxy terminus and from the carboxy to the amino terminus, any of the following combinations of cleavage sites: for example, uPA and MMP, uPA and matriptase, MMP and matriptase, matriptase and MMP and uPA. In some embodiments, the linker further comprises one or more spacer sequences at either or both ends of one or more of each protease cleavage site to provide flexibility. In some embodiments, the linker further comprises one or more of a GS sequence, e.g. a GS sequence as disclosed herein (e.g. any one or more of SEQ ID NOs: 52, 54, 57), at either or both ends of one or more of each protease cleavage site. In some embodiments, the CD45-gate CAR comprises one or more of the linkers disclosed herein (e.g. SEQ ID NOs: 7-10, 99-102, 172-177, 108-120), e.g. 1, 2, 3, 4, 5, 15, or any intervening integer value or any range between any two such values. The linkers can be placed, e.g., between the CD45 recruiting domain, e.g., an anti-CD45 scFv, and an antigen binding domain, e.g., an antigen binding domain of a CAR. In some embodiments, the CD45 recruiting domain comprises an anti-CD45 antibody such as scFv, and the linkers can be placed between VL and VH or between VH and VL of the CD45 binding domain. In some embodiments, the antigen binding domain comprises 53B6 as disclosed herein. In some embodiments, the GS sequence provides a spacer sequence that creates flexibility between functional domains. In some embodiments, either region or both regions flanking the protease cleavage site, or the spacer sequence providing flexibility is not a GS sequence, as many flexible sequences that may function equally well are known in the art. See for example, Whitlow M. et al., An improved linker for single-chain Fv with reduced aggregation and enhanced proteolytic stability. Protein Eng. 1993 November; 6 (8): 989-95. doi: 10.1093/protein/6.8.989. PMID: 8309948. In some embodiments, the spacer sequence creates flexibility and does not interfere with the activities of each functional domain. In some embodiments, the linker can be any suitable length, e.g. from 15-100 amino acids, 30-100 amino acids, 20-80 amino acids, 20-50 amino acids, or 25, 30, 35, 40, 45, 50, or 55 amino acids in length, or about 25, about 30, about 35, about 40, about 45, about 50, or about 55 amino acids in length.

In some embodiments, the protease cleavage site or one or more of the multiple protease cleavage sites is recognized by one or more of a serine protease, a cysteine-type lysosomal protease, an aspartyl-type lysosomal protease and a metalloproteinase. In some embodiments, a protease that recognizes the protease cleavage site, or that recognizes one or more of the multiple protease cleavage sites, is one or more of a serine protease, a cysteine-type lysosomal protease, an aspartyl-type lysosomal protease and a metalloproteinase.

In some embodiments, the linker comprises between 1 and 15 or between 1 and 10 or 1, 2, 3, 4, 5, 6, 7, 8, 9 or 10 cleavage sites, each one of which may be the same as or different from one or more of the other cleavage sites, and/or the number of proteases that recognize the protease cleavage site or sites is between 1 and 15 or between 1 and 10 or is 1, 2, 3, 4, 5, 6, 7, 8, 9 or 10.

In some embodiments, the linker comprises an amino GS peptide, a peptide comprising a protease cleavage site, and a carboxy GS peptide. In some embodiments, the linker comprises the amino acid sequence of SEQ ID NO: 9, which comprises an amino GS peptide (SEQ ID NO: 52), a cleavage peptide (e.g. SEQ ID NO: 53) comprising a protease cleavage site, and a carboxy GS peptide (SEQ ID NO: 54). In some embodiments, the linker comprises the same amino and carboxy GS peptides and a different cleavage peptide comprising the same or a different protease cleavage site.

In some embodiments, the linker of the protease-activating CD45-gate CAR disclosed herein comprises the amino acid sequence of any one or more of SEQ ID NOs: 9, 10, 102, 172-177, and 108-120. In some embodiments, the linker comprises one or more protease cleavage sites having an amino acid sequence selected from the group consisting of SEQ ID NOs: 32, 89-98 and 103-104, optionally wherein the number of protease cleavage sites is 1, 2, 3, 4, 5, 6, 7, 8, 9, 10, or between 1 and 15, and, when the linker comprises more than one protease cleavage site, each protease cleavage site is independently the same as or different from one or more of the other protease cleavage sites.

In some embodiments, the linker of the protease-activating CD45-gate CAR disclosed herein comprises, on either or both ends of, and contiguous with, one or more of the protease cleavage sites, one or more sets of contiguous GS sequences, wherein each GS sequence has an amino acid sequence of GS, GGS, GGGS (SEQ ID NO: 184), or GGGGS (SEQ ID NO:178), and the sets of contiguous GS sequences comprise GS sequences in any combination, optionally wherein a set of contiguous GS sequences comprises any combination of 1, 2, 3, 4, 5, 6, 7, 8, 9 or 10 GS sequences or comprises between 1 and 15 GS sequences in any combination. Exemplary embodiments of linkers comprising such sets of contiguous GS sequences include SEQ ID NOs: 9, 10, 102, 172-177, and 107-120.

In some embodiments, the protease-activating CD45-gate CAR comprises more than one linker comprising a protease cleavage site, wherein each linker has an amino acid sequence independently selected from SEQ ID NOs: 9, 10, 102, 172-177, and 107-120, and optionally wherein the number of linkers is 2, 3, 4, 5, or an integer value between 2 and 10. Exemplary embodiments of protease-activating CD45-gate CARs comprising more than one linker include SEQ ID NOs: 125-128 and 142-145.

In some embodiments, the extracellular domain comprises a stalk domain between the antigen binding domain and the transmembrane domain.

In some embodiments, the intracellular signaling domain comprises an activating domain. In some embodiments, the intracellular signaling domain comprises a costimulatory domain. In some embodiments, the intracellular signaling domain comprises an activating domain and a costimulatory domain. In some embodiments, the intracellular signaling domain comprises an activating domain such as an ITAM-containing domain. In some embodiments, the intracellular signaling domain comprises the CD3 zeta intracellular domain. In some embodiments, the intracellular signaling domain comprises a CD3 zeta domain that comprises the amino acid sequence of SEQ ID NO: 34 or a fragment thereof.

In some embodiments, an intracellular signaling domain for use in a protease-activating CD45-gate CAR can be the cytoplasmic sequences of, for example without limitation, the T cell receptor and co-receptors that act in concert to initiate signal transduction following antigen receptor engagement, as well as any derivative or variant of these sequences and any synthetic sequence that has the same functional capability.

In some embodiments, the intracellular domain comprises or further comprises at least one costimulatory domain. In some embodiments, the at least one costimulatory domain is a cytoplasmic signaling region or domain of CD28, OX-40, 4-1BB/CD137, CD2, CD7, CD27, CD30, CD40, programmed death-1 (PD-1), inducible T cell costimulator (ICOS), lymphocyte function-associated antigen-1 (LFA-1 (CD1 la/CD 18), CD3 gamma, CD3 delta, CD3 epsilon, CD247, CD276 (B7-H3), LIGHT, (TNFSF14), NKG2C, Ig alpha (CD79a), DAP-10, Fc gamma receptor, MHC class I molecule, TNF receptor proteins, an Immunoglobulin protein, cytokine receptor, integrins, Signaling Lymphocytic Activation Molecules (SLAM proteins), activating NK cell receptors, BTLA, a Toll ligand receptor, ICAM-1, B7-H3, CDS, ICAM-1, GITR, BAFFR, LIGHT, HVEM (LIGHTR), KIRDS2, SLAMF7, NKp80 (KLRFI), NKp44, NKp30, NKp46, CD19, CD4, CD8alpha, CD8beta, IL-2R beta, IL-2R gamma, IL-7R alpha, ITGA4, VLA1, CD49a, ITGA4, CD49D, ITGA6, VLA-6, CD49f, ITGAD, CD1 Id, ITGAE, CD103, ITGAL, CD1 la, LFA-1, IT GAM, CD1 lb, ITGAX, CD1 lc, ITGB1, CD29, ITGB2, CD 18, LFA-1, ITGB7, NKG2D, TNFR2, TRANCE/RANKL, DNAMI (CD226), SLAMF4 (CD244, 2B4), CD84, CD96 (Tactile), CEACAM1, CRT AM, Ly9 (CD229), CD 160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD 19a, a ligand that specifically binds with CD83, or any combination thereof.

In some embodiments, the protease-activating CD45-gate CAR comprises a signal sequence. In some embodiments, the signal sequence is the CD8 signal sequence. In some embodiments, the signal sequence comprises the amino acid sequence of SEQ ID NOs: 1 or 44.

In some embodiments, the amino acid sequence of the protease-activating CD45-gate CAR is or comprises the amino acid sequence of any one of SEQ ID NOs: 16-31 and 121-170, or is or comprises the amino acid sequence of any one of SEQ ID NOs: 16-31 and 121-170 but which lacks any one or more of: the signal sequence, hemagglutinin (HA) tag, and/or V5 tag.

In an alternative aspect of the protease-activating CD45-gate CAR disclosed herein, the CD45 recruiting domain itself comprises one or more than one protease cleavage sites (e.g. protease cleavage sites as disclosed herein), and the linker that joins the CD45 recruiting domain to the antigen binding domain either comprises no protease cleavage site or comprises one or more than one protease cleavage sites (e.g. protease cleavage sites as disclosed herein). In an embodiment, protease cleavage produces a CAR attached to a fragment of the CD45 protein, and the protease cleavage partially or wholly relieves the CD45-induced inhibition of the CAR's activity.

Also provided herein is a CAR that specifically recognizes and binds to MUC16. In an embodiment, the anti-MUC16 CAR comprises the amino acid sequence of any of SEQ ID NO: 11, 75, 76, 77, 78, and 79.

The present disclosure further provides a protease-activating CD45-gate T cell receptor (TCR). In an embodiment, the protease-activating CD45-gate TCR comprises a modified TCR α, β, γ, or δ polypeptide, wherein the modification comprises a CD45 recruiting domain joined by a protease-cleavable linker peptide to the amino terminus of the TCR α, β, γ, or δ polypeptide. In an embodiment, the protease-activating CD45-gate TCR comprises a modified single-chain TCR, wherein the modification comprises a CD45 recruiting domain joined by a protease-cleavable linker peptide to the amino terminus of a single-chain TCR. In an embodiment, the polypeptide is a CD45-TCR α, β, γ, or δ polypeptide. In an embodiment, the polypeptide is a CD45-single chain TCR. In another aspect, provided herein is a nucleic acid that encodes the CD45-gate TCR disclosed herein.

In another aspect, provided herein is a nucleic acid encoding the extracellular domain of the protease-activating CD45-gate CAR disclosed herein. In some embodiments, the encoded extracellular domain comprises a CD45 recruiting domain such as but not limited to a CD45 recruiting domain disclosed herein, the extracellular domain of a CAR such as but not limited to a CAR disclosed herein, and a linker connecting the carboxy terminus of the CD45 recruiting domain to the amino terminus of the CAR extracellular domain, such as but not limited to a linker disclosed herein.

In another aspect, provided herein is a nucleic acid that encodes the CD45-gate CAR disclosed herein.

Also provided herein is a nucleic acid that encodes a polypeptide that comprises the amino acid sequence of any of SEQ ID NOs: 11, 56, 75, 76, 77, 78, 79 and 121-170. In some embodiments, the polypeptide is an anti-MUC16 CAR as disclosed herein. In some embodiments, the polypeptide is a protease-activating CD45-gate CAR as disclosed herein.

In another aspect, provided herein is a vector comprising a nucleic acid as disclosed herein. In an embodiment, the vector is an expression vector. In an embodiment, the vector is a viral vector, a retroviral vector, a DNA vector, a plasmid, a RNA vector, an adenoviral vector, an adenovirus associated vector, a lentiviral vector, or any combination thereof.

In a further aspect, provided herein is an engineered cell e.g. an engineered immune cell comprising a protease-activating CD45-gate CAR as disclosed herein.

In a further aspect, provided herein is an engineered immune cell comprising a nucleic acid as disclosed herein.

In another aspect, provided herein is an engineered immune cell comprising a vector as disclosed herein.

In various embodiments, any of the engineered cells e.g. any of the engineered immune cells disclosed herein functionally express a protease-activating CD45-gate CAR as disclosed herein. In various embodiments, any of the engineered cells e.g. any of the engineered immune cells disclosed herein functionally express a protease-activating CD45-gate CAR that comprises the amino acid sequence of any of SEQ ID NOs: 11, 56, 75, 76, 77, 78, 79, and 121-170. Also provided herein are engineered cells e.g. engineered immune cells that functionally express a MUC16-specific CAR that comprises the amino acid sequence of any of SEQ ID NOs: 11, 56, 75, 76, 77, 78, 79 and 121-170. In various embodiments, any of the engineered cells e.g. any of the engineered immune cells disclosed herein is an isolated cell.

In various embodiments, the engineered cell e.g. engineered immune cell disclosed herein functionally expresses the protease-activating CD45-gate CAR disclosed herein from a nucleic acid encoding the protease-activating CD45-gate CAR. In various embodiments, the engineered cell e.g. engineered immune cell disclosed herein functionally expresses CD45, e.g. functionally expresses the cell's endogenous CD45 gene or functionally expresses CD45, or a fragment or variant of CD45, from an exogenous nucleic acid that encodes CD45 and that was introduced into the cell. In various embodiments, the engineered cell e.g. engineered immune cell disclosed herein functionally expresses the anti-MUC16 CAR disclosed herein from a nucleic acid encoding the anti-MUC16 CAR. In some embodiments, the anti-MUC16 CAR is a protease-activating CD45-gate MUC16CAR.

In various embodiments, the engineered immune cell disclosed herein is an engineered B cell, mast cell, myeloic-derived phagocyte, T cell e.g. an alpha/beta and/or gamma/delta T cell, tumor infiltrating lymphocyte (TIL), NK cell, TCR-expressing cell, dendritic cell, or NK-T cell. In some embodiments, the cell is and/or is derived from an autologous T cell. In some embodiments, the cell is and/or is derived from an allogeneic T cell or an iPSC (induced pluripotent stem cell). In some embodiments, the cell is and/or is derived from an iPSC-derived T cell.

In an embodiment, an engineered immune cell as disclosed herein comprises or further comprises one or more genomic modifications of one or more of the endogenous CD52 gene and the endogenous TCR genes, e.g. the TCRα gene. In an embodiment, an engineered immune cell as disclosed herein is a T cell.

In another aspect, provided herein is a population of immune cells comprising one or more of the engineered immune cells disclosed herein. In an embodiment, the population comprises about or at least about 1×10⁴, about or at least about 1×10⁵, about or at least about 1×10⁶, about or at least about 1×10⁷, or about or at least about 1×10⁸ engineered cells, e.g. engineered immune cells, as disclosed herein, optionally wherein the population does not comprise more than about 1×10¹⁰ or more than about 1×10⁹ or more than about 5×10⁹ engineered cells, e.g. engineered immune cells, as disclosed herein. In an embodiment, a population of immune cells as disclosed herein is enriched for the engineered immune cell as disclosed herein. In various embodiments, the population of immune cells is at least 50%, e.g. 50%, 55%, 60%, 65%, 70%, 75%, 80%, 85%, 90%, 95%, or more than 95% engineered cells that are T cells, e.g., alpha/beta T cells and gamma/delta T cells, B cells, natural killer (NK) cells, natural killer T (NKT) cells, mast cells, and/or myeloic-derived phagocytes. In various embodiments, the population of immune cells is at least 50%, e.g. 50%, 55%, 60%, 65%, 70%, 75%, 80%, 85%, 90%, 95%, or more than 95% engineered T cells.

In another aspect, provided herein is a composition comprising an engineered cell e.g. an engineered immune cell as disclosed herein or a population of cells as disclosed herein, and a pharmaceutically acceptable carrier.

Also provided herein is the use of any of the engineered immune cells e.g. engineered T cells provided herein in the manufacture of a medicament for the treatment of cancer or for inhibiting tumor growth or progression in a subject in need thereof. Also provided herein is any of the engineered immune cells e.g. engineered T cells provided herein for use as a medicament and for use in treating any disease or condition amenable to treatment by CAR T cell therapy. In some embodiments, the disease or condition is a cancer, autoimmune disease, or infection. In some embodiments, the disease or condition is a form of cancer.

In another aspect, provided herein is a method of treating a condition in a patient comprising administering to the patient an engineered immune cell as disclosed herein. In an embodiment of the method, the engineered immune cell is an allogeneic engineered immune cell derived from a donor other than the patient.

In another aspect, provided herein is a method of treating a condition in a patient comprising administering to the patient a population of engineered immune cells as disclosed herein. In an embodiment of the method, the engineered immune cells of the population are derived from one or more allogeneic immune cells from a donor other than the patient.

In another aspect, provided herein is a method of treating a condition in a patient comprising administering to the patient a pharmaceutical composition as disclosed herein. In an embodiment of the method, the composition comprises one or more engineered allogeneic immune cells derived from a donor other than the patient.

In embodiments of the methods of treating a condition in a patient disclosed herein, the condition may be any disease or condition amenable to treatment by CAR T cell therapy. In some embodiments, the disease or condition is a cancer, autoimmune disease, or infection. In some embodiments, the disease or condition is a form of cancer. In some embodiments, the cancer is a hematological malignancy or non-solid tumor. In some embodiments, the cancer is a solid cancer or solid tumor. In some embodiments, the cancer is a hematological malignancy that is acute lymphoblastic leukemia (ALL), acute myeloid leukemia (AML), chronic myelogenous leukemia (CML), chronic eosinophilic leukemia (CEL), myelodysplasia syndrome (MDS), non-Hodgkin's lymphoma (NHL), or multiple myeloma (MM). In some embodiments, the cancer is a solid cancer that is biliary cancer, bladder cancer, bone or soft tissue carcinoma, brain tumor, breast cancer, cervical cancer, colon cancer, colorectal adenocarcinoma, colorectal cancer, desmoid tumor, embryonal cancer, endometrial cancer, esophageal cancer, gastric cancer, gastric adenocarcinoma, glioblastoma multiforme, gynecological tumor, head and neck squamous cell carcinoma, hepatic cancer, lung cancer, malignant melanoma, osteosarcoma, ovarian cancer, pancreatic cancer, pancreatic ductal adenocarcinoma, primary astrocytic tumor, primary thyroid cancer, prostate cancer, renal cancer, renal cell carcinoma, rhabdomyosarcoma, skin cancer, soft tissue sarcoma, testicular germ-cell tumor, urothelial cancer, uterine sarcoma, or uterine cancer.

In another aspect, provided herein is a method of reducing on-target off-tumor toxicity of CAR T cells comprising administering to a patient a cell as disclosed herein, a population of cells as disclosed herein, or a composition as disclosed herein, wherein the on-target, off-tumor toxicity is lower than the on-target, off-tumor toxicity of control cells, a population of control cells or a composition of control cells, respectively, administered under the same conditions, wherein the control cells, population of control cells, or control cells of the composition comprise a CAR that lacks at least one of a CD45 recruiting domain and a linker that comprises a protease cleavage site.

In another aspect, provided herein is a method of increasing the efficiency of CAR T cells or CAR T cell therapy comprising administering to a patient a cell as disclosed herein, a population of cells as disclosed herein, or a composition as disclosed herein, wherein the efficiency is greater than the efficiency of control cells, a population of control cells or a composition of control cells, respectively, administered under the same conditions, wherein the control cells, population of control cells, or control cells of the composition comprise a CAR that lacks at least one of a CD45 recruiting domain and a linker that comprises a protease cleavage site.

In another aspect, provided herein is a method of increasing the efficacy of CAR T cell therapy comprising administering to a patient a cell as disclosed herein, a population of cells as disclosed herein, or a composition as disclosed herein, wherein the efficacy is greater than the efficacy of control cells, a population of control cells or a composition of control cells, respectively, administered under the same conditions, wherein the control cells, population of control cells, or control cells of the composition comprise a CAR that lacks at least one of a CD45 recruiting domain and a linker that comprises a protease cleavage site.

In another aspect, provided herein is a method of increasing the efficacy of CAR T cell therapy against a solid tumor comprising administering to a patient having a solid tumor a cell as disclosed herein, a population of cells as disclosed herein, or a composition as disclosed herein, wherein the efficacy against the solid tumor is greater than the efficacy against the solid tumor of control cells, a population of control cells or a composition of control cells, respectively, administered under the same conditions, wherein the control cells, population of control cells, or control cells of the composition comprise a CAR that lacks at least one of a CD45 recruiting domain and a linker that comprises a protease cleavage site.

In another aspect, provided herein is a method of reducing the incidence of side effects in CAR T cell therapy comprising administering to a patient a cell as disclosed herein, a population of cells as disclosed herein, or a composition as disclosed herein, wherein the incidence of side effects is lower than the incidence of side effects when control cells, a population of control cells or a composition of control cells, respectively, are administered under the same conditions, wherein the control cells, population of control cells, or control cells of the composition comprise a CAR that lacks at least one of a CD45 recruiting domain and a linker that comprises a protease cleavage site.

In another aspect, provided herein is a method of effecting reversible colocalization of CD45 and a CD45-gate CAR in a CAR T cell, the reversible colocalization comprising a CD45-gate CAR recruiting domain binding to a CD45 protein on a CAR T cell surface, resulting in colocalization, which binding and colocalization can be disrupted by a protease cleaving a protease cleavage site in the CD45-gate CAR's linker, thereby reversing the colocalization, the method comprising the steps of:
(a) providing a T cell that expresses CD45,
(b) introducing a nucleic acid encoding a protease-activating CD45-gate CAR as disclosed herein into the T cell,
(c) maintaining the T cell under conditions in which both CD45 and the CAR are functionally expressed at the cell surface,
resulting in a CD45-gate CAR recruiting domain binding to a CD45 protein on the CAR T cell surface and colocalization of the CAR and the CD45 protein, which colocalization can be reversed by a protease cleaving a protease cleavage site in the CD45-gate CAR's linker, wherein the cleaving produces a functional CAR no longer connected by the linker to the CD45-gate CAR recruiting domain.

In another aspect, provided herein is a method of effecting reversible reduction of CAR activity by a CD45 protein in a CAR T cell, the reversible reduction of CAR activity comprising a CD45-gate CAR recruiting domain binding to a CD45 protein on a CAR T cell surface, resulting in inactivation of the chimeric antigen receptor by the CD45, which inactivation can be disrupted by a protease cleaving a protease cleavage site in the CD45-gate CAR's linker, resulting in the disassociation of the chimeric antigen receptor and the CD45 protein and ending the inactivation of the chimeric antigen receptor by the CD45, the method comprising the steps of:
(a) providing a T cell that expresses CD45,
(b) introducing a nucleic acid encoding a CD45-gate CAR into the T cell,
(c) maintaining the T cell under conditions in which both CD45 and the CAR are functionally expressed at the cell surface,
resulting in a CD45-gate CAR recruiting domain binding to a CD45 protein on the CAR T cell surface, association of the CAR and the CD45 protein, and reduction of the CAR's activity by the CD45 protein, which activity reduction can be reversed by a protease cleaving a protease cleavage site in the CD45-gate CAR's linker, thereby ending the association of the CAR and the CD45 protein, and ending the CD45 protein's reduction of or inhibition of CAR activity, wherein the cleaving produces a functional CAR no longer connected by the linker to the CD45-gate CAR recruiting domain.

In another aspect, provided herein is a method of treating with CAR T cell therapy a patient who has a tumor characterized by a protease-rich tumor microenvironment, comprising administering to the patient a cell as disclosed herein, a population of cells as disclosed herein, or a composition as disclosed herein, wherein CAR T cell activity is lower outside the protease-rich tumor microenvironment than in the protease-rich tumor microenvironment.

In another aspect, provided herein is a method of regulating cytotoxic activity of a CAR T cell that comprises a CAR as disclosed herein, the method comprising inhibiting the cytotoxic activity of the CAR T cell by associating the CAR with CD45 of the CAR T cell, and activating the cytotoxic activity of the CAR T cell by subjecting and/or exposing the CAR T cell to a protease that recognizes and cleaves the protease cleavage site.

In embodiments of the methods of treating a condition in a patient disclosed herein, the cell, population of cells or composition can be administered to the subject on one occasion or can be administered to the subject on two or more occasions spaced at least about 1, 2, 3, 4, 5, 6, 7, or more days apart. In some embodiments, the disorder can be a viral disease, a bacterial disease, a cancer, an inflammatory disease, an immune disease, or an aging-associated disease.

Also provided herein are chimeric antigen receptors (CARs) that bind to MUC16 (“MUC16-specific CARs” or “anti-MUC16 CARs” or “MUC16 CARs”). It is demonstrated that the expression of MUC16 specific CARs in T cells is effective to activate the T cells upon contact with MUC16. The MUC16 specific CARs provided herein bind human MUC16 and exhibit cytotoxic activity upon contact with MUC16-expressing cells.

Accordingly, in another aspect, provided herein is a MUC16 specific chimeric antigen receptor (CAR) comprising an extracellular ligand-binding domain, a first transmembrane domain, and an intracellular signaling domain, wherein the extracellular domain comprises a single chain variable fragment (scFv) binding to the extracellular domain of MUC16.

The extracellular domain of MUC16 specific CARs comprises a scFv, wherein the scFv comprises a heavy chain variable (VH) region and a light chain variable (VL) region, the VH and VL regions each comprising three complementarity determining regions (CDRs) specific for MUC16.

In some embodiments, the VH region comprises (i) a VH complementarity determining region one (CDR1) having the amino acid sequence shown in SEQ ID NO: 60 or 63; (ii) a VH complementarity determining region two (CDR2) having the amino acid sequence shown in SEQ ID NO: 61 or 64; and (iii) a VH complementarity determining region three (CDR3) having the amino acid sequence shown in SEQ ID NO: 62 or 65; and/or the VL region comprises (i) a VL complementarity determining region one (CDR1) having the amino acid sequence shown in SEQ ID NO: 66 or 69; (ii) a VL complementarity determining region two (CDR2) having the amino acid sequence shown in SEQ ID NO: 67 or 70; and (iii) a VL complementarity determining region three (CDR3) having the amino acid sequence shown in SEQ ID NO: 68 or 71.

In some embodiments, provided are MUC16 specific CARs comprising a scFv, wherein the scFv comprises a VH region having the sequence shown in SEQ ID NO: 58.

In some embodiments, provided are MUC16 specific CARs comprising a scFV, wherein the scFv comprises a VL region having the sequence shown in SEQ ID NO: 59.

In some embodiments, provided are MUC16 specific CARs comprising a scFV comprising a VH region and a VL region, wherein the VH region has the sequence shown in SEQ ID NO: 58, and the VL region has the sequence shown in SEQ ID NO: 59.

In some embodiments, the VH region comprises the sequence shown in SEQ ID NO: 58, or a variant thereof with one or several conservative amino acid substitutions in residues that are not within a CDR and/or the VL region comprises the amino acid sequence shown in SEQ ID NO: 59, or a variant thereof with one or several amino acid substitutions in amino acids that are not within a CDR.

In some embodiments, the VH region comprises the amino acid sequence shown in SEQ ID NO: 58 and the VL region comprises the amino acid sequence shown in SEQ ID NO: 59.

In some embodiments, each CDR is defined in accordance with the Kabat definition, the Chothia definition, the combination of the Kabat definition and the Chothia definition, the AbM definition, or the contact definition of CDR.

In some embodiments, the MUC16 specific CAR intracellular signaling domain comprises a CD32 signalling domain. In some embodiments, the intracellular signaling domain comprises a 4-1BB domain. In some embodiments, the MUC16 specific CAR comprises a second intracellular signaling domain. In some embodiments, the second intracellular signaling domain comprises a 4-1BB domain.

In some embodiments, the MUC16 specific CARs disclosed herein may comprise a stalk domain between the extracellular ligand-binding domain and the first transmembrane domain. In some embodiments, the stalk domain is selected from the group consisting of: a CD8α hinge, an IgG1 hinge, and an FcγRIIIα hinge. In some embodiments, the stalk domain is a human CD8α hinge, a human IgG1 hinge, or a human FcγRIIIα hinge.

In some embodiments, the MUC16 specific CARs disclosed herein may comprise one or more epitopes or mimotopes specific for one or more monoclonal antibodies. In some embodiments, the one or more epitopes or mimotopes specific for a monoclonal antibody includes a CD20 epitope or mimotope. In some embodiments, the CD20 epitope or mimotope comprises the amino acid sequence shown in SEQ ID NO: 50 or SEQ ID NO: 51.

In some embodiments, the MUC16 specific CAR comprises the amino acid sequence shown in any of SEQ ID NO: 11, 75, 76, 77, 78, and 79, with or without a signal sequence. In some embodiments, the MUC16 specific CAR comprises the amino acid sequence shown in SEQ ID NO: 75, with or without a signal sequence. In some embodiments, the MUC16 specific CAR comprises the amino acid sequence shown in SEQ ID NO: 76, with or without a signal sequence.

In some embodiments, the first transmembrane domain comprises a CD8α chain transmembrane domain.

In some embodiments, the MUC16 specific CAR can comprise another extracellular ligand-binding domain which is not specific for MUC16.

In some embodiments, the extracellular ligand-binding domain(s), the first transmembrane domain, and intracellular signaling domain(s) are on a single polypeptide.

In some embodiments, the CAR can comprise a second transmembrane domain, wherein the first transmembrane domain and the extracellular ligand-binding domain(s) are on a first polypeptide, and wherein the second transmembrane domain and the intracellular signaling domain(s) are on a second polypeptide. In an exemplary embodiment, the first transmembrane domain comprises a transmembrane domain from the a chain of the high-affinity IgE receptor (FcεRI) and the second transmembrane domain comprises a transmembrane domain from the γ or β chain of FcεRI.

In some embodiments, the CAR can comprise a third polypeptide comprising a third transmembrane domain fused to an intracellular signaling domain from a co-stimulatory molecule, wherein the third transmembrane domain comprises a transmembrane domain from the γ or β chain of FcεRI.

In another aspect, provided herein is an isolated polynucleotide comprising a nucleic acid sequence encoding the MUC16 specific CAR described herein.

In another aspect, provided herein is an expression vector comprising the polynucleotide encoding the MUC16 specific CAR described herein.

In another aspect, provided herein is an engineered immune cell expressing at its cell surface membrane a MUC16 specific CAR described herein. In some embodiments, the engineered immune cell can comprise another CAR which is not specific for MUC16. In some embodiments, the engineered immune cell can comprise a polynucleotide encoding a suicide polypeptide. In some embodiments, the suicide polypeptide is RQR8.

In some embodiments, the engineered immune cell is derived from an inflammatory T-lymphocyte, a cytotoxic T-lymphocyte, a regulatory T-lymphocyte, or a helper T-lymphocyte. In some embodiments, the engineered immune cell is an inflammatory T-lymphocyte, a cytotoxic T-lymphocyte, a regulatory T-lymphocyte, or a helper T-lymphocyte.

In some embodiments, the engineered immune cell can comprise a disruption of one or more endogenous genes, wherein the endogenous gene encodes TCRα, TCRβ, CD52, glucocorticoid receptor (GR), deoxycytidine kinase (dCK), or an immune checkpoint protein such as for example programmed death-1 (PD-1).

In some embodiments, the engineered immune cell is obtained from a healthy donor. In some embodiments, the engineered immune cell is obtained from an individual afflicted with a disease or disorder.

In another aspect, provided herein is an engineered immune cell expressing at its cell surface membrane a MUC16 specific CAR as described herein for use as a medicament. In another aspect, provided herein is a MUC16 specific antibody for use as a medicament. In some embodiments, the medicament comprising the MUC16 specific CAR expressing immune cells or MUC16 specific antibodies is for use in treatment of a MUC16 associated disease or disorder.

In one aspect, provided herein is a population of cells comprising engineered immune cells expressing MUC16 specific CARs described herein, wherein (ii) said population of cells comprises a percentage of stem cell memory and central memory cells greater than 20%, 30% or 40%, and/or (iii) said population of cells achieves a percentage lysis of MUC16 expressing cells greater than 10%, 20%, 30% or 40%.

In another aspect, provided herein is a method of engineering an immune cell expressing any one of the MUC16 specific CARs described herein, the method comprising: providing an immune cell; and introducing into the cell at least one polynucleotide encoding said MUC16 specific CAR; whereby said immune cell expresses said MUC16 specific CAR.

In some embodiments, the method comprises providing an immune cell; introducing into the cell at least one polynucleotide encoding said MUC16 specific CAR; and introducing at least one polynucleotide encoding a CAR which is not specific for MUC16.

In another aspect, provided herein is a method of treating a subject suffering from a MUC16 associated disease or disorder, the method comprising: providing an immune cell expressing at the surface a MUC16 specific CAR as described herein; and administering said immune cells to said subject. The disclosure also provides methods of treating subjects suffering from a MUC16 associated disease or disorder, the method comprising providing MUC16 specific antibodies described herein and administering said antibodies to said subject.

In some embodiments, provided herein is a pharmaceutical composition comprising an engineered immune cell expressing MUC16 specific CARs as described herein. In other embodiments, the disclosure provides pharmaceutical compositions comprising any of the MUC16 specific antibodies described herein.

In another aspect, provided herein is a method of treating a condition associated with malignant cells expressing MUC16 in a subject comprising administering to a subject in need thereof an effective amount of the pharmaceutical composition comprising an engineered immune cell as described herein. In some embodiments, the condition is a cancer.

In another aspect, provided herein is a method of inhibiting tumor growth or progression in a subject who has malignant cells expressing MUC16, comprising administering to the subject in need thereof an effective amount of the pharmaceutical composition comprising an engineered immune cell as described herein or a MUC16 specific antibody as described herein to the subject.

In another aspect, provided herein is a method of inhibiting metastasis of malignant cells expressing MUC16 in a subject, comprising administering to the subject in need thereof an effective amount of the pharmaceutical composition comprising an engineered immune cell as described herein or a MUC16 specific antibody as described herein to the subject.

In another aspect, provided herein is a method of inducing tumor regression in a subject who has malignant cells expressing MUC16, comprising administering to the subject in need thereof an effective amount of the pharmaceutical composition comprising an engineered immune cell as described herein or a MUC16 specific antibody as described herein to the subject.

In some embodiments, any of the above methods further comprises administering one or more additional therapies, such as for example, a monoclonal antibody and/or a chemotherapeutic. In some embodiments, the monoclonal antibody can be, for example, an antibody that binds to a checkpoint inhibitor such as, for example, an anti-PD-1 antibody or an anti-PD-L1 antibody. In some embodiments, any of the above methods further comprises administering a Receptor Tyrosine Kinase inhibitor, an mTOR inhibitor, an epigenetic modulator, a proteasome inhibitor, an immunomodulatory agent such as lenalidomide, a Hedgehog inhibitor or an Isocitrate Dehydrogenase (IDH) inhibitors, to the subject.

### BRIEF DESCRIPTION OF THE DRAWINGS

FIGS. 1A-1B. FIG. 1A (left side) illustrates a potential mechanism of action for an exemplary protease-activating CD45-gate CAR. In the cell membrane of a T cell, for example, a linker peptide tethers (i.e. joins or connects) a CD45 recruiter domain to a CAR. The linker peptide comprises at least one protease site. The recruiter recognizes and binds to a CD45 protein, thus holding the CAR and CD45 protein in relatively close proximity to each other. This close proximity permits the CD45 to inactivate the CD3zeta domain. Thus, the CAR activity is reduced even when it binds to its target antigen, so long as the linker peptide is intact. When a protease cleaves the linker peptide (FIG. 1A, right side) (e.g. when the CAR T cell is in the environment of a tumor that expresses the protease that recognizes the protease site in the linker), the CAR is “released” from the CD45 and ceases to be inactivated by the CD45. The CAR T cell thus selectively becomes active primarily in the vicinity of its target tumor, thereby reducing on-target off-tumor toxicity. FIG. 1B presents schematic representations of the domains of exemplary CARs/CAR constructs. Upper: An exemplary CAR preprotein comprises a CD8 signal sequence and a CAR polypeptide. Lower: An exemplary protease-activating CD45-gate CAR preprotein comprises a CD8 signal sequence, optional hemagglutinin (HA) tag, CD45 recruiter domain, linker comprising glycine-serine (GS) rich sequences flanking a thrombin protease cleavage site, and a CAR.

FIGS. 2A-2B. CAR T cells comprising vectors encoding protease-activating CD45-gate CARs grow and express the transgenic protein. T cells were transduced with a vector that encodes a CAR that is not a protease-activating CD45-gate CAR (53B6, an anti-MUC16 CAR), with a vector that encodes a protease-activating CD45-gate CAR (CD45-Gate1-TMB-53B6, CD45-Gate2-TMB-53B6), or with a vector that encodes analogs of the protease-activating CD45-gate CARs that comprise a non-cleavable linker (CD45-Gate1-GS-53B6, CD45-Gate2-GS-53B6). The linker of the cleavable protein comprised: GS-region-thrombin cleavage site-V5 peptide-thrombin cleavage site-GS-region. The linker of the non-cleavable protein comprised: GS-region-V5 peptide-GS-region. FIG. 2A. Percentage of CAR+ cells at days 8 and 15 of production. FIG. 2B. Cell number at day 15 of production. NTD, not transduced.

FIG. 3A. Exposing the CAR T cells of FIGS. 2A-2B to thrombin resulted in reduced detection of the V5 tag in the cleavable CARs but not in the non-cleavable CARs. FIG. 3B. The ability of the CAR T cells of FIGS. 2A-2B to bind to soluble MUC16 was not significantly affected by the presence or absence of the CD45 gate.

FIGS. 4A-4B. Results of cytotoxicity assays used to assess inhibitory activity of CD45 gate. Assays were performed in R10 medium without thrombin or other exogenous protein present. FIG. 4A. Ovcar3 (MUC16^{High}), 1st round—48H cytotoxicity. FIG. 4B. Ovcar3 (MUC16^{High}), 2nd round—2×48H cytotoxicity. NTD: not transduced; Ovcar3 (MUC16^{High}): cell line that expresses a high density of the target antigen MUC16.

FIGS. 5A-5B. Results of cytotoxicity assays used to assess inhibitory activity of CD45 gate. Assays were performed in R10 medium without thrombin or other exogenous protein present. FIG. 5A. Cov644 (MUC16Low), 1st round—48H cytotoxicity. FIG. 5B. Cov644 (MUC16Low), 2nd round—2×48H cytotoxicity. NTD: not transduced; Cov644 (MUC16Low): cell line that expresses a low density of the target antigen MUC16.

FIGS. 6A-6B. Results of cytotoxicity assays used to assess reversibility of CD45 gate inhibitory activity. Assays were performed in serum-free medium. Black bars: no protease present. White bars: protease (thrombin) present. E:T: ratio of number of effector cells to number of target cells; NTD: not transduced.

FIG. 7A. 53B6 CAR T cells were successfully generated from two different T cell donors using lentiviral transfer vector containing the 53B6 scFv that targets the MUC16 protein. CAR+ T cell population was detected with recombinant MUC16 protein (SEA1-4) using flow cytometry. FIG. 7B. Phenotypes were assigned according to CD62L and CD45RO expression within the CAR+ T cell population as follows: stem cell memory (Tscm; CD45RO−/CD62L+), central memory (Tcm; CD45RO+/CD62L+), effector memory (Tem; CD45RO+/CD62L−), effector cells (Teff; CD45RO−/CD62L−). SEA=sperm protein, enterokinase, agrin; EV: empty vector.

FIGS. 8A-8C. 1×10⁷ luciferase-expressing ovarian target cells (OVCAR3, COV644, FUOV1) with different MUC16 surface protein expression levels (indicated in parentheses) were co-cultured with 53B6 CAR+ T cells for 72 hours at various Effector: Target (E:T) ratio. Empty vector (EV) control T cells were also included in the study. Percent target cell survival after being exposed to T cells was determined by comparing to target cells alone.

FIGS. 9A-9B. 1×10⁷ luciferase-expressing OVCAR3 target cells were co-cultured with 53B6 CAR+ T cells at Effector: Target ratio of 1:1 in a serial killing assay, where the T cells are exposed to fresh target cells every 2-3 days. Empty vector (EV) control T cells were also included in the study. Percent target cell survival after being exposed to T cells was determined by comparing to target cells alone. Data presented are the activity of 53B6 CAR T cells from two different donors.

FIGS. 10A-10B. 53B6 anti-MUC16 CAR T cells display anti-tumor activity against two different orthotopic ovarian tumor models. NSG mice (n=5) intra-peritoneally (IP) injected with 3×10⁶ luciferase-labeled OVCAR3 (FIG. 10A) or COV644 (FIG. 10B) cells received IP injection of 1×10⁶ or 3×10⁶ 53B6 CAR+ T cells, respectively, 14 days later. Tumor burden was monitored by bioluminescence, and the results demonstrated anti-tumor activity in both models. Empty-vector (EV) control T cells were also included in the study. LOD=Limit of Detection, based on bioluminescence imaging of non-tumor bearing mice.

FIG. 11. Schematic diagram of exemplary CD45-gate CAR constructs. TPS3, TPS4 etc. refer to linkers of various lengths (e.g. TPS6 20aa, TPS6 25aa, etc.) as shown in Table 2; MMP=matrix metalloproteinase; uPA=urokinase plasminogen activator (uPA); √ indicates presence of the indicated site; white rectangle between 53B6vL and 41BB ICD indicates the transmembrane and hinge region.

FIGS. 12A-12D. Production of new CD45-gate CAR T cells. CD45-gate CAR T cells containing a selective panel of TPS linkers were successfully generated and characterized using flow cytometry according to methods described in Example 2. Autoactivation and memory phenotypes of the CAR T cells were also determined using flow cytometry by analyzing the expression of CD45/41BB and CD45RO/CD62L on Day 9 and Day 14, respectively. FIG. 12A. The percentages of CAR transduction are comparable across all CD45-gate CAR T cells. FIG. 12B. The overall expansion of T cells remains similar among all the clones and is comparable to non-transduced control (NTD), indicting no obvious fratricide. FIG. 12C. Overall, CD45-gate CAR T cells show higher percentage of stem-cell memory subset (CD45RO−/CD62L+) than naked CAR T cells. FIG. 12D. CD45-gate CAR T cells exhibit less tonic signaling (e.g., CD25+/41BB+) than naked CAR T cells, suggesting a potential benefit of CD45-gate CARs in controlling T cell exhaustion. All cleavable linkers in FIGS. 12A-12D have a length of 45 amino acids and their sequences (e.g. TPS3 (45 aa), TPS4 (45aa), TPS6 (45 aa)) are set forth in Table 2. TPS3 (45aa) has the same amino acid sequence as SEQ ID NO:9, referred to in Table 2 as “GSTPS1 (45 aa)” (also known as “TPS1”).

FIGS. 13A-13D. CD45-gate CAR T cells containing different TPS linkers were generated. FIG. 13A. The percentages of CAR transduction are comparable for all TPS linkers except TPS10. FIG. 13B. CD45-gate does not cause obvious fratricide. FIG. 13C. CD45-gate CARs show more stem-cell memory phenotype than non-transduced T cells and T cells transduced with 53B6 (anti-MUC) CAR (non-CD45-gate). FIG. 13D. CD45-gate CARs show higher mean fluorescence intensity (MFI, top panel), but show less tonic signaling than T cells transduced with 53B6 (bottom panel). FIG. 13A shows the results of flow cytometry. All cleavable linkers in FIGS. 13A-13D have a length of 45 amino acids.

FIGS. 14A-14E. Generation and analysis of reagent cell lines. Target cells were developed in-house based on three cell lines that are positive for tumor-associated proteases, including an ovarian cancer cell line OVCAR3, a breast cancer cell line MDA-MB-231, and a lung carcinoma cell line NCI-H292. FIG. 14A. Flow cytometry results confirming the surface expression of matriptase (anti-matriptase antibody used at 3 μg/mL: R&D Systems Cat. No. BAF3946) for each cell line. Vertical axis: site scatter area (SSC-A). FIG. 14B. Surface-expressed matriptase activity was assessed through the detection of a V5 tag that is attached to the N-terminus of Muc16 CAR 53B6 via a matriptase cleavable linker TPS6 45aa, as described in Example 1. Minimal protease activity was detected in vitro despite matriptase expression, as evidenced by the fact that the V5 signal coincides with Muc16 CAR signal in OVCAR3, MDA MB 231, and H292. This indicates that V5 is not cleaved off by matriptase. Recombinant human matriptase cleaved the V5 signal. FIG. 14C. Validation of the expression of the target protein MUC16 by flow cytometry for two additional cell lines, H292-Muc16 and MDA MB 231-Muc16, for use as CAR target cells. FIG. 14D. ELISA kit (R7D, DUPA00) results, detecting soluble urokinase (uPA) secretion in conditioned media for each cell line. FIG. 14E. Summary table of target cells and protease expression.

FIGS. 15A-15F. TPS3 and TPS4 show response to endogenous proteases (−Matriptase) in a Jurkat NFAT reporter assay while TPS6 mainly responds to exogenous matriptase (+Matriptase). A Jurkat-NFAT-luciferase reporter cell line transduced with lentivirus encoding CD45-gate CARs was used to evaluate the activity of CD45-gate CARs in the presence of endogenous matriptase only (−Matriptase) or in the presence of exogenous matriptase (+Matriptase). Exposure to MUC16 positive target cells led to the activation of 53B6 CAR, while no activation was detected with a CD45-gate CAR with a non-cleavable linker. However, the activation can be restored via the cleavable linkers upon exposure to endogenous or exogenous proteases. TPS3 and TPS4 show response to endogenous proteases in a Jurkat NFAT reporter assay while TPS6 mainly responds to exogenous matriptase. All cleavable linkers in FIGS. 15A-15C have a length of 45 amino acids. The following construct names in FIGS. 15D-15F have the amino acid sequence of the following SEQ ID NOs, respectively: 1102 (2×TPS3): SEQ ID NO: 9; 1399 (TPS4): SEQ ID NO:137; 1401 (TPS6): SEQ ID NO:138; 1241 (2×TPS6): SEQ ID NO: 144; 1466 (2×TPS6): SEQ ID NO:153; 1467 (2×TPS6): SEQ ID NO:154; 1468 (2×TPS6): SEQ ID NO:155; 1469 (2×TPS6): SEQ ID NO:156. This shows that the CD45-gate functions with linkers of different lengths.

FIGS. 16A-16F. The short-term cytotoxicity of CD45-gate CAR T cells against target cell lines MDA-MB-231-Muc16, H292-Muc16, and OVCAR3 was evaluated using a nuclear GFP assay. Average fold change in target cell count and standard deviation are shown in each of FIGS. 16A-16F (E:T=3:1 for each cell line). Top panels: CD45-gate CARs with various TPS linkers showed a spectrum of activity in the presence of endogenous proteases, with some of the linkers (e.g., TPS4) exhibiting activity comparable to conventional 53B6 CAR. Bottom panels: all CD45-gate CARs with TPS linkers showed cytotoxic activities in the presence of exogenous matriptase. Incorporation of a non-cleavable GS linker in a CD45-gate CAR effectively inhibited CAR function. All cleavable linkers in FIGS. 16A-16F have a length of 45 amino acids.

FIGS. 17A-17B. CARs with TPS linkers maintained long-term cytotoxicity in the presence of endogenous/exogenous proteases. FIG. 17A. Activity measured in the absence of exogenous matriptase. FIG. 17B. Activity measured in the presence of exogenous matriptase. Activity ranking: TPS12>TPS4, TPS9, TPS11, TPS13>TPS3, TPS8>TPS6, TPS10. All cleavable linkers in FIGS. 17A-17B have a length of 45 amino acids.

FIGS. 18A-18C. Evaluation of CD45-gate CAR T cells (MDA-MB-231-Muc16) in a breast cancer xenograft model in 7-8 weeks old NSG mice. FIG. 18A. Schematic summary of the experimental scheme for in vivo analysis. FIG. 18B. CD45-Gate CARs with cleavable TPS3 or TPS4 linker showed anti-tumor activity while the CD45-Gate CAR with TPS6 linker did not show activity in this assay. CD45-gate CAR with non-cleavable GS linker had no CAR activity. FIG. 18C. Treatment with CD45-gate CAR T cells had no effect on the body weight of the mice and no toxicities were observed with any of the cells used. All cleavable linkers in FIGS. 18B-18C have a length of 45 amino acids.

FIGS. 19A-19C. Evaluation of CD45-gate CAR T cells (NCI-H292-MUC16) in a non-small cell lung cancer xenograft model in NShG mice. FIG. 19A. Schematic summary of the experimental scheme for in vivo analysis. FIG. 19B. CD45-Gate CAR with cleavable TPS4 linker showed anti-tumor activity. CD45-gate CAR with non-cleavable GS linker (αCD45-GS-53B6; this is the same construct as “GS non-cleavable” in FIGS. 12A-15C and 16A-18C; the non-cleavable linker has a length of 30 amino acids) had no CAR activity. FIG. 19C. Treatment with CD45-gate CAR T cells had no effect on the body weight of the mice and no toxicities were observed with any of the cells used. All cleavable linkers in FIGS. 19B-19C have a length of 45 amino acids.

FIGS. 20A-20B. Evaluation of other CD45 ligands in protease-activating CD45-gate CAR. T cells were transduced with a vector that encodes a CAR that is not a protease-activating CD45-Gate CAR (53B6, an anti-Muc16 CAR), or with a vector that encodes the antibody-based CD45-Gate CAR (CD45-GS45-53B6), and other alternative CD45-Gate CARs that comprise a non-cleavable linker (BTN3A1 (IgV)-GS45-53B6, BTN3A1-GS45-53B6, sec49k-GS45-53B6, UL11-GS45-53B6). The linker of the non-cleavable protein comprised a 45-mer G4S linker. Results of cytotoxicity assays used to assess inhibitory activity of CD45 gate. Assays were performed in R10 medium without thrombin or other exogenous protein present. FIG. 20A, OvCAR3 (Muc16^{High}) and FIG. 20B, Cov644 (Muc16^{Low}), 1st round—48H cytotoxicity. The constructs comprised the following domains as indicated by each construct's name: 53B6, which has the amino acid sequence of SEQ ID NO: 11; CD45, which has the amino acid sequence of SEQ ID NO: 3; GS45, which has the amino acid sequence of SEQ ID NO: 182; BTN3A1 (IgV), which has the amino acid sequence of SEQ ID NO: 14; BTN3A1, which has the amino acid sequence of SEQ ID NO: 183; sec49k, which has the amino acid sequence of SEQ ID NO: 12; UL11, which has the amino acid sequence of SEQ ID NO: 13. The constructs have the following amino acid sequences: BTN3A1 (IgV)-GS45-53B6: SEQ ID NO: 166; BTN3A1-GS45-53B6: SEQ ID NO: 168; sec49k-GS45-53B6: SEQ ID NO: 169; UL11-GS45-53B6: SEQ ID NO: 170.

### DETAILED DESCRIPTION

The present disclosure provides a protease-activating CD45-gate CAR e.g. a chimeric antigen receptor modified to comprise a removable CD45-recruiting domain, which specifically binds to CD45. The disclosure further provides related nucleic acids, engineered cells, compositions, and methods. The CD45 binding domain is linked, joined to or tethered to the antigen binding domain of a CAR by a peptide linker that comprises at least one protease cleavage site. The CAR's antigen binding domain can specifically bind to an antigen expressed by a target cell such as a tumor cell. Whilst not wishing to be bound by theory, it is hypothesized that when the protease-activating CD45-gate CAR is functionally expressed in a cell that also functionally expresses CD45, the protease-activating CD45-gate CAR will bind to the CD45 protein. This binding is thought to hold CD45 in close proximity to the protease-activating CD45-gate CAR. This is thought to permit the CD45, a phosphatase, to dephosphorylate the CAR's intracellular signalling domain, thereby at least partially suppressing the CAR's activity. When the CAR binds to its antigen under this condition, it is not activated to the same extent that it would have been in the absence of the CD45 gate. Thus the CAR is in a “suppressed” or “reduced activity” state. The protease-activating CD45-gate CAR's extracellular linker protease cleavage site can be recognized and cleaved by a protease present in the microenvironment of one or more tumor cells that the CAR targets. In this circumstance, the protease disconnects the CAR from the CD45 recruiting domain. This permits the CAR to disassociate from the CD45, thus ending the CD45-mediated suppression of the CAR's activation. Under this condition, when the CAR binds to its antigen, it can become activated up to the same extent that it would have been if it had never comprised a CD45 gate. Thus, the partial suppression of the CAR's activity is at least partially relieved, in other words the protease-activating CD45-gate CAR is at least partially activated, when the protease-activating CD45-gate CAR-expressing cell is in the presence of its target cell. The protease-activating CD45-gate CAR thus is expected to provide more selective CAR activation than a CAR that lacks the gate.

### General Techniques

The practice of the disclosure will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-1998) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987); PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995). Gene editing techniques using TALENs, CRISPR/Cas9, and megaTAL nucleases, for example, are within the skill of the art and explained fully in the literature, such as T. Gaj et al., Genome-Editing Technologies: Principles and Applications, Cold Spring Harb Perspect Biol 2016; 8: a023754 and citations therein, C. Sommer et al., Mol. Ther. 27:1126-38 (2019) (PMID: 31005597), C. Sommer et al., Mol. Ther. 28:2237-51 (PMID: 32592688).

### Definitions

As used herein “autologous” means that cells, a cell line, or population of cells used for treating subjects are originating from said subject.

As used herein “allogeneic” means that cells or population of cells used for treating subjects are not originating from said subject but from a donor.

As used herein, the term “endogenous” refers to any material from or produced inside an organism, cell, tissue or system.

As used herein, the term “exogenous” refers to any material introduced from or produced outside an organism, cell, tissue or system.

As used herein, “immune cell” refers to a cell of hematopoietic origin functionally involved in the initiation and/or execution of innate and/or adaptative immune response. Examples of immune cells include T cells, e.g., alpha/beta T cells and gamma/delta T cells, B cells, natural killer (NK) cells, natural killer T (NKT) cells, mast cells, and myeloic-derived phagocytes.

As used herein, the term “expression” refers to the transcription and/or translation of a particular nucleotide sequence driven by a promoter.

As used herein, “expression vector” refers to a vector comprising a recombinant polynucleotide comprising expression control sequences operatively linked to a nucleotide sequence to be expressed. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

As used herein, “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter).

As used herein, “expression control sequence” means a nucleic acid sequence that directs transcription of a nucleic acid. An expression control sequence can be a promoter, such as a constitutive or an inducible promoter, or an enhancer. The expression control sequence is operably linked to the nucleic acid sequence to be transcribed.

“Promoter” and “promoter sequence” are used interchangeably and refer to a DNA sequence capable of controlling the expression of a coding sequence or functional RNA. In general, a coding sequence is located 3′ to a promoter sequence. It is understood by those skilled in the art that different promoters may direct the expression of a gene in different tissues or cell types, or at different stages of development, or in response to different environmental or physiological conditions.

In any of the vectors of the present disclosure, the vector optionally comprises a promoter disclosed herein.

A “host cell” includes an individual cell or cell culture that can be or has been a recipient for vector(s) for incorporation of polynucleotide inserts. Host cells include progeny of a single host cell, and the progeny may not necessarily be completely identical (in morphology or in genomic DNA complement) to the original parent cell due to natural, accidental, or deliberate mutation. A host cell includes cells transfected in vivo with a polynucleotide(s) of this disclosure.

The term “extracellular ligand-binding domain” as used herein refers to an oligo- or polypeptide that is capable of binding a ligand. Preferably, the domain will be capable of interacting with a cell surface molecule. For example, the extracellular ligand-binding domain may be chosen to recognize a ligand that acts as a cell surface marker on target cells associated with a particular disease state. The term “stalk domain” is used herein to refer to any oligo- or polypeptide that functions to link the transmembrane domain to the extracellular ligand-binding domain. In particular, stalk domains are used to provide more flexibility and accessibility for the extracellular ligand-binding domain.

The term “intracellular signaling domain” refers to the portion of a protein which transduces the effector signal function signal and directs the cell to perform a specialized function.

A “co-stimulatory molecule” as used herein refers to the cognate binding partner on a T cell that specifically binds with a co-stimulatory ligand, thereby mediating a co-stimulatory response by the cell, such as, but not limited to proliferation. Co-stimulatory molecules include, but are not limited to an MHC class I molecule, BTLA and Toll ligand receptor. Examples of costimulatory molecules include CD27, CD28, CD8, 4-1 BB (CD137), OX40, CD30, CD40, PD-1, ICOS, lymphocyte function-associated antigen-1 (LFA-1), CD2, CD7, LIGHT, NKG2C, B7-H3 and a ligand that specifically binds with CD83 and the like.

A “co-stimulatory ligand” refers to a molecule on an antigen presenting cell that specifically binds a cognate co-stimulatory signal molecule on a T cell, thereby providing a signal which, in addition to the primary signal provided by, for instance, binding of a TCR/CD3 complex with an MHC molecule loaded with peptide, mediates a T cell response, including, but not limited to, proliferation activation, differentiation and the like. A co-stimulatory ligand can include but is not limited to CD7, B7-1 (CD80), B7-2 (CD86), PD-L1, PD-L2, 4-1 BBL, OX40L, inducible costimulatory ligand (ICOS-L), intercellular adhesion molecule (ICAM, CD30L, CD40, CD70, CD83, HLA-G, MICA, M1 CB, HVEM, lymphotoxin β receptor, 3/TR6, ILT3, ILT4, an agonist or antibody that binds Toll ligand receptor and a ligand that specifically binds with B7-H3. A co-stimulatory ligand also encompasses, inter alia, an antibody that specifically binds with a co-stimulatory molecule present on a T cell, such as but not limited to, CD27, CD28, 4-1 BB, OX40, CD30, CD40, PD-1, ICOS, lymphocyte function-associated antigen-1 (LFA-1), CD2, CD7, LTGHT, NKG2C, B7-H3, a ligand that specifically binds with CD83.

An “antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term encompasses not only intact polyclonal or monoclonal antibodies, but also antigen-binding fragments thereof (such as Fab, Fab′, F(ab′)2, and Fv), and any other modified configuration of the immunoglobulin molecule that comprises an antigen recognition site including, for example without limitation, single chain (scFv) and domain antibodies (including, for example, shark and camelid antibodies), and fusion proteins comprising an antibody. An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

The term “antigen-binding fragment” or “antigen binding portion” of an antibody, as used herein, refers to one or more fragments of an intact antibody that retain the ability to specifically bind to a given antigen. Antigen binding functions of an antibody can be performed by fragments of an intact antibody. Examples of binding fragments encompassed within the term “antigen binding fragment” of an antibody include Fab; Fab′; F(ab′)2; an Fd fragment consisting of the VH and CH1 domains; an Fv fragment consisting of the VL and VH domains of a single arm of an antibody; a single domain antibody (dAb) fragment (Ward et al., Nature 341:544-546, 1989), and an isolated complementarity determining region (CDR).

An antibody, an antibody conjugate, or a polypeptide that “specifically binds” to a target is a term well understood in the art, and methods to determine such specific binding are also well known in the art. A molecule is said to exhibit “specific binding” if it reacts or associates more frequently, more rapidly, with greater duration and/or with greater affinity with a particular cell or substance than it does with alternative cells or substances. An antibody “specifically binds” to a target if it binds with greater affinity, avidity, more readily, and/or with greater duration than it binds to other substances. It is also understood that by reading this definition, for example, an antibody (or moiety or epitope or mimotope) that specifically binds to a first target may or may not specifically bind to a second target. As such, “specific binding” does not necessarily require (although it can include) exclusive binding.

A “variable region” of an antibody refers to the variable region of the antibody light chain or the variable region of the antibody heavy chain, either alone or in combination. As known in the art, the variable regions of the heavy and light chain each consist of four framework regions (FR) connected by three complementarity determining regions (CDRs) also known as hypervariable regions. The CDRs in each chain are held together in close proximity by the FRs and, with the CDRs from the other chain, contribute to the formation of the antigen binding site of antibodies. There are at least two techniques for determining CDRs: (1) an approach based on cross-species sequence variability (i.e., Kabat et al. Sequences of Proteins of Immunological Interest, (5th ed., 1991, National Institutes of Health, Bethesda MD)); and (2) an approach based on crystallographic studies of antigen-antibody complexes (Al-lazikani et al., 1997, J. Molec. Biol. 273:927-948). As used herein, a CDR may refer to CDRs defined by either approach or by a combination of both approaches.

A “CDR” of a variable domain are amino acid residues within the variable region that are identified in accordance with the definitions of the Kabat, Chothia, the accumulation of both Kabat and Chothia, AbM, contact, and/or conformational definitions or any method of CDR determination well known in the art. Antibody CDRs may be identified as the hypervariable regions originally defined by Kabat et al. See, e.g., Kabat et al., 1992, Sequences of Proteins of Immunological Interest, 5th ed., Public Health Service, NIH, Washington D.C. The positions of the CDRs may also be identified as the structural loop structures originally described by Chothia and others. See, e.g., Chothia et al., Nature 342:877-883, 1989. Other approaches to CDR identification include the “AbM definition,” which is a compromise between Kabat and Chothia and is derived using Oxford Molecular's AbM antibody modeling software (now Accelrys®), or the “contact definition” of CDRs based on observed antigen contacts, set forth in MacCallum et al., J. Mol. Biol., 262:732-745, 1996. In another approach, referred to herein as the “conformational definition” of CDRs, the positions of the CDRs may be identified as the residues that make enthalpic contributions to antigen binding. See, e.g., Makabe et al., Journal of Biological Chemistry, 283:1 156-1 166, 2008. Still other CDR boundary definitions may not strictly follow one of the above approaches, but will nonetheless overlap with at least a portion of the Kabat CDRs, although they may be shortened or lengthened in light of prediction or experimental findings that particular residues or groups of residues or even entire CDRs do not significantly impact antigen binding. As used herein, a CDR may refer to CDRs defined by any approach known in the art, including combinations of approaches. The methods used herein may utilize CDRs defined according to any of these approaches. For any given embodiment containing more than one CDR, the CDRs may be defined in accordance with any of Kabat, Chothia, extended, AbM, contact, and/or conformational definitions.

Antibodies of the disclosure can be produced using techniques well known in the art, e.g., recombinant technologies, phage display technologies, synthetic technologies or combinations of such technologies or other technologies readily known in the art (see, for example, Jayasena, S. D., Clin. Chem., 45:1628-50, 1999 and Fellouse, F. A., et al, J. Mol. Biol., 373 (4): 924-40, 2007).

As known in the art, “polynucleotide,” or “nucleic acid,” as used interchangeably herein, refer to chains of nucleotides of any length, and include DNA and RNA. The nucleotides can be deoxyribonucleotides, ribonucleotides, modified nucleotides or bases, and/or their analogs, or any substrate that can be incorporated into a chain by DNA or RNA polymerase. A polynucleotide may comprise modified nucleotides, such as methylated nucleotides and their analogs. If present, modification to the nucleotide structure may be imparted before or after assembly of the chain. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl phosphonates, phosphotriesters, phosphoamidates, carbamates, etc.) and with charged linkages (e.g., phosphorothioates, phosphorodithioates, etc.), those containing pendant moieties, such as, for example, proteins (e.g., nucleases, toxins, antibodies, signal peptides, poly-L-lysine, etc.), those with intercalators (e.g., acridine, psoralen, etc.), those containing chelators (e.g., metals, radioactive metals, boron, oxidative metals, etc.), those containing alkylators, those with modified linkages (e.g., alpha anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha- or beta-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR2 (“amidate”), P(O)R, P(O)OR′, CO or CH2 (“formacetal”), in which each R or R′ is independently H or substituted or unsubstituted alkyl (1-20 C) optionally containing an ether (—O—) linkage, aryl, alkenyl, cycloalkyl, cycloalkenyl or araldyl. Not all linkages in a polynucleotide need be identical. The preceding description applies to all polynucleotides referred to herein, including RNA and DNA.

As used herein, “transfection” refers to the uptake of exogenous or heterologous RNA or DNA by a cell. A cell has been “transfected” by exogenous or heterologous RNA or DNA when such RNA or DNA has been introduced inside the cell. A cell has been “transformed” by exogenous or heterologous RNA or DNA when the transfected RNA or DNA effects a phenotypic change. The transforming RNA or DNA can be integrated (covalently linked) into chromosomal DNA making up the genome of the cell.

As used herein, “transformation” refers to the transfer of a nucleic acid fragment into the genome of a host organism, resulting in genetically stable inheritance. Host organisms containing the transformed nucleic acid fragments are referred to as “transgenic” or “recombinant” or “transformed” organisms.

As used herein, “substantially pure” refers to material which is at least 50% pure (i.e., free from contaminants), more preferably, at least 90% pure, more preferably, at least 95% pure, yet more preferably, at least 98% pure, and most preferably, at least 99% pure. The term “compete”, as used herein with regard to an antibody, means that a first antibody, or an antigen binding fragment (or portion) thereof, binds to an epitope or mimotope in a manner sufficiently similar to the binding of a second antibody, or an antigen binding portion thereof, such that the result of binding of the first antibody with its cognate epitope or mimotope is detectably decreased in the presence of the second antibody compared to the binding of the first antibody in the absence of the second antibody. The alternative, where the binding of the second antibody to its epitope or mimotope is also detectably decreased in the presence of the first antibody, can, but need not be the case. That is, a first antibody can inhibit the binding of a second antibody to its epitope or mimotope without that second antibody inhibiting the binding of the first antibody to its respective epitope or mimotope. However, where each antibody detectably inhibits the binding of the other antibody with its cognate epitope or mimotope or ligand, whether to the same, greater, or lesser extent, the antibodies are said to “cross-compete” with each other for binding of their respective epitope(s) or mimotope(s). Both competing and cross-competing antibodies are encompassed by the disclosure. Regardless of the mechanism by which such competition or cross-competition occurs (e.g., steric hindrance, conformational change, or binding to a common epitope or mimotope, or portion thereof), the skilled artisan would appreciate, based upon the teachings provided herein, that such competing and/or cross-competing antibodies are encompassed and can be useful for the methods disclosed herein.

As used herein, “treatment” is an approach for obtaining beneficial or desired clinical results. For purposes of this disclosure, beneficial or desired clinical results include, but are not limited to, one or more of the following: reducing the proliferation of (or destroying) neoplastic or cancerous cells, inhibiting metastasis of neoplastic cells, shrinking or decreasing the size of tumor, remission of a disease (e.g., cancer), decreasing symptoms resulting from a disease (e.g., cancer), increasing the quality of life of those suffering from a disease (e.g., cancer), decreasing the dose of other medications required to treat a disease (e.g., cancer), delaying the progression of a disease (e.g., cancer), curing a disease (e.g., cancer), and/or prolong survival of subjects having a disease (e.g., cancer).

“Ameliorating” means a lessening or improvement of one or more symptoms as compared to not administering a treatment. “Ameliorating” also includes shortening or reduction in duration of a symptom. As used herein, an “effective dosage” or “effective amount” of drug, compound, or pharmaceutical composition is an amount sufficient to effect any one or more beneficial or desired results. For prophylactic use, beneficial or desired results include eliminating or reducing the risk, lessening the severity, or delaying the outset of the disease, including biochemical, histological and/or behavioral symptoms of the disease, its complications and intermediate pathological phenotypes presenting during development of the disease. For therapeutic use, beneficial or desired results include clinical results such as reducing incidence or amelioration of one or more symptoms of various diseases or conditions (such as for example cancer), decreasing the dose of other medications required to treat the disease, enhancing the effect of another medication, and/or delaying the progression of the disease. An effective dosage can be administered in one or more administrations. For purposes of this disclosure, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

As used herein, a “subject” is any mammal, e.g a human, or a monkey. Mammals include, but are not limited to, farm animals, sport animals, pets, primates, horses, dogs, cats, mice and rats. In an exemplary embodiment, the subject is a human. In an exemplary embodiment, the subject is a monkey, e.g. a cynomolgus monkey.

As used herein, “vector” means a construct, which is capable of delivering, and, preferably, expressing, one or more gene(s) or sequence(s) of interest in a host cell. Examples of vectors include, but are not limited to, viral vectors, naked DNA or RNA expression vectors, plasmid, cosmid or phage vectors, DNA or RNA expression vectors associated with cationic condensing agents, DNA or RNA expression vectors encapsulated in liposomes, and certain eukaryotic cells, such as producer cells.

As used herein, “pharmaceutically acceptable carrier” or “pharmaceutically acceptable excipient” includes any material which, when combined with an active ingredient, allows the ingredient to retain biological activity and is non-reactive with the subject's immune system. Examples include, but are not limited to, any of the standard pharmaceutical carriers such as a phosphate buffered saline solution, water, emulsions such as oil/water emulsion, and various types of wetting agents. Preferred diluents for aerosol or parenteral administration are phosphate buffered saline (PBS) or normal (0.9%) saline. Compositions of the disclosure comprising such carriers are formulated by well-known conventional methods (see, for example, Remington's Pharmaceutical Sciences, 18th edition, A. Gennaro, ed., Mack Publishing Co., Easton, PA, 1990; and Remington, The Science and Practice of Pharmacy 21 st Ed. Mack Publishing, 2005).

As used herein, “alloreactivity” refers to the ability of T cells to recognize MHC complexes that were not encountered during thymic development. Alloreactivity manifests itself clinically as host versus graft rejection and graft versus host disease.

Reference to “about” a value or parameter herein includes (and describes) embodiments that are directed to that value or parameter per se. For example, description referring to “about X” includes description of “X.” Numeric ranges are inclusive of the numbers defining the range.

It is understood that wherever embodiments are described herein with the language “comprising,” otherwise analogous embodiments described in terms of “consisting of” and/or “consisting essentially of” are also provided.

Where aspects or embodiments of the disclosure are described in terms of a Markush group or other grouping of alternatives, the disclosure encompasses not only the entire group listed as a whole, but each member of the group individually and all possible subgroups of the main group, but also the main group absent one or more of the group members. The disclosure also envisages the explicit exclusion of one or more of any of the group members in the claimed disclosure.

Unless otherwise defined, all technical and scientific terms used herein have the same meaning as commonly understood by one of ordinary skill in the art to which this disclosure belongs. In case of conflict, the present specification, including definitions, will control. Throughout this specification and claims, the word “comprise,” or variations such as “comprises” or “comprising” will be understood to imply the inclusion of a stated integer or group of integers but not the exclusion of any other integer or group of integers. Unless otherwise required by context, singular terms shall include pluralities and plural terms shall include the singular.

Exemplary methods and materials are described herein, although methods and materials similar or equivalent to those described herein can also be used in the practice or testing of the disclosure. The materials, methods, and examples are illustrative only and not intended to be limiting.

An “antigen binding protein” comprises one or more antigen binding domains. An “antigen binding domain” as used herein means any polypeptide that binds a specified target antigen. In some embodiments, the antigen binding domain binds to an antigen on a tumor cell. In some embodiments, the antigen binding domain binds to an antigen on a cell involved in a hyperproliferative disease or to a viral or bacterial antigen.

Antigen binding domains include, but are not limited to, antibody binding regions that are immunologically functional fragments. The term “immunologically functional fragment” (or “fragment”) of an antigen binding domain is a species of antigen binding domain comprising a portion (regardless of how that portion is obtained or synthesized) of an antibody that lacks at least some of the amino acids present in a full-length chain, but which is still capable of specifically binding to a target antigen. Such fragments are biologically active in that they bind to the target antigen and can compete with other antigen binding domains, including intact antibodies, for binding to a given epitope or mimotope.

Immunologically functional immunoglobulin fragments include, but are not limited to, scFv fragments, Fab fragments (Fab′, F(ab′)2, and the like), one or more complementarity determining regions (“CDRs”), a diabody (heavy chain variable domain on the same polypeptide as a light chain variable domain, connected via a short peptide linker that is too short to permit pairing between the two domains on the same chain), domain antibodies, bivalent antigen binding domains (comprises two antigen binding sites), multispecific antigen binding domains, and single-chain antibodies. These fragments can be derived from any mammalian source, including but not limited to human, mouse, rat, camelid or rabbit. As will be appreciated by one of skill in the art, an antigen binding domain can include non-protein components.

The variable regions typically exhibit the same general structure of relatively conserved framework regions (FR) joined by the 3 hypervariable regions (CDRs). The CDRs from the two chains of each pair typically are aligned by the framework regions, which can enable binding to a specific epitope or mimotope. From N-terminal to C-terminal, both light and heavy chain variable regions typically comprise the domains FR1, CDR1, FR2, CDR2, FR3, CDR3 and FR4. By convention, CDR regions in the heavy chain are typically referred to as HC CDR1, CDR2, and CDR3. The CDR regions in the light chain are typically referred to as LC CDR1, CDR2, and CDR3.

In some embodiments, antigen binding domains comprise one or more complementarity binding regions (CDRs) present in the full-length light or heavy chain of an antibody, and in some embodiments comprise a single heavy chain and/or light chain or portion thereof. These fragments can be produced by recombinant DNA techniques or can be produced by enzymatic or chemical cleavage of antigen binding domains, including intact antibodies.

In some embodiments, the antigen binding domain is an antibody or fragment thereof, including one or more of the complementarity determining regions (CDRs) thereof. In some embodiments, the antigen binding domain is a single chain variable fragment (scFv), comprising light chain CDRs CDR1, CDR2 and CDR3, and heavy chain CDRs CDR1, CDR2 and CDR3.

The assignment of amino acids to each of the framework, CDR, and variable domains is typically in accordance with numbering schemes of Kabat numbering (see, e.g., Kabat et al. in Sequences of Proteins of Immunological Interest, 5th Ed., NIH Publication 91-3242, Bethesda Md. 1991), Chothia numbering (see, e.g., Chothia & Lesk, (1987), J Mol Biol 196:901-917; Al-Lazikani et al., (1997) J Mol Biol 273:927-948; Chothia et al., (1992) J Mol Biol 227:799-817; Tramontano et al., (1990) J Mol Biol 215 (1): 175-82; and U.S. Pat. No. 7,709,226), contact numbering, or the AbM scheme (Antibody Modeling program, Oxford Molecular).

In some embodiments, the antigen binding domain is a recombinant antigen receptor. The term “recombinant antigen receptor” as used herein refers broadly to a non-naturally occurring surface receptor that comprises an extracellular antigen-binding domain or an extracellular ligand-binding domain, a transmembrane domain and an intracellular domain. In some embodiments, the recombinant antigen receptor is a chimeric antigen receptor (CAR). Chimeric antigen receptors (CARs) are well-known in the art. A CAR is a fusion protein that comprises an extracellular domain comprising an antigen recognition moiety (also referred to herein as an antigen binding domain), a transmembrane domain and an intracellular domain comprising one or more T cell activation domains (see, e.g., Eshhar et al., Proc. Natl. Acad. Sci. USA, 90 (2): 720-724 (1993), and Sadelain et al., Curr. Opin. Immunol, 21 (2): 215-223 (2009)). A hinge region or domain typically is situated between the CAR's antigen recognition domain and the transmembrane domain.

In some embodiments, the intracellular domain of a recombinant antigen receptor comprises a co-stimulatory domain and/or an ITAM-containing domain. In some embodiments, the intracellular domain of a recombinant antigen receptor comprises an intracellular protein or a functional variant thereof (e.g., truncation(s), insertion(s), deletion(s) or substitution(s)).

The term “extracellular ligand-binding domain” or “extracellular antigen-binding domain” as used herein refers to a polypeptide that is capable of binding a ligand or an antigen or capable of interacting with a cell surface molecule, such as a ligand or a surface antigen. For example, the extracellular ligand-binding or antigen-binding domain may be chosen to recognize a ligand that acts as a cell surface marker on target cells associated with a particular disease state, e.g., a tumor-specific antigen. In some embodiments, the antigen-binding domain comprises an antibody, or an antigen binding fragment or an antigen binding portion of an antibody. In some embodiments, the antigen binding domain comprises an Fv or scFv, an Fab or scFab, an F(ab′)2 or a scF (ab′)2, an Fd, a monobody, an affibody, a camelid antibody, a VHH antibody, a single domain antibody, or a darpin. In some embodiments, the ligand-binding domain comprises a partner of a binding pair, such as a ligand that binds to a surface receptor, or an ectodomain of a surface receptor that binds to a ligand.

The terms “stalk domain” and “hinge domain” are used interchangeably herein to refer to any polypeptide that functions to link the transmembrane domain to the extracellular ligand-binding domain. In particular, stalk domains are used to provide more flexibility and accessibility for the extracellular ligand-binding domain.

The term “intracellular signaling domain” refers to the portion of a protein which transduces the effector signal function signal and directs the cell to perform a specialized function.

### Vectors

Expression vectors and administration of polynucleotide compositions are further described herein.

In another aspect, the disclosure provides a method of making any of the polynucleotides described herein.

Polynucleotides complementary to any such sequences are also encompassed by the disclosure. Polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the disclosure, and a polynucleotide may, but need not, be linked to other molecules and/or support materials.

Polynucleotides may comprise a native sequence (i.e., an endogenous sequence that encodes an antibody or a portion thereof) or may comprise a variant of such a sequence. Polynucleotide variants contain one or more substitutions, additions, deletions and/or insertions such that the immunoreactivity of the encoded polypeptide is not diminished, relative to a native immunoreactive molecule. The effect on the immunoreactivity of the encoded polypeptide may generally be assessed as described herein. Variants preferably exhibit at least about 70% identity, more preferably, at least about 80% identity, yet more preferably, at least about 90% identity, and most preferably, at least about 95% identity to a polynucleotide sequence that encodes a native antibody or a portion thereof. Two polynucleotide or polypeptide sequences are said to be “identical” if the sequence of nucleotides or amino acids in the two sequences is the same when aligned for maximum correspondence as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, or 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, WI), using default parameters. This program embodies several alignment schemes described in the following references: Dayhoff, M. O., 1978, A model of evolutionary change in proteins—Matrices for detecting distant relationships. In Dayhoff, M. O. (ed.) Atlas of Protein Sequence and Structure, National Biomedical Research Foundation, Washington DC Vol. 5, Suppl. 3, pp. 345-358; Hein J., 1990, Unified Approach to Alignment and Phylogenes pp. 626-645 Methods in Enzymology vol. 183, Academic Press, Inc., San Diego, CA; Higgins, D. G. and Sharp, P. M., 1989, CABIOS 5:151-153; Myers, E. W. and Muller W., 1988, CABIOS 4:1 1-17; Robinson, E. D., 1971, Comb. Theor. 1 1:105; Santou, N., Nes, M., 1987, Mol. Biol. Evol. 4:406-425; Sneath, P. H. A. and Sokal, R. R., 1973, Numerical Taxonomy the Principles and Practice of Numerical Taxonomy, Freeman Press, San Francisco, CA; Wilbur, W. J. and Lipman, D. J., 1983, Proc. Natl. Acad. Sci. USA 80:726-730.

Preferably, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e. the window size) and multiplying the results by 100 to yield the percentage of sequence identity.

Variants may also, or alternatively, be substantially homologous to a native gene, or a portion or complement thereof. Such polynucleotide variants are capable of hybridizing under moderately stringent conditions to a naturally occurring DNA sequence encoding a native antibody (or a complementary sequence).

Suitable “moderately stringent conditions” include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS.

As used herein, “highly stringent conditions” or “high stringency conditions” are those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/m\), 0.1% SDS, and 10% dextran sulfate at 42° C., with washes at 42° C. in 0.2×SSC (sodium chloride/sodium citrate) and 50% formamide at 55° C., followed by a high-stringency wash consisting of 0.1×SSC containing EDTA at 55° C. The skilled artisan will recognize how to adjust the temperature, ionic strength, etc. as necessary to accommodate factors such as probe length and the like.

It will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the disclosure. Further, alleles of the genes comprising the polynucleotide sequences provided herein are within the scope of the disclosure. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

The polynucleotides of this disclosure can be obtained using chemical synthesis, recombinant methods, or PCR. Methods of chemical polynucleotide synthesis are well known in the art and need not be described in detail herein. One of skill in the art can use the sequences provided herein and a commercial DNA synthesizer to produce a desired DNA sequence.

For preparing polynucleotides using recombinant methods, a polynucleotide comprising a desired sequence can be inserted into a suitable vector, and the vector in turn can be introduced into a suitable host cell for replication and amplification, as further discussed herein. Polynucleotides may be inserted into host cells by any means known in the art. Cells are transformed by introducing an exogenous polynucleotide by direct uptake, endocytosis, transfection, F-mating or electroporation. Once introduced, the exogenous polynucleotide can be maintained within the cell as a non-integrated vector (such as a plasmid) or integrated into the host cell genome. The polynucleotide so amplified can be isolated from the host cell by methods well known within the art. See, e.g., Sambrook et al., 1989.

Alternatively, PCR allows reproduction of DNA sequences. PCR technology is well known in the art and is described in U.S. Pat. Nos. 4,683,195, 4,800,159, 4,754,065 and 4,683,202, as well as PCR: The Polymerase Chain Reaction, Mullis et al. eds., Birkauswer Press, Boston, 1994.

RNA can be obtained by using the isolated DNA in an appropriate vector and inserting it into a suitable host cell. When the cell replicates and the DNA is transcribed into RNA, the RNA can then be isolated using methods well known to those of skill in the art, as set forth in Sambrook et al., 1989, supra, for example.

Suitable cloning vectors may be constructed according to standard techniques, or may be selected from a large number of cloning vectors available in the art. While the cloning vector selected may vary according to the host cell intended to be used, useful cloning vectors will generally have the ability to self-replicate, may possess a single target for a particular restriction endonuclease, and/or may carry genes for a marker that can be used in selecting clones containing the vector. Suitable examples include plasmids and bacterial viruses, e.g., pUC18, pUC19, Bluescript (e.g., pBS SK+) and its derivatives, mp18, mp19, pBR322, pMB9, ColE1, pCR1, RP4, phage DNAs, and shuttle vectors such as pSA3 and pAT28. These and many other cloning vectors are available from commercial vendors such as BioRad, Strategene, and Invitrogen.

Expression vectors generally are replicable polynucleotide constructs that contain a polynucleotide according to the disclosure. It is implied that an expression vector must be replicable in the host cells either as episomes or as an integral part of the chromosomal DNA. Suitable expression vectors include but are not limited to plasmids, viral vectors, including adenoviruses, adeno-associated viruses, retroviruses, cosmids, and expression vector(s) disclosed in PCT Publication No. WO 87/04462. Vector components may generally include, but are not limited to, one or more of the following: a signal sequence; an origin of replication; one or more marker genes; suitable transcriptional controlling elements (such as promoters, enhancers and terminator). For expression (i.e., translation), one or more translational controlling elements are also usually required, such as ribosome binding sites, translation initiation sites, and stop codons.

The vectors containing the polynucleotides of interest can be introduced into the host cell by any of a number of appropriate means, including electroporation, transfection employing calcium chloride, rubidium chloride, calcium phosphate, DEAE-dextran, or other substances; microprojectile bombardment; lipofection; and infection (e.g., where the vector is an infectious agent such as vaccinia virus). The choice of introducing vectors or polynucleotides will often depend on features of the host cell.

A polynucleotide encoding a protease-activating CD45-gate CAR disclosed herein, its extracellular domain, or another fragment of the protease-activating CD45-gate CAR may exist in an expression cassette or expression vector (e.g., a plasmid for introduction into a bacterial host cell, or a viral vector such as a baculovirus vector for transfection of an insect host cell, or a plasmid or viral vector such as a lentivirus for transfection of a mammalian host cell). In some embodiments, a polynucleotide or vector can include a nucleic acid sequence encoding ribosomal skip sequences such as, for example without limitation, a sequence encoding a 2A peptide. 2A peptides, which were identified in the Aphthovirus subgroup of picornaviruses, cause a ribosomal “skip” from one codon to the next without the formation of a peptide bond between the two amino acids encoded by the codons (see (Donnelly and Elliott 2001; Atkins, Wills et al. 2007; Doronina, Wu et al. 2008)). By “codon” is meant three nucleotides on an mRNA (or on the sense strand of a DNA molecule) that are translated by a ribosome into one amino acid residue. Thus, two polypeptides can be synthesized from a single, contiguous open reading frame within an mRNA when the polypeptides are separated by a 2A oligopeptide sequence that is in frame. Such ribosomal skip mechanisms are well known in the art and are known to be used by several vectors for the expression of several proteins encoded by a single messenger RNA.

To direct transmembrane polypeptides into the secretory pathway of a host cell, in some embodiments, a secretory signal sequence (also known as a leader sequence, prepro sequence or pre sequence) is provided in a polynucleotide sequence or vector sequence. The secretory signal sequence is operably linked to the transmembrane nucleic acid sequence, i.e., the two sequences are joined in the correct reading frame and positioned to direct the newly synthesized polypeptide into the secretory pathway of the host cell. Secretory signal sequences are commonly positioned 5′ to the nucleic acid sequence encoding the polypeptide of interest, although certain secretory signal sequences may be positioned elsewhere in the nucleic acid sequence of interest (see, e.g., Welch et al., U.S. Pat. No. 5,037,743; Holland et al., U.S. Pat. No. 5,143,830). Those skilled in the art will recognize that, in view of the degeneracy of the genetic code, considerable sequence variation is possible among these polynucleotide molecules. In some embodiments, nucleic acid sequences of the disclosure are codon-optimized for expression in mammalian cells, preferably for expression in human cells. Codon-optimization refers to the exchange in a sequence of interest of codons that are generally rare in highly expressed genes of a given species for codons that are generally frequent in highly expressed genes of such species, such codons encoding the same amino acids as the codons that are being exchanged.

Methods of preparing immune cells for use in immunotherapy are provided herein. In some embodiments, the methods comprise introducing a protease-activating CD45-gate CAR into immune cells, and expanding the cells. In some embodiments, the disclosure relates to a method of engineering an immune cell comprising: providing an immune cell and expressing at the surface of the cell at least one protease-activating CD45-gate CAR. In some embodiments, the method comprises: transfecting the cell with at least one polynucleotide encoding a protease-activating CD45-gate CAR, and expressing the at least one polynucleotide in the cell.

In some embodiments, the polynucleotide encoding the protease-activating CD45-gate CAR is present in an expression vector for stable expression in the cells. In some embodiments, the polynucleotide is present in a viral vector for stable expression in the cells. In some embodiments, the viral vector may be, for example, a lentiviral vector or adenoviral vector.

In some embodiments, polynucleotides encoding polypeptides according to the present disclosure can be mRNA which is introduced directly into the cells, for example by electroporation. In some embodiments, cytoPulse technology can be used to transiently permeabilize living cells for delivery of material into the cells. Parameters can be modified in order to determine conditions for high transfection efficiency with minimal mortality.

Also provided herein are methods of transfecting an immune cell e.g. a T cell. In some embodiments, the method comprises: contacting a T cell with RNA and applying to the T cell an agile pulse sequence consisting of: (a) an electrical pulse with a voltage range from about 2250 to 3000 V per centimeter; (b) a pulse width of 0.1 ms; (c) a pulse interval of about 0.2 to 10 ms between the electrical pulses of step (a) and (b); (d) an electrical pulse with a voltage range from about 2250 to 3000 V per centimeter with a pulse width of about 100 ms and a pulse interval of about 100 ms between the electrical pulse of step (b) and the first electrical pulse of step (c); and (e) four electrical pulses with a voltage of about 325 V with a pulse width of about 0.2 ms and a pulse interval of 2 ms between each of 4 electrical pulses. In some embodiments, a method of transfecting a T cell comprises contacting said T cell with RNA and applying to the T cell an agile pulse sequence comprising: (a) an electrical pulse with a voltage of about 2250, 2300, 2350, 2400, 2450, 2500, 2550, 2400, 2450, 2500, 2600, 2700, 2800, 2900 or 3000V per centimeter; (b) a pulse width of 0.1 ms; (c) and a pulse interval of about 0.2, 0.5, 1, 2, 3, 4, 5, 6, 7, 8, 9 or 10 ms between the electrical pulses of step (a) and (b); (d) one electrical pulse with a voltage range from about 2250 to 3000 V per centimeter, e.g. of 2250, 2300, 2350, 2400, 2450, 2500, 2550, 2400, 2450, 2500, 2600, 2700, 2800, 2900 or 3000V per centimeter with a pulse width of 100 ms and a pulse interval of 100 ms between the electrical pulse of step (b) and the first electrical pulse of step (c); and (e) 4 electrical pulses with a voltage of about 325 V with a pulse width of about 0.2 ms and a pulse interval of about 2 ms between each of 4 electrical pulses. Any values included in the value range described above are disclosed in the present application. Electroporation medium can be any suitable medium known in the art. In some embodiments, the electroporation medium has conductivity in a range spanning about 0.01 to about 1.0 milliSiemens.

In some embodiments, the method can further comprise a step of genetically modifying a cell by inactivating or reducing the expression level of at least one gene expressing, for example without limitation, a component of the TCR, a target for an immunosuppressive agent, an HLA gene, and/or an immune checkpoint protein such as, for example, PDCD1 or CTLA-4. By inactivating a gene it is intended that the gene of interest is not expressed in a functional protein form. In some embodiments, the gene to be inactivated is selected from the group consisting of, for example without limitation, TCRα, TCRβ, β2-microglobulin (“β2m”), CD52, GR, deoxycytidine kinase (DCK), PD-1, and CTLA-4. In some embodiments the method comprises inactivating or reducing the expression level of one or more genes by introducing into the cells a rare-cutting endonuclease able to selectively inactivate a gene by selective DNA cleavage. In some embodiments the rare-cutting endonuclease can be, for example, a transcription activator-like effector nuclease (TALE-nuclease or TALEN), a megaTAL nuclease or a Cas9 endonuclease.

In another aspect, a step of genetically modifying immune cells e.g. T cells can comprise: modifying immune cells e.g. T cells by inactivating at least one gene expressing a target for an immunosuppressive agent, and; expanding the cells, optionally in the presence of the immunosuppressive agent. An immunosuppressive agent is an agent that suppresses immune function by one of several mechanisms of action. An immunosuppressive agent can diminish the extent and/or voracity of an immune response. Non-limiting examples of immunosuppressive agents include calcineurin inhibitors, targets of rapamycin, interleukin-2 α-chain blockers, inhibitors of inosine monophosphate dehydrogenase, inhibitors of dihydrofolic acid reductase, corticosteroids, and immunosuppressive antimetabolites. Some cytotoxic immunosuppressants act by inhibiting DNA synthesis. Others may act through activation of T cells or by inhibiting the activation of helper cells. The methods according to the disclosure allow conferring immunosuppressive resistance to e.g. T cells for immunotherapy by inactivating the target of the immunosuppressive agent in the T cells. As non-limiting examples, targets for an immunosuppressive agent can be a receptor for an immunosuppressive agent such as for example without limitation CD52, glucocorticoid receptor (GR), FKBP family gene members, and cyclophilin family gene members.

Compositions and methods for expressing a protease-activating CD45-gate CAR are provide herein. Also provided are uses of such compositions and methods for improving the functional activities of immune cells e.g. T cells, such as CAR-T cells. The methods and compositions provided herein are useful for improving activation specificity and therapeutic efficacy of immune cells e.g. T cells such as CAR-T cells.

Immune cells e.g. T cells provided herein express a protease-activating CD45-gate CAR as disclosed herein. Advantageously, the immune cells provided herein exhibit improved in vivo activation specificity relative to cells that express a non-gated CAR e.g. that express the same or comparable CAR except it lacks a functional protease cleavage site.

### Protease-Activating CD45-Gate CAR

In one aspect, provided herein is a protease-activating CD45-gate chimeric antigen receptor (CD45-gate CAR) comprising an extracellular domain, a transmembrane domain, and an intracellular domain, wherein the extracellular domain comprises:
a CD45 recruiting domain,
an antigen binding domain, and
a linker connecting the carboxy terminus of the CD45 recruiting domain to the amino terminus of the antigen binding domain, wherein the linker comprises at least one protease cleavage site that is recognized by a protease,
and further wherein the intracellular domain comprises at least one signaling domain that can be reversibly inactivated by CD45.

In some embodiments, the extracellular domain comprises a stalk domain that joins the extracellular domain to the transmembrane domain.

In some embodiments, the intracellular signaling domain comprises an activating domain. In some embodiments, the intracellular signaling domain comprises a costimulatory domain. In some embodiments, the intracellular signaling domain comprises an activating domain and a costimulatory domain. In some embodiments, the intracellular signaling domain comprises an activating domain such as an ITAM-containing domain. In some embodiments, the intracellular signaling domain comprises the CD3 zeta intracellular domain. In some embodiments, the intracellular signaling domain comprises a CD3 zeta domain that comprises the amino acid sequence of SEQ ID NO: 34 or a fragment thereof.

In some embodiments the intracellular signaling domain comprises one, two or three ITAM domains selected from the group consisting of wildtype or variants of a CD3γ ITAM, a CD3δ ITAM, a CD3ε ITAM, a CD3ξ1 ITAM, a CD3ζ2 ITAM, and a CD3ζ3 ITAM, provided that, if three ITAM domains are selected that are wildtype ITAM domains, the three ITAM domains are not CD351, CD322 and CD33; see U.S. Provisional Patent Appl. No. 63/054,701, incorporated herein by reference in its entirety.

In some embodiments, the intracellular domain comprises or further comprises at least one costimulatory domain. In some embodiments, the at least one costimulatory domain is a signaling region of CD28, OX-40, 4-1BB/CD137, CD2, CD7, CD27, CD30, CD40, programmed death-1 (PD-1), inducible T cell costimulator (ICOS), lymphocyte function-associated antigen-1 (LFA-1 (CD1 la/CD 18), CD3 gamma, CD3 delta, CD3 epsilon, CD247, CD276 (B7-H3), LIGHT, (TNFSF14), NKG2C, Ig alpha (CD79a), DAP-10, Fc gamma receptor, MHC class I molecule, TNF receptor proteins, an Immunoglobulin protein, cytokine receptor, integrins, Signaling Lymphocytic Activation Molecules (SLAM proteins), activating NK cell receptors, BTLA, a Toll ligand receptor, ICAM-1, B7-H3, CDS, ICAM-1, GITR, BAFFR, LIGHT, HVEM (LIGHTR), KIRDS2, SLAMF7, NKp80 (KLRFI), NKp44, NKp30, NKp46, CD19, CD4, CD8alpha, CD8beta, IL-2R beta, IL-2R gamma, IL-7R alpha, ITGA4, VLA1, CD49a, ITGA4, CD49D, ITGA6, VLA-6, CD49f, ITGAD, CD1 Id, ITGAE, CD103, ITGAL, CD1 la, LFA-1, IT GAM, CD1 lb, ITGAX, CD1 lc, ITGB1, CD29, ITGB2, CD 18, LFA-1, ITGB7, NKG2D, TNFR2, TRANCE/RANKL, DNAMI (CD226), SLAMF4 (CD244, 2B4), CD84, CD96 (Tactile), CEACAM1, CRT AM, Ly9 (CD229), CD 160 (BY55), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, CD 19a, a ligand that specifically binds with CD83, or any combination thereof.

In some embodiments, the CD45 recruiting domain comprises one or more of an anti-CD45 antibody antigen binding domain, an anti-CD45 NANOBODY®, an anti-CD45 scFv, a truncated viral protein binder of CD45, an anti-CD45 Fab, an anti-CD45 camelid VHH, a CD45-binding protein, or a truncated endogenous CD45 binder. In some embodiments, the CD45 recruiting domain comprises one or more of truncated UL11, truncated sec49K, and truncated BTN3A1.

In some embodiments, the linker comprises one protease cleavage site. In some embodiments, the protease cleavage site comprises any of the amino acid sequences of SEQ ID NO: 32 and SEQ ID NOs: 89-102 (Table 2), which comprise (e.g. SEQ ID NOs: 99-102), or can be modified to comprise (e.g. any of SEQ ID NOs: 89-98), a GS linker at the amino and/or carboxy terminus when incorporated into the linker. In some embodiments, the linker comprises multiple protease cleavage sites, such as two, three, four, five, six or more protease cleavage sites, e.g. up to ten or up to fifteen protease cleavage sites, and each cleavage site is the same as or different from any of the other cleavage sites. In some embodiments, the linker is or comprises the amino acid sequence of any one or more of (e.g. 2, 3, 4, 5 copies of) of SEQ ID NOs: 99-102, 172-177, and 108-120. In some embodiments, the linker comprises one copy or more than one copy (e.g. 2, 3, 4, 5, 6, 10 or 15) of the amino acid sequence of any of SEQ ID NOs: 89-98, 103 and 104.

In some embodiments, the protease cleavage site or one or more of the multiple protease cleavage sites is recognized by one or more of thrombin, trypsin, plasmin, prostate-specific antigen (PSA), urokinase plasminogen activator (uPA), urokinase plasminogen activator receptor (uPAR), matrix metalloproteinase (MMP), matriptase (MT-SP1), legumain, a disintegrin and metalloproteinase (ADAM), and transmembrane Serine Protease (TMPRSS). Alternatively stated, in some embodiments, the protease that recognizes the protease cleavage site, or that recognizes one or more of the multiple protease cleavage sites, is one or more of thrombin, trypsin, plasmin, prostate-specific antigen (PSA), urokinase plasminogen activator (uPA), urokinase plasminogen activator receptor (uPAR), matrix metalloproteinase (MMP), matriptase (MT-SP1), legumain, a disintegrin and metalloproteinase (ADAM), transmembrane Serine Protease (TMPRSS), Granzyme B, activated protein C, Caspase, Cathepsin, Chymase, Elastase, Guanidinobenzoatase, HtrAl, Human Neutrophil Elastase, Lactoferrin, Marapsin, NS3/4A, PACE4, tissue plasminogen activator (tPA), DESC1, DPP-4, FAP, Hepsin, Matriptase-2, secretase, kallikrein-related peptidase (KLK), and tryptase, or a serine protease, a cysteine-type lysosomal protease, a metalloproteinase, a coagulation factor protease, or an aspartyl-type lysosomal protease. In some embodiments, the protease cleavage site comprises an amino acid sequence that is cleaved by a protease or type of protease listed herein. In some embodiments, the protease cleavage site or one or more of the multiple protease cleavage sites is recognized by an endogenous protease. In some embodiments, the protease that recognizes the protease cleavage site, or that recognizes one or more of the multiple protease cleavage sites, is an endogenous protease.

In some embodiments, the protease cleavage site or one or more of the multiple protease cleavage sites is recognized by one or more of a serine protease, a cysteine-type lysosomal protease, an aspartyl-type lysosomal protease and a metalloproteinase. In some embodiments, the protease that recognizes the protease cleavage site, or that recognizes one or more of the multiple protease cleavage sites, is one or more of a serine protease, a cysteine-type lysosomal protease, an aspartyl-type lysosomal protease and a metalloproteinase.

In some embodiments, the linker comprises an amino GS peptide, a peptide comprising a protease cleavage site, and a carboxy GS peptide. In some embodiments, the linker comprises the amino acid sequence of SEQ ID NO: 9, which comprises an amino GS peptide (SEQ ID NO: 52), a TPS cleavage peptide (SEQ ID NO: 53) comprising a protease cleavage site, and a carboxy GS peptide (SEQ ID NO: 54). In some embodiments, the linker comprises the same amino and carboxy GS peptides and a different cleavage peptide comprising the same or a (LHF) different protease cleavage site. Information on additional exemplary linkers is set forth in the tables below, which show the linker name, SEQ ID NO:, protease cleavage sites that each linker comprises and their corresponding SEQ ID NOs:, and the length (in amino acids (“a.a.”) of each linker. For example, the linker TPS4 45aa (SEQ ID NO: 107) comprises an MMP cleavage site having an amino acid sequence of SEQ ID NO: 93 and a matriptase cleavage site having an amino acid sequence of SEQ ID NO: 91. As shown in Table 2, TPS4 45aa (SEQ ID NO: 107) further comprises a GS peptide at the amino and carboxy termini. Additional linker information can be found at, for example, M. Geiger et al., Protease-activation using anti-idiotypic masks enables tumor specificity of a folate receptor 1-T cell bispecific antibody, Nature Commun 11:3196 (2020); E. J. Kwon et al., Ultrasensitive tumour-penetrating nanosensors of protease activity, Nature Biomed Eng 1:0054 (2017); WO 2016/118629 A1, US 20170204139A1; WO 2013/130683 A2, US 2013 0165389A1.

TABLE 1A

| | Matriptase | | |
|---|---|---|---|
| Linker | site | MMP site | Length |
| TPS1 45aa | LSGRSDNH* | SPLGLAGS | 45 a.a. |
| (SEQ ID | (SEQ ID | (SEQ ID | |
| NO: 9) | NO: 97) | NO: 103) | |
| TPS3 45aa | LSGRSDNH* | SPLGLAGS | 45 a.a. |
| (SEQ ID | (SEQ ID | (SEQ ID | |
| NO: 9) | NO: 97) | NO: 103) | |
| TPS6 45aa | PMAKK | | 45 a.a. |
| (SEQ ID | (SEQ ID | | |
| NO: 113) | NO: 95) | | |

*The sequence “LSGRSDNH” (SEQ ID NO 97) can be cleaved by both matriptase and uPA.

TABLE 1B

| | | Matriptase | |
|---|---|---|---|
| Linker | MMP site | site | Length |
| TPS4 45aa | VHMPLGFLGP | RQARVVNG | 45 a.a. |
| (SEQ ID | (SEQ ID | (SEQ ID | |
| NO: 107) | NO: 93) | NO: 91) | |
| TPS8 45aa | SPLGLAGS | PMAKK | 45 a.a. |
| (SEQ ID | (SEQ ID | (SEQ ID | |
| NO: 115) | NO: 103) | NO: 95) | |
| TPS9 45aa | VHMPLGFLGP | PMAKK | 45 a.a. |
| (SEQ ID | (SEQ ID | (SEQ ID | |
| NO: 116) | NO: 93) | NO: 95) | |
| TPS10 45aa | PLGVRGK | PMAKK | 45 a.a. |
| (SEQ ID | (SEQ ID | (SEQ ID | |
| NO: 117) | NO: 104) | NO: 95) | |
| TPS11 45aa | SPLGLAGS | RQARVVNG | 45 a.a. |
| (SEQ ID | (SEQ ID | (SEQ ID | |
| NO: 118) | NO: 103) | NO: 91) | |
| TPS12 45aa | VHMPLGFLGP | RQARVVNG | 45 a.a. |
| (SEQ ID | (SEQ ID | (SEQ ID | |
| NO: 119) | NO: 93) | NO: 91) | |
| TPS13 45aa | PLGVRGK | RQARVVNG | 45 a.a. |
| (SEQ ID | (SEQ ID | (SEQ ID | |
| NO: 120) | NO: 104) | NO: 91) | |

In some embodiments, the antigen binding domain specifically binds BCMA, MUC16 (also known as CA125), EGFR, EGFRVIII, MUC1, Flt-3, WT-1, CD20, CD23, CD30, CD38, CD70, CD33, CD133, MHC-WT1, TSPAN10, MHC-PRAME, MHC-NY-ESO1, HER2 (ERBB2), CAIX (Carbonic anhydrase IX), LIV1, ADAM10, CHRNA2, LeY, NKG2D, CS1, CD44v6, ROR1, CD19, Claudin-18.2 (Claudin-18A2, or Claudin18 isoform 2), PSCA, DLL3 (Delta-like protein 3, Drosophila Delta homolog 3, Delta3), Mud 7 (Mucin17, Muc3, Muc3), FAP alpha (Fibroblast Activation Protein alpha), Ly6G6D (Lymphocyte antigen 6 complex locus protein G6d, c6orf23, G6D, MEGT1, NG25), PSMA, MSLN, or RNF43 (E3 ubiquitin-protein ligase RNF43, RING finger protein 43). CARs and/or antibodies that target the antigens are disclosed, for example, in the following: BCMA-WO201616630, WO2020150339, WO2019196713, WO2016014565, WO2017025038; MUC16: U.S. Pat. No. 9,169,328, WO2016149368, WO2020023888; EGFRvIII: WO2017125830, WO2016016341; Flt3: WO2018222935, WO2020010284, WO2017173410; CD20: WO2018145649, WO2020010235, WO2020123691; CD38: WO2017025323; CD70: WO2019152742, WO2018152181; CD33: WO2016014576; CD133: WO2018072025; CS1: WO2019030240; ROR1: WO2016115559; CD19: WO2002077029, U.S. Pat. No. 11,077,144; Claudin: WO2018006882, WO2021008463; DLL3: WO2020180591; WT1: US20160152725A1, U.S. Pat. No. 7,622,119B2; CD23: U.S. Pat. No. 6,011,138A, CN1568198A; CD30: U.S. Pat. Nos. 10,815,301B2, 10,808,035B2; PRAME: US20180148503A1, WO2020186204A1; LIV1: US20200231699A1; NKG2D: WO2021179353A1, US20210269501A1; FAP Alpha: US20200246383A1, US20210115102A1; PSMA: US20210277141A1, WO2020108646A1; MSLN: CN109680002A, CN109628492A.

In some embodiments, the antigen binding domain specifically binds MUC16. In some embodiments, the antigen binding domain specifically binds MUC16 and the protease-activating CD45-gate CAR comprises the amino acid sequence of any of SEQ ID NOs: 11, 56, 75, 76, 77, 78, 79, and 121-170, with or without a signal sequence. In some embodiments, the antigen binding domain specifically binds MUC16 and comprises the amino acid sequence of SEQ ID NO: 75 or 76, with or without a signal sequence. In some embodiments, the antigen binding domain specifically binds to an antigen expressed by a certain type of tumor and the protease cleavage sites can be recognized and cleaved by a protease that the same type of tumor expresses. In some embodiments, the protease-activating CD45-gate CAR both (1) can specifically recognize and bind to an antigen expressed by a tumor and (2) comprises a CD45-CAR linker that can be cleaved by a protease that the tumor secretes and/or that is present and/or active in the tumor microenvironment.

In some embodiments, the antigen binding domain specifically binds to a breast cancer tumor antigen or a colorectal cancer tumor antigen and one or more of the at least one protease cleavage sites can be recognized and cleaved by uPA. In some embodiments, the protease-activating CD45-gate CAR's antigen binding domain specifically recognizes and binds to an antigen characteristic of any of cervical, breast, ovarian and colorectal cancers and at least one of its protease cleavage sites can be recognized and cleaved by MMP-2 and/or MMP-9. In some embodiments, the protease-activating CD45-gate CAR's antigen binding domain specifically recognizes and binds to an antigen characteristic of any of breast and ovarian cancers and at least one of its protease cleavage sites can be recognized and cleaved by matriptase.

In some embodiments, the protease-activating CD45-gate CAR comprises a signal sequence. In some embodiments, the signal sequence is the CD8 signal sequence. In some embodiments, the signal sequence comprises the amino acid sequence of SEQ ID NOs: 1 or 44.

Also provided herein is a CAR that specifically recognizes and binds to MUC16 (also known as CA125). In an embodiment, the anti-MUC16 CAR comprises the amino acid sequence of any of SEQ ID NOs: 11, 56, 75, 76, 77, 78, 79, and 121-170, or a variant thereof that does not comprise a signal sequence, or a conservative variant thereof.

MUC16 has been found to be an overexpressed antigen in several cancers, including ovarian, breast, pancreatic, non-small-cell lung cancer, intrahepatic cholangiocarcinoma-mass forming type, adenocarcinoma of the uterine cervix, and adenocarcinoma of the gastric tract. See, e.g., Haridas et al., FASEB J. 28:4183-99 (2014). The present disclosure therefore provides, in some embodiments, a method of treating any of ovarian cancer, breast cancer, pancreatic cancer, non-small-cell lung cancer, intrahepatic cholangiocarcinoma-mass forming type, adenocarcinoma of the uterine cervix, and adenocarcinoma of the gastric tract comprising administering to a patient who has that condition one or more engineered immune cells that functionally express a MUC16-specific CAR disclosed herein e.g. one comprising the amino acid sequence of any of SEQ ID NOs: 11, 56, 75, 76, 77, 78, 79, and 121-170, and variants thereof described herein or that functionally express a protease-activating CD45-gate MUC16-specific CAR disclosed herein (e.g. one comprising the amino acid sequence of SEQ ID NOs: 11, 16, 18, 20-31, 56, 75, 76, 77, 78, 79, or 121-170, or a variant thereof described herein).

In various embodiments, the protease-activating CD45-gate CAR protein disclosed herein comprises the amino acid sequence of, e.g., any one or more of SEQ ID NOs: 1-178 and conservative variants thereof. In certain embodiments, the protease-activating CD45-gate CAR comprises an amino acid sequence which comprises at least 70%, for example at least 80%, or at least 90%, 95%, 97%, or 99% sequence identity with any one or more of SEQ ID NOs: 1-178.

In some embodiments, the protease-activating CD45-gate CAR disclosed herein comprises any one of the amino acid sequences of SEQ ID NOs: 11, 16, 17 (control sequence-contains no protease cleavage site), 18, 19 (control sequence-contains no protease cleavage site), 20, 21, 22, 23, 24, 25, 26, 27, 28, 29, 30, 31, 56, 75, 76, 77, 78, 79, and 121-170. Some of these sequences contain one or both of the HA (tag) (SEQ ID NO: 2) and V5 peptide motif (SEQ ID NO: 55). In some embodiments of the protease-activating CD45-gate CAR of the disclosure, either or both of the HA (tag) and V5 peptide motif, and/or the signal peptide, are excluded from the protease-activating CD45-gate CAR. The present disclosure therefore provides a protease-activating CD45-gate CAR that comprises a variant of the amino acid sequence of any one of SEQ ID NOs: 11, 16-31, 56, 75-79 and 121-170 wherein the variant excludes (does not comprise) the amino acid sequence of the HA tag of SEQ ID NO: 2 and/or the amino acid sequence of the V5 peptide motif of SEQ ID NO: 55 and/or the amino acid sequence of CD8ss SEQ ID NO: 1, or an amino acid sequence which comprises at least 70%, for example at least 80%, or at least 90%, 95%, 97%, 99% or 100% sequence identity therewith. Some embodiments of the protease-activating CD45-gate CAR disclosed herein contain one, two, or all three of the HA (tag) (SEQ ID NO: 2), V5 peptide motif (SEQ ID NO: 55) and CD8 signal sequence (SEQ ID NO: 1).

In some embodiments, the protease-activating CD45-gate CAR comprises a CD45 recruiting domain, a CAR, and a linker connecting the carboxy terminus of the CD45 recruiting domain to the amino terminus of the antigen binding domain of the CAR. In some embodiments, the protease-activating CD45-gate CAR comprises a CD45 recruiting domain, a CAR, and a linker connecting the carboxy terminus of the CD45 recruiting domain to the amino terminus of the antigen binding domain of the CAR wherein the CAR comprises the amino acid sequence of SEQ ID NO: 11.

In some embodiments, the protease-activating CD45-gate CAR extracellular CD45 recruiting domain comprises the amino acid sequence of SEQ ID NOs: 3, 4, 12, 13, 14, or 15.

In some embodiments, the protease-activating CD45-gate CAR extracellular CD45 recruiting domain together with the protease-cleavable linker comprises the amino acid sequence of SEQ ID NOs: 5 or 6.

In some embodiments, the protease-activating CD45-gate CAR extracellular protease-cleavable linker comprises the amino acid sequence of SEQ ID NOs: 8, 9, or 10 and/or the protease cleavage site having the amino acid sequence of SEQ ID NOs: 32 (thrombin cleavage site) or 53 (TPS cleavage site) and/or either or both of SEQ ID NOs: 52 or 54 (sequences flanking the cleavage site). In some embodiments, the protease-activating CD45-gate CAR comprises a non-cleavable linker; in some embodiments, the non-cleavable linker comprises the amino acid sequence of SEQ ID NO: 7.

In some embodiments, the protease-activating CD45-gate CAR extracellular antigen binding domain comprises the amino acid sequence of SEQ ID NO: 56.

In some embodiments, the protease-activating CD45-gate CAR transmembrane domain comprises the amino acid sequence of SEQ ID NO: 39. In some embodiments, the CAR's transmembrane and intracellular domain comprises the amino acid sequence of SEQ ID NO: 41.

In some embodiments, the protease-activating CD45-gate CAR intracellular domain comprises one or more of the amino acid sequences of SEQ ID NOs: 33, 40 and 43.

In some embodiments, the protease-activating CD45-gate CAR hinge domain comprises the amino acid sequence of SEQ ID NOs: 36, 37 or 38.

In some embodiments, the protease-activating CD45-gate CAR disclosed herein comprises an N-terminal signal sequence. In some embodiments, the signal sequence has the amino acid sequence of SEQ ID NOs: 1 or 44. In some embodiments, a nucleic acid that encodes the protease-activating CD45-gate CAR encodes the signal sequence, and, during processing within the cell, the signal sequence is removed, leaving the remainder of the encoded amino acid sequence in the mature form of the protein. This mature form of protease-activating CD45-gate CAR is within the scope of the disclosure. In some embodiments, the protease-activating CD45-gate CAR disclosed herein does not comprise an N-terminal signal sequence.

In another aspect, provided herein is a nucleic acid encoding the extracellular domain of the protease-activating CD45-gate CAR disclosed herein. In another aspect, provided herein is a nucleic acid that encodes the protease-activating CD45-gate CAR disclosed herein. In certain embodiments, a nucleic acid of the disclosure encodes a protease-activating CD45-gate CAR that comprises an amino acid sequence which comprises at least 70%, for example at least 80%, or at least 90%, 95%, 97%, or 99% sequence identity with one or more of the amino acid sequences of SEQ ID NOs: 1-56. In certain embodiments, a nucleic acid of the disclosure encodes a protease-activating CD45-gate CAR that comprises an amino acid sequence which comprises at least 70%, for example at least 80%, or at least 90%, 95%, 97%, or 99% sequence identity with the amino acid sequence of SEQ ID NOs: 11, 16, 17 (control sequence-contains no protease cleavage site), 18, 19 (control sequence-contains no protease cleavage site), 20, 21, 22, 23, 24, 25, 26, 27, 28, 29, 30, 31, 56, 75, 76, 77, 78, 79, and 121-170, or a variant thereof that does not comprise an HA tag and/or a V5 sequence.

Also provided herein is a nucleic acid that encodes the amino acid sequence of any of SEQ ID NOs: 11, 56, 75, 76, 77, 78, 79, and 121-170. Also provided herein is a nucleic acid that encodes the amino acid sequence of SEQ ID NO: 56. In certain embodiments, a nucleic acid of the disclosure encodes an anti-MUC16 CAR that comprises an amino acid sequence which comprises at least 70%, for example at least 80%, or at least 90%, 95%, 97%, or 99% sequence identity with the amino acid sequence of SEQ ID NOs: 11, 56, 75, 76, 77, 78, 79, and 121-170.

In another aspect, provided herein is a vector comprising the nucleic acid disclosed herein. In an embodiment, the vector is an expression vector. In an embodiment, the vector is a viral vector, a retroviral vector, a DNA vector, a plasmid, a RNA vector, an adenoviral vector, an adenovirus associated vector, a lentiviral vector, or any combination thereof.

In a further aspect, provided herein is an engineered cell e.g. an engineered immune cell comprising the protease-activating CD45-gate CAR disclosed herein. In a further aspect, provided herein is an engineered immune cell comprising the nucleic acid disclosed herein. In another aspect, provided herein is an engineered immune cell comprising the vector disclosed herein.

In another aspect, an immune cell e.g. T cell of the disclosure comprises e.g. expresses a polypeptide that consists of or comprises one or more amino acid sequences listed in Table 2. In some embodiments, an immune cell e.g. T cell of the disclosure comprises e.g. expresses a nucleic acid that encodes one or more amino acid sequences listed in Table 2.

In various embodiments, any of the engineered cells e.g. any of the engineered immune cells disclosed herein functionally express the protease-activating CD45-gate CAR disclosed herein. In various embodiments, any of the engineered cells e.g. any of the engineered immune cells disclosed herein functionally express a protease-activating CD45-gate CAR that comprises the amino acid sequence of SEQ ID NO: 11, 56, 75, 76, 77, 78, 79, and 121-170 or a variant thereof that lacks an N-terminal signal sequence. Also provided herein are engineered cells e.g. engineered immune cells that functionally express a CAR that comprises the amino acid sequence of SEQ ID NO: 11, 56, 75, 76, 77, 78, 79, and 121-170 or a variant thereof that lacks an N-terminal signal sequence. In various embodiments, any of the engineered cells e.g. any of the engineered immune cells disclosed herein is an isolated cell.

In various embodiments, the engineered cell e.g. engineered immune cell disclosed herein functionally expresses the protease-activating CD45-gate CAR disclosed herein from a nucleic acid encoding the protease-activating CD45-gate CAR. In various embodiments, the engineered cell e.g. engineered immune cell disclosed herein functionally expresses CD45, e.g. functionally expresses the cell's endogenous CD45 gene or functionally expresses CD45 from an exogenous nucleic acid that encodes CD45 and was introduced into cell.

TABLE 2
Exemplary Protein Sequences

| SEQ ID NO: | Name | Amino Acid Sequence |
|---|---|---|
| 1 | CD8ss | MALPVTALLLPLALLLHAARP |
| 2 | HA(tag) | GYPYDVPDYA |
| 3 | CD45-Gate1 | DIVMTQTPASVSEPVGGSVTIKCQASQSFYNLLAWYQQKPGQPPKLLIYDASDL |
| | (4131) | ASGVPSRFKGSGSGTDFTLTISDLECADAAAYYCQSADGSSYAFGGGTEVVVKG |
| | | GGGGSGGGGSGGGGSGGGGSQEQLEESGGGLVKPEGSLTLTCTASGVSFSSSYW |
| | | IYWVRQAPGKGLEWIACIYTGSSGSTYYASWAKGRFTVSETSSTTVTLQMTSLT |
| | | AADTATYFCARASAWTYGMDLWGPGTLVTVSS |
| 4 | CD45-Gate2 | DIVMTQTPASVSEPVGGTVTIMCQASQSISNWLAWYQQKPGQPPKLLIYQASKL |
| | (4122) | ASGVPSRFKGSGSGTEYTLTISDLECADAATYYCQSYYDSGSNVFFAFGGGTKV |
| | | VVEGGGGGSGGGGSGGGGSGGGGSLSLEESGGDLVKPGASLTLTCTASGFSFSA |
| | | GYWICWVRQAPGKGLEWIACTYAGRSGSTYYANWVNGRFTIPKTSSTTVTLQMT |
| | | SLSGADTASYFCARGNAGVAVGALWGPGTLVTVSS |
| 5 | CD45-Gate1 | DIVMTQTPASVSEPVGGSVTIKCQASQSFYNLLAWYQQKPGQPPKLLIYDASDL |
| | withTPS | ASGVPSRFKGSGSGTDFTLTISDLECADAAAYYCQSADGSSYAFGGGTEVVVKG |
| | | GGGGSGGGGSLSGRSDNHSPLGLAGSGGGGSGGGGSQEQLEESGGGLVKPEGSL |
| | | TLTCTASGVSFSSSYWIYWVRQAPGKGLEWIACIYTGSSGSTYYASWAKGRFTV |
| | | SETSSTTVTLQMTSLTAADTATYFCARASAWTYGMDLWGPGTLVTVSS |
| 6 | CD45-Gate2 | DIVMTQTPASVSEPVGGTVTIMCQASQSISNWLAWYQQKPGQPPKLLIYQASKL |
| | withTPS | ASGVPSRFKGSGSGTEYTLTISDLECADAATYYCQSYYDSGSNVFFAFGGGTKV |
| | | VVEGGGGGSGGGGSLSGRSDNHSPLGLAGSGGGGSGGGGSLSLEESGGDLVKPG |
| | | ASLTLTCTASGFSFSAGYWICWVRQAPGKGLEWIACTYAGRSGSTYYANWVNGR |
| | | FTIPKTSSTTVTLQMTSLSGADTASYFCARGNAGVAVGALWGPGTLVTVSS |
| 171 | CD45-Gate3 | DILLTQSPATLSLSPGERATLSCRASQNIGTSIQWYQQKPGQAPRLLIRSSSES |
| | (RB/RO) | ISGIPSRFSGSGSGTDFTLTISSLEPEDFAVYYCQQSNTWPFTFGQGTKLEIKG |
| | | GGGSGGGGSGGGGSGGGGSEVQLVESGAEVKKPGASVKVSCKASGYTFTNYITH |
| | | WVKQEPGQGLEWIGYFNPYNHGTKYNEKFKGRATLTADKSISTAYMELSSLRSE |
| | | DTAVYYCARSGPYAWFDTWGQGTTVTVSS |
| 11 | MUC16 CAR1 | QVQLQESGPGLVKPSETLSLTCTVSGGSISYYSWTWVRQPAGQGLEWIGRIHIS |
| | (53B6) | GVTNHNPSLKSRVSMSIDTSRTQFSLRLTSVTAADTAVYFCARSGGTYSAFDIW |
| | | GQGTMVTVSSGGGGSGGGGSGGGGSGGGGSEIVLTQSPGTLSLSPGERSTLSCR |
| | | ASQSFTSNYLAWYQQRPGQAPRLLIYGASTRAIGIPDRFSGSGSGTDFTLTISR |
| | | LEPEDFAVYYCQQYGSSPWTFGQGTKVEIKTTTPAPRPPTPAPTIASQPLSLRP |
| | | EACRPAAGGAVHTRGLDFACDIYIWAPLAGTCGVLLLSLVITLYCKRGRKKLLY |
| | | IFKQPFMRPVQTTQEEDGCSCRFPEEEEGGCELRVKFSRSADAPAYQQGQNQLY |
| | | NELNLGRREEYDVLDKRRGRDPEMGGKPRRKNPQEGLYNELQKDKMAEAYSEIG |
| | | MKGERRRGKGHDGLYQGLSTATKDTYDALHMQALPPR |
| 12 | Sec49k | GFHTINATWWANITLVGPPDTPVTWYDTQGLWFCNGSRVKNPQIRHTCNDQNLT |
| | | LIHVNKTYERTYMGYNRQGTKKEDYKVVVIPPPPATVKPQPEPEYVEVYMGENK |
| | | TLEGPPGTPVTWFNQDGKKFCEGEKVLHPEFNHTCDKQNLILLFVNFTHDGAYL |
| | | GYNHQGTQRTHYEVTVLDLFPDSGQMKIENHSEETEQKNDEHHNWQKQGGQKQG |
| | | GQKTNQTKVNDRRKTAQKRPSKLKPATIEAMLVTVTAGSNLTLVGPKAEGKVTW |
| | | FDGDLKRPCEPNYRLRHECNNQNLTLINVTKDYEGTYYGTNDKDEGKRYRVKVN |
| | | TTNSQSVKIQPYTRQTTPDQEHKFELQFETNGNYDSKIP |
| 13 | UL11 | HDACIPVVGKIGTNVTLNAVDFHPGDHVRWSYGPGGAGYMLCVYTGSWTEYKKP |
| | | DIIFKCLSNNSLLLINVTVNYTNTYRTLTSLNNWVHNQHHHKFPGWNLDTCYSL |
| | | TVNENGTFPTTTTKKPTTTTRTTTTTTTKKTTTTRTTTAAKKTTISTTHHKHSS |
| | | PKKSSTPNSHVEHHVGFEATAAETPLQPSPQHQHVATH |
| 14 | BTN3A1-IgV | QFSVLGPSGPILAMVGEDADLPCHLEPTMSAETMELKWVSSSLRQVVNVYADGK |
| | | EVEDRQSAPYRGRTSILRDGITAGKAALRIHNVTASDSGKYLCYFQDGDFYEKA |
| | | LVELKVA |
| 15 | BTN3A1-IgC | ALGSDLHVDVKGYKDGGIHLECRSTGWYPQPQIQWSNNKGENIPTVEAPVVADG |
| | | VGLYAVAASVIMRGSSGEGVSCTIRSSLLGLEKTASISIADPFFRSAQRWIAAL |
| | | AGT |
| 183 | BTN3A1 | QFSVLGPSGPILAMVGEDADLPCHLFPTMSAETMELKWVSSSLRQVVNVYADGK |
| | | EVEDRQSAPYRGRTSILRDGITAGKAALRIHNVTASDSGKYLCYFQDGDFYEKA |
| | | LVELKVAALGSDLHVDVKGYKDGGIHLECRSTGWYPQPQIQWSNNKGENIPTVE |
| | | APVVADGVGLYAVAASVIMRGSSGEGVSCTIRSSLLGLEKTASISIADPFFRSA |
| | | QRWIAALAGT |
| 16 | 0975-N-HA- | MALPVTALLLPLALLLHAARPGYPYDVPDYADIVMTQTPASVSEPVGGSVTIKC |
| | 4131scFv-V5- | QASQSFYNLLAWYQQKPGQPPKLLIYDASDLASGVPSRFKGSGSGTDFTLTISD |
| | 2G4STMB-53B6 | LECADAAAYYCQSADGSSYAFGGGTEVVVKGGGGGSGGGGSGGGGSGGGGSQEQ |
| | | LEESGGGLVKPEGSLTLTCTASGVSFSSSYWIYWVRQAPGKGLEWIACIYTGSS |
| | | GSTYYASWAKGRFTVSETSSTTVTLQMTSLTAADTATYFCARASAWTYGMDLWG |
| | | PGTLVTVSSGGGGSGGGGSLVPRGSKPIPNPLLGLDSTLVPRGSGGGGSGGGGS |
| | | QVQLQESGPGLVKPSETLSLTCTVSGGSISYYSWTWVRQPAGQGLEWIGRIHIS |
| | | GVTNHNPSLKSRVSMSIDTSRTQFSLRLTSVTAADTAVYFCARSGGTYSAFDIW |
| | | GQGTMVTVSSGGGGSGGGGSGGGGSGGGGSEIVLTQSPGTLSLSPGERSTLSCR |
| | | ASQSFTSNYLAWYQQRPGQAPRLLIYGASTRAIGIPDRFSGSGSGTDFTLTISR |
| | | LEPEDFAVYYCQQYGSSPWTFGQGTKVEIKTTTPAPRPPTPAPTIASQPLSLRP |
| | | EACRPAAGGAVHTRGLDFACDIYIWAPLAGTCGVLLLSLVITLYCKRGRKKLLY |
| | | IFKQPFMRPVQTTQEEDGCSCRFPEEEEGGCELRVKFSRSADAPAYQQGQNQLY |
| | | NELNLGRREEYDVLDKRRGRDPEMGGKPRRKNPQEGLYNELQKDKMAEAYSEIG |
| | | MKGERRRGKGHDGLYQGLSTATKDTYDALHMQALPPR |
| 17 | 0976-N-HA- | MALPVTALLLPLALLLHAARPGYPYDVPDYADIVMTQTPASVSEPVGGSVTIKC |
| | 4131scFv-V5- | QASQSFYNLLAWYQQKPGQPPKLLIYDASDLASGVPSRFKGSGSGTDFTLTISD |
| | 3G4S-53B6 | LECADAAAYYCQSADGSSYAFGGGTEVVVKGGGGGSGGGGSGGGGSGGGGSQEQ |
| | | LEESGGGLVKPEGSLTLTCTASGVSFSSSYWIYWVRQAPGKGLEWIACIYTGSS |
| | | GSTYYASWAKGRFTVSETSSTTVTLQMTSLTAADTATYFCARASAWTYGMDLWG |
| | | PGTLVTVSSGGGGSGGGGSGGGSGSKPIPNPLLGLDSTGGGSGSGGGGSGGGGS |
| | | QVQLQESGPGLVKPSETLSLTCTVSGGSISYYSWTWVRQPAGQGLEWIGRIHIS |
| | | GVTNHNPSLKSRVSMSIDTSRTQFSLRLTSVTAADTAVYFCARSGGTYSAFDIW |
| | | GQGTMVTVSSGGGGSGGGGSGGGGSGGGGSEIVLTQSPGTLSLSPGERSTLSCR |
| | | ASQSFTSNYLAWYQQRPGQAPRLLIYGASTRAIGIPDRFSGSGSGTDFTLTISR |
| | | LEPEDFAVYYCQQYGSSPWTFGQGTKVEIKTTTPAPRPPTPAPTIASQPLSLRP |
| | | EACRPAAGGAVHTRGLDFACDIYIWAPLAGTCGVLLLSLVITLYCKRGRKKLLY |
| | | IFKQPFMRPVQTTQEEDGCSCRFPEEEEGGCELRVKFSRSADAPAYQQGQNQLY |
| | | NELNLGRREEYDVLDKRRGRDPEMGGKPRRKNPQEGLYNELQKDKMAEAYSEIG |
| | | MKGERRRGKGHDGLYQGLSTATKDTYDALHMQALPPR |
| 18 | 0977-N-HA- | MALPVTALLLPLALLLHAARPGYPYDVPDYADIVMTQTPASVSEPVGGTVTIMC |
| | 4122scFv-V5- | QASQSISNWLAWYQQKPGQPPKLLIYQASKLASGVPSRFKGSGSGTEYTLTISD |
| | 2G4STMB-53B6 | LECADAATYYCQSYYDSGSNVFFAFGGGTKVVVEGGGGGSGGGGSGGGGSGGGG |
| | | SLSLEESGGDLVKPGASLTLTCTASGFSFSAGYWICWVRQAPGKGLEWIACTYA |
| | | GRSGSTYYANWVNGRFTIPKTSSTTVTLQMTSLSGADTASYFCARGNAGVAVGA |
| | | LWGPGTLVTVSSGGGGSGGGGSLVPRGSKPIPNPLLGLDSTLVPRGSGGGGSGG |
| | | GGSQVQLQESGPGLVKPSETLSLTCTVSGGSISYYSWTWVRQPAGQGLEWIGRI |
| | | HISGVTNHNPSLKSRVSMSIDTSRTQFSLRLTSVTAADTAVYFCARSGGTYSAF |
| | | DIWGQGTMVTVSSGGGGSGGGGSGGGGSGGGGSEIVLTQSPGTLSLSPGERSTL |
| | | SCRASQSFTSNYLAWYQQRPGQAPRLLIYGASTRAIGIPDRFSGSGSGTDFTLT |
| | | ISRLEPEDFAVYYCQQYGSSPWTFGQGTKVEIKTTTPAPRPPTPAPTIASQPLS |
| | | LRPEACRPAAGGAVHTRGLDFACDIYIWAPLAGTCGVLLLSLVITLYCKRGRKK |
| | | LLYIFKQPFMRPVQTTQEEDGCSCRFPEEEEGGCELRVKFSRSADAPAYQQGQN |
| | | QLYNELNLGRREEYDVLDKRRGRDPEMGGKPRRKNPQEGLYNELQKDKMAEAYS |
| | | EIGMKGERRRGKGHDGLYQGLSTATKDTYDALHMQALPPR |
| 19 | 0978-N-HA- | MALPVTALLLPLALLLHAARPGYPYDVPDYADIVMTQTPASVSEPVGGTVTIMC |
| | 4122scFv-V5- | QASQSISNWLAWYQQKPGQPPKLLIYQASKLASGVPSRFKGSGSGTEYTLTISD |
| | 3G4S-53B6 | LECADAATYYCQSYYDSGSNVFFAFGGGTKVVVEGGGGGSGGGGSGGGGSGGGG |
| | | SLSLEESGGDLVKPGASLTLTCTASGFSFSAGYWICWVRQAPGKGLEWIACTYA |
| | | GRSGSTYYANWVNGRFTIPKTSSTTVTLQMTSLSGADTASYFCARGNAGVAVGA |
| | | LWGPGTLVTVSSGGGGSGGGGSGGGSGSKPIPNPLLGLDSTGGGSGSGGGGSGG |
| | | GGSQVQLQESGPGLVKPSETLSLTCTVSGGSISYYSWTWVRQPAGQGLEWIGRI |
| | | HISGVTNHNPSLKSRVSMSIDTSRTQFSLRLTSVTAADTAVYFCARSGGTYSAF |
| | | DIWGQGTMVTVSSGGGGSGGGGSGGGGSGGGGSEIVLTQSPGTLSLSPGERSTL |
| | | SCRASQSFTSNYLAWYQQRPGQAPRLLIYGASTRAIGIPDRFSGSGSGTDFTLT |
| | | ISRLEPEDFAVYYCQQYGSSPWTFGQGTKVEIKTTTPAPRPPTPAPTIASQPLS |
| | | LRPEACRPAAGGAVHTRGLDFACDIYIWAPLAGTCGVLLLSLVITLYCKRGRKK |
| | | LLYIFKQPFMRPVQTTQEEDGCSCRFPEEEEGGCELRVKFSRSADAPAYQQGQN |
| | | QLYNELNLGRREEYDVLDKRRGRDPEMGGKPRRKNPQEGLYNELQKDKMAEAYS |
| | | EIGMKGERRRGKGHDGLYQGLSTATKDTYDALHMQALPPR |
| 20 | 01082-N-HA- | MALPVTALLLPLALLLHAARPGYPYDVPDYADIVMTQTPASVSEPVGGSVTIKC |
| | 4131scFv- | QASQSFYNLLAWYQQKPGQPPKLLIYDASDLASGVPSRFKGSGSGTDFTLTISD |
| | TPS1-53B6 | LECADAAAYYCQSADGSSYAFGGGTEVVVKGGGGGSGGGGSGGGGSGGGGSQEQ |
| | | LEESGGGLVKPEGSLTLTCTASGVSFSSSYWIYWVRQAPGKGLEWIACIYTGSS |
| | | GSTYYASWAKGRFTVSETSSTTVTLQMTSLTAADTATYFCARASAWTYGMDLWG |
| | | PGTLVTVSSGGGGSGGGGSGGGSGLSGRSDNHSPLGLAGSGGGSGGGGSGGGGS |
| | | QVQLQESGPGLVKPSETLSLTCTVSGGSISYYSWTWVRQPAGQGLEWIGRIHIS |
| | | GVTNHNPSLKSRVSMSIDTSRTQFSLRLTSVTAADTAVYFCARSGGTYSAFDIW |
| | | GQGTMVTVSSGGGGSGGGGSGGGGSGGGGSEIVLTQSPGTLSLSPGERSTLSCR |
| | | ASQSFTSNYLAWYQQRPGQAPRLLIYGASTRAIGIPDRFSGSGSGTDFTLTISR |
| | | LEPEDFAVYYCQQYGSSPWTFGQGTKVEIKTTTPAPRPPTPAPTIASQPLSLRP |
| | | EACRPAAGGAVHTRGLDFACDIYIWAPLAGTCGVLLLSLVITLYCKRGRKKLLY |
| | | IFKQPFMRPVQTTQEEDGCSCRFPEEEEGGCELRVKFSRSADAPAYQQGQNQLY |
| | | NELNLGRREEYDVLDKRRGRDPEMGGKPRRKNPQEGLYNELQKDKMAEAYSEIG |
| | | MKGERRRGKGHDGLYQGLSTATKDTYDALHMQALPPR |
| 21 | 01092-N-HA- | MALPVTALLLPLALLLHAARPGYPYDVPDYADIVMTQTPASVSEPVGGTVTIMC |
| | 4122scFv- | QASQSISNWLAWYQQKPGQPPKLLIYQASKLASGVPSRFKGSGSGTEYTLTISD |
| | TPS1-53B6 | LECADAATYYCQSYYDSGSNVFFAFGGGTKVVVEGGGGGSGGGGSGGGGSGGGG |
| | | SLSLEESGGDLVKPGASLTLTCTASGFSFSAGYWICWVRQAPGKGLEWIACTYA |
| | | GRSGSTYYANWVNGRFTIPKTSSTTVTLQMTSLSGADTASYFCARGNAGVAVGA |
| | | LWGPGTLVTVSSGGGGSGGGGSGGGSGLSGRSDNHSPLGLAGSGGGSGGGGSGG |
| | | GGSQVQLQESGPGLVKPSETLSLTCTVSGGSISYYSWTWVRQPAGQGLEWIGRI |
| | | HISGVTNHNPSLKSRVSMSIDTSRTQFSLRLTSVTAADTAVYFCARSGGTYSAF |
| | | DIWGQGTMVTVSSGGGGSGGGGSGGGGSGGGGSEIVLTQSPGTLSLSPGERSTL |
| | | SCRASQSFTSNYLAWYQQRPGQAPRLLIYGASTRAIGIPDRFSGSGSGTDFTLT |
| | | ISRLEPEDFAVYYCQQYGSSPWTFGQGTKVEIKTTTPAPRPPTPAPTIASQPLS |
| | | LRPEACRPAAGGAVHTRGLDFACDIYIWAPLAGTCGVLLLSLVITLYCKRGRKK |
| | | LLYIFKQPFMRPVQTTQEEDGCSCRFPEEEEGGCELRVKFSRSADAPAYQQGQN |
| | | QLYNELNLGRREEYDVLDKRRGRDPEMGGKPRRKNPQEGLYNELQKDKMAEAYS |
| | | EIGMKGERRRGKGHDGLYQGLSTATKDTYDALHMQALPPR |
| 22 | 01098-N-HA- | MALPVTALLLPLALLLHAARPGYPYDVPDYADIVMTQTPASVSEPVGGSVTIKC |
| | 4131scFv-V5- | QASQSFYNLLAWYQQKPGQPPKLLIYDASDLASGVPSRFKGSGSGTDFTLTISD |
| | 2XTPS1-53B6 | LECADAAAYYCQSADGSSYAFGGGTEVVVKGGGGGSGGGGSGGGGSGGGGSQEQ |
| | | LEESGGGLVKPEGSLTLTCTASGVSFSSSYWIYWVRQAPGKGLEWIACIYTGSS |
| | | GSTYYASWAKGRFTVSETSSTTVTLQMTSLTAADTATYFCARASAWTYGMDLWG |
| | | PGTLVTVSSGGGGSLSGRSDNHSPLGLAGSKPIPNPLLGLDSTLSGRSDNHSPL |
| | | GLAGSGGGGSQVQLQESGPGLVKPSETLSLTCTVSGGSISYYSWTWVRQPAGQG |
| | | LEWIGRIHISGVTNHNPSLKSRVSMSIDTSRTQFSLRLTSVTAADTAVYFCARS |
| | | GGTYSAFDIWGQGTMVTVSSGGGGSGGGGSGGGGSGGGGSEIVLTQSPGTLSLS |
| | | PGERSTLSCRASQSFTSNYLAWYQQRPGQAPRLLIYGASTRAIGIPDRFSGSGS |
| | | GTDFTLTISRLEPEDFAVYYCQQYGSSPWTFGQGTKVEIKTTTPAPRPPTPAPT |
| | | IASQPLSLRPEACRPAAGGAVHTRGLDFACDIYIWAPLAGTCGVLLLSLVITLY |
| | | CKRGRKKLLYIFKQPFMRPVQTTQEEDGCSCRFPEEEEGGCELRVKFSRSADAP |
| | | AYQQGQNQLYNELNLGRREEYDVLDKRRGRDPEMGGKPRRKNPQEGLYNELQKD |
| | | KMAEAYSEIGMKGERRRGKGHDGLYQGLSTATKDTYDALHMQALPPR |
| 23 | 01099-N-HA- | MALPVTALLLPLALLLHAARPGYPYDVPDYADIVMTQTPASVSEPVGGTVTIMC |
| | 4122scFv-V5- | QASQSISNWLAWYQQKPGQPPKLLIYQASKLASGVPSRFKGSGSGTEYTLTISD |
| | 2XTPS1-53B6 | LECADAATYYCQSYYDSGSNVFFAFGGGTKVVVEGGGGGSGGGGSGGGGSGGGG |
| | | SLSLEESGGDLVKPGASLTLTCTASGFSFSAGYWICWVRQAPGKGLEWIACTYA |
| | | GRSGSTYYANWVNGRFTIPKTSSTTVTLQMTSLSGADTASYFCARGNAGVAVGA |
| | | LWGPGTLVTVSSGGGGSLSGRSDNHSPLGLAGSKPIPNPLLGLDSTLSGRSDNH |
| | | SPLGLAGSGGGGSQVQLQESGPGLVKPSETLSLTCTVSGGSISYYSWTWVRQPA |
| | | GQGLEWIGRIHISGVTNHNPSLKSRVSMSIDTSRTQFSLRLTSVTAADTAVYFC |
| | | ARSGGTYSAFDIWGQGTMVTVSSGGGGSGGGGSGGGGSGGGGSEIVLTQSPGTL |
| | | SLSPGERSTLSCRASQSFTSNYLAWYQQRPGQAPRLLIYGASTRAIGIPDRFSG |
| | | SGSGTDFTLTISRLEPEDFAVYYCQQYGSSPWTFGQGTKVEIKTTTPAPRPPTP |
| | | APTIASQPLSLRPEACRPAAGGAVHTRGLDFACDIYIWAPLAGTCGVLLLSLVI |
| | | TLYCKRGRKKLLYIFKQPFMRPVQTTQEEDGCSCRFPEEEEGGCELRVKFSRSA |
| | | DAPAYQQGQNQLYNELNLGRREEYDVLDKRRGRDPEMGGKPRRKNPQEGLYNEL |
| | | QKDKMAEAYSEIGMKGERRRGKGHDGLYQGLSTATKDTYDALHMQALPPR |
| 24 | 01102-N-HA- | MALPVTALLLPLALLLHAARPGYPYDVPDYADIVMTQTPASVSEPVGGSVTIKC |
| | 4131-VL-RS- | QASQSFYNLLAWYQQKPGQPPKLLIYDASDLASGVPSRFKGSGSGTDFTLTISD |
| | VH-TPS1-53B6 | LECADAAAYYCQSADGSSYAFGGGTEVVVKGGGGGSGGGGSLSGRSDNHSPLGL |
| | | AGSGGGGSGGGGSQEQLEESGGGLVKPEGSLTLTCTASGVSFSSSYWIYWVRQA |
| | | PGKGLEWIACIYTGSSGSTYYASWAKGRFTVSETSSTTVTLQMTSLTAADTATY |
| | | FCARASAWTYGMDLWGPGTLVTVSSGGGGSGGGGSGGGSGLSGRSDNHSPLGLA |
| | | GSGGGSGGGGSGGGGSQVQLQESGPGLVKPSETLSLTCTVSGGSISYYSWTWVR |
| | | QPAGQGLEWIGRIHISGVTNHNPSLKSRVSMSIDTSRTQFSLRLTSVTAADTAV |
| | | YFCARSGGTYSAFDIWGQGTMVTVSSGGGGSGGGGSGGGGSGGGGSEIVLTQSP |
| | | GTLSLSPGERSTLSCRASQSFTSNYLAWYQQRPGQAPRLLIYGASTRAIGIPDR |
| | | FSGSGSGTDFTLTISRLEPEDFAVYYCQQYGSSPWTFGQGTKVEIKTTTPAPRP |
| | | PTPAPTIASQPLSLRPEACRPAAGGAVHTRGLDFACDIYIWAPLAGTCGVLLLS |
| | | LVITLYCKRGRKKLLYIFKQPFMRPVQTTQEEDGCSCRFPEEEEGGCELRVKFS |
| | | RSADAPAYQQGQNQLYNELNLGRREEYDVLDKRRGRDPEMGGKPRRKNPQEGLY |
| | | NELQKDKMAEAYSEIGMKGERRRGKGHDGLYQGLSTATKDTYDALHMQALPPR |
| 25 | 01103-N-HA- | MALPVTALLLPLALLLHAARPGYPYDVPDYADIVMTQTPASVSEPVGGTVTIMC |
| | 4122-VL-RS- | QASQSISNWLAWYQQKPGQPPKLLIYQASKLASGVPSRFKGSGSGTEYTLTISD |
| | VH-TPS1-53B6 | LECADAATYYCQSYYDSGSNVFFAFGGGTKVVVEGGGGGSGGGGSLSGRSDNHS |
| | | PLGLAGSGGGGSGGGGSLSLEESGGDLVKPGASLTLTCTASGFSFSAGYWICWV |
| | | RQAPGKGLEWIACTYAGRSGSTYYANWVNGRFTIPKTSSTTVTLQMTSLSGADT |
| | | ASYFCARGNAGVAVGALWGPGTLVTVSSGGGGSGGGGSGGGSGLSGRSDNHSPL |
| | | GLAGSGGGSGGGGSGGGGSQVQLQESGPGLVKPSETLSLTCTVSGGSISYYSWT |
| | | WVRQPAGQGLEWIGRIHISGVTNHNPSLKSRVSMSIDTSRTQFSLRLTSVTAAD |
| | | TAVYFCARSGGTYSAFDIWGQGTMVTVSSGGGGSGGGGSGGGGSGGGGSEIVLT |
| | | QSPGTLSLSPGERSTLSCRASQSFTSNYLAWYQQRPGQAPRLLIYGASTRAIGI |
| | | PDRFSGSGSGTDFTLTISRLEPEDFAVYYCQQYGSSPWTFGQGTKVEIKTTTPA |
| | | PRPPTPAPTIASQPLSLRPEACRPAAGGAVHTRGLDFACDIYIWAPLAGTCGVL |
| | | LLSLVITLYCKRGRKKLLYIFKQPFMRPVQTTQEEDGCSCRFPEEEEGGCELRV |
| | | KFSRSADAPAYQQGQNQLYNELNLGRREEYDVLDKRRGRDPEMGGKPRRKNPQE |
| | | GLYNELQKDKMAEAYSEIGMKGERRRGKGHDGLYQGLSTATKDTYDALHMQALP |
| | | PR |
| 26 | 01112-N-HA- | MALPVTALLLPLALLLHAARPGYPYDVPDYADIVMTQTPASVSEPVGGSVTIKC |
| | 4131-VL-TMB- | QASQSFYNLLAWYQQKPGQPPKLLIYDASDLASGVPSRFKGSGSGTDFTLTISD |
| | VH-V5- | LECADAAAYYCQSADGSSYAFGGGTEVVVKGGGGGSGGGGSLVPRGSGGGGSGG |
| | 2G4STMB-53B6 | GGSQEQLEESGGGLVKPEGSLTLTCTASGVSFSSSYWIYWVRQAPGKGLEWIAC |
| | | IYTGSSGSTYYASWAKGRFTVSETSSTTVTLQMTSLTAADTATYFCARASAWTY |
| | | GMDLWGPGTLVTVSSGGGGSGGGGSLVPRGSKPIPNPLLGLDSTLVPRGSGGGG |
| | | SGGGGSQVQLQESGPGLVKPSETLSLTCTVSGGSISYYSWTWVRQPAGQGLEWI |
| | | GRIHISGVTNHNPSLKSRVSMSIDTSRTQFSLRLTSVTAADTAVYFCARSGGTY |
| | | SAFDIWGQGTMVTVSSGGGGSGGGGSGGGGSGGGGSEIVLTQSPGTLSLSPGER |
| | | STLSCRASQSFTSNYLAWYQQRPGQAPRLLIYGASTRAIGIPDRFSGSGSGTDF |
| | | TLTISRLEPEDFAVYYCQQYGSSPWTFGQGTKVEIKTTTPAPRPPTPAPTIASQ |
| | | PLSLRPEACRPAAGGAVHTRGLDFACDIYIWAPLAGTCGVLLLSLVITLYCKRG |
| | | RKKLLYIFKQPFMRPVQTTQEEDGCSCRFPEEEEGGCELRVKFSRSADAPAYQQ |
| | | GQNQLYNELNLGRREEYDVLDKRRGRDPEMGGKPRRKNPQEGLYNELQKDKMAE |
| | | AYSEIGMKGERRRGKGHDGLYQGLSTATKDTYDALHMQALPPR |
| 27 | 01113-N-HA- | MALPVTALLLPLALLLHAARPGYPYDVPDYADIVMTQTPASVSEPVGGTVTIMC |
| | 4122-VL-TMB- | QASQSISNWLAWYQQKPGQPPKLLIYQASKLASGVPSRFKGSGSGTEYTLTISD |
| | VH-V5- | LECADAATYYCQSYYDSGSNVFFAFGGGTKVVVEGGGGGSGGGGSLVPRGSGGG |
| | 2G4STMB-53B6 | GSGGGGSLSLEESGGDLVKPGASLTLTCTASGFSFSAGYWICWVRQAPGKGLEW |
| | | IACTYAGRSGSTYYANWVNGRFTIPKTSSTTVTLQMTSLSGADTASYFCARGNA |
| | | GVAVGALWGPGTLVTVSSGGGGSGGGGSLVPRGSKPIPNPLLGLDSTLVPRGSG |
| | | GGGSGGGGSQVQLQESGPGLVKPSETLSLTCTVSGGSISYYSWTWVRQPAGQGL |
| | | EWIGRIHISGVTNHNPSLKSRVSMSIDTSRTQFSLRLTSVTAADTAVYFCARSG |
| | | GTYSAFDIWGQGTMVTVSSGGGGSGGGGSGGGGSGGGGSEIVLTQSPGTLSLSP |
| | | GERSTLSCRASQSFTSNYLAWYQQRPGQAPRLLIYGASTRAIGIPDRFSGSGSG |
| | | TDFTLTISRLEPEDFAVYYCQQYGSSPWTFGQGTKVEIKTTTPAPRPPTPAPTI |
| | | ASQPLSLRPEACRPAAGGAVHTRGLDFACDIYIWAPLAGTCGVLLLSLVITLYC |
| | | KRGRKKLLYIFKQPFMRPVQTTQEEDGCSCRFPEEEEGGCELRVKFSRSADAPA |
| | | YQQGQNQLYNELNLGRREEYDVLDKRRGRDPEMGGKPRRKNPQEGLYNELQKDK |
| | | MAEAYSEIGMKGERRRGKGHDGLYQGLSTATKDTYDALHMQALPPR |
| 28 | 01121-N-HA- | MALPVTALLLPLALLLHAARPGYPYDVPDYAGFHTINATWWANITLVGPPDTPV |
| | sec49k-V5- | TWYDTQGLWFCNGSRVKNPQIRHTCNDQNLTLIHVNKTYERTYMGYNRQGTKKE |
| | 2G4STMB-53B6 | DYKVVVIPPPPATVKPQPEPEYVFVYMGENKTLEGPPGTPVTWFNQDGKKFCEG |
| | | EKVLHPEFNHTCDKQNLILLFVNFTHDGAYLGYNHQGTQRTHYEVTVLDLFPDS |
| | | GQMKIENHSEETEQKNDEHHNWQKQGGQKQGGQKTNQTKVNDRRKTAQKRPSKL |
| | | KPATIEAMLVTVTAGSNLTLVGPKAEGKVTWEDGDLKRPCEPNYRLRHECNNQN |
| | | LTLINVTKDYEGTYYGTNDKDEGKRYRVKVNTTNSQSVKIQPYTRQTTPDQEHK |
| | | FELQFETNGNYDSKIPGGGGSGGGGSLVPRGSKPIPNPLLGLDSTLVPRGSGGG |
| | | GSGGGGSQVQLQESGPGLVKPSETLSLTCTVSGGSISYYSWTWVRQPAGQGLEW |
| | | IGRIHISGVTNHNPSLKSRVSMSIDTSRTQFSLRLTSVTAADTAVYFCARSGGT |
| | | YSAFDIWGQGTMVTVSSGGGGSGGGGSGGGGSGGGGSEIVLTQSPGTLSLSPGE |
| | | RSTLSCRASQSFTSNYLAWYQQRPGQAPRLLIYGASTRAIGIPDRFSGSGSGTD |
| | | FTLTISRLEPEDFAVYYCQQYGSSPWTFGQGTKVEIKTTTPAPRPPTPAPTIAS |
| | | QPLSLRPEACRPAAGGAVHTRGLDFACDIYIWAPLAGTCGVLLLSLVITLYCKR |
| | | GRKKLLYIFKQPFMRPVQTTQEEDGCSCRFPEEEEGGCELRVKFSRSADAPAYQ |
| | | QGQNQLYNELNLGRREEYDVLDKRRGRDPEMGGKPRRKNPQEGLYNELQKDKMA |
| | | EAYSEIGMKGERRRGKGHDGLYQGLSTATKDTYDALHMQALPPR |
| 29 | 01122-N-HA- | MALPVTALLLPLALLLHAARPGYPYDVPDYAHDACIPVVGKIGTNVTLNAVDFH |
| | UL11-V5- | PGDHVRWSYGPGGAGYMLCVYTGSWTEYKKPDIIFKCLSNNSLLLINVTVNYTN |
| | 2G4STMB-53B6 | TYRTLTSLNNWVHNQHHHKFPGWNLDTCYSLTVNENGTEPTTTTKKPTTTTRTT |
| | | TTTTTKKTTTTRTTTAAKKTTISTTHHKHSSPKKSSTPNSHVEHHVGFEATAAE |
| | | TPLQPSPQHQHVATHGGGGSGGGGSLVPRGSKPIPNPLLGLDSTLVPRGSGGGG |
| | | SGGGGSQVQLQESGPGLVKPSETLSLTCTVSGGSISYYSWTWVRQPAGQGLEWI |
| | | GRIHISGVTNHNPSLKSRVSMSIDTSRTQFSLRLTSVTAADTAVYFCARSGGTY |
| | | SAFDIWGQGTMVTVSSGGGGSGGGGSGGGGSGGGGSEIVLTQSPGTLSLSPGER |
| | | STLSCRASQSFTSNYLAWYQQRPGQAPRLLIYGASTRAIGIPDRFSGSGSGTDF |
| | | TLTISRLEPEDFAVYYCQQYGSSPWTFGQGTKVEIKTTTPAPRPPTPAPTIASQ |
| | | PLSLRPEACRPAAGGAVHTRGLDFACDIYIWAPLAGTCGVLLLSLVITLYCKRG |
| | | RKKLLYIFKQPFMRPVQTTQEEDGCSCRFPEEEEGGCELRVKFSRSADAPAYQQ |
| | | GQNQLYNELNLGRREEYDVLDKRRGRDPEMGGKPRRKNPQEGLYNELQKDKMAE |
| | | AYSEIGMKGERRRGKGHDGLYQGLSTATKDTYDALHMQALPPR |
| 30 | 01207-N-HA- | MALPVTALLLPLALLLHAARPGYPYDVPDYAQFSVLGPSGPILAMVGEDADLPC |
| | IgV-V5- | HLEPTMSAETMELKWVSSSLRQVVNVYADGKEVEDRQSAPYRGRTSILRDGITA |
| | 2G4STMB-53B6 | GKAALRIHNVTASDSGKYLCYFQDGDFYEKALVELKVAGGGGSGGGGSLVPRGS |
| | | KPIPNPLLGLDSTLVPRGSGGGGSGGGGSQVQLQESGPGLVKPSETLSLTCTVS |
| | | GGSISYYSWTWVRQPAGQGLEWIGRIHISGVTNHNPSLKSRVSMSIDTSRTQFS |
| | | LRLTSVTAADTAVYFCARSGGTYSAFDIWGQGTMVTVSSGGGGSGGGGSGGGGS |
| | | GGGGSEIVLTQSPGTLSLSPGERSTLSCRASQSFTSNYLAWYQQRPGQAPRLLI |
| | | YGASTRAIGIPDRFSGSGSGTDFTLTISRLEPEDFAVYYCQQYGSSPWTFGQGT |
| | | KVEIKTTTPAPRPPTPAPTIASQPLSLRPEACRPAAGGAVHTRGLDFACDIYIW |
| | | APLAGTCGVLLLSLVITLYCKRGRKKLLYIFKQPFMRPVQTTQEEDGCSCRFPE |
| | | EEEGGCELRVKFSRSADAPAYQQGQNQLYNELNLGRREEYDVLDKRRGRDPEMG |
| | | GKPRRKNPQEGLYNELQKDKMAEAYSEIGMKGERRRGKGHDGLYQGLSTATKDT |
| | | YDALHMQALPPR |
| 31 | 01208-N-HA- | MALPVTALLLPLALLLHAARPGYPYDVPDYAALGSDLHVDVKGYKDGGIHLECR |
| | IgC-V5- | STGWYPQPQIQWSNNKGENIPTVEAPVVADGVGLYAVAASVIMRGSSGEGVSCT |
| | 2G4STMB-53B6 | IRSSLLGLEKTASISIADPFFRSAQRWIAALAGTGGGGSGGGGSLVPRGSKPIP |
| | | NPLLGLDSTLVPRGSGGGGSGGGGSQVQLQESGPGLVKPSETLSLTCTVSGGSI |
| | | SYYSWTWVRQPAGQGLEWIGRIHISGVTNHNPSLKSRVSMSIDTSRTQFSLRLT |
| | | SVTAADTAVYFCARSGGTYSAFDIWGQGTMVTVSSGGGGSGGGGSGGGGSGGGG |
| | | SEIVLTQSPGTLSLSPGERSTLSCRASQSFTSNYLAWYQQRPGQAPRLLIYGAS |
| | | TRAIGIPDRFSGSGSGTDFTLTISRLEPEDFAVYYCQQYGSSPWTFGQGTKVEI |
| | | KTTTPAPRPPTPAPTIASQPLSLRPEACRPAAGGAVHTRGLDFACDIYIWAPLA |
| | | GTCGVLLLSLVITLYCKRGRKKLLYIFKQPFMRPVQTTQEEDGCSCRFPEEEEG |
| | | GCELRVKFSRSADAPAYQQGQNQLYNELNLGRREEYDVLDKRRGRDPEMGGKPR |
| | | RKNPQEGLYNELQKDKMAEAYSEIGMKGERRRGKGHDGLYQGLSTATKDTYDAL |
| | | HMQALPPR |
| 33 | 4-1BB intra- | KRGRKKLLYIFKQPFMRPVQTTQEEDGCSCRFPEEEEGGCEL |
| | cellular | |
| | signaling | |
| | domain (ISD) | |
| 34 | CD3ζ intra- | RVKFSRSADAPAYQQGQNQLYNELNLGRREEYDVLDKRRGRDPEMGGKPRRKNP |
| | cellular | QEGLYNELQKDKMAEAYSEIGMKGERRRGKGHDGLYQGLSTATKDTYDALHMQA |
| | signaling | LPPR |
| | domain (ISD) | |
| 35 | CD28-IC (CD28 | RSKRSRGGHSDYMNMTPRRPGPTRKHYQPYAPPRDFAAYRS |
| | co- | |
| | stimulatory | |
| | domain) | |
| 36 | FcγRIIIα | GLWSTISSFEPPGYQ |
| | hinge | |
| 37 | CD8α hinge | TTTPAPRPPTPTIASQPLSLRPEACRPAAGGAVRTRGLDFACD |
| 38 | IgG1 hinge | EPKSPDKTHTCPPCPAPPVAGPSVFLFPPKPKDTLMTkRTPEVTCVVVDVSHED |
| | | PEVKFNWYVDGVEVHNAETKPREEQYNSTYRVVSVLTVLHQDWLNGKEYNCKVS |
| | | NKALPAPIEKTISKAKGQPREPQVITLPPSRDELTKNOVSLTCLVKGFIPSDIA |
| | | VEWESNGQPENNYKTTPPVLDSDGSFFLYSKLTVDKSRWQQGNVFSCSVMHEAL |
| | | HNHYTOKSLSLSPGK |
| 39 | CD8α | IYIWAPLAGTCGVLLLSLVITLYG |
| | transmembrane | |
| | (TM) domain | |
| 40 | CD3ζ intra- | RVKFSRSADAPAYQQGQNQLYNELNLGRREEYDVIDKRRGRDPEMGGKPRRKNP |
| | cellular | QEGLYNELQKDKMAEAYSEIGMKGERRRGKGEDGLYQGLSTATKDTYDALHMQA |
| | signaling | LPPP |
| | domain (ISD) | |
| 41 | FcεRI α-TM-IC | FFIPLLVVILFAVDTGLFISTQQQVTFLLKIKRTRKGERLLNPHPKPNTKNN |
| | (FcεRI α | |
| | chain | |
| | transmembrane | |
| | and intra- | |
| | cellular | |
| | domain) | |
| 42 | FcεRiβ-ΔITAM | MDTESNRRANLALPQEPSSVPAFEVLEISPQEVSSGRELKSASSPPLHTWLTVL |
| | (FcεRi β | KKEQEFLGVTQILTAMICLCFGTVVCSVLDISHIEGDIFSSFKAGYPFWGAIFF |
| | chain without | SISGMLSIISERRNATYLVRGSLGANTASSIAGGTGITILIINLKKSLAYIHIH |
| | ITAM) | SCQKFFETKCFMSFSTEIVVMMLFLTILGLGSAVSLTICGAGEELKGNKVPE |
| 43 | CD23-IC (CD28 | RSKRSRGGHSDYMNMTPRRPGPTPKHYQPIPPPDFAAYRS |
| | co- | |
| | stimulatory | |
| | domain) | |
| 44 | FgεRIγ-SP | MIPAVVILLLLLVEQAAA |
| | (signal | |
| | peptide) | |
| 45 | FcεRI γ-ΔITAM | LGEPQLCYILDAILFLIGIVLTLLICRLKIQVRKAAITSYEKS |
| | (FcεRI γ | |
| | chain without | |
| | ITAM) | |
| 46 | GSG-P2A (GSG- | GSGATNFSLLKQAGDVEENPGP |
| | P2A ribosomal | |
| | skip poly- | |
| | peptide) | |
| 47 | GSG-T2A (GSG- | GSGERGSLLTCGDVEENPGP |
| | T2A ribosomal | |
| | skip poly- | |
| | peptide) | |
| 48 | safety switch | CPYSNPSLCSGGGGSELPTQGTFSNVSTNVSPAKPTTTACPYSNPSLCSGGGGS |
| | | PAPRPPTPAPTIASQPLSLRPEACRPAAGGAVHTRGLDFACDIYIWAPLAGTCG |
| | | VLLLSLVITLYCNHRNRRRVCKCPRPVV |
| 49 | safety | MGTSLLCWMALCLLGADHADACPYSNPSLCSGGGGSELPTQGTFSNVSTNVSPA |
| | switch-amino | KPTTTACPYSNPSLCSGGGGSPAPRPPTPAPTIASQPLSLRPEACRPAAGGAVH |
| | term. | TRGLDFACDIYIWAPLAGTCGVLLLSLVITLYCNHRNRRRVCKCPRPVV |
| 50 | CD20 mimotope | CPYSNPSLC |
| 51 | 2xCD20 | GSGGGGSCPYSNPSLCSGGGGSCPYSNPSLCSGGGGS |
| | mimotope | |
| 52 | aminoGS | GGGGSGGGGSGGGSG |
| 54 | carboxyGS | GGGSGGGGSGGGGS |
| 55 | V5 | KPIPNPLLGLDST |
| 56 | Leader-53B6 | MALPVTALLLPLALLLHAARPQVQLQESGPGLVKPSETLSLTCTVSGGSISYYS |
| | scEv | WTWVRQPAGQGLEWIGRIHISGVTNHNPSLKSRVSMSIDTSRTQFSLRLTSVTA |
| | | ADTAVYFCARSGGTYSAFDIWGQGTMVTVSSGGGGSGGGGSGGGGSGGGGSEIV |
| | | LTQSPGTLSLSPGERSTLSCRASQSFTSNYLAWYQQRPGQAPRLLIYGASTRAI |
| | | GIPDRFSGSGSGTDFTLTISRLEPEDFAVYYCQQYGSSPWTFGQGTKVEIK |
| 57 | GSlinker | GGGGSGGGGSGGGGS |
| 178 | GS sequence | GGGGS |
| | (1) | |
| 181 | GS45 (GS | GGGGSGGGGSGGGSGSGGGGSGGGSGGGSGGGSGSGGGGSGGGGS |
| | sequence 2) | |
| 182 | GS45 (3) | GGGGSGGGGSGGGSGGGSGSGGGGSGGGGSGGGGSGGGGSGGGGS |

PROTEASE CLEAVAGE SITES:

| | | |
|---|---|---|
| 32 | TMB cleavage | LVPRGS |
| | site | |
| 53 | TPS cleavage | LSGRSDNHSPLGLAGS |
| 89 | MMP2/9/MTSP | VHMPLGFLGPRQARVVNG |
| | site (1) | |
| 90 | MMP2/9/MTSP | VHMPLGFLGPGSARVVNG |
| | site (2) | |
| 91 | MTSP | RQARVVNG |
| | (matriptase) | |
| | site (1) | |
| 92 | MTSPsite (2) | RQARVGSG |
| 93 | MMP2/9 (1) | VHMPLGFLGP |
| 94 | MMP2/9 (2) | VHMPLSFLGP |
| 95 | MTSP (3) | PMAKK |
| 96 | MTSP (4) | PMAKGS |
| 97 | MTSP (5) | LSGRSDNH |
| 98 | MTSP (6) | LSGRSDSH |
| 103 | MMP (site3) | SPLGLAGS |
| 104 | MMP (site 4) | PLGVRGK |
| 105 | MTSP (7) | PMAKG |
| 106 | MTSP (8) | GSARVVNG |

LINKER SEQUENCES WITH PROTEASE CLEAVAGE SITES

| | | |
|---|---|---|
| 7 | GS (45aa) | GGGGSGGGGSGGGSGSKPIPNPLLGLDSTGGGSGSGGGGSGGGGS |
| 8 | GSTMB (45aa) | GGGGSGGGGSLVPRGSKPIPNPLLGLDSTLVPRGSGGGGSGGGGS |
| 9 | GSTPS1 (45aa) | GGGGSGGGGSGGGSGLSGRSDNHSPLGLAGSGGGSGGGGSGGGGS |
| | (also | |
| | referred to | |
| | as TPS1 | |
| | (45aa); same | |
| | sequence as | |
| | TPS3 (45aa) | |
| | and TPS2 | |
| | (45aa)) | |
| 10 | GSTPS2 (55aa) | GGGGSLSGRSDNHSPLGLAGSKPIPNPLLGLDSTLSGRSDNHSPLGLAGSGGGG |
| | (also | S |
| | referred to | |
| | as “TPS2 | |
| | (55aa)”) | |
| 99 | TPS4 (25aa) | GGGGSVHMPLGFLGPRQARVVNGGS |
| 100 | TPS5 (25aa) | GGGGSVHMPLGFLGPGSARVVNGGS |
| 101 | TPS6 (25aa) | GGGGSGGGGSPMAKKGGGGSGGGGS |
| 102 | TPS7 (25aa) | GGGGSGGGGSPMAKGSGGGSGGGGS |
| 107 | TPS4 (45aa) | GGGGSGGGGSGGGGSVHMPLGFLGPRQARVVNGGSGGGGSGGGGS |
| 172 | TPS8 (25aa) | GSSPLGLAGSGGGGSGGGGSPMAKK |
| 173 | TPS9 (25aa) | GGSVHMPLGFLGPGGSGGGSPMAKK |
| 174 | TPS10 (25aa) | GGSPLGVRGKGGGGSGGGGSPMAKK |
| 175 | TPS11 (25aa) | GGSSPLGLAGSGSGGGSRQARVVNG |
| 176 | TPS12 (25aa) | GGSVHMPLGFLGPGGGSRQARVVNG |
| 177 | TPS13 (25aa) | GGSPLGVRGKGGSGGGSRQARVVNG |
| 108 | TPS5 45aa | GGGGSGGGGSGGGGSVHMPLGFLGPGSARVVNGGSGGGGSGGGGS |
| 109 | TPS6 20aa | GGGGSPMAKKGGGGSGGGGS |
| 110 | TPS6 25aa | GGGGSGGGGSPMAKKGGGGSGGGGS |
| 111 | TPS6 30aa | GGGGSGGGGSPMAKKGGGGSGGGGSGGGGS |
| 112 | TPS6 35aa | GGGGSGGGGSGGGGSPMAKKGGGGSGGGGSGGGGS |
| 113 | TPS6 45aa | GGGGSGGGGSGGGGSGGGGSPMAKKGGGGSGGGGSGGGGSGGGGS |
| 114 | TPS7 45aa | GGGGSGGGGSGGGGSGGGGSPMAKGSGGGSGGGGSGGGGSGGGGS |
| 115 | TPS8 45aa | GGGGSGGGGSGSSPLGLAGSGGGGSGGGGSPMAKKGGGGSGGGGS |
| 116 | TPS9 45aa | GGGGSGGGGSGGSVHMPLGFLGPGGSGGGSPMAKKGGGGSGGGGS |
| 117 | TPS10 45aa | GGGGSGGGGSGGSPLGVRGKGGGGSGGGGSPMAKKGGGGSGGGGS |
| 118 | TPS11 45aa | GGGGSGGGGSGGSSPLGLAGSGSGGGSRQARVVNGGGGGSGGGGS |
| 119 | TPS12 45aa | GGGGSGGGGSGGSVHMPLGFLGPGGGSRQARVVNGGGGGSGGGGS |
| 120 | TPS13 45aa | GGGGSGGGGSGGSPLGVRGKGGSGGGSRQARVVNGGGGGSGGGGS |

Exemplary protease-activating CD45-gate MUC16-specific CARs

| | | |
|---|---|---|
| 121 | N-HA- | MALPVTALLLPLALLLHAARPGYPYDVPDYADIVMTQTPASVSEPVGGSVTIKC |
| | 4131scFv- | QASQSFYNLLAWYQQKPGQPPKLLIYDASDLASGVPSRFKGSGSGTDFTLTISD |
| | TPS4-53B6 | LECADAAAYYCQSADGSSYAFGGGTEVVVKGGGGGSGGGGSGGGGSGGGGSQEQ |
| | | LEESGGGLVKPEGSLTLTCTASGVSFSSSYWIYWVRQAPGKGLEWIACIYTGSS |
| | | GSTYYASWAKGRFTVSETSSTTVTLQMTSLTAADTATYFCARASAWTYGMDLWG |
| | | PGTLVTVSSGGGGSGGGGSGGGGSVHMPLGFLGPRQARVVNGGSGGGGSGGGGS |
| | | QVQLQESGPGLVKPSETLSLTCTVSGGSISYYSWTWVRQPAGQGLEWIGRIHIS |
| | | GVTNHNPSLKSRVSMSIDTSRTQFSLRLTSVTAADTAVYFCARSGGTYSAFDIW |
| | | GQGTMVTVSSGGGGSGGGGSGGGGSGGGGSEIVLTQSPGTLSLSPGERSTLSCR |
| | | ASQSFTSNYLAWYQQRPGQAPRLLIYGASTRAIGIPDRFSGSGSGTDFTLTISR |
| | | LEPEDFAVYYCQQYGSSPWTFGQGTKVEIKTTTPAPRPPTPAPTIASQPLSLRP |
| | | EACRPAAGGAVHTRGLDFACDIYIWAPLAGTCGVLLLSLVITLYCKRGRKKLLY |
| | | IFKQPFMRPVQTTQEEDGCSCRFPEEEEGGCELRVKFSRSADAPAYQQGQNQLY |
| | | NELNLGRREEYDVLDKRRGRDPEMGGKPRRKNPQEGLYNELQKDKMAEAYSEIG |
| | | MKGERRRGKGHDGLYQGLSTATKDTYDALHMQALPPR |
| 122 | N-HA- | MALPVTALLLPLALLLHAARPGYPYDVPDYADIVMTQTPASVSEPVGGSVTIKC |
| | 4131scFv- | QASQSFYNLLAWYQQKPGQPPKLLIYDASDLASGVPSRFKGSGSGTDFTLTISD |
| | TPS5-53B6 | LECADAAAYYCQSADGSSYAFGGGTEVVVKGGGGGSGGGGSGGGGSGGGGSQEQ |
| | | LEESGGGLVKPEGSLTLTCTASGVSFSSSYWIYWVRQAPGKGLEWIACIYTGSS |
| | | GSTYYASWAKGRFTVSETSSTTVTLQMTSLTAADTATYFCARASAWTYGMDLWG |
| | | PGTLVTVSSGGGGSGGGGSGGGGSVHMPLGFLGPGSARVVNGGSGGGGSGGGGS |
| | | QVQLQESGPGLVKPSETLSLTCTVSGGSISYYSWTWVRQPAGQGLEWIGRIHIS |
| | | GVTNHNPSLKSRVSMSIDTSRTQFSLRLTSVTAADTAVYFCARSGGTYSAFDIW |
| | | GQGTMVTVSSGGGGSGGGGSGGGGSGGGGSEIVLTQSPGTLSLSPGERSTLSCR |
| | | ASQSFTSNYLAWYQQRPGQAPRLLIYGASTRAIGIPDRFSGSGSGTDFTLTISR |
| | | LEPEDFAVYYCQQYGSSPWTFGQGTKVEIKTTTPAPRPPTPAPTIASQPLSLRP |
| | | EACRPAAGGAVHTRGLDFACDIYIWAPLAGTCGVLLLSLVITLYCKRGRKKLLY |
| | | IFKQPFMRPVQTTQEEDGCSCRFPEEEEGGCELRVKFSRSADAPAYQQGQNQLY |
| | | NELNLGRREEYDVLDKRRGRDPEMGGKPRRKNPQEGLYNELQKDKMAEAYSEIG |
| | | MKGERRRGKGHDGLYQGLSTATKDTYDALHMQALPPR |
| 123 | N-HA- | MALPVTALLLPLALLLHAARPGYPYDVPDYADIVMTQTPASVSEPVGGSVTIKC |
| | 4131scFv- | QASQSFYNLLAWYQQKPGQPPKLLIYDASDLASGVPSRFKGSGSGTDFTLTISD |
| | TPS6-53B6 | LECADAAAYYCQSADGSSYAFGGGTEVVVKGGGGGSGGGGSGGGGSGGGGSQEQ |
| | | LEESGGGLVKPEGSLTLTCTASGVSFSSSYWIYWVRQAPGKGLEWIACIYTGSS |
| | | GSTYYASWAKGRFTVSETSSTTVTLQMTSLTAADTATYFCARASAWTYGMDLWG |
| | | PGTLVTVSSGGGGSGGGGSGGGGSGGGGSPMAKKGGGGSGGGGSGGGGSGGGGS |
| | | QVQLQESGPGLVKPSETLSLTCTVSGGSISYYSWTWVRQPAGQGLEWIGRIHIS |
| | | GVTNHNPSLKSRVSMSIDTSRTQFSLRLTSVTAADTAVYFCARSGGTYSAFDIW |
| | | GQGTMVTVSSGGGGSGGGGSGGGGSGGGGSEIVLTQSPGTLSLSPGERSTLSCR |
| | | ASQSFTSNYLAWYQQRPGQAPRLLIYGASTRAIGIPDRFSGSGSGTDFTLTISR |
| | | LEPEDFAVYYCQQYGSSPWTFGQGTKVEIKTTTPAPRPPTPAPTIASQPLSLRP |
| | | EACRPAAGGAVHTRGLDFACDIYIWAPLAGTCGVLLLSLVITLYCKRGRKKLLY |
| | | IFKQPFMRPVQTTQEEDGCSCRFPEEEEGGCELRVKFSRSADAPAYQQGQNQLY |
| | | NELNLGRREEYDVLDKRRGRDPEMGGKPRRKNPQEGLYNELQKDKMAEAYSEIG |
| | | MKGERRRGKGHDGLYQGLSTATKDTYDALHMQALPPR |
| 124 | N-HA- | MALPVTALLLPLALLLHAARPGYPYDVPDYADIVMTQTPASVSEPVGGSVTIKC |
| | 4131scFv- | QASQSFYNLLAWYQQKPGQPPKLLIYDASDLASGVPSRFKGSGSGTDFTLTISD |
| | TPS7-53B6 | LECADAAAYYCQSADGSSYAFGGGTEVVVKGGGGGSGGGGSGGGGSGGGGSQEQ |
| | | LEESGGGLVKPEGSLTLTCTASGVSFSSSYWIYWVRQAPGKGLEWIACIYTGSS |
| | | GSTYYASWAKGRFTVSETSSTTVTLQMTSLTAADTATYFCARASAWTYGMDLWG |
| | | PGTLVTVSSGGGGSGGGGSGGGGSGGGGSPMAKGSGGGSGGGGSGGGGSGGGGS |
| | | QVQLQESGPGLVKPSETLSLTCTVSGGSISYYSWTWVRQPAGQGLEWIGRIHIS |
| | | GVTNHNPSLKSRVSMSIDTSRTQFSLRLTSVTAADTAVYFCARSGGTYSAFDIW |
| | | GQGTMVTVSSGGGGSGGGGSGGGGSGGGGSEIVLTQSPGTLSLSPGERSTLSCR |
| | | ASQSFTSNYLAWYQQRPGQAPRLLIYGASTRAIGIPDRFSGSGSGTDFTLTISR |
| | | LEPEDFAVYYCQQYGSSPWTFGQGTKVEIKTTTPAPRPPTPAPTIASQPLSLRP |
| | | EACRPAAGGAVHTRGLDFACDIYIWAPLAGTCGVLLLSLVITLYCKRGRKKLLY |
| | | IFKQPFMRPVQTTQEEDGCSCRFPEEEEGGCELRVKFSRSADAPAYQQGQNQLY |
| | | NELNLGRREEYDVLDKRRGRDPEMGGKPRRKNPQEGLYNELQKDKMAEAYSEIG |
| | | MKGERRRGKGHDGLYQGLSTATKDTYDALHMQALPPR |
| 125 | N-HA- | MALPVTALLLPLALLLHAARPGYPYDVPDYADIVMTQTPASVSEPVGGSVTIKC |
| | 4131scFv- | QASQSFYNLLAWYQQKPGQPPKLLIYDASDLASGVPSRFKGSGSGTDFTLTISD |
| | 2XTPS4-53B6 | LECADAAAYYCQSADGSSYAFGGGTEVVVKGGGGGSGGGGSVHMPLGFLGPRQA |
| | | RVVNGGSGGGGSQEQLEESGGGLVKPEGSLTLTCTASGVSFSSSYWIYWVRQAP |
| | | GKGLEWIACIYTGSSGSTYYASWAKGRFTVSETSSTTVTLQMTSLTAADTATYF |
| | | CARASAWTYGMDLWGPGTLVTVSSGGGGSGGGGSGGGGSVHMPLGFLGPRQARV |
| | | VNGGSGGGGSGGGGSQVQLQESGPGLVKPSETLSLTCTVSGGSISYYSWTWVRQ |
| | | PAGQGLEWIGRIHISGVTNHNPSLKSRVSMSIDTSRTQFSLRLTSVTAADTAVY |
| | | FCARSGGTYSAFDIWGQGTMVTVSSGGGGSGGGGSGGGGSGGGGSEIVLTQSPG |
| | | TLSLSPGERSTLSCRASQSFTSNYLAWYQQRPGQAPRLLIYGASTRAIGIPDRF |
| | | SGSGSGTDFTLTISRLEPEDFAVYYCQQYGSSPWTFGQGTKVEIKTTTPAPRPP |
| | | TPAPTIASQPLSLRPEACRPAAGGAVHTRGLDFACDIYIWAPLAGTCGVLLLSL |
| | | VITLYCKRGRKKLLYIFKQPFMRPVQTTQEEDGCSCRFPEEEEGGCELRVKFSR |
| | | SADAPAYQQGQNQLYNELNLGRREEYDVLDKRRGRDPEMGGKPRRKNPQEGLYN |
| | | ELQKDKMAEAYSEIGMKGERRRGKGHDGLYQGLSTATKDTYDALHMQALPPR |
| 126 | N-HA- | MALPVTALLLPLALLLHAARPGYPYDVPDYADIVMTQTPASVSEPVGGSVTIKC |
| | 4131scFv- | QASQSFYNLLAWYQQKPGQPPKLLIYDASDLASGVPSRFKGSGSGTDFTLTISD |
| | 2XTPS5-53B6 | LECADAAAYYCQSADGSSYAFGGGTEVVVKGGGGGSGGGGSVHMPLGFLGPGSA |
| | | RVVNGGSGGGGSQEQLEESGGGLVKPEGSLTLTCTASGVSFSSSYWIYWVRQAP |
| | | GKGLEWIACIYTGSSGSTYYASWAKGRFTVSETSSTTVTLQMTSLTAADTATYF |
| | | CARASAWTYGMDLWGPGTLVTVSSGGGGSGGGGSGGGGSVHMPLGFLGPGSARV |
| | | VNGGSGGGGSGGGGSQVQLQESGPGLVKPSETLSLTCTVSGGSISYYSWTWVRQ |
| | | PAGQGLEWIGRIHISGVTNHNPSLKSRVSMSIDTSRTQFSLRLTSVTAADTAVY |
| | | FCARSGGTYSAFDIWGQGTMVTVSSGGGGSGGGGSGGGGSGGGGSEIVLTQSPG |
| | | TLSLSPGERSTLSCRASQSFTSNYLAWYQQRPGQAPRLLIYGASTRAIGIPDRF |
| | | SGSGSGTDFTLTISRLEPEDFAVYYCQQYGSSPWTFGQGTKVEIKTTTPAPRPP |
| | | TPAPTIASQPLSLRPEACRPAAGGAVHTRGLDFACDIYIWAPLAGTCGVLLLSL |
| | | VITLYCKRGRKKLLYIFKQPFMRPVQTTQEEDGCSCRFPEEEEGGCELRVKFSR |
| | | SADAPAYQQGQNQLYNELNLGRREEYDVLDKRRGRDPEMGGKPRRKNPQEGLYN |
| | | ELQKDKMAEAYSEIGMKGERRRGKGHDGLYQGLSTATKDTYDALHMQALPPR |
| 127 | N-HA- | MALPVTALLLPLALLLHAARPGYPYDVPDYADIVMTQTPASVSEPVGGSVTIKC |
| | 4131scFv- | QASQSFYNLLAWYQQKPGQPPKLLIYDASDLASGVPSRFKGSGSGTDFTLTISD |
| | 2XTP56-53B6 | LECADAAAYYCQSADGSSYAFGGGTEVVVKGGGGGSGGGGSGGGGSPMAKKGGG |
| | | GSGGGGSGGGGSQEQLEESGGGLVKPEGSLTLTCTASGVSFSSSYWIYWVRQAP |
| | | GKGLEWIACIYTGSSGSTYYASWAKGRFTVSETSSTTVTLQMTSLTAADTATYF |
| | | CARASAWTYGMDLWGPGTLVTVSSGGGGSGGGGSGGGGSGGGGSPMAKKGGGGS |
| | | GGGGSGGGGSGGGGSQVQLQESGPGLVKPSETLSLTCTVSGGSISYYSWTWVRQ |
| | | PAGQGLEWIGRIHISGVTNHNPSLKSRVSMSIDTSRTQFSLRLTSVTAADTAVY |
| | | FCARSGGTYSAFDIWGQGTMVTVSSGGGGSGGGGSGGGGSGGGGSEIVLTQSPG |
| | | TLSLSPGERSTLSCRASQSFTSNYLAWYQQRPGQAPRLLIYGASTRAIGIPDRF |
| | | SGSGSGTDFTLTISRLEPEDFAVYYCQQYGSSPWTFGQGTKVEIKTTTPAPRPP |
| | | TPAPTIASQPLSLRPEACRPAAGGAVHTRGLDFACDIYIWAPLAGTCGVLLLSL |
| | | VITLYCKRGRKKLLYIFKQPFMRPVQTTQEEDGCSCRFPEEEEGGCELRVKFSR |
| | | SADAPAYQQGQNQLYNELNLGRREEYDVLDKRRGRDPEMGGKPRRKNPQEGLYN |
| | | ELQKDKMAEAYSEIGMKGERRRGKGHDGLYQGLSTATKDTYDALHMQALPPR |
| 128 | N-HA- | MALPVTALLLPLALLLHAARPGYPYDVPDYADIVMTQTPASVSEPVGGSVTIKC |
| | 4131scFv- | QASQSFYNLLAWYQQKPGQPPKLLIYDASDLASGVPSRFKGSGSGTDFTLTISD |
| | 2XTP57-53B6 | LECADAAAYYCQSADGSSYAFGGGTEVVVKGGGGGSGGGGSGGGGSPMAKGSGG |
| | | GSGGGGSGGGGSQEQLEESGGGLVKPEGSLTLTCTASGVSFSSSYWIYWVRQAP |
| | | GKGLEWIACIYTGSSGSTYYASWAKGRFTVSETSSTTVTLQMTSLTAADTATYF |
| | | CARASAWTYGMDLWGPGTLVTVSSGGGGSGGGGSGGGGSGGGGSPMAKGSGGGS |
| | | GGGGSGGGGSGGGGSQVQLQESGPGLVKPSETLSLTCTVSGGSISYYSWTWVRQ |
| | | PAGQGLEWIGRIHISGVTNHNPSLKSRVSMSIDTSRTQFSLRLTSVTAADTAVY |
| | | FCARSGGTYSAFDIWGQGTMVTVSSGGGGSGGGGSGGGGSGGGGSEIVLTQSPG |
| | | TLSLSPGERSTLSCRASQSFTSNYLAWYQQRPGQAPRLLIYGASTRAIGIPDRF |
| | | SGSGSGTDFTLTISRLEPEDFAVYYCQQYGSSPWTFGQGTKVEIKTTTPAPRPP |
| | | TPAPTIASQPLSLRPEACRPAAGGAVHTRGLDFACDIYIWAPLAGTCGVLLLSL |
| | | VITLYCKRGRKKLLYIFKQPFMRPVQTTQEEDGCSCRFPEEEEGGCELRVKFSR |
| | | SADAPAYQQGQNQLYNELNLGRREEYDVLDKRRGRDPEMGGKPRRKNPQEGLYN |
| | | ELQKDKMAEAYSEIGMKGERRRGKGHDGLYQGLSTATKDTYDALHMQALPPR |
| 129 | N-HA-BTN3A1- | MALPVTALLLPLALLLHAARPGYPYDVPDYAQFSVLGPSGPILAMVGEDADLPC |
| | V5-2G4STMB- | HLFPTMSAETMELKWVSSSLRQVVNVYADGKEVEDRQSAPYRGRTSILRDGITA |
| | 53B6 | GKAALRIHNVTASDSGKYLCYFQDGDFYEKALVELKVAALGSDLHVDVKGYKDG |
| | | GIHLECRSTGWYPQPQIQWSNNKGENIPTVEAPVVADGVGLYAVAASVIMRGSS |
| | | GEGVSCTIRSSLLGLEKTASISIADPFFRSAQRWIAALAGTGGGGSGGGGSLVP |
| | | RGSKPIPNPLLGLDSTLVPRGSGGGGSGGGGSQVQLQESGPGLVKPSETLSLTC |
| | | TVSGGSISYYSWTWVRQPAGQGLEWIGRIHISGVTNHNPSLKSRVSMSIDTSRT |
| | | QFSLRLTSVTAADTAVYFCARSGGTYSAFDIWGQGTMVTVSSGGGGSGGGGSGG |
| | | GGSGGGGSEIVLTQSPGTLSLSPGERSTLSCRASQSFTSNYLAWYQQRPGQAPR |
| | | LLIYGASTRAIGIPDRFSGSGSGTDFTLTISRLEPEDFAVYYCQQYGSSPWTFG |
| | | QGTKVEIKTTTPAPRPPTPAPTIASQPLSLRPEACRPAAGGAVHTRGLDFACDI |
| | | YIWAPLAGTCGVLLLSLVITLYCKRGRKKLLYIFKQPFMRPVQTTQEEDGCSCR |
| | | FPEEEEGGCELRVKFSRSADAPAYQQGQNQLYNELNLGRREEYDVLDKRRGRDP |
| | | EMGGKPRRKNPQEGLYNELQKDKMAEAYSEIGMKGERRRGKGHDGLYQGLSTAT |
| | | KDTYDALHMQALPPR |
| 130 | N-HA-RORB-V5- | MALPVTALLLPLALLLHAARPGYPYDVPDYADILLTQSPATLSLSPGERATLSC |
| | 2G4STMB-53B6 | RASQNIGTSIQWYQQKPGQAPRLLIRSSSESISGIPSRFSGSGSGTDFTLTISS |
| | | LEPEDFAVYYCQQSNTWPFTFGQGTKLEIKGGGGSGGGGSGGGGSGGGGSEVQL |
| | | VESGAEVKKPGASVKVSCKASGYTFTNYIIHWVKQEPGQGLEWIGYFNPYNHGT |
| | | KYNEKFKGRATLTADKSISTAYMELSSLRSEDTAVYYCARSGPYAWFDTWGQGT |
| | | TVTVSSGGGGSGGGGSLVPRGSKPIPNPLLGLDSTLVPRGSGGGGSGGGGSQVQ |
| | | LQESGPGLVKPSETLSLTCTVSGGSISYYSWTWVRQPAGQGLEWIGRIHISGVT |
| | | NHNPSLKSRVSMSIDTSRTQFSLRLTSVTAADTAVYFCARSGGTYSAFDIWGQG |
| | | TMVTVSSGGGGSGGGGSGGGGSGGGGSEIVLTQSPGTLSLSPGERSTLSCRASQ |
| | | SFTSNYLAWYQQRPGQAPRLLIYGASTRAIGIPDRFSGSGSGTDFTLTISRLEP |
| | | EDFAVYYCQQYGSSPWTFGQGTKVEIKTTTPAPRPPTPAPTIASQPLSLRPEAC |
| | | RPAAGGAVHTRGLDFACDIYIWAPLAGTCGVLLLSLVITLYCKRGRKKLLYIFK |
| | | QPFMRPVQTTQEEDGCSCRFPEEEEGGCELRVKFSRSADAPAYQQGQNQLYNEL |
| | | NLGRREEYDVLDKRRGRDPEMGGKPRRKNPQEGLYNELQKDKMAEAYSEIGMKG |
| | | ERRRGKGHDGLYQGLSTATKDTYDALHMQALPPR |
| 131 | N-HA-RORB-V5- | MALPVTALLLPLALLLHAARPGYPYDVPDYADILLTQSPATLSLSPGERATLSC |
| | 3G45-53B6 | RASQNIGTSIQWYQQKPGQAPRLLIRSSSESISGIPSRFSGSGSGTDFTLTISS |
| | | LEPEDFAVYYCQQSNTWPFTFGQGTKLEIKGGGGSGGGGSGGGGSGGGGSEVQL |
| | | VESGAEVKKPGASVKVSCKASGYTFTNYIIHWVKQEPGQGLEWIGYFNPYNHGT |
| | | KYNEKFKGRATLTADKSISTAYMELSSLRSEDTAVYYCARSGPYAWFDTWGQGT |
| | | TVTVSSGGGGSGGGGSGGGSGSKPIPNPLLGLDSTGGGSGSGGGGSGGGGSQVQ |
| | | LQESGPGLVKPSETLSLTCTVSGGSISYYSWTWVRQPAGQGLEWIGRIHISGVT |
| | | NHNPSLKSRVSMSIDTSRTQFSLRLTSVTAADTAVYFCARSGGTYSAFDIWGQG |
| | | TMVTVSSGGGGSGGGGSGGGGSGGGGSEIVLTQSPGTLSLSPGERSTLSCRASQ |
| | | SFTSNYLAWYQQRPGQAPRLLIYGASTRAIGIPDRFSGSGSGTDFTLTISRLEP |
| | | EDFAVYYCQQYGSSPWTFGQGTKVEIKTTTPAPRPPTPAPTIASQPLSLRPEAC |
| | | RPAAGGAVHTRGLDFACDIYIWAPLAGTCGVLLLSLVITLYCKRGRKKLLYIFK |
| | | QPFMRPVQTTQEEDGCSCRFPEEEEGGCELRVKFSRSADAPAYQQGQNQLYNEL |
| | | NLGRREEYDVLDKRRGRDPEMGGKPRRKNPQEGLYNELQKDKMAEAYSEIGMKG |
| | | ERRRGKGHDGLYQGLSTATKDTYDALHMQALPPR |
| 132 | N-HA-RORB- | MALPVTALLLPLALLLHAARPGYPYDVPDYADILLTQSPATLSLSPGERATLSC |
| | TPS4-53B6 | RASQNIGTSIQWYQQKPGQAPRLLIRSSSESISGIPSRFSGSGSGTDFTLTISS |
| | | LEPEDFAVYYCQQSNTWPFTFGQGTKLEIKGGGGSGGGGSGGGGSGGGGSEVQL |
| | | VESGAEVKKPGASVKVSCKASGYTFTNYIIHWVKQEPGQGLEWIGYFNPYNHGT |
| | | KYNEKFKGRATLTADKSISTAYMELSSLRSEDTAVYYCARSGPYAWFDTWGQGT |
| | | TVTVSSGGGGSGGGGSGGGGSVHMPLGELGPRQARVVNGGSGGGGSGGGGSQVQ |
| | | LQESGPGLVKPSETLSLTCTVSGGSISYYSWTWVRQPAGQGLEWIGRIHISGVT |
| | | NHNPSLKSRVSMSIDTSRTQFSLRLTSVTAADTAVYFCARSGGTYSAFDIWGQG |
| | | TMVTVSSGGGGSGGGGSGGGGSGGGGSEIVLTQSPGTLSLSPGERSTLSCRASQ |
| | | SFTSNYLAWYQQRPGQAPRLLIYGASTRAIGIPDRFSGSGSGTDFTLTISRLEP |
| | | EDFAVYYCQQYGSSPWTFGQGTKVEIKTTTPAPRPPTPAPTIASQPLSLRPEAC |
| | | RPAAGGAVHTRGLDFACDIYIWAPLAGTCGVLLLSLVITLYCKRGRKKLLYIFK |
| | | QPFMRPVQTTQEEDGCSCRFPEEEEGGCELRVKFSRSADAPAYQQGQNQLYNEL |
| | | NLGRREEYDVLDKRRGRDPEMGGKPRRKNPQEGLYNELQKDKMAEAYSEIGMKG |
| | | ERRRGKGHDGLYQGLSTATKDTYDALHMQALPPR |
| 133 | N-HA-RORB- | MALPVTALLLPLALLLHAARPGYPYDVPDYADILLTQSPATLSLSPGERATLSC |
| | TPS6-53B6 | RASQNIGTSIQWYQQKPGQAPRLLIRSSSESISGIPSRFSGSGSGTDFTLTISS |
| | | LEPEDFAVYYCQQSNTWPFTFGQGTKLEIKGGGGSGGGGSGGGGSGGGGSEVQL |
| | | VESGAEVKKPGASVKVSCKASGYTFTNYIIHWVKQEPGQGLEWIGYFNPYNHGT |
| | | KYNEKFKGRATLTADKSISTAYMELSSLRSEDTAVYYCARSGPYAWFDTWGQGT |
| | | TVTVSSGGGGSGGGGSGGGGSGGGGSPMAKKGGGGSGGGGSGGGGSGGGGSQVQ |
| | | LQESGPGLVKPSETLSLTCTVSGGSISYYSWTWVRQPAGQGLEWIGRIHISGVT |
| | | NHNPSLKSRVSMSIDTSRTQFSLRLTSVTAADTAVYFCARSGGTYSAFDIWGQG |
| | | TMVTVSSGGGGSGGGGSGGGGSGGGGSEIVLTQSPGTLSLSPGERSTLSCRASQ |
| | | SFTSNYLAWYQQRPGQAPRLLIYGASTRAIGIPDRFSGSGSGTDFTLTISRLEP |
| | | EDFAVYYCQQYGSSPWTFGQGTKVEIKTTTPAPRPPTPAPTIASQPLSLRPEAC |
| | | RPAAGGAVHTRGLDFACDIYIWAPLAGTCGVLLLSLVITLYCKRGRKKLLYIFK |
| | | QPFMRPVQTTQEEDGCSCRFPEEEEGGCELRVKFSRSADAPAYQQGQNQLYNEL |
| | | NLGRREEYDVLDKRRGRDPEMGGKPRRKNPQEGLYNELQKDKMAEAYSEIGMKG |
| | | ERRRGKGHDGLYQGLSTATKDTYDALHMQALPPR |
| 134 | N-HA-RORB- | MALPVTALLLPLALLLHAARPGYPYDVPDYADILLTQSPATLSLSPGERATLSC |
| | 2XTP54-53B6 | RASQNIGTSIQWYQQKPGQAPRLLIRSSSESISGIPSRFSGSGSGTDFTLTISS |
| | | LEPEDFAVYYCQQSNTWPFTFGQGTKLEIKGGGGSGGGGSVHMPLGFLGPRQAR |
| | | VVNGGSGGGGSEVQLVESGAEVKKPGASVKVSCKASGYTFTNYIIHWVKQEPGQ |
| | | GLEWIGYFNPYNHGTKYNEKFKGRATLTADKSISTAYMELSSLRSEDTAVYYCA |
| | | RSGPYAWFDTWGQGTTVTVSSGGGGSGGGGSGGGGSVHMPLGELGPRQARVVNG |
| | | GSGGGGSGGGGSQVQLQESGPGLVKPSETLSLTCTVSGGSISYYSWTWVRQPAG |
| | | QGLEWIGRIHISGVTNHNPSLKSRVSMSIDTSRTQFSLRLTSVTAADTAVYFCA |
| | | RSGGTYSAFDIWGQGTMVTVSSGGGGSGGGGSGGGGSGGGGSEIVLTQSPGTLS |
| | | LSPGERSTLSCRASQSFTSNYLAWYQQRPGQAPRLLIYGASTRAIGIPDRFSGS |
| | | GSGTDFTLTISRLEPEDFAVYYCQQYGSSPWTFGQGTKVEIKTTTPAPRPPTPA |
| | | PTIASQPLSLRPEACRPAAGGAVHTRGLDFACDIYIWAPLAGTCGVLLLSLVIT |
| | | LYCKRGRKKLLYIFKQPFMRPVQTTQEEDGCSCRFPEEEEGGCELRVKFSRSAD |
| | | APAYQQGQNQLYNELNLGRREEYDVLDKRRGRDPEMGGKPRRKNPQEGLYNELQ |
| | | KDKMAEAYSEIGMKGERRRGKGHDGLYQGLSTATKDTYDALHMQALPPR |
| 135 | N-HA-RORB- | MALPVTALLLPLALLLHAARPGYPYDVPDYADILLTQSPATLSLSPGERATLSC |
| | 2XTP56-53B6 | RASQNIGTSIQWYQQKPGQAPRLLIRSSSESISGIPSRFSGSGSGTDFTLTISS |
| | | LEPEDFAVYYCQQSNTWPFTFGQGTKLEIKGGGGSGGGGSGGGGSPMAKKGGGG |
| | | SGGGGSGGGGSEVQLVESGAEVKKPGASVKVSCKASGYTFTNYIIHWVKQEPGQ |
| | | GLEWIGYFNPYNHGTKYNEKFKGRATLTADKSISTAYMELSSLRSEDTAVYYCA |
| | | RSGPYAWFDTWGQGTTVTVSSGGGGSGGGGSGGGGSGGGGSPMAKKGGGGSGGG |
| | | GSGGGGSGGGGSQVQLQESGPGLVKPSETLSLTCTVSGGSISYYSWTWVRQPAG |
| | | QGLEWIGRIHISGVTNHNPSLKSRVSMSIDTSRTQFSLRLTSVTAADTAVYFCA |
| | | RSGGTYSAFDIWGQGTMVTVSSGGGGSGGGGSGGGGSGGGGSEIVLTQSPGTLS |
| | | LSPGERSTLSCRASQSFTSNYLAWYQQRPGQAPRLLIYGASTRAIGIPDRFSGS |
| | | GSGTDFTLTISRLEPEDFAVYYCQQYGSSPWTFGQGTKVEIKTTTPAPRPPTPA |
| | | PTIASQPLSLRPEACRPAAGGAVHTRGLDFACDIYIWAPLAGTCGVLLLSLVIT |
| | | LYCKRGRKKLLYIFKQPFMRPVQTTQEEDGCSCRFPEEEEGGCELRVKFSRSAD |
| | | APAYQQGQNQLYNELNLGRREEYDVLDKRRGRDPEMGGKPRRKNPQEGLYNELQ |
| | | KDKMAEAYSEIGMKGERRRGKGHDGLYQGLSTATKDTYDALHMQALPPR |
| 136 | V5-GS20- | MALPVTALLLPLALLLHAARPGKPIPNPLLGLDSTGGGGSGGGGSGGGGSGGGG |
| | 4131scFv- | SDIVMTQTPASVSEPVGGSVTIKCQASQSFYNLLAWYQQKPGQPPKLLIYDASD |
| | TPS1-53B6 | LASGVPSRFKGSGSGTDFTLTISDLECADAAAYYCQSADGSSYAFGGGTEVVVK |
| | | GGGGGSGGGGSGGGGSGGGGSQEQLEESGGGLVKPEGSLTLTCTASGVSFSSSY |
| | | WIYWVRQAPGKGLEWIACIYTGSSGSTYYASWAKGRFTVSETSSTTVTLQMTSL |
| | | TAADTATYFCARASAWTYGMDLWGPGTLVTVSSGGGGSGGGGSGGGSGLSGRSD |
| | | NHSPLGLAGSGGGSGGGGSGGGGSQVQLQESGPGLVKPSETLSLTCTVSGGSIS |
| | | YYSWTWVRQPAGQGLEWIGRIHISGVTNHNPSLKSRVSMSIDTSRTQFSLRLTS |
| | | VTAADTAVYFCARSGGTYSAFDIWGQGTMVTVSSGGGGSGGGGSGGGGSGGGGS |
| | | EIVLTQSPGTLSLSPGERSTLSCRASQSFTSNYLAWYQQRPGQAPRLLIYGAST |
| | | RAIGIPDRFSGSGSGTDFTLTISRLEPEDFAVYYCQQYGSSPWTFGQGTKVEIK |
| | | TTTPAPRPPTPAPTIASQPLSLRPEACRPAAGGAVHTRGLDFACDIYIWAPLAG |
| | | TCGVLLLSLVITLYCKRGRKKLLYIFKQPFMRPVQTTQEEDGCSCRFPEEEEGG |
| | | CELRVKFSRSADAPAYQQGQNQLYNELNLGRREEYDVLDKRRGRDPEMGGKPRR |
| | | KNPQEGLYNELQKDKMAEAYSEIGMKGERRRGKGHDGLYQGLSTATKDTYDALH |
| | | MQALPPR |
| 137 | V5-GS20- | MALPVTALLLPLALLLHAARPGKPIPNPLLGLDSTGGGGSGGGGSGGGGSGGGG |
| | 4131scFv- | SDIVMTQTPASVSEPVGGSVTIKCQASQSFYNLLAWYQQKPGQPPKLLIYDASD |
| | TPS4-53B6 | LASGVPSRFKGSGSGTDFTLTISDLECADAAAYYCQSADGSSYAFGGGTEVVVK |
| | | GGGGGSGGGGSGGGGSGGGGSQEQLEESGGGLVKPEGSLTLTCTASGVSFSSSY |
| | | WIYWVRQAPGKGLEWIACIYTGSSGSTYYASWAKGRFTVSETSSTTVTLQMTSL |
| | | TAADTATYFCARASAWTYGMDLWGPGTLVTVSSGGGGSGGGGSGGGGSVHMPLG |
| | | FLGPRQARVVNGGSGGGGSGGGGSQVQLQESGPGLVKPSETLSLTCTVSGGSIS |
| | | YYSWTWVRQPAGQGLEWIGRIHISGVTNHNPSLKSRVSMSIDTSRTQFSLRLTS |
| | | VTAADTAVYFCARSGGTYSAFDIWGQGTMVTVSSGGGGSGGGGSGGGGSGGGGS |
| | | EIVLTQSPGTLSLSPGERSTLSCRASQSFTSNYLAWYQQRPGQAPRLLIYGAST |
| | | RAIGIPDRFSGSGSGTDFTLTISRLEPEDFAVYYCQQYGSSPWTFGQGTKVEIK |
| | | TTTPAPRPPTPAPTIASQPLSLRPEACRPAAGGAVHTRGLDFACDIYIWAPLAG |
| | | TCGVLLLSLVITLYCKRGRKKLLYIFKQPFMRPVQTTQEEDGCSCRFPEEEEGG |
| | | CELRVKFSRSADAPAYQQGQNQLYNELNLGRREEYDVLDKRRGRDPEMGGKPRR |
| | | KNPQEGLYNELQKDKMAEAYSEIGMKGERRRGKGHDGLYQGLSTATKDTYDALH |
| | | MQALPPR |
| 138 | V5-G520- | MALPVTALLLPLALLLHAARPGKPIPNPLLGLDSTGGGGSGGGGSGGGGSGGGG |
| | 4131scFv- | SDIVMTQTPASVSEPVGGSVTIKCQASQSFYNLLAWYQQKPGQPPKLLIYDASD |
| | TPS6-53B6 | LASGVPSRFKGSGSGTDFTLTISDLECADAAAYYCQSADGSSYAFGGGTEVVVK |
| | | GGGGGSGGGGSGGGGSGGGGSQEQLEESGGGLVKPEGSLTLTCTASGVSFSSSY |
| | | WIYWVRQAPGKGLEWIACIYTGSSGSTYYASWAKGRFTVSETSSTTVTLQMTSL |
| | | TAADTATYFCARASAWTYGMDLWGPGTLVTVSSGGGGSGGGGSGGGGSGGGGSP |
| | | MAKKGGGGSGGGGSGGGGSGGGGSQVQLQESGPGLVKPSETLSLTCTVSGGSIS |
| | | YYSWTWVRQPAGQGLEWIGRIHISGVTNHNPSLKSRVSMSIDTSRTQFSLRLTS |
| | | VTAADTAVYFCARSGGTYSAFDIWGQGTMVTVSSGGGGSGGGGSGGGGSGGGGS |
| | | EIVLTQSPGTLSLSPGERSTLSCRASQSFTSNYLAWYQQRPGQAPRLLIYGAST |
| | | RAIGIPDRFSGSGSGTDFTLTISRLEPEDFAVYYCQQYGSSPWTFGQGTKVEIK |
| | | TTTPAPRPPTPAPTIASQPLSLRPEACRPAAGGAVHTRGLDFACDIYIWAPLAG |
| | | TCGVLLLSLVITLYCKRGRKKLLYIFKQPFMRPVQTTQEEDGCSCRFPEEEEGG |
| | | CELRVKFSRSADAPAYQQGQNQLYNELNLGRREEYDVLDKRRGRDPEMGGKPRR |
| | | KNPQEGLYNELQKDKMAEAYSEIGMKGERRRGKGHDGLYQGLSTATKDTYDALH |
| | | MQALPPR |
| 139 | V5-G520- | MALPVTALLLPLALLLHAARPGKPIPNPLLGLDSTGGGGSGGGGSGGGGSGGGG |
| | 4131scFv- | SDIVMTQTPASVSEPVGGSVTIKCQASQSFYNLLAWYQQKPGQPPKLLIYDASD |
| | 2XTPS6-53B6 | LASGVPSRFKGSGSGTDFTLTISDLECADAAAYYCQSADGSSYAFGGGTEVVVK |
| | | GGGGGSGGGGSGGGGSPMAKKGGGGSGGGGSGGGGSQEQLEESGGGLVKPEGSL |
| | | TLTCTASGVSFSSSYWIYWVRQAPGKGLEWIACIYTGSSGSTYYASWAKGRFTV |
| | | SETSSTTVTLQMTSLTAADTATYFCARASAWTYGMDLWGPGTLVTVSSGGGGSG |
| | | GGGSGGGGSGGGGSPMAKKGGGGSGGGGSGGGGSGGGGSQVQLQESGPGLVKPS |
| | | ETLSLTCTVSGGSISYYSWTWVRQPAGQGLEWIGRIHISGVTNHNPSLKSRVSM |
| | | SIDTSRTQFSLRLTSVTAADTAVYFCARSGGTYSAFDIWGQGTMVTVSSGGGGS |
| | | GGGGSGGGGSGGGGSEIVLTQSPGTLSLSPGERSTLSCRASQSFTSNYLAWYQQ |
| | | RPGQAPRLLIYGASTRAIGIPDRFSGSGSGTDFTLTISRLEPEDFAVYYCQQYG |
| | | SSPWTFGQGTKVEIKTTTPAPRPPTPAPTIASQPLSLRPEACRPAAGGAVHTRG |
| | | LDFACDIYIWAPLAGTCGVLLLSLVITLYCKRGRKKLLYIFKQPFMRPVQTTQE |
| | | EDGCSCRFPEEEEGGCELRVKFSRSADAPAYQQGQNQLYNELNLGRREEYDVLD |
| | | KRRGRDPEMGGKPRRKNPQEGLYNELQKDKMAEAYSEIGMKGERRRGKGHDGLY |
| | | QGLSTATKDTYDALHMQALPPR |
| 140 | V5-GS20- | MALPVTALLLPLALLLHAARPGKPIPNPLLGLDSTGGGGSGGGGSGGGGSGGGG |
| | 4131scFv- | SDIVMTQTPASVSEPVGGSVTIKCQASQSFYNLLAWYQQKPGQPPKLLIYDASD |
| | G545-53B6 | LASGVPSRFKGSGSGTDFTLTISDLECADAAAYYCQSADGSSYAFGGGTEVVVK |
| | | GGGGGSGGGGSGGGGSGGGGSQEQLEESGGGLVKPEGSLTLTCTASGVSFSSSY |
| | | WIYWVRQAPGKGLEWIACIYTGSSGSTYYASWAKGRFTVSETSSTTVTLQMTSL |
| | | TAADTATYFCARASAWTYGMDLWGPGTLVTVSSGGGGSGGGGSGGGSGSGGGGS |
| | | GGGSGGGSGGGSGSGGGGSGGGGSQVQLQESGPGLVKPSETLSLTCTVSGGSIS |
| | | YYSWTWVRQPAGQGLEWIGRIHISGVTNHNPSLKSRVSMSIDTSRTQFSLRLTS |
| | | VTAADTAVYFCARSGGTYSAFDIWGQGTMVTVSSGGGGSGGGGSGGGGSGGGGS |
| | | EIVLTQSPGTLSLSPGERSTLSCRASQSFTSNYLAWYQQRPGQAPRLLIYGAST |
| | | RAIGIPDRFSGSGSGTDFTLTISRLEPEDFAVYYCQQYGSSPWTFGQGTKVEIK |
| | | TTTPAPRPPTPAPTIASQPLSLRPEACRPAAGGAVHTRGLDFACDIYIWAPLAG |
| | | TCGVLLLSLVITLYCKRGRKKLLYIFKQPFMRPVQTTQEEDGCSCRFPEEEEGG |
| | | CELRVKFSRSADAPAYQQGQNQLYNELNLGRREEYDVLDKRRGRDPEMGGKPRR |
| | | KNPQEGLYNELQKDKMAEAYSEIGMKGERRRGKGHDGLYQGLSTATKDTYDALH |
| | | MQALPPR |
| 141 | 4131scFv-V5- | MALPVTALLLPLALLLHAARPGYPYDVPDYADIVMTQTPASVSEPVGGSVTIKC |
| | 2XTPS1-53B6 | QASQSFYNLLAWYQQKPGQPPKLLIYDASDLASGVPSRFKGSGSGTDFTLTISD |
| | | LECADAAAYYCQSADGSSYAFGGGTEVVVKGGGGGSGGGGSLSGRSDNHSPLGL |
| | | AGSGGGGSGGGGSQEQLEESGGGLVKPEGSLTLTCTASGVSFSSSYWIYWVRQA |
| | | PGKGLEWIACIYTGSSGSTYYASWAKGRFTVSETSSTTVTLQMTSLTAADTATY |
| | | FCARASAWTYGMDLWGPGTLVTVSSGGGGSGGGGSKPIPNPLLGLDSTGGGSGL |
| | | SGRSDNHSPLGLAGSGGGSGGGGSGGGGSQVQLQESGPGLVKPSETLSLTCTVS |
| | | GGSISYYSWTWVRQPAGQGLEWIGRIHISGVTNHNPSLKSRVSMSIDTSRTQFS |
| | | LRLTSVTAADTAVYFCARSGGTYSAFDIWGQGTMVTVSSGGGGSGGGGSGGGGS |
| | | GGGGSEIVLTQSPGTLSLSPGERSTLSCRASQSFTSNYLAWYQQRPGQAPRLLI |
| | | YGASTRAIGIPDRFSGSGSGTDFTLTISRLEPEDFAVYYCQQYGSSPWTFGQGT |
| | | KVEIKTTTPAPRPPTPAPTIASQPLSLRPEACRPAAGGAVHTRGLDFACDIYIW |
| | | APLAGTCGVLLLSLVITLYCKRGRKKLLYIFKQPFMRPVQTTQEEDGCSCRFPE |
| | | EEEGGCELRVKFSRSADAPAYQQGQNQLYNELNLGRREEYDVLDKRRGRDPEMG |
| | | GKPRRKNPQEGLYNELQKDKMAEAYSEIGMKGERRRGKGHDGLYQGLSTATKDT |
| | | YDALHMQALPPR |
| 142 | 4131scFv-V5- | MALPVTALLLPLALLLHAARPGYPYDVPDYADIVMTQTPASVSEPVGGSVTIKC |
| | 2XTPS4-53B6 | QASQSFYNLLAWYQQKPGQPPKLLIYDASDLASGVPSRFKGSGSGTDFTLTISD |
| | | LECADAAAYYCQSADGSSYAFGGGTEVVVKGGGGGSGGGGSVHMPLGELGPRQA |
| | | RVVNGGSGGGGSQEQLEESGGGLVKPEGSLTLTCTASGVSFSSSYWIYWVRQAP |
| | | GKGLEWIACIYTGSSGSTYYASWAKGRFTVSETSSTTVTLQMTSLTAADTATYF |
| | | CARASAWTYGMDLWGPGTLVTVSSGGGGSGGGGSKPIPNPLLGLDSTGGGGSVH |
| | | MPLGFLGPRQARVVNGGSGGGGSGGGGSQVQLQESGPGLVKPSETLSLTCTVSG |
| | | GSISYYSWTWVRQPAGQGLEWIGRIHISGVTNHNPSLKSRVSMSIDTSRTQFSL |
| | | RLTSVTAADTAVYFCARSGGTYSAFDIWGQGTMVTVSSGGGGSGGGGSGGGGSG |
| | | GGGSEIVLTQSPGTLSLSPGERSTLSCRASQSFTSNYLAWYQQRPGQAPRLLIY |
| | | GASTRAIGIPDRFSGSGSGTDFTLTISRLEPEDFAVYYCQQYGSSPWTFGQGTK |
| | | VEIKTTTPAPRPPTPAPTIASQPLSLRPEACRPAAGGAVHTRGLDFACDIYIWA |
| | | PLAGTCGVLLLSLVITLYCKRGRKKLLYIFKQPFMRPVQTTQEEDGCSCRFPEE |
| | | EEGGCELRVKFSRSADAPAYQQGQNQLYNELNLGRREEYDVLDKRRGRDPEMGG |
| | | KPRRKNPQEGLYNELQKDKMAEAYSEIGMKGERRRGKGHDGLYQGLSTATKDTY |
| | | DALHMQALPPR |
| 143 | 4131scFv-V5- | MALPVTALLLPLALLLHAARPGYPYDVPDYADIVMTQTPASVSEPVGGSVTIKC |
| | 2XTPS5-53B6 | QASQSFYNLLAWYQQKPGQPPKLLIYDASDLASGVPSRFKGSGSGTDFTLTISD |
| | | LECADAAAYYCQSADGSSYAFGGGTEVVVKGGGGGSGGGGSVHMPLGELGPGSA |
| | | RVVNGGSGGGGSQEQLEESGGGLVKPEGSLTLTCTASGVSFSSSYWIYWVRQAP |
| | | GKGLEWIACIYTGSSGSTYYASWAKGRFTVSETSSTTVTLQMTSLTAADTATYF |
| | | CARASAWTYGMDLWGPGTLVTVSSGGGGSGGGGSKPIPNPLLGLDSTGGGGSVH |
| | | MPLGFLGPGSARVVNGGSGGGGSGGGGSQVQLQESGPGLVKPSETLSLTCTVSG |
| | | GSISYYSWTWVRQPAGQGLEWIGRIHISGVTNHNPSLKSRVSMSIDTSRTQFSL |
| | | RLTSVTAADTAVYFCARSGGTYSAFDIWGQGTMVTVSSGGGGSGGGGSGGGGSG |
| | | GGGSEIVLTQSPGTLSLSPGERSTLSCRASQSFTSNYLAWYQQRPGQAPRLLIY |
| | | GASTRAIGIPDRFSGSGSGTDFTLTISRLEPEDFAVYYCQQYGSSPWTFGQGTK |
| | | VEIKTTTPAPRPPTPAPTIASQPLSLRPEACRPAAGGAVHTRGLDFACDIYIWA |
| | | PLAGTCGVLLLSLVITLYCKRGRKKLLYIFKQPFMRPVQTTQEEDGCSCRFPEE |
| | | EEGGCELRVKFSRSADAPAYQQGQNQLYNELNLGRREEYDVLDKRRGRDPEMGG |
| | | KPRRKNPQEGLYNELQKDKMAEAYSEIGMKGERRRGKGHDGLYQGLSTATKDTY |
| | | DALHMQALPPR |
| 144 | 4131scFv-V5- | MALPVTALLLPLALLLHAARPGYPYDVPDYADIVMTQTPASVSEPVGGSVTIKC |
| | 2XTPS6-53B6 | QASQSFYNLLAWYQQKPGQPPKLLIYDASDLASGVPSRFKGSGSGTDFTLTISD |
| | | LECADAAAYYCQSADGSSYAFGGGTEVVVKGGGGGSGGGGSGGGGSPMAKKGGG |
| | | GSGGGGSGGGGSQEQLEESGGGLVKPEGSLTLTCTASGVSFSSSYWIYWVRQAP |
| | | GKGLEWIACIYTGSSGSTYYASWAKGRFTVSETSSTTVTLQMTSLTAADTATYF |
| | | CARASAWTYGMDLWGPGTLVTVSSGGGGSGGGGSKPIPNPLLGLDSTGGGGSPM |
| | | AKKGGGGSGGGGSGGGGSGGGGSQVQLQESGPGLVKPSETLSLTCTVSGGSISY |
| | | YSWTWVRQPAGQGLEWIGRIHISGVTNHNPSLKSRVSMSIDTSRTQFSLRLTSV |
| | | TAADTAVYFCARSGGTYSAFDIWGQGTMVTVSSGGGGSGGGGSGGGGSGGGGSE |
| | | IVLTQSPGTLSLSPGERSTLSCRASQSFTSNYLAWYQQRPGQAPRLLIYGASTR |
| | | AIGIPDRFSGSGSGTDFTLTISRLEPEDFAVYYCQQYGSSPWTFGQGTKVEIKT |
| | | TTPAPRPPTPAPTIASQPLSLRPEACRPAAGGAVHTRGLDFACDIYIWAPLAGT |
| | | CGVLLLSLVITLYCKRGRKKLLYIFKQPFMRPVQTTQEEDGCSCRFPEEEEGGC |
| | | ELRVKFSRSADAPAYQQGQNQLYNELNLGRREEYDVLDKRRGRDPEMGGKPRRK |
| | | NPQEGLYNELQKDKMAEAYSEIGMKGERRRGKGHDGLYQGLSTATKDTYDALHM |
| | | QALPPR |
| 145 | 4131scFv-V5- | MALPVTALLLPLALLLHAARPGYPYDVPDYADIVMTQTPASVSEPVGGSVTIKC |
| | 2XTPS7-53B6 | QASQSFYNLLAWYQQKPGQPPKLLIYDASDLASGVPSRFKGSGSGTDFTLTISD |
| | | LECADAAAYYCQSADGSSYAFGGGTEVVVKGGGGGSGGGGSGGGGSPMAKGSGG |
| | | GSGGGGSGGGGSQEQLEESGGGLVKPEGSLTLTCTASGVSFSSSYWIYWVRQAP |
| | | GKGLEWIACIYTGSSGSTYYASWAKGRFTVSETSSTTVTLQMTSLTAADTATYF |
| | | CARASAWTYGMDLWGPGTLVTVSSGGGGSGGGGSKPIPNPLLGLDSTGGGGSPM |
| | | AKGSGGGSGGGGSGGGGSGGGGSQVQLQESGPGLVKPSETLSLTCTVSGGSISY |
| | | YSWTWVRQPAGQGLEWIGRIHISGVTNHNPSLKSRVSMSIDTSRTQFSLRLTSV |
| | | TAADTAVYFCARSGGTYSAFDIWGQGTMVTVSSGGGGSGGGGSGGGGSGGGGSE |
| | | IVLTQSPGTLSLSPGERSTLSCRASQSFTSNYLAWYQQRPGQAPRLLIYGASTR |
| | | AIGIPDRFSGSGSGTDFTLTISRLEPEDFAVYYCQQYGSSPWTFGQGTKVEIKT |
| | | TTPAPRPPTPAPTIASQPLSLRPEACRPAAGGAVHTRGLDFACDIYIWAPLAGT |
| | | CGVLLLSLVITLYCKRGRKKLLYIFKQPFMRPVQTTQEEDGCSCRFPEEEEGGC |
| | | ELRVKFSRSADAPAYQQGQNQLYNELNLGRREEYDVLDKRRGRDPEMGGKPRRK |
| | | NPQEGLYNELQKDKMAEAYSEIGMKGERRRGKGHDGLYQGLSTATKDTYDALHM |
| | | QALPPR |
| 146 | N-HA- | MALPVTALLLPLALLLHAARPGYPYDVPDYADIVMTQTPASVSEPVGGSVTIKC |
| | 4131scFv- | QASQSFYNLLAWYQQKPGQPPKLLIYDASDLASGVPSRFKGSGSGTDFTLTISD |
| | G530-53B6 | LECADAAAYYCQSADGSSYAFGGGTEVVVKGGGGGSGGGGSGGGGSGGGGSQEQ |
| | | LEESGGGLVKPEGSLTLTCTASGVSFSSSYWIYWVRQAPGKGLEWIACIYTGSS |
| | | GSTYYASWAKGRFTVSETSSTTVTLQMTSLTAADTATYFCARASAWTYGMDLWG |
| | | PGTLVTVSSGGGGSGGGGSGGGSGGGSGSGGGGSGGGGSQVQLQESGPGLVKPS |
| | | ETLSLTCTVSGGSISYYSWTWVRQPAGQGLEWIGRIHISGVTNHNPSLKSRVSM |
| | | SIDTSRTQFSLRLTSVTAADTAVYFCARSGGTYSAFDIWGQGTMVTVSSGGGGS |
| | | GGGGSGGGGSGGGGSEIVLTQSPGTLSLSPGERSTLSCRASQSFTSNYLAWYQQ |
| | | RPGQAPRLLIYGASTRAIGIPDRFSGSGSGTDFTLTISRLEPEDFAVYYCQQYG |
| | | SSPWTFGQGTKVEIKTTTPAPRPPTPAPTIASQPLSLRPEACRPAAGGAVHTRG |
| | | LDFACDIYIWAPLAGTCGVLLLSLVITLYCKRGRKKLLYIFKQPFMRPVQTTQE |
| | | EDGCSCRFPEEEEGGCELRVKFSRSADAPAYQQGQNQLYNELNLGRREEYDVLD |
| | | KRRGRDPEMGGKPRRKNPQEGLYNELQKDKMAEAYSEIGMKGERRRGKGHDGLY |
| | | QGLSTATKDTYDALHMQALPPR |
| 147 | N-HA- | MALPVTALLLPLALLLHAARPGYPYDVPDYADIVMTQTPASVSEPVGGSVTIKC |
| | 4131scFv- | QASQSFYNLLAWYQQKPGQPPKLLIYDASDLASGVPSRFKGSGSGTDFTLTISD |
| | GS20-53B6 | LECADAAAYYCQSADGSSYAFGGGTEVVVKGGGGGSGGGGSGGGGSGGGGSQEQ |
| | | LEESGGGLVKPEGSLTLTCTASGVSFSSSYWIYWVRQAPGKGLEWIACIYTGSS |
| | | GSTYYASWAKGRFTVSETSSTTVTLQMTSLTAADTATYFCARASAWTYGMDLWG |
| | | PGTLVTVSSGGGGSGGGGSGGGSGGGGGSQVQLQESGPGLVKPSETLSLTCTVS |
| | | GGSISYYSWTWVRQPAGQGLEWIGRIHISGVTNHNPSLKSRVSMSIDTSRTQFS |
| | | LRLTSVTAADTAVYFCARSGGTYSAFDIWGQGTMVTVSSGGGGSGGGGSGGGGS |
| | | GGGGSEIVLTQSPGTLSLSPGERSTLSCRASQSFTSNYLAWYQQRPGQAPRLLI |
| | | YGASTRAIGIPDRFSGSGSGTDFTLTISRLEPEDFAVYYCQQYGSSPWTFGQGT |
| | | KVEIKTTTPAPRPPTPAPTIASQPLSLRPEACRPAAGGAVHTRGLDFACDIYIW |
| | | APLAGTCGVLLLSLVITLYCKRGRKKLLYIFKQPFMRPVQTTQEEDGCSCRFPE |
| | | EEEGGCELRVKFSRSADAPAYQQGQNQLYNELNLGRREEYDVLDKRRGRDPEMG |
| | | GKPRRKNPQEGLYNELQKDKMAEAYSEIGMKGERRRGKGHDGLYQGLSTATKDT |
| | | YDALHMQALPPR |
| 148 | N-HA- | MALPVTALLLPLALLLHAARPGYPYDVPDYADIVMTQTPASVSEPVGGSVTIKC |
| | 4131scFv- | QASQSFYNLLAWYQQKPGQPPKLLIYDASDLASGVPSRFKGSGSGTDFTLTISD |
| | GS10-53B6 | LECADAAAYYCQSADGSSYAFGGGTEVVVKGGGGGSGGGGSGGGGSGGGGSQEQ |
| | | LEESGGGLVKPEGSLTLTCTASGVSFSSSYWIYWVRQAPGKGLEWIACIYTGSS |
| | | GSTYYASWAKGRFTVSETSSTTVTLQMTSLTAADTATYFCARASAWTYGMDLWG |
| | | PGTLVTVSSGGGGSGGGGSQVQLQESGPGLVKPSETLSLTCTVSGGSISYYSWT |
| | | WVRQPAGQGLEWIGRIHISGVTNHNPSLKSRVSMSIDTSRTQFSLRLTSVTAAD |
| | | TAVYFCARSGGTYSAFDIWGQGTMVTVSSGGGGSGGGGSGGGGSGGGGSEIVLT |
| | | QSPGTLSLSPGERSTLSCRASQSFTSNYLAWYQQRPGQAPRLLIYGASTRAIGI |
| | | PDRFSGSGSGTDFTLTISRLEPEDFAVYYCQQYGSSPWTFGQGTKVEIKTTTPA |
| | | PRPPTPAPTIASQPLSLRPEACRPAAGGAVHTRGLDFACDIYIWAPLAGTCGVL |
| | | LLSLVITLYCKRGRKKLLYIFKQPFMRPVQTTQEEDGCSCRFPEEEEGGCELRV |
| | | KFSRSADAPAYQQGQNQLYNELNLGRREEYDVLDKRRGRDPEMGGKPRRKNPQE |
| | | GLYNELQKDKMAEAYSEIGMKGERRRGKGHDGLYQGLSTATKDTYDALHMQALP |
| | | PR |
| 149 | V5-GS20- | MALPVTALLLPLALLLHAARPGKPIPNPLLGLDSTGGGGSGGGGSGGGGSGGGG |
| | 4131scFv- | SDIVMTQTPASVSEPVGGSVTIKCQASQSFYNLLAWYQQKPGQPPKLLIYDASD |
| | TPS6-35-53B6 | LASGVPSRFKGSGSGTDFTLTISDLECADAAAYYCQSADGSSYAFGGGTEVVVK |
| | | GGGGGSGGGGSGGGGSGGGGSQEQLEESGGGLVKPEGSLTLTCTASGVSFSSSY |
| | | WIYWVRQAPGKGLEWIACIYTGSSGSTYYASWAKGRFTVSETSSTTVTLQMTSL |
| | | TAADTATYFCARASAWTYGMDLWGPGTLVTVSSGGGGSGGGGSGGGGSPMAKKG |
| | | GGGSGGGGSGGGGSQVQLQESGPGLVKPSETLSLTCTVSGGSISYYSWTWVRQP |
| | | AGQGLEWIGRIHISGVTNHNPSLKSRVSMSIDTSRTQFSLRLTSVTAADTAVYF |
| | | CARSGGTYSAFDIWGQGTMVTVSSGGGGSGGGGSGGGGSGGGGSEIVLTQSPGT |
| | | LSLSPGERSTLSCRASQSFTSNYLAWYQQRPGQAPRLLIYGASTRAIGIPDRES |
| | | GSGSGTDFTLTISRLEPEDFAVYYCQQYGSSPWTFGQGTKVEIKTTTPAPRPPT |
| | | PAPTIASQPLSLRPEACRPAAGGAVHTRGLDFACDIYIWAPLAGTCGVLLLSLV |
| | | ITLYCKRGRKKLLYIFKQPFMRPVQTTQEEDGCSCRFPEEEEGGCELRVKFSRS |
| | | ADAPAYQQGQNQLYNELNLGRREEYDVLDKRRGRDPEMGGKPRRKNPQEGLYNE |
| | | LQKDKMAEAYSEIGMKGERRRGKGHDGLYQGLSTATKDTYDALHMQALPPR |
| 150 | V5-G520- | MALPVTALLLPLALLLHAARPGKPIPNPLLGLDSTGGGGSGGGGSGGGGSGGGG |
| | 4131scFv- | SDIVMTQTPASVSEPVGGSVTIKCQASQSFYNLLAWYQQKPGQPPKLLIYDASD |
| | TPS6-30-53B6 | LASGVPSRFKGSGSGTDFTLTISDLECADAAAYYCQSADGSSYAFGGGTEVVVK |
| | | GGGGGSGGGGSGGGGSGGGGSQEQLEESGGGLVKPEGSLTLTCTASGVSFSSSY |
| | | WIYWVRQAPGKGLEWIACIYTGSSGSTYYASWAKGRFTVSETSSTTVTLQMTSL |
| | | TAADTATYFCARASAWTYGMDLWGPGTLVTVSSGGGGSGGGGSPMAKKGGGGSG |
| | | GGGSGGGGSQVQLQESGPGLVKPSETLSLTCTVSGGSISYYSWTWVRQPAGQGL |
| | | EWIGRIHISGVTNHNPSLKSRVSMSIDTSRTQFSLRLTSVTAADTAVYFCARSG |
| | | GTYSAFDIWGQGTMVTVSSGGGGSGGGGSGGGGSGGGGSEIVLTQSPGTLSLSP |
| | | GERSTLSCRASQSFTSNYLAWYQQRPGQAPRLLIYGASTRAIGIPDRFSGSGSG |
| | | TDFTLTISRLEPEDFAVYYCQQYGSSPWTFGQGTKVEIKTTTPAPRPPTPAPTI |
| | | ASQPLSLRPEACRPAAGGAVHTRGLDFACDIYIWAPLAGTCGVLLLSLVITLYC |
| | | KRGRKKLLYIFKQPFMRPVQTTQEEDGCSCRFPEEEEGGCELRVKFSRSADAPA |
| | | YQQGQNQLYNELNLGRREEYDVLDKRRGRDPEMGGKPRRKNPQEGLYNELQKDK |
| | | MAEAYSEIGMKGERRRGKGHDGLYQGLSTATKDTYDALHMQALPPR |
| 151 | V5-GS20- | MALPVTALLLPLALLLHAARPGKPIPNPLLGLDSTGGGGSGGGGSGGGGSGGGG |
| | 4131scFv- | SDIVMTQTPASVSEPVGGSVTIKCQASQSFYNLLAWYQQKPGQPPKLLIYDASD |
| | TPS6-25-53B6 | LASGVPSRFKGSGSGTDFTLTISDLECADAAAYYCQSADGSSYAFGGGTEVVVK |
| | | GGGGGSGGGGSGGGGSGGGGSQEQLEESGGGLVKPEGSLTLTCTASGVSFSSSY |
| | | WIYWVRQAPGKGLEWIACIYTGSSGSTYYASWAKGRFTVSETSSTTVTLQMTSL |
| | | TAADTATYFCARASAWTYGMDLWGPGTLVTVSSGGGGSGGGGSPMAKKGGGGSG |
| | | GGGSQVQLQESGPGLVKPSETLSLTCTVSGGSISYYSWTWVRQPAGQGLEWIGR |
| | | IHISGVTNHNPSLKSRVSMSIDTSRTQFSLRLTSVTAADTAVYFCARSGGTYSA |
| | | FDIWGQGTMVTVSSGGGGSGGGGSGGGGSGGGGSEIVLTQSPGTLSLSPGERST |
| | | LSCRASQSFTSNYLAWYQQRPGQAPRLLIYGASTRAIGIPDRFSGSGSGTDFTL |
| | | TISRLEPEDFAVYYCQQYGSSPWTFGQGTKVEIKTTTPAPRPPTPAPTIASQPL |
| | | SLRPEACRPAAGGAVHTRGLDFACDIYIWAPLAGTCGVLLLSLVITLYCKRGRK |
| | | KLLYIFKQPFMRPVQTTQEEDGCSCRFPEEEEGGCELRVKFSRSADAPAYQQGQ |
| | | NQLYNELNLGRREEYDVLDKRRGRDPEMGGKPRRKNPQEGLYNELQKDKMAEAY |
| | | SEIGMKGERRRGKGHDGLYQGLSTATKDTYDALHMQALPPR |
| 152 | V5-GS20- | MALPVTALLLPLALLLHAARPGKPIPNPLLGLDSTGGGGSGGGGSGGGGSGGGG |
| | 4131scFv- | SDIVMTQTPASVSEPVGGSVTIKCQASQSFYNLLAWYQQKPGQPPKLLIYDASD |
| | TPS6-20-53B6 | LASGVPSRFKGSGSGTDFTLTISDLECADAAAYYCQSADGSSYAFGGGTEVVVK |
| | | GGGGGSGGGGSGGGGSGGGGSQEQLEESGGGLVKPEGSLTLTCTASGVSFSSSY |
| | | WIYWVRQAPGKGLEWIACIYTGSSGSTYYASWAKGRFTVSETSSTTVTLQMTSL |
| | | TAADTATYFCARASAWTYGMDLWGPGTLVTVSSGGGGSPMAKKGGGGSGGGGSQ |
| | | VQLQESGPGLVKPSETLSLTCTVSGGSISYYSWTWVRQPAGQGLEWIGRIHISG |
| | | VTNHNPSLKSRVSMSIDTSRTQFSLRLTSVTAADTAVYFCARSGGTYSAFDIWG |
| | | QGTMVTVSSGGGGSGGGGSGGGGSGGGGSEIVLTQSPGTLSLSPGERSTLSCRA |
| | | SQSFTSNYLAWYQQRPGQAPRLLIYGASTRAIGIPDRFSGSGSGTDFTLTISRL |
| | | EPEDFAVYYCQQYGSSPWTFGQGTKVEIKTTTPAPRPPTPAPTIASQPLSLRPE |
| | | ACRPAAGGAVHTRGLDFACDIYIWAPLAGTCGVLLLSLVITLYCKRGRKKLLYI |
| | | FKQPFMRPVQTTQEEDGCSCRFPEEEEGGCELRVKFSRSADAPAYQQGQNQLYN |
| | | ELNLGRREEYDVLDKRRGRDPEMGGKPRRKNPQEGLYNELQKDKMAEAYSEIGM |
| | | KGERRRGKGHDGLYQGLSTATKDTYDALHMQALPPR |
| 153 | V5-G520- | MALPVTALLLPLALLLHAARPGKPIPNPLLGLDSTGGGGSGGGGSGGGGSGGGG |
| | 4131scFv- | SDIVMTQTPASVSEPVGGSVTIKCQASQSFYNLLAWYQQKPGQPPKLLIYDASD |
| | 2XTPS6-35- | LASGVPSRFKGSGSGTDFTLTISDLECADAAAYYCQSADGSSYAFGGGTEVVVK |
| | 53B6 | GGGGGSGGGGSGGGGSPMAKKGGGGSGGGGSGGGGSQEQLEESGGGLVKPEGSL |
| | | TLTCTASGVSFSSSYWIYWVRQAPGKGLEWIACIYTGSSGSTYYASWAKGRFTV |
| | | SETSSTTVTLQMTSLTAADTATYFCARASAWTYGMDLWGPGTLVTVSSGGGGSG |
| | | GGGSGGGGSPMAKKGGGGSGGGGSGGGGSQVQLQESGPGLVKPSETLSLTCTVS |
| | | GGSISYYSWTWVRQPAGQGLEWIGRIHISGVTNHNPSLKSRVSMSIDTSRTQFS |
| | | LRLTSVTAADTAVYFCARSGGTYSAFDIWGQGTMVTVSSGGGGSGGGGSGGGGS |
| | | GGGGSEIVLTQSPGTLSLSPGERSTLSCRASQSFTSNYLAWYQQRPGQAPRLLI |
| | | YGASTRAIGIPDRFSGSGSGTDFTLTISRLEPEDFAVYYCQQYGSSPWTFGQGT |
| | | KVEIKTTTPAPRPPTPAPTIASQPLSLRPEACRPAAGGAVHTRGLDFACDIYIW |
| | | APLAGTCGVLLLSLVITLYCKRGRKKLLYIFKQPFMRPVQTTQEEDGCSCRFPE |
| | | EEEGGCELRVKFSRSADAPAYQQGQNQLYNELNLGRREEYDVLDKRRGRDPEMG |
| | | GKPRRKNPQEGLYNELQKDKMAEAYSEIGMKGERRRGKGHDGLYQGLSTATKDT |
| | | YDALHMQALPPR |
| 154 | V5-G520- | MALPVTALLLPLALLLHAARPGKPIPNPLLGLDSTGGGGSGGGGSGGGGSGGGG |
| | 4131scFv- | SDIVMTQTPASVSEPVGGSVTIKCQASQSFYNLLAWYQQKPGQPPKLLIYDASD |
| | 2XTPS6-30- | LASGVPSRFKGSGSGTDFTLTISDLECADAAAYYCQSADGSSYAFGGGTEVVVK |
| | 53B6 | GGGGGSGGGGSGGGGSPMAKKGGGGSGGGGSGGGGSQEQLEESGGGLVKPEGSL |
| | | TLTCTASGVSFSSSYWIYWVRQAPGKGLEWIACIYTGSSGSTYYASWAKGRFTV |
| | | SETSSTTVTLQMTSLTAADTATYFCARASAWTYGMDLWGPGTLVTVSSGGGGSG |
| | | GGGSPMAKKGGGGSGGGGSGGGGSQVQLQESGPGLVKPSETLSLTCTVSGGSIS |
| | | YYSWTWVRQPAGQGLEWIGRIHISGVTNHNPSLKSRVSMSIDTSRTQFSLRLTS |
| | | VTAADTAVYFCARSGGTYSAFDIWGQGTMVTVSSGGGGSGGGGSGGGGSGGGGS |
| | | EIVLTQSPGTLSLSPGERSTLSCRASQSFTSNYLAWYQQRPGQAPRLLIYGAST |
| | | RAIGIPDRFSGSGSGTDFTLTISRLEPEDFAVYYCQQYGSSPWTFGQGTKVEIK |
| | | TTTPAPRPPTPAPTIASQPLSLRPEACRPAAGGAVHTRGLDFACDIYIWAPLAG |
| | | TCGVLLLSLVITLYCKRGRKKLLYIFKQPFMRPVQTTQEEDGCSCRFPEEEEGG |
| | | CELRVKFSRSADAPAYQQGQNQLYNELNLGRREEYDVLDKRRGRDPEMGGKPRR |
| | | KNPQEGLYNELQKDKMAEAYSEIGMKGERRRGKGHDGLYQGLSTATKDTYDALH |
| | | MQALPPR |
| 155 | V5-GS20- | MALPVTALLLPLALLLHAARPGKPIPNPLLGLDSTGGGGSGGGGSGGGGSGGGG |
| | 4131scFv- | SDIVMTQTPASVSEPVGGSVTIKCQASQSFYNLLAWYQQKPGQPPKLLIYDASD |
| | 2XTPS6-25- | LASGVPSRFKGSGSGTDFTLTISDLECADAAAYYCQSADGSSYAFGGGTEVVVK |
| | 53B6 | GGGGGSGGGGSGGGGSPMAKKGGGGSGGGGSGGGGSQEQLEESGGGLVKPEGSL |
| | | TLTCTASGVSFSSSYWIYWVRQAPGKGLEWIACIYTGSSGSTYYASWAKGRFTV |
| | | SETSSTTVTLQMTSLTAADTATYFCARASAWTYGMDLWGPGTLVTVSSGGGGSG |
| | | GGGSPMAKKGGGGSGGGGSQVQLQESGPGLVKPSETLSLTCTVSGGSISYYSWT |
| | | WVRQPAGQGLEWIGRIHISGVTNHNPSLKSRVSMSIDTSRTQFSLRLTSVTAAD |
| | | TAVYFCARSGGTYSAFDIWGQGTMVTVSSGGGGSGGGGSGGGGSGGGGSEIVLT |
| | | QSPGTLSLSPGERSTLSCRASQSFTSNYLAWYQQRPGQAPRLLIYGASTRAIGI |
| | | PDRFSGSGSGTDFTLTISRLEPEDFAVYYCQQYGSSPWTFGQGTKVEIKTTTPA |
| | | PRPPTPAPTIASQPLSLRPEACRPAAGGAVHTRGLDFACDIYIWAPLAGTCGVL |
| | | LLSLVITLYCKRGRKKLLYIFKQPFMRPVQTTQEEDGCSCRFPEEEEGGCELRV |
| | | KFSRSADAPAYQQGQNQLYNELNLGRREEYDVLDKRRGRDPEMGGKPRRKNPQE |
| | | GLYNELQKDKMAEAYSEIGMKGERRRGKGHDGLYQGLSTATKDTYDALHMQALP |
| | | PR |
| 156 | V5-GS20- | MALPVTALLLPLALLLHAARPGKPIPNPLLGLDSTGGGGSGGGGSGGGGSGGGG |
| | 4131scFv- | SDIVMTQTPASVSEPVGGSVTIKCQASQSFYNLLAWYQQKPGQPPKLLIYDASD |
| | 2XTP56-20- | LASGVPSRFKGSGSGTDFTLTISDLECADAAAYYCQSADGSSYAFGGGTEVVVK |
| | 53B6 | GGGGGSGGGGSGGGGSPMAKKGGGGSGGGGSGGGGSQEQLEESGGGLVKPEGSL |
| | | TLTCTASGVSFSSSYWIYWVRQAPGKGLEWIACIYTGSSGSTYYASWAKGRFTV |
| | | SETSSTTVTLQMTSLTAADTATYFCARASAWTYGMDLWGPGTLVTVSSGGGGSP |
| | | MAKKGGGGSGGGGSQVQLQESGPGLVKPSETLSLTCTVSGGSISYYSWTWVRQP |
| | | AGQGLEWIGRIHISGVTNHNPSLKSRVSMSIDTSRTQFSLRLTSVTAADTAVYF |
| | | CARSGGTYSAFDIWGQGTMVTVSSGGGGSGGGGSGGGGSGGGGSEIVLTQSPGT |
| | | LSLSPGERSTLSCRASQSFTSNYLAWYQQRPGQAPRLLIYGASTRAIGIPDRES |
| | | GSGSGTDFTLTISRLEPEDFAVYYCQQYGSSPWTFGQGTKVEIKTTTPAPRPPT |
| | | PAPTIASQPLSLRPEACRPAAGGAVHTRGLDFACDIYIWAPLAGTCGVLLLSLV |
| | | ITLYCKRGRKKLLYIFKQPFMRPVQTTQEEDGCSCRFPEEEEGGCELRVKFSRS |
| | | ADAPAYQQGQNQLYNELNLGRREEYDVLDKRRGRDPEMGGKPRRKNPQEGLYNE |
| | | LQKDKMAEAYSEIGMKGERRRGKGHDGLYQGLSTATKDTYDALHMQALPPR |
| 157 | V5-GS20- | MALPVTALLLPLALLLHAARPGKPIPNPLLGLDSTGGGGSGGGGSGGGGSGGGG |
| | 4131scFv- | SDIVMTQTPASVSEPVGGSVTIKCQASQSFYNLLAWYQQKPGQPPKLLIYDASD |
| | TPS8-53B6 | LASGVPSRFKGSGSGTDFTLTISDLECADAAAYYCQSADGSSYAFGGGTEVVVK |
| | | GGGGGSGGGGSGGGGSGGGGSQEQLEESGGGLVKPEGSLTLTCTASGVSFSSSY |
| | | WIYWVRQAPGKGLEWIACIYTGSSGSTYYASWAKGRFTVSETSSTTVTLQMTSL |
| | | TAADTATYFCARASAWTYGMDLWGPGTLVTVSSGGGGSGGGGSGSSPLGLAGSG |
| | | GGGSGGGGSPMAKKGGGGSGGGGSQVQLQESGPGLVKPSETLSLTCTVSGGSIS |
| | | YYSWTWVRQPAGQGLEWIGRIHISGVTNHNPSLKSRVSMSIDTSRTQFSLRLTS |
| | | VTAADTAVYFCARSGGTYSAFDIWGQGTMVTVSSGGGGSGGGGSGGGGSGGGGS |
| | | EIVLTQSPGTLSLSPGERSTLSCRASQSFTSNYLAWYQQRPGQAPRLLIYGAST |
| | | RAIGIPDRFSGSGSGTDFTLTISRLEPEDFAVYYCQQYGSSPWTFGQGTKVEIK |
| | | TTTPAPRPPTPAPTIASQPLSLRPEACRPAAGGAVHTRGLDFACDIYIWAPLAG |
| | | TCGVLLLSLVITLYCKRGRKKLLYIFKQPFMRPVQTTQEEDGCSCRFPEEEEGG |
| | | CELRVKFSRSADAPAYQQGQNQLYNELNLGRREEYDVLDKRRGRDPEMGGKPRR |
| | | KNPQEGLYNELQKDKMAEAYSEIGMKGERRRGKGHDGLYQGLSTATKDTYDALH |
| | | MQALPPR |
| 158 | V5-GS20- | MALPVTALLLPLALLLHAARPGKPIPNPLLGLDSTGGGGSGGGGSGGGGSGGGG |
| | 4131scFv- | SDIVMTQTPASVSEPVGGSVTIKCQASQSFYNLLAWYQQKPGQPPKLLIYDASD |
| | TPS9-53B6 | LASGVPSRFKGSGSGTDFTLTISDLECADAAAYYCQSADGSSYAFGGGTEVVVK |
| | | GGGGGSGGGGSGGGGSGGGGSQEQLEESGGGLVKPEGSLTLTCTASGVSFSSSY |
| | | WIYWVRQAPGKGLEWIACIYTGSSGSTYYASWAKGRFTVSETSSTTVTLQMTSL |
| | | TAADTATYFCARASAWTYGMDLWGPGTLVTVSSGGGGSGGGGSGGSVHMPLGEL |
| | | GPGGSGGGSPMAKKGGGGSGGGGSQVQLQESGPGLVKPSETLSLTCTVSGGSIS |
| | | YYSWTWVRQPAGQGLEWIGRIHISGVTNHNPSLKSRVSMSIDTSRTQFSLRLTS |
| | | VTAADTAVYFCARSGGTYSAFDIWGQGTMVTVSSGGGGSGGGGSGGGGSGGGGS |
| | | EIVLTQSPGTLSLSPGERSTLSCRASQSFTSNYLAWYQQRPGQAPRLLIYGAST |
| | | RAIGIPDRFSGSGSGTDFTLTISRLEPEDFAVYYCQQYGSSPWTFGQGTKVEIK |
| | | TTTPAPRPPTPAPTIASQPLSLRPEACRPAAGGAVHTRGLDFACDIYIWAPLAG |
| | | TCGVLLLSLVITLYCKRGRKKLLYIFKQPFMRPVQTTQEEDGCSCRFPEEEEGG |
| | | CELRVKFSRSADAPAYQQGQNQLYNELNLGRREEYDVLDKRRGRDPEMGGKPRR |
| | | KNPQEGLYNELQKDKMAEAYSEIGMKGERRRGKGHDGLYQGLSTATKDTYDALH |
| | | MQALPPR |
| 159 | V5-GS20- | MALPVTALLLPLALLLHAARPGKPIPNPLLGLDSTGGGGSGGGGSGGGGSGGGG |
| | 4131scFv- | SDIVMTQTPASVSEPVGGSVTIKCQASQSFYNLLAWYQQKPGQPPKLLIYDASD |
| | TPS10-53B6 | LASGVPSRFKGSGSGTDFTLTISDLECADAAAYYCQSADGSSYAFGGGTEVVVK |
| | | GGGGGSGGGGSGGGGSGGGGSQEQLEESGGGLVKPEGSLTLTCTASGVSFSSSY |
| | | WIYWVRQAPGKGLEWIACIYTGSSGSTYYASWAKGRFTVSETSSTTVTLQMTSL |
| | | TAADTATYFCARASAWTYGMDLWGPGTLVTVSSGGGGSGGGGSGGSPLGVRGKG |
| | | GGGSGGGGSPMAKKGGGGSGGGGSQVQLQESGPGLVKPSETLSLTCTVSGGSIS |
| | | YYSWTWVRQPAGQGLEWIGRIHISGVTNHNPSLKSRVSMSIDTSRTQFSLRLTS |
| | | VTAADTAVYFCARSGGTYSAFDIWGQGTMVTVSSGGGGSGGGGSGGGGSGGGGS |
| | | EIVLTQSPGTLSLSPGERSTLSCRASQSFTSNYLAWYQQRPGQAPRLLIYGAST |
| | | RAIGIPDRFSGSGSGTDFTLTISRLEPEDFAVYYCQQYGSSPWTFGQGTKVEIK |
| | | TTTPAPRPPTPAPTIASQPLSLRPEACRPAAGGAVHTRGLDFACDIYIWAPLAG |
| | | TCGVLLLSLVITLYCKRGRKKLLYIFKQPFMRPVQTTQEEDGCSCRFPEEEEGG |
| | | CELRVKFSRSADAPAYQQGQNQLYNELNLGRREEYDVLDKRRGRDPEMGGKPRR |
| | | KNPQEGLYNELQKDKMAEAYSEIGMKGERRRGKGHDGLYQGLSTATKDTYDALH |
| | | MQALPPR |
| 160 | V5-GS20- | MALPVTALLLPLALLLHAARPGKPIPNPLLGLDSTGGGGSGGGGSGGGGSGGGG |
| | 4131scFv- | SDIVMTQTPASVSEPVGGSVTIKCQASQSFYNLLAWYQQKPGQPPKLLIYDASD |
| | TPS11-53B6 | LASGVPSRFKGSGSGTDFTLTISDLECADAAAYYCQSADGSSYAFGGGTEVVVK |
| | | GGGGGSGGGGSGGGGSGGGGSQEQLEESGGGLVKPEGSLTLTCTASGVSFSSSY |
| | | WIYWVRQAPGKGLEWIACIYTGSSGSTYYASWAKGRFTVSETSSTTVTLQMTSL |
| | | TAADTATYFCARASAWTYGMDLWGPGTLVTVSSGGGGSGGGGSGGSSPLGLAGS |
| | | GSGGGSRQARVVNGGGGGSGGGGSQVQLQESGPGLVKPSETLSLTCTVSGGSIS |
| | | YYSWTWVRQPAGQGLEWIGRIHISGVTNHNPSLKSRVSMSIDTSRTQFSLRLTS |
| | | VTAADTAVYFCARSGGTYSAFDIWGQGTMVTVSSGGGGSGGGGSGGGGSGGGGS |
| | | EIVLTQSPGTLSLSPGERSTLSCRASQSFTSNYLAWYQQRPGQAPRLLIYGAST |
| | | RAIGIPDRFSGSGSGTDFTLTISRLEPEDFAVYYCQQYGSSPWTFGQGTKVEIK |
| | | TTTPAPRPPTPAPTIASQPLSLRPEACRPAAGGAVHTRGLDFACDIYIWAPLAG |
| | | TCGVLLLSLVITLYCKRGRKKLLYIFKQPFMRPVQTTQEEDGCSCRFPEEEEGG |
| | | CELRVKFSRSADAPAYQQGQNQLYNELNLGRREEYDVLDKRRGRDPEMGGKPRR |
| | | KNPQEGLYNELQKDKMAEAYSEIGMKGERRRGKGHDGLYQGLSTATKDTYDALH |
| | | MQALPPR |
| 161 | V5-G520- | MALPVTALLLPLALLLHAARPGKPIPNPLLGLDSTGGGGSGGGGSGGGGSGGGG |
| | 4131scFv- | SDIVMTQTPASVSEPVGGSVTIKCQASQSFYNLLAWYQQKPGQPPKLLIYDASD |
| | TPS12-53B6 | LASGVPSRFKGSGSGTDFTLTISDLECADAAAYYCQSADGSSYAFGGGTEVVVK |
| | | GGGGGSGGGGSGGGGSGGGGSQEQLEESGGGLVKPEGSLTLTCTASGVSFSSSY |
| | | WIYWVRQAPGKGLEWIACIYTGSSGSTYYASWAKGRFTVSETSSTTVTLQMTSL |
| | | TAADTATYFCARASAWTYGMDLWGPGTLVTVSSGGGGSGGGGSGGSVHMPLGFL |
| | | GPGGGSRQARVVNGGGGGSGGGGSQVQLQESGPGLVKPSETLSLTCTVSGGSIS |
| | | YYSWTWVRQPAGQGLEWIGRIHISGVTNHNPSLKSRVSMSIDTSRTQFSLRLTS |
| | | VTAADTAVYFCARSGGTYSAFDIWGQGTMVTVSSGGGGSGGGGSGGGGSGGGGS |
| | | EIVLTQSPGTLSLSPGERSTLSCRASQSFTSNYLAWYQQRPGQAPRLLIYGAST |
| | | RAIGIPDRFSGSGSGTDFTLTISRLEPEDFAVYYCQQYGSSPWTFGQGTKVEIK |
| | | TTTPAPRPPTPAPTIASQPLSLRPEACRPAAGGAVHTRGLDFACDIYIWAPLAG |
| | | TCGVLLLSLVITLYCKRGRKKLLYIFKQPFMRPVQTTQEEDGCSCRFPEEEEGG |
| | | CELRVKFSRSADAPAYQQGQNQLYNELNLGRREEYDVLDKRRGRDPEMGGKPRR |
| | | KNPQEGLYNELQKDKMAEAYSEIGMKGERRRGKGHDGLYQGLSTATKDTYDALH |
| | | MQALPPR |
| 162 | V5-G520- | MALPVTALLLPLALLLHAARPGKPIPNPLLGLDSTGGGGSGGGGSGGGGSGGGG |
| | 4131scFv- | SDIVMTQTPASVSEPVGGSVTIKCQASQSFYNLLAWYQQKPGQPPKLLIYDASD |
| | TPS13-53B6 | LASGVPSRFKGSGSGTDFTLTISDLECADAAAYYCQSADGSSYAFGGGTEVVVK |
| | | GGGGGSGGGGSGGGGSGGGGSQEQLEESGGGLVKPEGSLTLTCTASGVSFSSSY |
| | | WIYWVRQAPGKGLEWIACIYTGSSGSTYYASWAKGRFTVSETSSTTVTLQMTSL |
| | | TAADTATYFCARASAWTYGMDLWGPGTLVTVSSGGGGSGGGGSGGSPLGVRGKG |
| | | GSGGGSRQARVVNGGGGGSGGGGSQVQLQESGPGLVKPSETLSLTCTVSGGSIS |
| | | YYSWTWVRQPAGQGLEWIGRIHISGVTNHNPSLKSRVSMSIDTSRTQFSLRLTS |
| | | VTAADTAVYFCARSGGTYSAFDIWGQGTMVTVSSGGGGSGGGGSGGGGSGGGGS |
| | | EIVLTQSPGTLSLSPGERSTLSCRASQSFTSNYLAWYQQRPGQAPRLLIYGAST |
| | | RAIGIPDRFSGSGSGTDFTLTISRLEPEDFAVYYCQQYGSSPWTFGQGTKVEIK |
| | | TTTPAPRPPTPAPTIASQPLSLRPEACRPAAGGAVHTRGLDFACDIYIWAPLAG |
| | | TCGVLLLSLVITLYCKRGRKKLLYIFKQPFMRPVQTTQEEDGCSCRFPEEEEGG |
| | | CELRVKFSRSADAPAYQQGQNQLYNELNLGRREEYDVLDKRRGRDPEMGGKPRR |
| | | KNPQEGLYNELQKDKMAEAYSEIGMKGERRRGKGHDGLYQGLSTATKDTYDALH |
| | | MQALPPR |
| 163 | V5-G520- | MALPVTALLLPLALLLHAARPGKPIPNPLLGLDSTGGGGSGGGGSGGGGSGGGG |
| | 4131scFv- | SDIVMTQTPASVSEPVGGSVTIKCQASQSFYNLLAWYQQKPGQPPKLLIYDASD |
| | G545-53B6 | LASGVPSRFKGSGSGTDFTLTISDLECADAAAYYCQSADGSSYAFGGGTEVVVK |
| | | GGGGGSGGGGSGGGGSGGGGSQEQLEESGGGLVKPEGSLTLTCTASGVSFSSSY |
| | | WIYWVRQAPGKGLEWIACIYTGSSGSTYYASWAKGRFTVSETSSTTVTLQMTSL |
| | | TAADTATYFCARASAWTYGMDLWGPGTLVTVSSGGGGSGGGGSGGGSGGGSGSG |
| | | GGGSGGGGSGGGGSGGGGSGGGGSQVQLQESGPGLVKPSETLSLTCTVSGGSIS |
| | | YYSWTWVRQPAGQGLEWIGRIHISGVTNHNPSLKSRVSMSIDTSRTQFSLRLTS |
| | | VTAADTAVYFCARSGGTYSAFDIWGQGTMVTVSSGGGGSGGGGSGGGGSGGGGS |
| | | EIVLTQSPGTLSLSPGERSTLSCRASQSFTSNYLAWYQQRPGQAPRLLIYGAST |
| | | RAIGIPDRFSGSGSGTDFTLTISRLEPEDFAVYYCQQYGSSPWTFGQGTKVEIK |
| | | TTTPAPRPPTPAPTIASQPLSLRPEACRPAAGGAVHTRGLDFACDIYIWAPLAG |
| | | TCGVLLLSLVITLYCKRGRKKLLYIFKQPFMRPVQTTQEEDGCSCRFPEEEEGG |
| | | CELRVKFSRSADAPAYQQGQNQLYNELNLGRREEYDVLDKRRGRDPEMGGKPRR |
| | | KNPQEGLYNELQKDKMAEAYSEIGMKGERRRGKGHDGLYQGLSTATKDTYDALH |
| | | MQALPPR |
| 164 | v5-G520- | MALPVTALLLPLALLLHAARPGKPIPNPLLGLDSTGGGGSGGGGSGGGGSGGGG |
| | 4131scFv- | SDIVMTQTPASVSEPVGGSVTIKCQASQSFYNLLAWYQQKPGQPPKLLIYDASD |
| | G535-53B6 | LASGVPSRFKGSGSGTDFTLTISDLECADAAAYYCQSADGSSYAFGGGTEVVVK |
| | | GGGGGSGGGGSGGGGSGGGGSQEQLEESGGGLVKPEGSLTLTCTASGVSFSSSY |
| | | WIYWVRQAPGKGLEWIACIYTGSSGSTYYASWAKGRFTVSETSSTTVTLQMTSL |
| | | TAADTATYFCARASAWTYGMDLWGPGTLVTVSSGGGSGGGSGSGGGGSGGGGSG |
| | | GGGSGGGGSGGGGSQVQLQESGPGLVKPSETLSLTCTVSGGSISYYSWTWVRQP |
| | | AGQGLEWIGRIHISGVTNHNPSLKSRVSMSIDTSRTQFSLRLTSVTAADTAVYF |
| | | CARSGGTYSAFDIWGQGTMVTVSSGGGGSGGGGSGGGGSGGGGSEIVLTQSPGT |
| | | LSLSPGERSTLSCRASQSFTSNYLAWYQQRPGQAPRLLIYGASTRAIGIPDRFS |
| | | GSGSGTDFTLTISRLEPEDFAVYYCQQYGSSPWTFGQGTKVEIKTTTPAPRPPT |
| | | PAPTIASQPLSLRPEACRPAAGGAVHTRGLDFACDIYIWAPLAGTCGVLLLSLV |
| | | ITLYCKRGRKKLLYIFKQPFMRPVQTTQEEDGCSCRFPEEEEGGCELRVKFSRS |
| | | ADAPAYQQGQNQLYNELNLGRREEYDVLDKRRGRDPEMGGKPRRKNPQEGLYNE |
| | | LQKDKMAEAYSEIGMKGERRRGKGHDGLYQGLSTATKDTYDALHMQALPPR |
| 165 | V5-GS20- | MALPVTALLLPLALLLHAARPGKPIPNPLLGLDSTGGGGSGGGGSGGGGSGGGG |
| | 4131scFv- | SDIVMTQTPASVSEPVGGSVTIKCQASQSFYNLLAWYQQKPGQPPKLLIYDASD |
| | GS25-53B6 | LASGVPSRFKGSGSGTDFTLTISDLECADAAAYYCQSADGSSYAFGGGTEVVVK |
| | | GGGGGSGGGGSGGGGSGGGGSQEQLEESGGGLVKPEGSLTLTCTASGVSFSSSY |
| | | WIYWVRQAPGKGLEWIACIYTGSSGSTYYASWAKGRFTVSETSSTTVTLQMTSL |
| | | TAADTATYFCARASAWTYGMDLWGPGTLVTVSSGGGGSGGGGSGGGGSGGGGSG |
| | | GGGSQVQLQESGPGLVKPSETLSLTCTVSGGSISYYSWTWVRQPAGQGLEWIGR |
| | | IHISGVTNHNPSLKSRVSMSIDTSRTQFSLRLTSVTAADTAVYFCARSGGTYSA |
| | | FDIWGQGTMVTVSSGGGGSGGGGSGGGGSGGGGSEIVLTQSPGTLSLSPGERST |
| | | LSCRASQSFTSNYLAWYQQRPGQAPRLLIYGASTRAIGIPDRFSGSGSGTDFTL |
| | | TISRLEPEDFAVYYCQQYGSSPWTFGQGTKVEIKTTTPAPRPPTPAPTIASQPL |
| | | SLRPEACRPAAGGAVHTRGLDFACDIYIWAPLAGTCGVLLLSLVITLYCKRGRK |
| | | KLLYIFKQPFMRPVQTTQEEDGCSCRFPEEEEGGCELRVKFSRSADAPAYQQGQ |
| | | NQLYNELNLGRREEYDVLDKRRGRDPEMGGKPRRKNPQEGLYNELQKDKMAEAY |
| | | SEIGMKGERRRGKGHDGLYQGLSTATKDTYDALHMQALPPR |
| 166 | N-HA-IgV- | MALPVTALLLPLALLLHAARPGYPYDVPDYAQFSVLGPSGPILAMVGEDADLPC |
| | G545-53B6 | HLFPTMSAETMELKWVSSSLRQVVNVYADGKEVEDRQSAPYRGRTSILRDGITA |
| | | GKAALRIHNVTASDSGKYLCYFQDGDFYEKALVELKVAGGGGSGGGGSGGGSGG |
| | | GSGSGGGGSGGGGSGGGGSGGGGSGGGGSQVQLQESGPGLVKPSETLSLTCTVS |
| | | GGSISYYSWTWVRQPAGQGLEWIGRIHISGVTNHNPSLKSRVSMSIDTSRTQFS |
| | | LRLTSVTAADTAVYFCARSGGTYSAFDIWGQGTMVTVSSGGGGSGGGGSGGGGS |
| | | GGGGSEIVLTQSPGTLSLSPGERSTLSCRASQSFTSNYLAWYQQRPGQAPRLLI |
| | | YGASTRAIGIPDRFSGSGSGTDFTLTISRLEPEDFAVYYCQQYGSSPWTFGQGT |
| | | KVEIKTTTPAPRPPTPAPTIASQPLSLRPEACRPAAGGAVHTRGLDFACDIYIW |
| | | APLAGTCGVLLLSLVITLYCKRGRKKLLYIFKQPFMRPVQTTQEEDGCSCRFPE |
| | | EEEGGCELRVKFSRSADAPAYQQGQNQLYNELNLGRREEYDVLDKRRGRDPEMG |
| | | GKPRRKNPQEGLYNELQKDKMAEAYSEIGMKGERRRGKGHDGLYQGLSTATKDT |
| | | YDALHMQALPPR |
| 167 | N-HA-IgC- | MALPVTALLLPLALLLHAARPGYPYDVPDYAALGSDLHVDVKGYKDGGIHLECR |
| | G545-53B6 | STGWYPQPQIQWSNNKGENIPTVEAPVVADGVGLYAVAASVIMRGSSGEGVSCT |
| | | IRSSLLGLEKTASISIADPFFRSAQRWIAALAGTGGGGSGGGGSGGGSGGGSGS |
| | | GGGGSGGGGSGGGGSGGGGSGGGGSQVQLQESGPGLVKPSETLSLTCTVSGGSI |
| | | SYYSWTWVRQPAGQGLEWIGRIHISGVTNHNPSLKSRVSMSIDTSRTQFSLRLT |
| | | SVTAADTAVYFCARSGGTYSAFDIWGQGTMVTVSSGGGGSGGGGSGGGGSGGGG |
| | | SEIVLTQSPGTLSLSPGERSTLSCRASQSFTSNYLAWYQQRPGQAPRLLIYGAS |
| | | TRAIGIPDRFSGSGSGTDFTLTISRLEPEDFAVYYCQQYGSSPWTFGQGTKVEI |
| | | KTTTPAPRPPTPAPTIASQPLSLRPEACRPAAGGAVHTRGLDFACDIYIWAPLA |
| | | GTCGVLLLSLVITLYCKRGRKKLLYIFKQPFMRPVQTTQEEDGCSCRFPEEEEG |
| | | GCELRVKFSRSADAPAYQQGQNQLYNELNLGRREEYDVLDKRRGRDPEMGGKPR |
| | | RKNPQEGLYNELQKDKMAEAYSEIGMKGERRRGKGHDGLYQGLSTATKDTYDAL |
| | | HMQALPPR |
| 168 | N-HA-BTN3A1- | MALPVTALLLPLALLLHAARPGYPYDVPDYAQFSVLGPSGPILAMVGEDADLPC |
| | G545-53B6 | HLFPTMSAETMELKWVSSSLRQVVNVYADGKEVEDRQSAPYRGRTSILRDGITA |
| | | GKAALRIHNVTASDSGKYLCYFQDGDFYEKALVELKVAALGSDLHVDVKGYKDG |
| | | GIHLECRSTGWYPQPQIQWSNNKGENIPTVEAPVVADGVGLYAVAASVIMRGSS |
| | | GEGVSCTIRSSLLGLEKTASISIADPFFRSAQRWIAALAGTGGGGSGGGGSGGG |
| | | SGGGSGSGGGGSGGGGSGGGGSGGGGSGGGGSQVQLQESGPGLVKPSETLSLTC |
| | | TVSGGSISYYSWTWVRQPAGQGLEWIGRIHISGVTNHNPSLKSRVSMSIDTSRT |
| | | QFSLRLTSVTAADTAVYFCARSGGTYSAFDIWGQGTMVTVSSGGGGSGGGGSGG |
| | | GGSGGGGSEIVLTQSPGTLSLSPGERSTLSCRASQSFTSNYLAWYQQRPGQAPR |
| | | LLIYGASTRAIGIPDRFSGSGSGTDFTLTISRLEPEDFAVYYCQQYGSSPWTFG |
| | | QGTKVEIKTTTPAPRPPTPAPTIASQPLSLRPEACRPAAGGAVHTRGLDFACDI |
| | | YIWAPLAGTCGVLLLSLVITLYCKRGRKKLLYIFKQPFMRPVQTTQEEDGCSCR |
| | | FPEEEEGGCELRVKFSRSADAPAYQQGQNQLYNELNLGRREEYDVLDKRRGRDP |
| | | EMGGKPRRKNPQEGLYNELQKDKMAEAYSEIGMKGERRRGKGHDGLYQGLSTAT |
| | | KDTYDALHMQALPPR |
| 169 | N-HA-sec49k- | MALPVTALLLPLALLLHAARPGYPYDVPDYAGFHTINATWWANITLVGPPDTPV |
| | GS45-53B6 | TWYDTQGLWFCNGSRVKNPQIRHTCNDQNLTLIHVNKTYERTYMGYNRQGTKKE |
| | | DYKVVVIPPPPATVKPQPEPEYVFVYMGENKTLEGPPGTPVTWFNQDGKKFCEG |
| | | EKVLHPEFNHTCDKQNLILLFVNFTHDGAYLGYNHQGTQRTHYEVTVLDLFPDS |
| | | GQMKIENHSEETEQKNDEHHNWQKQGGQKQGGQKTNQTKVNDRRKTAQKRPSKL |
| | | KPATIEAMLVTVTAGSNLTLVGPKAEGKVTWFDGDLKRPCEPNYRLRHECNNQN |
| | | LTLINVTKDYEGTYYGTNDKDEGKRYRVKVNTTNSQSVKIQPYTRQTTPDQEHK |
| | | FELQFETNGNYDSKIPGGGGSGGGGSGGGSGGGSGSGGGGSGGGGSGGGGSGGG |
| | | GSGGGGSQVQLQESGPGLVKPSETLSLTCTVSGGSISYYSWTWVRQPAGQGLEW |
| | | IGRIHISGVTNHNPSLKSRVSMSIDTSRTQFSLRLTSVTAADTAVYFCARSGGT |
| | | YSAFDIWGQGTMVTVSSGGGGSGGGGSGGGGSGGGGSEIVLTQSPGTLSLSPGE |
| | | RSTLSCRASQSFTSNYLAWYQQRPGQAPRLLIYGASTRAIGIPDRFSGSGSGTD |
| | | FTLTISRLEPEDFAVYYCQQYGSSPWTFGQGTKVEIKTTTPAPRPPTPAPTIAS |
| | | QPLSLRPEACRPAAGGAVHTRGLDFACDIYIWAPLAGTCGVLLLSLVITLYCKR |
| | | GRKKLLYIFKQPFMRPVQTTQEEDGCSCRFPEEEEGGCELRVKFSRSADAPAYQ |
| | | QGQNQLYNELNLGRREEYDVLDKRRGRDPEMGGKPRRKNPQEGLYNELQKDKMA |
| | | EAYSEIGMKGERRRGKGHDGLYQGLSTATKDTYDALHMQALPPR |
| 170 | N-HA-UL11- | MALPVTALLLPLALLLHAARPGYPYDVPDYAHDACIPVVGKIGTNVTLNAVDFH |
| | GS45-53B6 | PGDHVRWSYGPGGAGYMLCVYTGSWTEYKKPDIIFKCLSNNSLLLINVTVNYTN |
| | | TYRTLTSLNNWVHNQHHHKFPGWNLDTCYSLTVNENGTFPTTTTKKPTTTTRTT |
| | | TTTTTKKTTTTRTTTAAKKTTISTTHHKHSSPKKSSTPNSHVEHHVGFEATAAE |
| | | TPLQPSPQHQHVATHGGGGSGGGGSGGGSGGGSGSGGGGSGGGGSGGGGSGGGG |
| | | SGGGGSQVQLQESGPGLVKPSETLSLTCTVSGGSISYYSWTWVRQPAGQGLEWI |
| | | GRIHISGVTNHNPSLKSRVSMSIDTSRTQFSLRLTSVTAADTAVYFCARSGGTY |
| | | SAFDIWGQGTMVTVSSGGGGSGGGGSGGGGSGGGGSEIVLTQSPGTLSLSPGER |
| | | STLSCRASQSFTSNYLAWYQQRPGQAPRLLIYGASTRAIGIPDRFSGSGSGTDF |
| | | TLTISRLEPEDFAVYYCQQYGSSPWTFGQGTKVEIKTTTPAPRPPTPAPTIASQ |
| | | PLSLRPEACRPAAGGAVHTRGLDFACDIYIWAPLAGTCGVLLLSLVITLYCKRG |
| | | RKKLLYIFKQPFMRPVQTTQEEDGCSCRFPEEEEGGCELRVKFSRSADAPAYQQ |
| | | GQNQLYNELNLGRREEYDVLDKRRGRDPEMGGKPRRKNPQEGLYNELQKDKMAE |
| | | AYSEIGMKGERRRGKGHDGLYQGLSTATKDTYDALHMQALPPR |

### MUC16-Specific CARs

In another aspect, provided herein is a CAR that specifically binds to MUC16, wherein the CAR comprises an extracellular ligand-binding domain comprising: a VH region having the sequence shown in SEQ ID NO: 58, and/or a VL region having the sequence shown in SEQ ID NO: 59. In some embodiments, the VH and VL are linked together by a flexible linker. In some embodiments, a flexible linker comprises the amino acid sequence shown in SEQ ID NO: 57 ((GGGGS) 3).

In another aspect, provided is a MUC16 specific chimeric antigen receptor (CAR) comprising an extracellular ligand-binding domain, a first transmembrane domain, and an intracellular signaling domain, wherein the extracellular domain comprises a single chain Fv fragment (scFv) comprising a heavy chain variable (VH) region and a light chain variable (VL) region, wherein the VH region comprises (i) a VH complementarity determining region one (CDR1) having the amino acid sequence shown in SEQ ID NO:60 or 63; (ii) a VH complementarity determining region two (CDR2) having the amino acid sequence shown in SEQ ID NO: 61 or 64; and (iii) a VH complementarity determining region three (CDR3) having the amino acid sequence shown in SEQ ID NO:62 or 65.

In another aspect, provided is a MUC16 specific chimeric antigen receptor (CAR) comprising an extracellular ligand-binding domain, a first transmembrane domain, and an intracellular signaling domain, wherein the extracellular domain comprises a single chain Fv fragment (scFv) comprising a heavy chain variable (VH) region and a light chain variable (VL) region, wherein the VL region comprises (i) a VL complementarity determining region one (CDR1) having the amino acid sequence shown in SEQ ID NO: 66 or 69; (ii) a VL complementarity determining region two (CDR2) having the amino acid sequence shown in SEQ ID NO: 67 or 70; and (iii) a VL complementarity determining region three (CDR3) having the amino acid sequence shown in SEQ ID NO: 68 or 71.

In another aspect, provided is a MUC16 specific chimeric antigen receptor (CAR) comprising an extracellular ligand-binding domain, a first transmembrane domain, and an intracellular signaling domain, wherein the extracellular domain comprises a single chain Fv fragment (scFv) comprising a heavy chain variable (VH) region and a light chain variable (VL) region, wherein (a) the VH region comprises (i) a VH complementarity determining region one (CDR1) having the amino acid sequence shown in SEQ ID NO: 60 or 63; (ii) a VH complementarity determining region two (CDR2) having the amino acid sequence shown in SEQ ID NO: 61 or 64; and (iii) a VH complementarity determining region three (CDR3) having the amino acid sequence shown in SEQ ID NO: 62 or 65; and (b) the VL region comprises (i) a VL complementarity determining region one (CDR1) having the amino acid sequence shown in SEQ ID NO: 66; (ii) a VL complementarity determining region two (CDR2) having the amino acid sequence shown in SEQ ID NO:67; and (iii) a VL complementarity determining region three (CDR3) having the amino acid sequence shown in SEQ ID NO: 68.

Also provided herein are CDR portions of antigen binding domains of antibodies to MUC16 or CDR portions of extracellular ligand-binding domains of CARs to MUC16 (including Chothia, Kabat CDRs, and CDR contact regions). Determination of CDR regions is well within the skill of the art. It is understood that in some embodiments, CDRs can be a combination of the Kabat and Chothia CDR (also termed “combined CRs” or “extended CDRs”). In some embodiments, the CDRs are the Kabat CDRs. In other embodiments, the CDRs are the Chothia CDRs. In other words, in embodiments with more than one CDR, the CDRs may be any of Kabat, Chothia, combination CDRs, or combinations thereof.

TABLE 3
53B6 MUC16 CAR component sequences

| | |
|---|---|
| | SEQ |
| Clone | ID NO: |

VH Sequence

| | | |
|---|---|---|
| 53B6-VH | QVQLQESGPGLVKPSETLSLTCTVSGGSISYYSWTWVRQPA | 58 |
| | GQGLEWIGRIHISGVTNHNPSLKSRVSMSIDTSRTQFSLRL | |
| | TSVTAADTAVYFCARSGGTYSAFDIWGQGTMVTVSS | |

VL sequence

| | | |
|---|---|---|
| 53B6-VL | EIVLTQSPGTLSLSPGERSTLSCRASQSFTSNYLAWYQQRP | 59 |
| | GQAPRLLIYGASTRAIGIPDRFSGSGSGTDFTLTISRLEPE | |
| | DFAVYYCQQYGSSPWTFGQGTKVEIK | |

CDRs for 53B6 VH (Kabat definition)

| | | |
|---|---|---|
| CDR1 | YYSWT | 60 |
| CDR2 | RIHISGVTNHNPSLKS | 61 |
| CDR3 | SGGTYSAFDI | 62 |

CDRs for 53B6 VH (Chothia definition)

| | | |
|---|---|---|
| CDR1 | SGGSISYY | 63 |
| CDR2 | HISGV | 64 |
| CDR3 | SGGTYSAFDI | 65 |

CDRs for 53B6 VL (Kabat definition)

| | | |
|---|---|---|
| CDR1 | RASQSFTSNYLA | 66 |
| CDR2 | GASTRAI | 67 |
| CDR3 | QQYGSSPWT | 68 |

CDRs for 53B6 VL (Chothia definition)

| | | |
|---|---|---|
| CDR1 | RASQSFTSNYLA | 69 |
| CDR2 | GASTRAI | 70 |
| CDR3 | QQYGSSPWT | 71 |

CAR Amino Acid Sequences

| | | |
|---|---|---|
| CD8α signal | MALPVTALLLPLALLLHAARP | 1 |
| sequence | | |
| GS linker 2 | GGGGSGGGGSGGGGSGGGGS | 72 |
| CD8α hinge | TTTPAPRPPTPAPTIASQPLSLRPEACRPAAGGAVHTRGLD | 73 |
| and | FACDIYIWAPLAGTCGVLLLSLVIT | |
| transmembrane | | |
| regions | | |
| 41BB | KRGRKKLLYIFKQPFMRPVQTTQEEDGCSCRFPEEEEGGCE | 33 |
| cytoplasmic | L | |
| signaling | | |
| domain | | |
| CD3ζ | RVKFSRSADAPAYQQGQNQLYNELNLGRREEYDVLDKRRGR | 34 |
| cytoplasmic | DPEMGGKPRRKNPQEGLYNELQKDKMAEAYSEIGMKGERRR | |
| signaling | GKGHDGLYQGLSTATKDTYDALHMQALPPR | |
| domain | | |

Mimotope sequences

| | | |
|---|---|---|
| Rituximab | | |
| Mimotope | CPYSNPSLC | 50 |
| QBEND-10 | | |
| Epitope | ELPTQGTFSNVSTNVSPAKPTTTA | 74 |
| Epitope | ELPTQGTFSNVSTNVS | 179 |

TABLE 4
53B6 CAR sequences

| Clone | | | SEQ ID |
|---|---|---|---|
| Name | Name/Component | Sequence | NO: |
| 53B6 | CD8α signal | MALPVTALLLPLALLLHAARPQVQLQESGPGLVKPS | 75 |
| | sequence, 53B6 | ETLSLTCTVSGGSISYYSWTWVRQPAGQGLEWIGRI | |
| | scFv, CD8α | HISGVTNHNPSLKSRVSMSIDTSRTQFSLRLTSVTA | |
| | hinge and | ADTAVYFCARSGGTYSAFDIWGQGTMVTVSSGGGGS | |
| | transmembrane | GGGGSGGGGSGGGGSEIVLTQSPGTLSLSPGERSTL | |
| | regions, 41BB | SCRASQSFTSNYLAWYQQRPGQAPRLLIYGASTRAI | |
| | cytoplasmic | GIPDRFSGSGSGTDFTLTISRLEPEDFAVYYCQQYG | |
| | signaling | SSPWTFGQGTKVEIKTTTPAPRPPTPAPTIASQPLS | |
| | domain, CD3ζ | LRPEACRPAAGGAVHTRGLDFACDIYIWAPLAGTCG | |
| | cytoplasmic | VLLLSLVITLYCKRGRKKLLYIFKQPFMRPVQTTQE | |
| | signaling | EDGCSCRFPEEEEGGCELRVKFSRSADAPAYQQGQN | |
| | domain | QLYNELNLGRREEYDVLDKRRGRDPEMGGKPRRKNP | |
| | | QEGLYNELQKDKMAEAYSEIGMKGERRRGKGHDGLY | |
| | | QGLSTATKDTYDALHMQALPPR | |
| 53B6- | CD8α signal | MALPVTALLLPLALLLHAARPQVQLQESGPGLVKPS | 76 |
| SR2 | sequence, 53B6 | ETLSLTCTVSGGSISYYSWTWVRQPAGQGLEWIGRI | |
| | scFv, CD20 | HISGVTNHNPSLKSRVSMSIDTSRTQFSLRLTSVTA | |
| | mimotope, CD20 | ADTAVYFCARSGGTYSAFDIWGQGTMVTVSSGGGGS | |
| | mimotope, CD8α | GGGGSGGGGSGGGGSEIVLTQSPGTLSLSPGERSTL | |
| | hinge and | SCRASQSFTSNYLAWYQQRPGQAPRLLIYGASTRAI | |
| | transmembrane | GIPDRFSGSGSGTDFTLTISRLEPEDFAVYYCQQYG | |
| | regions, 41BB | SSPWTFGQGTKVEIKGSGGGGSCPYSNPSLCSGGGG | |
| | cytoplasmic | SCPYSNPSLCSGGGGSTTTPAPRPPTPAPTIASQPL | |
| | signaling | SLRPEACRPAAGGAVHTRGLDFACDIYIWAPLAGTC | |
| | domain, CD3ζ | GVLLLSLVITLYCKRGRKKLLYIFKQPFMRPVQTTQ | |
| | cytoplasmic | EEDGCSCRFPEEEEGGCELRVKFSRSADAPAYQQGQ | |
| | signaling | NQLYNELNLGRREEYDVLDKRRGRDPEMGGKPRRKN | |
| | domain | PQEGLYNELQKDKMAEAYSEIGMKGERRRGKGHDGL | |
| | | YQGLSTATKDTYDALHMQALPPR | |
| 53B6- | CD8α signal | MALPVTALLLPLALLLHAARPGGGGSCPYSNPSLCG | 77 |
| RSR | sequence, CD20 | GGGSQVQLQESGPGLVKPSETLSLTCTVSGGSISYY | |
| | mimotope, 53B6 | SWTWVRQPAGQGLEWIGRIHISGVTNHNPSLKSRVS | |
| | scFv, CD20 | MSIDTSRTQFSLRLTSVTAADTAVYFCARSGGTYSA | |
| | mimotope, CD8α | FDIWGQGTMVTVSSGGGGSGGGGSGGGGSGGGGSEI | |
| | hinge and | VLTQSPGTLSLSPGERSTLSCRASQSFTSNYLAWYQ | |
| | transmembrane | QRPGQAPRLLIYGASTRAIGIPDRFSGSGSGTDFTL | |
| | regions, 41BB | TISRLEPEDFAVYYCQQYGSSPWTFGQGTKVEIKGG | |
| | cytoplasmic | GGSCPYSNPSLCTTTPAPRPPTPAPTIASQPLSLRP | |
| | signaling | EACRPAAGGAVHTRGLDFACDIYIWAPLAGTCGVLL | |
| | domain, CD3ζ | LSLVITLYCKRGRKKLLYIFKQPFMRPVQTTQEEDG | |
| | cytoplasmic | CSCRFPEEEEGGCELRVKFSRSADAPAYQQGQNQLY | |
| | signaling | NELNLGRREEYDVLDKRRGRDPEMGGKPRRKNPQEG | |
| | domain | LYNELQKDKMAEAYSEIGMKGERRRGKGHDGLYQGL | |
| | | STATKDTYDALHMQALPPR | |
| 53B6- | CD8α signal | MALPVTALLLPLALLLHAARPGGGGSCPYSNPSLCG | 78 |
| R2S | sequence, CD20 | GGGSCPYSNPSLCGGGGSQVQLQESGPGLVKPSETL | |
| | mimotope, CD20 | SLTCTVSGGSISYYSWTWVRQPAGQGLEWIGRIHIS | |
| | mimotope, 53B6 | GVTNHNPSLKSRVSMSIDTSRTQFSLRLTSVTAADT | |
| | scFv, CD8α | AVYFCARSGGTYSAFDIWGQGTMVTVSSGGGGSGGG | |
| | hinge and | GSGGGGSGGGGSEIVLTQSPGTLSLSPGERSTLSCR | |
| | transmembrane | ASQSFTSNYLAWYQQRPGQAPRLLIYGASTRAIGIP | |
| | regions, 41BB | DRFSGSGSGTDFTLTISRLEPEDFAVYYCQQYGSSP | |
| | cytoplasmic | WTFGQGTKVEIKTTTPAPRPPTPAPTIASQPLSLRP | |
| | signaling | EACRPAAGGAVHTRGLDFACDIYIWAPLAGTCGVLL | |
| | domain, CD3ζ | LSLVITLYCKRGRKKLLYIFKQPFMRPVQTTQEEDG | |
| | cytoplasmic | CSCRFPEEEEGGCELRVKFSRSADAPAYQQGQNQLY | |
| | signaling | NELNLGRREEYDVLDKRRGRDPEMGGKPRRKNPQEG | |
| | domain | LYNELQKDKMAEAYSEIGMKGERRRGKGHDGLYQGL | |
| | | STATKDTYDALHMQALPPR | |
| 53B6- | CD8α signal | MALPVTALLLPLALLLHAARPGGGGSCPYSNPSLCS | 79 |
| QR3 | sequence, CD20 | GGGGSGGGGSQVQLQESGPGLVKPSETLSLTCTVSG | |
| | mimotope, 53B6 | GSISYYSWTWVRQPAGQGLEWIGRIHISGVTNHNPS | |
| | scFv, CD20 | LKSRVSMSIDTSRTQFSLRLTSVTAADTAVYFCARS | |
| | mimotope, | GGTYSAFDIWGQGTMVTVSSGGGGSGGGGSGGGGSG | |
| | QBEND-10 | GGGSEIVLTQSPGTLSLSPGERSTLSCRASQSFTSN | |
| | epitope, CD20 | YLAWYQQRPGQAPRLLIYGASTRAIGIPDRFSGSGS | |
| | mimotope, | GTDFTLTISRLEPEDFAVYYCQQYGSSPWTFGQGTK | |
| | hinge and | VEIKGSGGGGSCPYSNPSLCSGGGGSELPTQGTFSN | |
| | transmembrane | VSTNVSPAKPTTTACPYSNPSLCTTTPAPRPPTPAP | |
| | regions of | TIASQPLSLRPEACRPAAGGAVHTRGLDFACDIYIW | |
| | human CD8α | APLAGTCGVLLLSLVITLYCKRGRKKLLYIFKQPFM | |
| | molecule, 41BB | RPVQTTQEEDGCSCRFPEEEEGGCELLRVKFSRSAD | |
| | signaling | APAYQQGQNQLYNELNLGRREEYDVLDKRRGRDPEM | |
| | domain, CD3ζ | GGKPRRKNPQEGLYNELQKDKMAEAYSEIGMKGERR | |
| | signaling | RGKGHDGLYQGLSTATKDTYDALHMQALPPR | |
| | domain | | |
| 53B6- | CD8α signal | MALPVTALLLPLALLLHAARPGGGGSCPYSNPSLCG | 180 |
| RSR (2) | sequence, CD20 | GGGSQVQLQESGPGLVKPSETLSLTCTVSGGSISYY | |
| | mimotope, 53B6 | SWTWVRQPAGQGLEWIGRIHISGVTNHNPSLKSRVS | |
| | scFv, CD20 | MSIDTSRTQFSLRLTSVTAADTAVYFCARSGGTYSA | |
| | mimotope, CD8α | FDIWGQGTMVTVSSGGGGSGGGGSGGGGSGGGGSEI | |
| | hinge and | VLTQSPGTLSLSPGERSTLSCRASQSFTSNYLAWYQ | |
| | transmembrane | QRPGQAPRLLIYGASTRAIGIPDRFSGSGSGTDFTL | |
| | regions, 41BB | TISRLEPEDFAVYYCQQYGSSPWTFGQGTKVEIKGG | |
| | cytoplasmic | GGSCPYSNPSLCGGGGSTTTPAPRPPTPAPTIASQP | |
| | signaling | LSLRPEACRPAAGGAVHTRGLDFACDIYIWAPLAGT | |
| | domain, CD3ζ | CGVLLLSLVITLYCKRGRKKLLYIFKQPFMRPVQTT | |
| | cytoplasmic | QEEDGCSCRFPEEEEGGCELRVKFSRSADAPAYQQG | |
| | signaling | QNQLYNELNLGRREEYDVLDKRRGRDPEMGGKPRRK | |
| | domain | NPQEGLYNELQKDKMAEAYSEIGMKGERRRGKGHDG | |
| | | LYQGLSTATKDTYDALHMQALPPR | |

The disclosure encompasses modifications to the CARs and polypeptides shown in Table 4, including functionally equivalent CARs having modifications which do not significantly affect their properties and variants which have enhanced or decreased activity and/or affinity. For example, the amino acid sequence may be mutated to obtain an antibody with the desired binding affinity to MUC16. Modification of polypeptides is routine practice in the art and need not be described in detail herein. Examples of modified polypeptides include polypeptides with conservative substitutions of amino acid residues, one or more deletions or additions of amino acids which do not significantly deleteriously change the functional activity, or which mature (enhance) the affinity of the polypeptide for its ligand, or use of chemical analogs.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to an epitope or mimotope tag. Other insertional variants of the antibody molecule include the fusion to the N- or C-terminus of the antibody of an enzyme or a polypeptide which increases the half-life of the antibody in the blood circulation.

Substitution variants have at least one amino acid residue in the antibody molecule removed and a different residue inserted in its place. The sites of greatest interest for substitutional mutagenesis include the hypervariable regions, but FR alterations are also contemplated. Conservative substitutions are shown in Table 6 under the heading of “conservative substitutions.” If such substitutions result in a change in biological activity, then more substantial changes, denominated “exemplary substitutions” in Table 6, or as further described below in reference to amino acid classes, may be introduced and the products screened.

In some embodiments, provided herein is a CAR, which specifically binds to MUC16, wherein the CAR comprises a VH region comprising a sequence shown in SEQ ID NO: 58; and/or a VL region comprising a sequence shown in SEQ ID NO: 59.

In some embodiments, the provided herein are CARs comprising CDR portions of antibodies to MUC16 antibodies based on CDR contact regions. CDR contact regions are regions of an antibody that imbue specificity to the antibody for an antigen. In general, CDR contact regions include the residue positions in the CDRs and Vernier zones which are constrained in order to maintain proper loop structure for the antibody to bind a specific antigen. See, e.g., Makabe et al., J. Biol. Chem., 283:1156-1166, 2007. Determination of CDR contact regions is well within the skill of the art.

The binding affinity (K_{D}) of the ligand binding domain of the MUC16 specific CAR as described herein to MUC16 (such as human MUC16 (e.g., Uniprot accession number: Q8WXI7)) can be for example about 0.1 to about 1000 nM, for example between about 0.5 nM to about 500 nM, or for example between about 1 nM to about 250 nM. In some embodiments, the binding affinity is about any of 1000 nm, 750 nm, 500 nm, 400 nm, 300 nm, 250 nm, 200 nM, 100 nM, 90 nM, 80 nM, 70 nM, 60 nM, 50 nM, 45 nM, 40 nM, 35 nM, 30 nM, 25 nM, 20 nM, 19 nm, 18 nm, 17 nm, 16 nm, 15 nM, 10 nM, 8 nM, 7.5 nM, 7 nM, 6.5 nM, 6 nM, 5.5 nM, 5 nM, 4 nM, 3 nM, 2 nM, 1 nM, 0.5 nM, 0.3 nM or 0.1 nM.

In some embodiments, the binding affinity is less than about any of 1000 nm, 900 nm, 800 nm, 250 nM, 200 nM, 100 nM, 50 nM, 30 nM, 20 nM, 10 nM, 7.5 nM, 7 nM, 6.5 nM, 6 nM, 5 nM.

### Monoclonal Antibody-Specific Epitopes and Mimotopes

In some embodiments, the extracellular domain of any one of the MUC16 specific CARs disclosed herein may comprise one or more epitopes or mimotopes specific for (i.e., specifically recognized by) a monoclonal antibody. These epitopes or mimotopes are also referred to herein as mAb-specific epitopes or mimotopes. In these embodiments, the extracellular domain comprises the VH and VL polypeptides that specifically bind to MUC16 and one or more epitopes or mimotopes that bind to one or more monoclonal antibodies (mAbs). CARs comprising the mAb-specific epitopes or mimotopes can be single-chain or multi-chain.

The inclusion of epitopes or mimotopes specific for monoclonal antibodies in the extracellular domain of the CARs described herein allows sorting and depletion of engineered immune cells expressing the CARs. In some embodiments, this feature also promotes recovery of endogenous MUC16 expressing cells that were depleted by administration of engineered immune cells expressing the CARs.

Accordingly, in some embodiments, the present disclosure relates to a method for sorting and/or depleting the engineered immune cells endowed with the CARs comprising mAb-specific epitopes or mimotopes and a method for promoting recovery of endogenous MUC16 expressing cells, such as bone marrow progenitor cells.

Several epitope- or mimotope-monoclonal antibody couples can be used to generate CARs comprising monoclonal antibody specific epitopes or mimotopes; in particular, those already approved for medical use, such as CD20 epitope or mimotope/rituximab as a non-limiting example.

In some embodiments, the monoclonal antibody specific for the epitope or mimotope may be conjugated with a cytotoxic drug. It is also possible to promote CDC cytotoxicity by using engineered antibodies on which are grafted component(s) of the complement system. In some embodiments, activation of the CAR-T cells can be modulated by depleting the cells using an antibody which recognizes the epitope or mimotope.

The disclosure also encompasses methods for sorting the engineered immune cells endowed with the MUC16 specific CARs expressing the mAb-specific epitope(s) or mimotope(s) and therapeutic methods where the activation of the engineered immune cells endowed with these CARs is modulated by depleting the cells using an antibody that targets the external ligand binding domain of said CARs.

CARs comprising one or more epitopes or mimotopes specifically recognized by a monoclonal antibody are disclosed in WO2016/120216, which is hereby incorporated by reference in its entirety. The one or more epitopes or mimotopes can be selected from any number of epitopes and mimotopes known in the art. In some embodiments, the one or more epitopes or mimotopes can be a target of a monoclonal antibody approved for medical use, such as, for example without limitation, the CD20 epitope or mimotope recognized by rituximab. In some embodiments, the one or more epitopes or mimotopes comprises any one or more of the amino acid sequences shown in SEQ ID NOs: 50, 51 and 74.

In some embodiments, the epitope or mimotope can be located between the scFv and the hinge of a CAR. In some embodiments, two instances of the same epitope or mimotope, separated by linkers, may be used in the CAR. For example, a polypeptide comprising 2 copies of the mimotope shown in SEQ ID NO: 50, separated by linkers, as shown in GSGGGGSCPYSNPSLCSGGGGSCPYSNPSLCSGGGGS (SEQ ID NO: 51), can be used within a CAR, located between the light chain variable region and the hinge.

In some embodiments, the extracellular binding domain of the CAR comprising the VH and VL polypeptides and the mAb-specific epitope(s) or mimotope(s) may have different structures depending on the position of insertion of the epitope(s) or mimotope(s) and the use of linkers. For example, the extracellular binding domain of the MUC16 specific CAR comprising mAb-specific epitopes or mimotopes may have one of the following structures (a mimotope may substitute for an epitope in the following structures):

V₁-L₁-V₂-(L)ₓ-Epitope1-(L)ₓ-;
V₁-L₁-V₂-(L)ₓ-Epitope1-(L)ₓ-Epitope2-(L)ₓ-;
V₁-L₁-V₂-(L)ₓ-Epitope1-(L)ₓ-Epitope2-(L)ₓ-
Epitope3-(L)ₓ-;
(L)ₓ-Epitope1-(L)ₓ-V₁-L₁-V₂;
(L)ₓ-Epitope1-(L)ₓ-Epitope2-(L)ₓ-V₁-L₁-V₂;
Epitopel-(L)ₓ-Epitope2-(L)ₓ-Epitope3-(L)ₓ-V₁-L₁-
V₂;
(L)ₓ-Epitope1-(L)ₓ-V₁-L₁-V₂-(L)ₓ-Epitope2-(L)ₓ;
(L)ₓ-Epitope1-(L)ₓ-V₁-L₁-V₂-(L)ₓ-Epitope2-(L)ₓ-
Epitope3-(L)ₓ-;
(L)ₓ-Epitope1-(L)ₓ-V₁-L₁-V₂-(L)ₓ-Epitope2-(L)ₓ-
Epitope3-(L)ₓ-Epitope4-(L)ₓ-;
(L)ₓ-Epitope1-(L)ₓ-Epitope2-(L)ₓ-V₁-L₁-V₂-(L)ₓ-
Epitope3-(L)ₓ-;
(L)ₓ-Epitopel-(L)ₓ-Epitope2-(L)ₓ-V₁-L₁-V₂-(L)ₓ-
Epitope3-(L)ₓ-Epitope4-(L)ₓ-;
V₁-(L)ₓ-Epitope1-(L)ₓ-V₂;
V₁-(L)ₓ-Epitope1-(L)ₓ-V₂-(L)ₓ-Epitope2-(L)ₓ;
V₁-(L)ₓ-Epitope1-(L)ₓ-V₂-(L)ₓ-Epitope2-(L)ₓ-
Epitope3-(L)ₓ;
V₁-(L)ₓ-Epitope1-(L)ₓ-V₂-(L)ₓ-Epitope2-(L)ₓ-
Epitope3-(L)ₓ-Epitope4-(L)ₓ;
(L)ₓ-Epitopel-(L)ₓ-V₁-(L)ₓ-Epitope2-(L)ₓ-V₂;
or,
(L)ₓ-Epitope1-(L)ₓ-V₁-(L)ₓ-Epitope2-(L)ₓ-V₂-(L)ₓ-
Epitope3-(L)ₓ;

wherein,
V₁ is V_{L} and V₂ is V_{H} or V₁ is V_{H} and V₂ is V_{L};
L₁ is a linker suitable to link the V_{H} chain to the V_{L} chain;
L is a linker comprising glycine and serine residues, and each occurrence of L in the extracellular binding domain can be identical or different to other occurrence of L in the same extracellular binding domain, for example SGGGG (SEQ ID NO: 185), GGGGS (SEQ ID NO: 178) or SGGGGS (SEQ ID NO: 186) (all of which occur, e.g., in SEQ ID NO:52), and,
x is 0 or 1 and each occurrence of x is selected independently from the others; and,
Epitope 1, Epitope 2, Epitope 3 and Epitope 4 are mAb-specific epitopes and can be identical or different.

In some embodiments, the extracellular binding domain of the CAR comprises the following sequence:
V₁-L₁-V₂-(L)ₓ-Epitope1-(L)ₓ-Epitope2-(L)ₓ-; or,
(L)ₓ-Epitope1-(L)ₓ-V₁-L₁-V₂-(L)ₓ-Epitope2-(L)ₓ-Epitope3-(L)ₓ-Epitope4-(L)ₓ-, wherein V1, V2, L₁, L, x and Epitope 1, Epitope 2, Epitope 3 and Epitope 4 are as defined above.

In some embodiments, any one of the MUC16 specific CARs disclosed herein may comprise one or more mAb-specific epitopes or mimotopes selected from a CD52 epitope or mimotope, a CD20 epitope or mimotope, a CD3 epitope or mimotope, a CD41 epitope or mimotope, a CD25 epitope or mimotope, a CD30 epitope or mimotope, an EGFR epitope or mimotope, a TNFα epitope or mimotope, a VEGF epitope or mimotope, a complement protein C5 epitope or mimotope, a CD1 la epitope or mimotope, a CD33 epitope or mimotope, an alpha-4 integrin epitope or mimotope, an IgE Fc region epitope or mimotope, an RSV protein F epitope or mimotope, an IL-6 receptor epitope or mimotope, a HER2 receptor epitope or mimotope, an integrin α₄β₇ epitope or mimotope, a BAFF (B-cell activatin factor) epitope or mimotope, an IL-1β epitope or mimotope, a RANKL epitope or mimotope, a CTLA4 epitope or mimotope, a CD34 epitope or mimotope, an IL-12 epitope or mimotope, and/or an IL-23 epitope or mimotope.

In some embodiments, the MUC16 specific CARs disclosed herein may comprise one or more mAb-specific epitopes or mimotopes selected from epitopes and mimotopes specifically recognized by alemtuzumab, ibritumomab tiuxetan, muromonab-CD3, tositumomab, abciximab, basiliximab, brentuximab vedotin, cetuximab, infliximab, rituximab, bevacizumab, certolizumab pegol, daclizumab, eculizumab, efalizumab, gemtuzumab, natalizumab, omalizumab, palivizumab, ranibizumab, tocilizumab, trastuzumab, vedolizumab, adalimumab, belimumab, canakinumab, denosumab, golimumab, ipilimumab, ofatumumab, panitumumab, QBEND-10 and/or ustekinumab.

In some embodiments, the MUC16 specific CARs comprise one or more mAb-specific epitopes or mimotopes selected from the epitopes and mimotopes disclosed in Table 5:

TABLE 5
Examples of mAb-specific epitopes and mimotopes
that can be used in the extracellular binding
domain of the MUC16 specific CARs of the
disclosure such as for example mimotopes and
epitopes with their corresponding mAb.
Rituximab

| | | |
|---|---|---|
| Mimotope | SEQ ID NO: 50 | CPYSNPSLC |

Palivizumab

| | | |
|---|---|---|
| Epitope | SEQ ID NO: 80 | NSELLSLINDMP |
| | | ITNDQKKLMSNN |

Cetuximab

| | | |
|---|---|---|
| Mimotope 1 | SEQ ID NO: 81 | CQFDLSTRRLKC |
| Mimotope 2 | SEQ ID NO: 82 | CQYNLSSRALKC |
| Mimotope 3 | SEQ ID NO: 83 | CVWQRWQKSYVC |
| Mimotope 4 | SEQ ID NO: 84 | CMWDRFSRWYKC |

Nivolumab

| | | |
|---|---|---|
| Epitope 1 | SEQ ID NO: 85 | SFVLNWYRMSPS |
| | | NQTDKLAAFPED |
| | | R |
| Epitope 2 | SEQ ID NO: 86 | SGTYLCGAISLA |
| | | PKAQIKE |

QBEND-10

| | | |
|---|---|---|
| Epitope | SEQ ID NO: 87 | ELPTQGTFSNVS |
| | | TNVSPAKPTTTA |

Alemtuzumab

| | | |
|---|---|---|
| Epitope | SEQ ID NO: 88 | GQNDTSQTSSPS |

The intracellular signaling domain of a CAR as disclosed herein is responsible for intracellular signaling following the binding of extracellular ligand-binding domain to the target resulting in the activation of the immune cell and immune response. The intracellular signaling domain has the ability to activate at least one of the normal effector functions of the immune cell in which the CAR is expressed. For example, the effector function of a T cell can be a cytolytic activity or helper activity including the secretion of cytokines.

### Expression of Proteins in Cells and Related Embodiments

In some embodiments, an immune cell e.g. T cell of the disclosure comprises and/or expresses a polypeptide that comprises any one or more of the sequences listed in Tables 1-3 (SEQ ID NOs: 1-79). In some embodiments, an immune cell e.g. T cell of the disclosure comprises and/or expresses a nucleic acid e.g. a vector that encodes such a polypeptide.

In some embodiments, an immune cell e.g. T cell of the disclosure comprises and/or expresses a polypeptide that comprises the amino acid sequence of MUC16 CAR1 (53B6) (SEQ ID NO: 11). In some embodiments, an immune cell e.g. T cell of the disclosure comprises and/or expresses a polypeptide that comprises the amino acid sequence of Sec49k (SEQ ID NO: 12). In some embodiments, an immune cell e.g. T cell of the disclosure comprises and/or expresses a polypeptide that comprises the amino acid sequence of UL11 (SEQ ID NO: 13). In some embodiments, an immune cell e.g. T cell of the disclosure comprises and/or expresses a polypeptide that comprises the amino acid sequence of BTN3A1-IgV (SEQ ID NO: 14). In some embodiments, an immune cell e.g. T cell of the disclosure comprises and/or expresses a polypeptide that comprises the amino acid sequence of BTN3A1-IgC (SEQ ID NO: 15).

In some embodiments, an immune cell e.g. T cell of the disclosure comprises and/or expresses a nucleic acid e.g. a vector that encodes a protease-activating CD45-gate CAR disclosed herein. In some embodiments, an immune cell e.g. T cell of the disclosure comprises and/or expresses a nucleic acid e.g. a vector that comprises a nucleic acid sequence that encodes the amino acid sequence of a MUC16 CAR (53B6) (any of SEQ ID NOs: 11, 56, 75, 76, 77, 78, 79, and 121-170). In some embodiments, an immune cell e.g. T cell of the disclosure comprises and/or expresses a nucleic acid e.g. a vector that comprises a nucleic acid sequence that encodes the amino acid sequence of Sec49k (SEQ ID NO: 12). In some embodiments, an immune cell e.g. T cell of the disclosure comprises and/or expresses a nucleic acid e.g. a vector that comprises a nucleic acid sequence that encodes the amino acid sequence of UL11 (SEQ ID NO: 13). In some embodiments, an immune cell e.g. T cell of the disclosure comprises and/or expresses a nucleic acid e.g. a vector that comprises a nucleic acid sequence that encodes the amino acid sequence of BTN3A1-IgV (SEQ ID NO: 14). In some embodiments, an immune cell e.g. T cell of the disclosure comprises and/or expresses a nucleic acid e.g. a vector that comprises a nucleic acid sequence that encodes the amino acid sequence of BTN3A1-IgC (SEQ ID NO: 15).

The disclosure encompasses modifications to the proteins of the disclosure embodiments shown in Table 2, including functionally equivalent proteins having modifications which do not significantly affect their properties and variants which have enhanced or decreased activity and/or affinity. Modification of polypeptides is routine practice in the art and need not be described in detail herein. Examples of modified polypeptides include polypeptides with conservative substitutions of amino acid residues, one or more deletions or additions of amino acids which do not significantly deleteriously change the functional activity, or which mature (enhance) the affinity of the polypeptide for its ligand, or use of chemical analogs.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue or the antibody fused to an epitope or mimotope tag.

Substitution variants have at least one amino acid residue in the protein removed and a different residue inserted in its place. Conservative substitutions are shown in Table 6 under the heading of “conservative substitutions.” If such substitutions result in a change in biological activity, then more substantial changes, denominated “exemplary substitutions” in Table 6, or as further described below in reference to amino acid classes, may be introduced and the products screened.

TABLE 6
Amino Acid Substitutions

| Original residue | | |
|---|---|---|
| (naturally occurring | Conservative | |
| amino acid) | substitutions | Exemplary subsitutions |
| Ala (A) | Val | Val; Leu; Ile |
| Arg (R) | Lys | Lys; Gln; Asn |
| Asn (N) | Gln | Gln; His; Asp; Lys; Arg |
| Asp (D) | Glu | Glu; Asn |
| Cys (C) | Ser | Ser; Ala |
| Gln (Q) | Asn | Asn; Glu |
| Glu (E) | Asp | Asp; Gln |
| Gly (G) | Ala | Ala |
| His (H) | Arg | Asn; Gln; Lys; Arg |
| Ile (I) | Leu | Leu; Val; Met; Ala; |
| | | Phe; Norleucine |
| Leu (L) | Ile | Norleucine; Ile; Val; |
| | | Met; Ala; Phe |
| Lys (K) | Arg | Arg; Gln; Asn |
| Met (M) | Leu | Leu; Phe; Ile |
| Phe (F) | Tyr | Leu; Val; Ile; Ala; Tyr |
| Pro (P) | Ala | Ala |
| Ser (S) | Thr | Thr |
| Thr (T) | Ser | Ser |
| Trp (W) | Tyr | Tyr; Phe |
| Tyr (Y) | Phe | Trp; Phe; Thr; Ser |
| Val (V) | Leu | Ile; Leu; Met; |
| | | Phe; Ala; Norleucine |

Protease-activating CD45-gate CAR protein and protease-activating CD45-gate CAR protein derivatives may be synthesized in situ in the cell after introduction of polynucleotides encoding the proteins into the cell. Alternatively, protease-activating CD45-gate CAR protein and protease-activating CD45-gate CAR protein derivative proteins may be produced outside of cells, and then introduced into cells. Methods for introducing a polynucleotide construct into cells are known in the art. In some embodiments, stable transformation methods can be used to integrate the polynucleotide construct into the genome of the cell. In other embodiments, transient transformation methods can be used to transiently express the polynucleotide construct, and the polynucleotide construct not integrated into the genome of the cell. In other embodiments, virus-mediated methods can be used. The polynucleotides may be introduced into a cell by any suitable means such as for example, recombinant viral vectors (e.g. retroviruses e.g. lentiviruses, adenoviruses), liposomes, and the like. Transient transformation methods include, for example without limitation, microinjection, electroporation or particle bombardment. Polynucleotides may be included in vectors, such as for example plasmid vectors or viral vectors.

In some embodiments, an immune cell e.g. T cell of the disclosure can comprise at least one protease-activating CD45-gate CAR protein or protease-activating CD45-gate CAR protein derivative. In some embodiments, an immune cell e.g. T cell can comprise at least one protease-activating CD45-gate CAR protein or protease-activating CD45-gate CAR protein derivative and one or more additional CARs (gated or not gated), and each CAR may comprise different extracellular ligand-binding domains.

In some embodiments of an immune cell e.g. T cell provided herein, a CAR that the T cell expresses can comprise an extracellular ligand-binding domain (e.g., a single chain variable fragment (scFv)), a transmembrane domain, and an intracellular signaling domain. In some embodiments, the extracellular ligand-binding domain, transmembrane domain, and intracellular signaling domain are in one polypeptide, i.e., in a single chain. Multichain CARs and polypeptides are also provided herein. In some embodiments, the multichain CARs comprise: a first polypeptide comprising a transmembrane domain and at least one extracellular ligand-binding domain, and a second polypeptide comprising a transmembrane domain and at least one intracellular signaling domain, wherein the polypeptides assemble together to form a multichain CAR. The CAR can be modified as described herein to comprise a CD45 recruiting domain that is linked to the amino terminus of the CAR's extracellular ligand-binding domain.

The extracellular ligand-binding domain specifically binds to a target of interest. In some embodiments, the target of interest can be any molecule of interest, including, for example, without limitation, BCMA, MUC16 (also known as CA125), EGFR, EGFRVIII, MUC1, Flt-3, WT-1, CD20, CD23, CD30, CD38, CD70, CD33, CD133, MHC-WT1, TSPAN10, MHC-PRAME, MHC-NY-ESO1, HER2 (ERBB2), CAIX (Carbonic anhydrase IX), LIV1, ADAM10, CHRNA2, LeY, NKG2D, CS1, CD44v6, ROR1, CD19, Claudin-18.2 (Claudin-18A2, or Claudin18 isoform 2), PSCA, DLL3 (Delta-like protein 3, Drosophila Delta homolog 3, Delta3), Mud 7 (Mucin17, Muc3, Muc3), FAP alpha (Fibroblast Activation Protein alpha), Ly6G6D (Lymphocyte antigen 6 complex locus protein God, c6orf23, G6D, MEGT1, NG25), PSMA, MSLN, or RNF43 (E3 ubiquitin-protein ligase RNF43, RING finger protein 43).

In some embodiments, the extracellular ligand-binding domain comprises an scFv comprising the light chain variable (V_{L}) region and the heavy chain variable (VH) region of a target antigen specific monoclonal antibody joined by a flexible linker. Single chain variable region fragments are made by linking light and/or heavy chain variable regions by using a short linking peptide (Bird et al., Science 242:423-426, 1988). An example of a linking peptide is the GS linker having the amino acid sequence (GGGGS) 3 (SEQ ID NO: 57), which bridges approximately 3.5 nm between the carboxy terminus of one variable region and the amino terminus of the other variable region. Linkers of other sequences have been designed and used (Bird et al., 1988, supra). In general, linkers can be short, flexible polypeptides and preferably comprised of about 20 or fewer amino acid residues. Linkers can in turn be modified for additional functions, such as attachment of drugs or attachment to solid supports. The single chain variants can be produced either recombinantly or synthetically. For synthetic production of scFv, an automated synthesizer can be used. For recombinant production of scFv, a suitable plasmid or other vector containing a polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

In some embodiments, a flexible linker as described herein is modified to comprise a protease cleavage site and the modified linker connects the CD45 recruiting domain of the protease-activating CD45-gate CAR described herein to the antigen binding domain of the protease-activating CD45-gate CAR. An example of such a modified linker comprises the amino acid sequence of SEQ ID NO: 9.

The intracellular signaling domain of a protease-activating CD45-gate CAR according to the disclosure is responsible for intracellular signaling following the binding of extracellular ligand-binding domain to the target resulting in the activation of the immune cell and immune response. The intracellular signaling domain has the ability to activate at least one of the normal effector functions of the immune cell in which the protease-activating CD45-gate CAR is expressed. For example, the effector function of a T cell can be a cytolytic activity or helper activity including the secretion of cytokines.

In some embodiments, an intracellular signaling domain for use in a protease-activating CD45-gate CAR can be the cytoplasmic sequences of, for example without limitation, the T cell receptor and co-receptors that act in concert to initiate signal transduction following antigen receptor engagement, as well as any derivative or variant of these sequences and any synthetic sequence that has the same functional capability. Intracellular signaling domains comprise two distinct classes of cytoplasmic signaling sequences: those that initiate antigen-dependent primary activation, and those that act in an antigen-independent manner to provide a secondary or co-stimulatory signal. Primary cytoplasmic signaling sequences can comprise signaling motifs which are known as immunoreceptor tyrosine-based activation motifs or ITAMs. ITAMs are well defined signaling motifs found in the intracytoplasmic tail of a variety of receptors that serve as binding sites for syk/zap70 class tyrosine kinases. Examples of ITAM used in the protease-activating CD45-gate CARs of the disclosure can include as non-limiting examples those derived from TCRξ, FcRγ, FcRβ, FcRε, CD3γ, CD3δ, CD3ε, CD5, CD22, CD79a, CD79b and CD66d. In some embodiments, the intracellular signaling domain of the CAR can comprise the CD3ξ signaling domain (e.g. comprising or consisting of the amino acid sequence of SEQ ID NO: 34).

In some embodiments the intracellular signaling domain of the protease-activating CD45-gate CAR of the disclosure comprises a domain of a co-stimulatory molecule. In some embodiments, the intracellular signaling domain of a protease-activating CD45-gate CAR of the disclosure comprises a part of a co-stimulatory molecule selected from the group consisting of a fragment of 41BB (GenBank: AAA53133, e.g. comprising or consisting of the amino acid sequence of SEQ ID NO: 33) and CD28 (NP_006130.1, e.g. comprising or consisting of the amino acid sequence of SEQ ID NO: 35). In some embodiments, the intracellular signaling domain of the CAR of the disclosure comprises an amino acid sequence which comprises at least 70%, preferably at least 80%, more preferably at least 90%, 95%, 97%, or 99% sequence identity with one or more of the amino acid sequences shown in SEQ ID NOs: 33-35.

CARs generally are expressed on the surface membrane of the cell. Thus, the protease-activating CD45-gate CAR disclosed herein can comprise a transmembrane domain. Suitable transmembrane domains for a protease-activating CD45-gate CAR disclosed herein have the ability to (a) be expressed at the surface of a cell, for example an immune cell such as, for example without limitation, lymphocyte cells (e.g. T cells) or Natural killer (NK) cells, and (b) interact with the ligand-binding domain and intracellular signaling domain for directing a cellular response of an immune cell against a predefined target cell. The transmembrane domain can be derived either from a natural or from a synthetic source. The transmembrane domain can be derived from any membrane-bound or transmembrane protein. As non-limiting examples, the transmembrane polypeptide can be a domain of the T cell receptor such as α, β, γ or δ polypeptide constituting CD3 complex, IL-2 receptor e.g. p55 (α chain), p75 (β chain or γ chain), subunit chain of Fc receptors, in particular Fcγ receptor III or CD proteins. Alternatively, the transmembrane domain can be synthetic and can comprise predominantly hydrophobic residues such as leucine and valine. In some embodiments said transmembrane domain is derived from the human CD8α chain (e.g., NP_001139345.1). The protease-activating CD45-gate CAR can further comprise a stalk domain between the extracellular ligand-binding domain and said transmembrane domain. A stalk domain may comprise up to 300 amino acids, for example, from 10 to 100 amino acids or 25 to 50 amino acids. The stalk region may be derived from all or part of naturally occurring molecules, such as from all or part of the extracellular region of CD8, CD4, or CD28, or from all or part of an antibody constant region. Alternatively the stalk domain may be a synthetic sequence that corresponds to a naturally occurring stalk sequence, or may be an entirely synthetic stalk sequence. In some embodiments said stalk domain is a part of human CD8α chain (e.g., NP_001139345.1). In another particular embodiment, the transmembrane domain comprises a part of the human CD8α chain. In some embodiments, protease-activating CD45-gate CARs disclosed herein can comprise a CD45 recruiting domain, a protease-cleavable linker, an extracellular ligand-binding domain, CD8α human stalk and transmembrane domains, the CD3ξ signaling domain, and 4-1BB signaling domain. In some embodiments, a nucleic acid encoding a protease-activating CD45-gate CAR as disclosed herein can be introduced into an immune cell as a transgene via a vector e.g. a plasmid vector. In some embodiments, the vector e.g. plasmid vector can also contain, for example, a selection marker which provides for identification and/or selection of cells which received the vector.

Protease-activating CD45-gate CAR polypeptides may be synthesized in situ in the cell after introduction of polynucleotides encoding the protease-activating CD45-gate CAR polypeptides into the cell. Alternatively, protease-activating CD45-gate CAR polypeptides may be produced outside of cells, and then introduced into cells. Methods for introducing a polynucleotide construct into cells are known in the art. In some embodiments, stable transformation methods can be used to integrate the polynucleotide construct into the genome of the cell. In other embodiments, transient transformation methods can be used to transiently express the polynucleotide construct, and the polynucleotide construct not integrated into the genome of the cell. In other embodiments, virus-mediated methods can be used. The polynucleotides may be introduced into a cell by any suitable means such as for example, recombinant viral vectors (e.g. retroviruses (e.g. lentiviruses), adenoviruses), liposomes, and the like. Transient transformation methods include, for example without limitation, microinjection, electroporation or particle bombardment. Polynucleotides may be included in vectors, such as for example plasmid vectors or viral vectors.

Methods of generating engineered immune cells expressing any of the protease-activating CD45-gate CARs provided herein is described in WO/2016/166630, incorporated by reference in its entirety.

Also provided herein are immune cells e.g. T cells such as isolated T cells obtained according to any one of the methods described herein and modified e.g. engineered to comprise and express the nucleic acids, vectors and polypeptides disclosed herein. Any immune cell capable of expressing heterologous DNAs can be used for the purpose of expressing the protease-activating CD45-gate CAR protein or protease-activating CD45-gate CAR protein derivative and optionally any additional CAR of interest. In some embodiments, the immune cell is a T cell. In some embodiments, an immune cell can be derived from, for example without limitation, a stem cell. The stem cells can be adult stem cells, non-human embryonic stem cells, more particularly non-human stem cells, cord blood stem cells, progenitor cells, bone marrow stem cells, induced pluripotent stem cells, totipotent stem cells or hematopoietic stem cells. Representative human cells are CD34+ cells. The isolated cell can also be a dendritic cell, killer dendritic cell, a mast cell, a NK-cell, a B-cell or a T cell selected from the group consisting of inflammatory T-lymphocytes, cytotoxic T-lymphocytes, regulatory T-lymphocytes or helper T-lymphocytes. In some embodiments, the cell can be derived from the group consisting of CD4+T-lymphocytes and CD8+T-lymphocytes.

In some embodiments, the engineered immune cell disclosed herein is an engineered B cell, mast cell, myeloic-derived phagocyte, T cell e.g. an alpha/beta and/or gamma/delta T cell, tumor infiltrating lymphocyte (TIL), NK cell, TCR-expressing cell, dendritic cell, or NK-T cell. In some embodiments, the cell is and/or is derived from an autologous T cell. In some embodiments, the cell is and/or is derived from an allogeneic T cell.

In some embodiments, the immune cells e.g. T cells such as isolated T cells are further modified e.g. genetically modified by methods described herein (e.g. known gene editing techniques that employ, for example, TALENs, CRISPR/Cas9, or megaTAL nucleases).

Prior to expansion and genetic modification, a source of cells can be obtained from a subject through a variety of non-limiting methods. Cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In some embodiments, any number of T cell lines available and known to those skilled in the art, may be used. In some embodiments, cells can be derived from a healthy donor, from a subject diagnosed with cancer or from a subject diagnosed with an infection. In some embodiments, cells can be part of a mixed population of cells which present different phenotypic characteristics.

In some embodiments, an isolated cell or engineered immune cell according to the present disclosure comprises one inactivated gene selected from the group consisting of CD52, GR, PD-1, CTLA-4, LAG3, Tim3, BTLA, BY55, TIGIT, B7H5, LAIR1, SIGLEC10, 2B4, HLA, TCRα and TCRβ and/or expresses a CAR, a multi-chain CAR and/or a pTα transgene. In some embodiments, an isolated cell comprises polynucleotides encoding polypeptides comprising a multi-chain CAR. In some embodiments, the isolated cell according to the present disclosure comprises two inactivated genes selected from the group consisting of: CD52 and GR, CD52 and TCRα, CDR52 and TCRβ, GR and TCRα, GR and TCRβ, TCRα and TCRβ, PD-1 and TCRα, PD-1 and TCRβ, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ and/or expresses a CAR (e.g. the protease-activating CD45-gate CAR of the present disclosure or the anti-MUC16 CAR of the present disclosure), a multi-chain CAR and a pTα transgene.

Gene inactivation can be carried out by methods practiced by those with skill in the art. The methods include, but are not limited to gene inactivation by use of zinc fingers, TALEN®s, and CRISPR/Cas-based system.

In some embodiments, the protease-activating CD45-gate CAR-containing immune cell has an inactivated CD52 gene. In some embodiments only one copy of the CD52 gene is inactivated.

In some embodiments, the protease-activating CD45-gate CAR-containing immune cell has an inactivated TCRα gene.

In some embodiments, the protease-activating CD45-gate CAR-containing immune cell has an inactivated TCRβ gene.

In some embodiments, TALEN® is used for gene inactivation. In such embodiments, the efficiency of gene inactivation with TALEN® is not 100%, and resulting TCRαβ-negative T-cells are enriched by depleting residual TCRαβ-positive T cells before cryopreservation. However, CD52-negative cells are not purified, resulting in a cell product with varying frequencies of CD52-negative cells, typically between 60-80%. Accordingly in some embodiments, the genotype of the protease-activating CD45-gate CAR T cells of the disclosure is protease-activating CD45-gate CAR+_TCRαβ−_CD52+/−T-cells.

In some embodiments, TCR is rendered not functional in the cells according to the disclosure by inactivating TCRα gene and/or TCRβ gene(s). In some embodiments, a method to obtain modified cells derived from an individual is provided, wherein the cells can proliferate independently of the major histocompatibility complex (MHC) signaling pathway. Modified cells, which can proliferate independently of the MHC signaling pathway, susceptible to be obtained by this method are encompassed in the scope of the present disclosure. Modified cells disclosed herein can be used in for treating subjects in need thereof against Host versus Graft (HvG) rejection and Graft versus Host Disease (GvHD); therefore in the scope of the present disclosure is a method of treating subjects in need thereof against Host versus Graft (HvG) rejection and Graft versus Host Disease (GvHD) comprising treating said subject by administering to said subject an effective amount of modified cells comprising inactivated TCRα and/or TCRβ genes.

In some embodiments, the immune cells are engineered to be resistant to one or more chemotherapy drugs. The chemotherapy drug can be, for example, a purine nucleotide analogue (PNA), thus making the immune cell suitable for cancer treatment combining adoptive immunotherapy and chemotherapy. Exemplary PNAs include, for example, clofarabine, fludarabine, and cytarabine, alone or in combination. PNAs are metabolized by deoxycytidine kinase (dCK) into mono-, di-, and tri-phosphate PNA. Their tri-phosphate forms compete with ATP for DNA synthesis, act as pro-apoptotic agents, and are potent inhibitors of ribonucleotide reductase (RNR), which is involved in trinucleotide production. Provided herein are protease-activating CD45-gate CAR-T cells comprising an inactivated dCK gene. In some embodiments, the dCK knockout cells are made by transfection of T cells using polynucleotides encoding specific TAL-nuclease directed against dCK genes by, for example, electroporation of mRNA. The dCK knockout protease-activating CD45-gate CAR-T cells are resistant to PNAs, including for example clorofarabine and/or fludarabine, and maintain T cell cytotoxic activity toward cells that express the ligand of the protease-activating CD45-gate CAR.

In some embodiments, isolated cells or cell lines of the disclosure can comprise a pTα or a functional variant thereof. In some embodiments, an isolated cell or cell line can be further genetically modified by inactivating the TCRα gene.

In some embodiments, the CAR-T cell (e.g. protease-activating CD45-gate CAR-expressing T cell) comprises a polynucleotide encoding a safety switch, such as for example RQR8. See, e.g., WO2013153391A, which is hereby incorporated by reference in its entirety. In CAR-T cells comprising the polynucleotide, the safety switch polypeptide is expressed at the surface of a CAR-T cell. In some embodiments, the safety switch polypeptide comprises the amino acid sequence shown in SEQ ID NO: 48.

(SEQ ID NO: 48)
CPYSNPSLCSGGGGSELPTQGTFSNVSTNVSPAKPTTTACPYSNPSLCSG
GGGSPAPRPPTPAPTIASQPLSLRPEACRPAAGGAVHTRGLDFACDIYIW
APLAGTCGVLLLSLVITLYCNHRNRRRVCKCPRPVV

The safety switch polypeptide may also comprise a signal peptide at the amino terminus. In some embodiments, the safety switch polypeptide comprises the amino acid sequence shown in SEQ ID NO: 49.

(SEQ ID NO: 49)
MGTSLLCWMALCLLGADHADACPYSNPSLCSGGGGSELPTQGTFSNVSTN
VSPAKPTTTACPYSNPSLCSGGGGSPAPRPPTPAPTIASQPLSLRPEACR
PAAGGAVHTRGLDFACDIYIWAPLAGTCGVLLLSLVITLYCNHRNRRRVC
KCPRPVV

When the safety switch polypeptide is expressed at the surface of a CAR-T cell, binding of rituximab to the R epitopes or mimotopes of the polypeptide causes lysis of the cell. More than one molecule of rituximab may bind per polypeptide expressed at the cell surface. Each R epitope or mimotope of the polypeptide may bind a separate molecule of rituximab. Deletion of protease-activating CD45-gate CAR-T cells may occur in vivo, for example by administering rituximab to a subject. The decision to delete the transferred cells may arise from undesirable effects being detected in the subject which are attributable to the transferred cells, such as for example, when unacceptable levels of toxicity are detected.

In some embodiments, the CAR-T cell comprises a selected epitope or mimotope within the scFv or extracellular domain (e.g. stalk domain) of the protease-activating CD45-gate CAR, the selected epitope or mimotope having a specificity to be recognized by a specific antibody. See, e.g., PCT application PCT/EP2016/051467, WO2016/120216, “mAb-DRIVEN CHIMERIC ANTIGEN RECEPTOR SYSTEMS FOR SORTING/DEPLETING ENGINEERED IMMUNE CELLS,” filed on Jan. 25, 2016, which is hereby incorporated by reference in its entirety. Such an epitope or mimotope facilitates sorting and/or depleting the CAR-T cells. The epitope or mimotope can be selected from any number of epitopes and mimotopes known in the art. In some embodiments, the epitope or mimotope can be a target of a monoclonal antibody approved for medical use, such as, for example without limitation, a CD20 epitope or mimotope recognized by rituximab. In some embodiments, the epitope or mimotope comprises the amino acid sequence shown in SEQ ID NO: 50.

(SEQ ID NO: 50)
CPYSNPSLC

In some embodiments, the epitope or mimotope is located within the CAR. For example without limitation, the epitope or mimotope can be located between the scFv and the hinge of a CAR. In some embodiments, two instances of the same epitope or mimotope, separate by linkers, may be used in the CAR. For example, the polypeptide comprising the amino acid sequence shown in SEQ ID NO: 51 can be used within a CAR, located between the light chain variable region and the hinge.

(SEQ ID NO: 51)
GSGGGGSCPYSNPSLCSGGGGSCPYSNPSLCSGGGGS

In some embodiments, the epitope- or mimotope-specific antibody may be conjugated with a cytotoxic drug. It is also possible to promote CDC cytotoxicity by using engineered antibodies on which are grafted component(s) of the complement system. In some embodiments, activation of the CAR-T cells can be modulated by depleting the cells using an antibody which recognizes the epitope or mimotope.

Adverse events also may be minimized by transducing the immune cells (containing one or more CARs) with a suicide gene. It may also be desired to incorporate an inducible “on” or “accelerator” switch into the immune cells. Suitable techniques include use of inducible caspase-9 (U.S. Appl. 2011/0286980) or a thymidine kinase, before, after or at the same time, as the cells are transduced with the CAR construct of the present disclosure. Additional methods for introducing suicide genes and/or “on” switches include TALENS, zinc fingers, RNAi, siRNA, shRNA, antisense technology, and other techniques known in the art.

In accordance with the disclosure, additional on-off or other types of control switch techniques may be incorporated herein. These techniques may employ the use of dimerization domains and optional activators of such domain dimerization. These techniques include, e.g., those described by Wu et al., Science 2014 350 (6258) utilizing FKBP/Rapalog dimerization systems in certain cells, the contents of which are incorporated by reference herein in their entirety. Additional dimerization technology is described in, e.g., Fegan et al. Chem. Rev. 2010, 110, 3315-3336 as well as U.S. Pat. Nos. 5,830,462; 5,834,266; 5,869,337; and 6,165,787, the contents of which are also incorporated by reference herein in their entirety. Additional dimerization pairs may include cyclosporine-A/cyclophilin, receptor, estrogen/estrogen receptor (optionally using tamoxifen), glucocorticoids/glucocorticoid receptor, tetracycline/tetracycline receptor, vitamin D/vitamin D receptor. Further examples of dimerization technology can be found in e.g., WO 2014/127261, WO 2015/090229, US 2014/0286987, US2015/0266973, US2016/0046700, U.S. Pat. No. 8,486,693, US 2014/0171649, and US 2012/0130076, the contents of which are further incorporated by reference herein in their entirety.

Also provided herein are cell lines obtained from a modified e.g. transformed immune cell e.g. T cell according to any of the methods described herein. In some embodiments, an immune cell e.g. T cell according to the disclosure comprises a polynucleotide encoding a protease-activating CD45-gate CAR protein or protease-activating CD45-gate CAR protein derivative. In some embodiments, an immune cell e.g. T cell according to the disclosure comprises a polynucleotide encoding a protease-activating CD45-gate CAR protein or protease-activating CD45-gate CAR protein derivative and a polynucleotide encoding an additional CAR.

The immune cells e.g. T cells of the disclosure can be activated and expanded, either prior to or after modification of the cells, using methods as generally described, for example without limitation, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005. Immune cells e.g. T cells can be expanded in vitro or in vivo. Generally, the immune cells of the disclosure can be expanded, for example, by contact with an agent that stimulates a CD3 TCR complex and a co-stimulatory molecule on the surface of the immune cells to create an activation signal for the cell. For example, chemicals such as calcium ionophore A23187, phorbol 12-myristate 13-acetate (PMA), or mitogenic lectins like phytohemagglutinin (PHA) can be used to create an activation signal for the immune cell e.g. T cell.

In some embodiments, T cell populations may be stimulated in vitro by contact with, for example, an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. Conditions appropriate for T cell culture include an appropriate medium (e.g., Minimal Essential Media or RPMI Media 1640 or, X-vivo 5, (Lonza)) that may contain factors necessary for proliferation and viability, including serum (e.g., fetal bovine or human serum), interleukin-2 (IL-2), insulin, IFN-γ, IL-4, IL-7, GM-CSF, IL-10, IL-2, IL-15, a TGFβ, and TNF, or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM V, DMEM, MEM, α-MEM, F-12, X-Vivo 10, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO2). Immune cells e.g. T cells that have been exposed to varied stimulation times may exhibit different characteristics.

In some embodiments, the cells of the disclosure can be expanded by co-culturing with tissue or cells. The cells can also be expanded in vivo, for example in the subject's blood after administrating the cell into the subject.

In another aspect, the disclosure provides compositions (such as pharmaceutical compositions) comprising any of the cells of the disclosure or any of the populations of cells of the disclosure. In some embodiments, the composition comprises a T cell comprising a polynucleotide encoding a protease-activating CD45-gate CAR protein or protease-activating CD45-gate CAR protein derivative, which may further comprise a polynucleotide encoding a second CAR. The compositions comprise, for example, an immune cell e.g. T cell of the disclosure, e.g. an immune cell that expresses a protease-activating CD45-gate CAR and/or protease-activating CD45-gate CAR protein derivative, or comprise a population of cells that comprises an immune cell e.g. T cell of the disclosure, and one or more pharmaceutically acceptable carriers or excipients.

In some embodiments, primary cells isolated from a donor are manipulated as described herein to provide a population of cells of which a subpopulation (e.g. a proportion less than 100%, such as 10%, 20%, 30%) of the resulting cells comprise all of the desired modifications. Such a resulting population comprising a mixture of cells that comprise all of the modifications and cells that do not can be used in the methods of treatment of the disclosure and to prepare the compositions of the disclosure. Alternatively, this population of cells (the “starting population”) can be manipulated by known methods e.g. cell sorting and/or expansion of cells that have the desired modifications, to provide a population of cells that is enriched for those cells comprising one or more of the desired modifications (e.g. enriched for cells that express the desired antigen binding protein, for cells that express a protease-activating CD45-gate CAR protein and/or protease-activating CD45-gate CAR protein derivative), that is, that comprises a higher percentage of such modified cells than did the starting population. The population enriched for the modified cells can then be used in the methods of treatment of the disclosure and to prepare the compositions of the disclosure, for example. In some embodiments, the enriched population of cells contains or contains at least 40%, 50%, 60%, 70%, 80%, 90%, 95%, or 99% cells that have one or more of the modifications. In other embodiments, the proportion of cells of the enriched population of cells that comprise one or more of the modifications is at least 30% higher than the proportion of cells of the starting population of cells that comprise the desired modifications.

In some embodiments, a population of immune cells comprises one or more of the engineered immune cells disclosed herein. In an embodiment, the population comprises about or at least about 1×10⁴, about or at least about 1×10⁵, about or at least about 1×10⁶, about or at least about 1×10⁷, or about or at least about 1×10⁸ engineered cells, e.g. engineered immune cells, as disclosed herein, optionally wherein the population does not comprise more than about 1×10¹⁰ or more than about 1×10⁹ or more than about 5×10⁹ engineered cells, e.g. engineered immune cells, as disclosed herein. In an embodiment, a population of immune cells as disclosed herein is enriched for the engineered immune cell as disclosed herein. In various embodiments, the population of immune cells is at least 50%, e.g. 50%, 55%, 60%, 65%, 70%, 75%, 80%, 85%, 90%, 95%, or more than 95% engineered cells that are T cells, e.g., alpha/beta T cells and gamma/delta T cells, B cells, natural killer (NK) cells, natural killer T (NKT) cells, mast cells, and/or myeloic-derived phagocytes. In various embodiments, the population of immune cells is at least 50%, e.g. 50%, 55%, 60%, 65%, 70%, 75%, 80%, 85%, 90%, 95%, or more than 95% engineered T cells.

### Methods of Treating

Immune cells e.g. T cells obtained by the methods described above, or cell lines derived from such immune cells or T cells, can be used as a medicament. In some embodiments, such a medicament can be used for treating a disorder such as for example a viral disease, a bacterial disease, a cancer, an inflammatory disease, an immune disease, an autoimmune disease, an infection, or an aging-associated disease. In some embodiments, the cancer is a hematological malignancy or non-solid tumor. In some embodiments, the cancer is a solid cancer or solid tumor. In some embodiments, the cancer is a solid cancer or solid tumor. In some embodiments, the cancer is a hematological malignancy that is acute lymphoblastic leukemia (ALL), acute myeloid leukemia (AML), chronic myelogenous leukemia (CML), chronic eosinophilic leukemia (CEL), myelodysplasia syndrome (MDS), non-Hodgkin's lymphoma (NHL), or multiple myeloma (MM). In some embodiments, the cancer is a solid cancer that is biliary cancer, bladder cancer, bone or soft tissue carcinoma, brain tumor, breast cancer, cervical cancer, colon cancer, colorectal adenocarcinoma, colorectal cancer, desmoid tumor, embryonal cancer, endometrial cancer, esophageal cancer, gastric cancer, gastric adenocarcinoma, glioblastoma multiforme, gynecological tumor, head and neck squamous cell carcinoma, hepatic cancer, lung cancer, malignant melanoma, osteosarcoma, ovarian cancer, pancreatic cancer, pancreatic ductal adenocarcinoma, primary astrocytic tumor, primary thyroid cancer, prostate cancer, renal cancer, renal cell carcinoma, rhabdomyosarcoma, skin cancer, soft tissue sarcoma, testicular germ-cell tumor, urothelial cancer, uterine sarcoma, or uterine cancer. In some embodiments, the cancer can be selected from the group consisting of gastric cancer, sarcoma, osteosarcoma, rhabdomyosarcoma, tissue sarcoma, uterine sarcoma, lymphoma, leukemia, head and neck cancer, thymic cancer, epithelial cancer, salivary cancer, liver cancer, stomach cancer, thyroid cancer, lung cancer, ovarian cancer, breast cancer, prostate cancer, esophageal cancer, pancreatic cancer, glioma, leukemia, multiple myeloma, renal cell carcinoma, bladder cancer, cervical cancer, choriocarcinoma, colon cancer, oral cancer, skin cancer, and melanoma. In some embodiments, the subject is a previously treated adult subject with locally advanced or metastatic melanoma, squamous cell head and neck cancer (SCHNC), ovarian carcinoma, sarcoma, or relapsed or refractory classic Hodgkin's Lymphoma (cHL). In an embodiment, the condition is ovarian cancer and the protease-activating CD45-gate CAR comprises the amino acid sequence of any of SEQ ID NOs: 11, 56, 75, 76, 77, 78, 79, and 121-170.

In some embodiments, immune cells e.g. T cells according to the disclosure, or cell line derived from the immune cells e.g. T cells, can be used in the manufacture of a medicament for treatment of a disorder in a subject in need thereof. In some embodiments, the disorder can be, for example, a cancer, an autoimmune disorder, or an infection.

Also provided herein are methods for treating subjects. In some embodiments the method comprises administering or providing an immune cell e.g. T cell of the disclosure to a subject in need thereof. In some embodiments, the method comprises a step of administering the immune cells e.g. T cells of the disclosure to a subject in need thereof.

In another aspect, provided herein is a method of treating a condition in a patient comprising administering to the patient an engineered immune cell as disclosed herein. In an embodiment of the method, the engineered immune cell is an allogeneic engineered immune cell derived from a donor other than the patient.

In another aspect, provided herein is a method of treating a condition in a patient comprising administering to the patient a population of immune cells as disclosed herein. In an embodiment of the method, the immune cells of the population are derived from one or more allogeneic immune cells from a donor other than the patient.

In another aspect, provided herein is a method of treating a condition in a patient comprising administering to the patient a pharmaceutical composition as disclosed herein. In an embodiment of the method, the composition comprises one or more engineered allogeneic immune cells derived from a donor other than the patient.

In some embodiments, immune cells e.g. T cells of the disclosure can undergo robust in vivo cell expansion and can persist for an extended amount of time. Methods of treatment of the disclosure can be ameliorating, curative or prophylactic. The method of the disclosure may be either part of an autologous immunotherapy or part of an allogenic immunotherapy treatment. The disclosure is particularly suitable for allogeneic immunotherapy. Immune cells e.g. T cells from donors can be transformed into non-alloreactive cells using standard protocols and reproduced as needed, thereby producing e.g. CAR-T cells which may be administered to one or several subjects. Such CAR-T cell therapy can be made available as an “off the shelf” therapeutic product.

In another aspect, the disclosure provides a method of inhibiting tumor growth or progression in a subject who has a tumor, comprising administering to the subject an effective amount of immune cells e.g. T cells as described herein. In another aspect, the disclosure provides a method of inhibiting or preventing metastasis of cancer cells in a subject, comprising administering to the subject in need thereof an effective amount of immune cells e.g. T cells as described herein. In another aspect, the disclosure provides a method of inducing tumor regression in a subject who has a tumor, comprising administering to the subject an effective amount of immune cells e.g. T cells as described herein.

In some embodiments, the immune cells e.g. T cells provided herein can be administered parenterally in a subject. In some embodiments, the subject is a human.

In some embodiments, the method can further comprise administering an effective amount of a second therapeutic agent. In some embodiments, the second therapeutic agent is, for example, crizotinib, palbociclib, an anti-CTLA4 antibody, an anti-4-1 BB antibody, a PD-1 antibody, or a PD-L1 antibody.

Also provided is the use of any of the immune cells e.g. T cells provided herein in the manufacture of a medicament for the treatment of cancer or for inhibiting tumor growth or progression in a subject in need thereof.

Also provided herein are uses and methods as described herein wherein the condition is ovarian cancer and the protease-activating CD45-gate CAR comprises the amino acid sequence of any of SEQ ID NOs: 11, 56, 75, 76, 77, 78, 79, and 121-170. Also provided herein are uses and methods as described herein wherein the condition is ovarian cancer and a CAR comprising the amino acid sequence of any of SEQ ID NOs: 11, 56, 75, 76, 77, 78, 79, and 121-170 is used in addition to or instead of the protease-activating CD45-gate CAR.

The degree to which the gate modulates the protease-activating CD45-gate CAR's activity can be assessed in various ways. One way is to measure the cytotoxicity of a T cell that expresses the protease-activating CD45-gate CAR in comparison to various controls, such as a T cell that expresses a protein that is the same as the protease-activating CD45-gate CAR but that lacks a protease cleavage site in the linker that connects the CD45 recruiting domain to the ligand binding domain (non-cleavable gate), a T cell that expresses a protein that comprises the same CAR but that has no CD45 recruiting domain (non-gated CAR), and a T cell that has not been transduced. The cytotoxicity of these cells can be measured by incubating them with target cells that express the ligand that the CAR antigen binding domain recognizes. The assay can be adjusted by varying the ratio of the effector CAR T cell to the target ligand-expressing cell. When the effector and target cells are incubated together under the appropriate conditions, while not wishing to be bound by theory, it is expected that the CAR T cells will exhibit high cytotoxicity and both the cleavable and non-cleavable CD45-gate CAR T cells will exhibit lower cytotoxicity because the gate at least partially inactivates the CAR. It is expected that the non-transduced cells will exhibit no cytotoxicity. This form of assay serves to confirm that, under the conditions of the assay, the gate can at least partially inactivate the CAR when the CAR's ligand is present but the protease is absent. It thus mimics the in vivo condition in which the protease-activating CD45-gate CAR T cell encounters its antigen off-tumor. In such an assay, in some embodiments, the protease-activating CD45-gate CAR cytotoxicity is reduced by or by at least about 20%, 30%, 40%, 50%, 60%, 70%, 80%, or 90% relative to the non-gated CAR. The degree of reduction observed depends on the conditions of the assay.

To mimic the in vivo condition in which the protease-activating CD45-gate CAR T cell encounters antigen at the tumor site, the same assay can be repeated in the presence of a protease that can cleave the linker that connects the CD45 recruiting domain to the ligand binding domain. It is expected that the protease will not affect the non-gated CAR T cells' cytotoxicity. It is also expected that the cleavable protease-activating CD45-gate CAR will show reduced cytotoxicity in the absence of the protease and will show higher cytotoxicity in the presence of the protease as compared with in the absence of the protease, because, while not wishing to be bound by theory, in the presence of the protease, the gate that inhibited CAR activation has been removed by cleavage of the linker. It is expected that the non-cleavable gate CAR cytotoxicity will not be affected by the presence of the protease. It is expected that its cytotoxicity will be reduced both in the absence of and in the presence of the protease, because, while not wishing to be bound by theory, the gate inhibits the CAR's activation under both conditions. In such an assay, in some embodiments, in the absence of protease, the protease-activating CD45-gate CAR cytotoxicity is reduced by or by at least about 20%, 30%, 40%, 50%, 60%, 70%, 80%, or 90% relative to the non-gated CAR and, in the presence of protease, the cells' cytotoxicity is significantly higher. The degrees of reduction and increase observed depend on the conditions of the assay.

In another aspect, provided herein is a method of reducing on-target off-tumor toxicity of CAR T cells comprising administering to a patient a cell as disclosed herein, a population of cells as disclosed herein, or a composition as disclosed herein, wherein the on-target, off-tumor toxicity is lower than the on-target, off-tumor toxicity of control cells, a population of control cells or a composition of control cells, respectively, administered under the same conditions, wherein the control cells, population of control cells, or control cells of the composition comprise a CAR that lacks at least one of a CD45 recruiting domain and a linker that comprises a protease cleavage site.

In another aspect, provided herein is a method of increasing the efficiency of CAR T cells or CAR T cell therapy comprising administering to a patient a cell as disclosed herein, a population of cells as disclosed herein, or a composition as disclosed herein, wherein the efficiency is greater than the efficiency of control cells, a population of control cells or a composition of control cells, respectively, administered under the same conditions, wherein the control cells, population of control cells, or control cells of the composition comprise a CAR that lacks at least one of a CD45 recruiting domain and a linker that comprises a protease cleavage site.

In another aspect, provided herein is a method of increasing the efficacy of CAR T cell therapy comprising administering to a patient a cell as disclosed herein, a population of cells as disclosed herein, or a composition as disclosed herein, wherein the efficacy is greater than the efficacy of control cells, a population of control cells or a composition of control cells, respectively, administered under the same conditions, wherein the control cells, population of control cells, or control cells of the composition comprise a CAR that lacks at least one of a CD45 recruiting domain and a linker that comprises a protease cleavage site.

In another aspect, provided herein is a method of increasing the efficacy of CAR T cell therapy against a solid tumor comprising administering to a patient having a solid tumor a cell as disclosed herein, a population of cells as disclosed herein, or a composition as disclosed herein, wherein the efficacy against the solid tumor is greater than the efficacy against the solid tumor of control cells, a population of control cells or a composition of control cells, respectively, administered under the same conditions, wherein the control cells, population of control cells, or control cells of the composition comprise a CAR that lacks at least one of a CD45 recruiting domain and a linker that comprises a protease cleavage site.

In another aspect, provided herein is a method of reducing the incidence of side effects in CAR T cell therapy comprising administering to a patient a cell as disclosed herein, a population of cells as disclosed herein, or a composition as disclosed herein, wherein the incidence of side effects is lower than the incidence of side effects when control cells, a population of control cells or a composition of control cells, respectively, are administered under the same conditions, wherein the control cells, population of control cells, or control cells of the composition comprise a CAR that lacks at least one of a CD45 recruiting domain and a linker that comprises a protease cleavage site.

In another aspect, provided herein is a method of effecting reversible colocalization of CD45 and a CD45-gate CAR in a CAR T cell, the reversible colocalization comprising a CD45-gate CAR recruiting domain binding to a CD45 protein on a CAR T cell surface, resulting in colocalization, which binding and colocalization can be disrupted by a protease cleaving a protease cleavage site in the CD45-gate CAR's linker, thereby reversing the colocalization, the method comprising the steps of:
(a) providing a T cell that expresses CD45,
(b) introducing a nucleic acid encoding a protease-activating CD45-gate CAR as disclosed herein into the T cell,
(c) maintaining the T cell under conditions in which both CD45 and the CAR are functionally expressed at the cell surface,
resulting in a CD45-gate CAR recruiting domain binding to a CD45 protein on the CAR T cell surface and colocalization of the CAR and the CD45 protein, which colocalization can be reversed by a protease cleaving a protease cleavage site in the CD45-gate CAR's linker, wherein the cleaving produces a functional CAR no longer connected by the linker to the CD45-gate CAR recruiting domain.

In another aspect, provided herein is a method of effecting reversible reduction of CAR activity by a CD45 protein in a CAR T cell, the reversible reduction of CAR activity comprising a CD45-gate CAR recruiting domain binding to a CD45 protein on a CAR T cell surface, resulting in inactivation of the chimeric antigen receptor by the CD45, which inactivation can be disrupted by a protease cleaving a protease cleavage site in the CD45-gate CAR's linker, resulting in the disassociation of the chimeric antigen receptor and the CD45 protein and ending the inactivation of the chimeric antigen receptor by the CD45, the method comprising the steps of:
(a) providing a T cell that expresses CD45,
(b) introducing a nucleic acid encoding a CD45-gate CAR into the T cell,
(c) maintaining the T cell under conditions in which both CD45 and the CAR are functionally expressed at the cell surface,
resulting in a CD45-gate CAR recruiting domain binding to a CD45 protein on the CAR T cell surface, association of the CAR and the CD45 protein, and reduction of the CAR's activity by the CD45 protein, which activity reduction can be reversed by a protease cleaving a protease cleavage site in the CD45-gate CAR's linker, thereby ending the association of the CAR and the CD45 protein, and ending the CD45 protein's reduction of CAR activity, wherein the cleaving produces a functional CAR no longer connected by the linker to the CD45-gate CAR recruiting domain.

In another aspect, provided herein is a method of treating with CAR T cell therapy a patient who has a tumor characterized by a protease-rich tumor microenvironment, comprising administering to the patient a cell as disclosed herein, a population of cells as disclosed herein, or a composition as disclosed herein, wherein CAR T cell activity is lower outside the protease-rich tumor microenvironment than in the protease-rich tumor microenvironment.

In another aspect, provided herein is a method of regulating cytotoxic activity of a CAR T cell that comprises a CAR as disclosed herein, the method comprising inhibiting the cytotoxic activity of the CAR T cell by associating the CAR with CD45 of the CAR T cell, and activating the cytotoxic activity of the CAR T cell by subjecting and/or exposing the CAR T cell to a protease that recognizes and cleaves the protease cleavage site.

In embodiments of the methods of treating a condition in a patient disclosed herein, the cell, population of cells or composition can be administered to the subject on one occasion or can be administered to the subject on two or more occasions spaced at least about 1, 2, 3, 4, 5, 6, 7, or more days apart. In some embodiments, the disorder can be a viral disease, a bacterial disease, a cancer, an inflammatory disease, an immune disease, or an aging-associated disease.

In some embodiments, the treatment can be in combination with one or more therapies against cancer selected from the group of antibodies therapy, chemotherapy, cytokines therapy, dendritic cell therapy, gene therapy, hormone therapy, laser light therapy and radiation therapy.

In some embodiments, treatment can be administered to or administrated into subjects undergoing an immunosuppressive treatment. Indeed, the disclosure may rely on cells or a population of cells which have been made resistant to at least one immunosuppressive agent due to the inactivation of a gene encoding a receptor for such immunosuppressive agent. In this aspect, the immunosuppressive treatment may help the selection and expansion of the T cells according to the disclosure within the subject.

The administration of the cells or population of cells according to the disclosure may be carried out in any convenient manner, including by aerosol inhalation, injection, ingestion, transfusion, implantation or transplantation. The compositions described herein may be administered to a subject subcutaneously, intradermally, intratumorally, intranodally, intramedullary, intramuscularly, by intravenous or intralymphatic injection, or intraperitoneally. In one embodiment, the cell compositions of the disclosure are administered by intravenous injection.

In some embodiments, the administration of the cells or population of cells can comprise administration of, for example, about 10⁴ to about 10⁹ cells per kg body weight including all integer values of cell numbers within those ranges. In some embodiments the administration of the cells or population of cells can comprise administration of about 10⁵ to about 10⁶ cells per kg body weight including all integer values of cell numbers within those ranges. The cells or population of cells can be administered in one or more doses. In some embodiments, an effective amount of cells can be administered as a single dose. In some embodiments, an effective amount of cells can be administered as more than one dose over a period time. Timing of administration is within the judgment of the managing physician and depends on the clinical condition of the subject. The cells or population of cells may be obtained from any source, such as a blood bank or a donor. While individual needs vary, determination of optimal ranges of effective amounts of a given cell type for a particular disease or conditions is within the skill of the art. An effective amount means an amount which provides a therapeutic or prophylactic benefit. The dosage administered will be dependent upon the age, health and weight of the recipient, the kind of concurrent treatment, if any, the frequency of treatment and the nature of the effect desired. In some embodiments, an effective amount of cells or composition comprising those cells are administered parenterally. In some embodiments, administration can be an intravenous administration. In some embodiments, administration can be directly done by injection within a tumor.

In some embodiments of the disclosure, cells are administered to a subject in conjunction with (e.g., before, simultaneously or following) any number of relevant treatment modalities, including but not limited to treatment with agents such as monoclonal antibody therapy, CCR2 antagonist (e.g., INC-8761), antiviral therapy, cidofovir and interleukin-2, Cytarabine (also known as ARA-C) or nataliziimab treatment for MS subjects or efaliztimab treatment for psoriasis subjects or other treatments for PML subjects. In some embodiments, protease-activating CD45-gate CAR-T cells are administered to a subject in conjunction with one or more of the following: an anti-PD-1 antibody (e.g., nivolumab, pembrolizumab, or PF-06801591), an anti-PD-L1 antibody (e.g., avelumab, atezolizumab, or durvalumab), an anti-OX40 antibody (e.g., PF-04518600), an anti-4-1 BB antibody (e.g., PF-05082566), an anti-MCSF antibody (e.g., PD-0360324), an anti-GITR antibody, and/or an anti-TIGIT antibody. In further embodiments, the immune cells e.g. T cells of the disclosure may be used in combination with chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludaribine, cyclosporin, FK506, rapamycin, mycoplienolic acid, steroids, FR901228, cytokines, and/or irradiation. These drugs inhibit either the calcium dependent phosphatase calcineurin (cyclosporine and FK506) or inhibit the p70S6 kinase that is important for growth factor induced signaling (rapamycin) (Henderson, Naya et al. Immunology. 1991 July; 73 (3): 316-321; Liu, Albers et al. Biochemistry 1992 Apr. 28; 31 (16): 3896-901; Bierer, Hollander et al. Curr Opin Immunol. 1993 October; 5 (5): 763-73).

In a further embodiment, the cell compositions of the disclosure are administered to a subject in conjunction with (e.g., before, simultaneously or following) bone marrow transplantation, T cell ablative therapy using either chemotherapy agents such as fludarabine, external-beam radiation therapy (XRT), cyclophosphamide, or antibodies such as OKT3 or CAMPATH. In some embodiments, the cell compositions of the disclosure are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of expanded immune cells of the disclosure. In some embodiments, expanded cells are administered before or following surgery.

### Kits

The disclosure also provides kits for use in the instant methods. Kits of the disclosure include one or more containers comprising a composition of the disclosure or an immune cell e.g. a T cell of the disclosure or a population of cells comprising an immune cell e.g. a T cell of the disclosure. In various embodiments, the immune cell e.g. T cell comprises one or more polynucleotide(s) encoding a protease-activating CD45-gate CAR protein as described herein or a protease-activating CD45-gate CAR protein derivative. The kit further comprises instructions for use in accordance with any of the methods of the disclosure described herein. Generally, these instructions comprise a description of administration of the composition, immune cell e.g. T cell or population of cells for the above described therapeutic treatments. In some embodiments the engineered immune cells are formulated in a solution comprising about 5% DMSO. Further, the engineered immune cells can be provided in a frozen state.

The instructions relating to the use of the kit components generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert (e.g., a paper sheet included in the kit), but machine-readable instructions (e.g., instructions carried on a magnetic or optical storage disk) are also acceptable.

The kits of this disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an immune cell e.g. T cell according to the disclosure. The container may further comprise a second pharmaceutically active agent.

Kits may optionally provide additional components such as buffers and interpretive information. Normally, the kit comprises a container and a label or package insert(s) on or associated with the container.

### EXAMPLES

The following examples are offered for illustrative purposes only, and are not intended to limit the scope of the disclosure in any way. Indeed, various modifications of the disclosure in addition to those shown and described herein will become apparent to those skilled in the art from the foregoing description and fall within the scope of the appended claims.

### Example 1: Synthesis and Demonstration of CD45-Gate CAR

### CD45-Gate CAR Structure

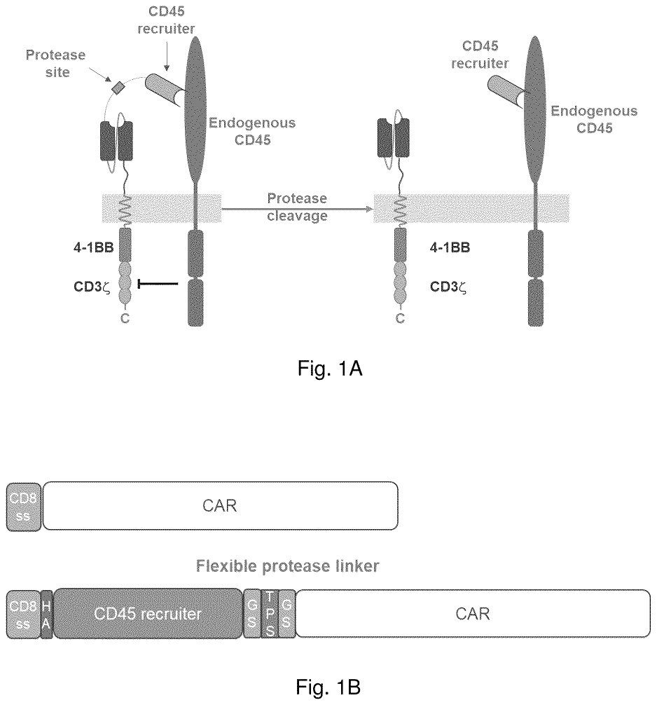

FIG. 1A shows a schematic representation of a protease-activating CD45-gate CAR. To investigate how the protease-activating CD45-gate can regulate the function of CAR T cells, we used a 2nd generation CAR (“53B6”) that specifically targets MUC16-expressing cells. A generic CAR protein is comprised of an extracellular target-binding domain, a transmembrane domain, and an intracellular signaling domain that, for example, comprises an activation domain (e.g. CD3 zeta cytoplasmic domain) and optionally also a costimulatory domain (e.g. 4-1BB signalling domain). In general, the extracellular domain of the CD45-gate CAR disclosed herein comprises, in addition, a CD45 recruiting domain (“recruiter”), fused via a flexible linker, which linker comprises, in certain embodiments, one or more protease-cleavable sites, to the N-terminus of a generic CAR, as illustrated in FIGS. 1A and 1B (bottom construct). As diagrammed in FIG. 1B (bottom construct), an additional N-HA tag (comprising the sequence YPYDVPDYA (SEQ ID NO: 187), a variant of SEQ ID NO: 2 without the initial G) was introduced as well for detection of the CD45-gate CAR, and the CD8 signal peptide was fused to the N-terminus of each protein to enable trafficking and surface expression. Non-gated, non-N-HA-tagged 53B6 (FIG. 1B, top construct) was used as a control in some of the experiments.

### CD45-Gate CAR Expression in T Cells

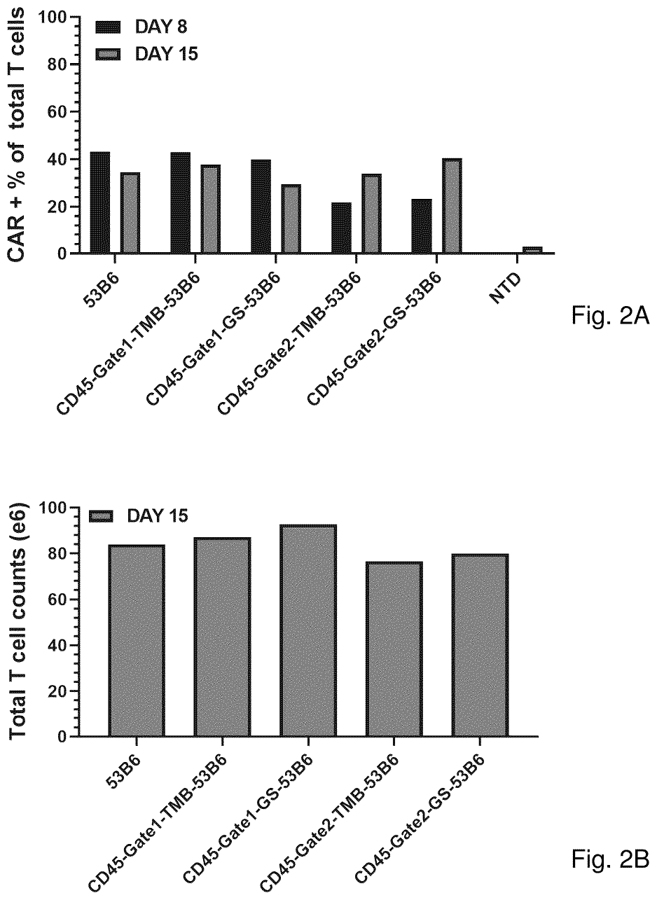

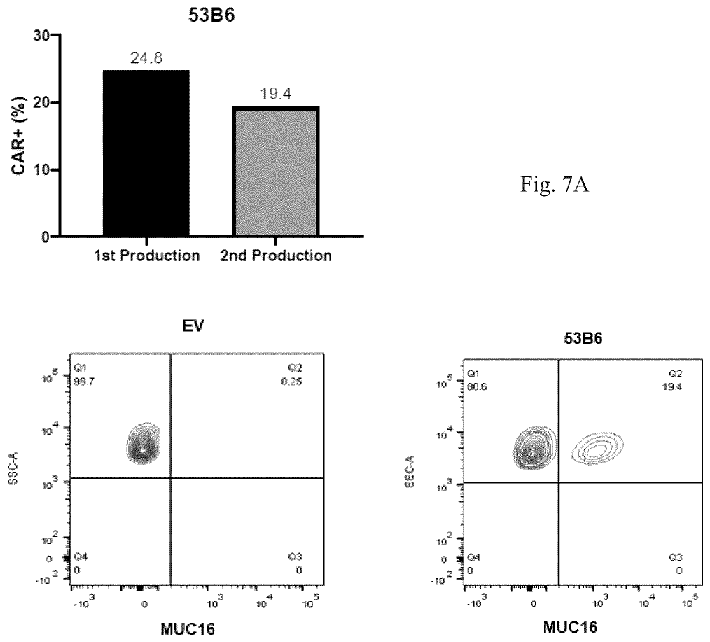

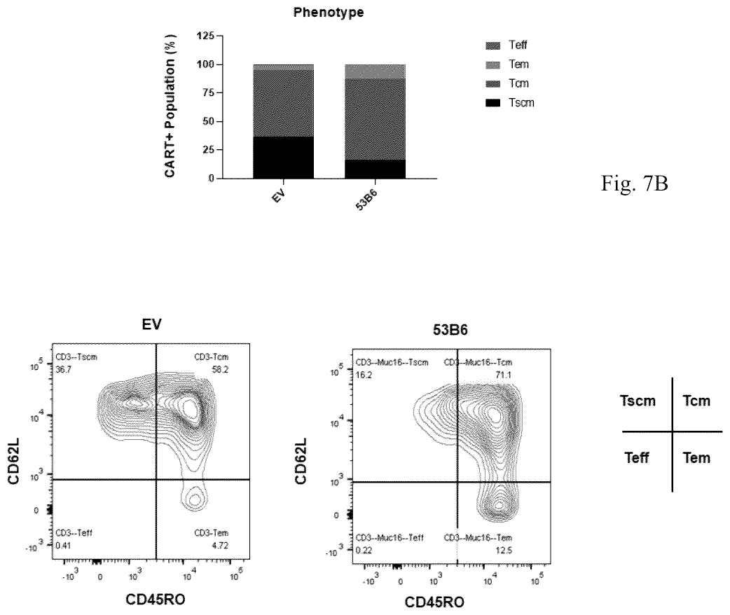

To evaluate CAR T cells that functionally express the protease-activating CD45-gate CAR, two variants of the CD45-gate were prepared, one comprising a first anti-CD45 scFv as the CD45 recruiter and the other comprising a second, different anti-CD45 scFv as the CD45 recruiter. A flexible protease-cleavable linker, which contained two thrombin protease cleavage sites flanking a V5 peptide motif, which can be used to detect the linker (SEQ ID NO: 8, GGGGSGGGGSLVPRGSKPIPNPLLGLDSTLVPRGSGGGGSGGGGS), joined the CD45 recruiter to the N-terminus of the CAR. These two protease-activating CD45-gate CAR clones are designated “CD45-Gate1-TMB-53B6” (comprising amino acid sequence of SEQ ID NO: 16) and “CD45-Gate2-TMB-53B6” (“TMB” indicating the thrombin-cleavable linker) (comprising amino acid sequence of SEQ ID NO: 18), where the exemplary anti-MUC16 CAR clone 53B6 was used to demonstrate the proof of concept of such a protease-activating CD45-gate CAR. In parallel, a non-cleavable linker of the same length (SEQ ID NO: 7, GGGGSGGGGSGGGSGSKPIPNPLLGLDSTGGGSGSGGGGSGGGGS) was used to generate additional control CARs, designated “CD45-Gate1-GS-53B6” (comprising amino acid sequence of SEQ ID NO: 17) and “CD45-Gate2-GS-53B6” (comprising amino acid sequence of SEQ ID NO: 19), both of which are not expected to be cleaved by a protease. CAR T cells expressing either the non-gate 53B6 CAR or the CD45-gate 53B6 CARs were generated with lentiviral vectors as previously described (14,15). In brief (and using the methods described in Example 2 below, CAR-T Production), primary T cells from a healthy donor were transduced with lentiviruses (MOI of 5) expressing the different CAR molecules and the percentage of CAR+ cells were recorded at day 8 and day 15. As the data in FIG. 2A show, CARs with the CD45-gate1 (“CD45-Gate1-TMB-53B6” and “CD45-Gate1-GS-53B6”) show CAR+ rate comparable to that of the 53B6 CAR throughout the production. The CD45-gate2 CARs (“CD45-Gate2-TMB-53B6” and “CD45-Gate2-GS-53B6”) exhibit lower transduction rate initially, but are able to match up to the CAR+ percentage in the end of the production (data for FIG. 2A were obtained as described below under Example 2 with respect to FIG. 7A, Flow Cytometry Analysis). The overall growth of T cells remains similar among all the clones (see FIG. 2B). These data together indicate that, although CD45 is expressed on the surface of CAR T cells, surprisingly the CD45-gate CAR can be produced and expressed in the T cells successfully without causing severe fratricide.

### CD45-Gate CAR Protease Cleavage and Ligand Binding

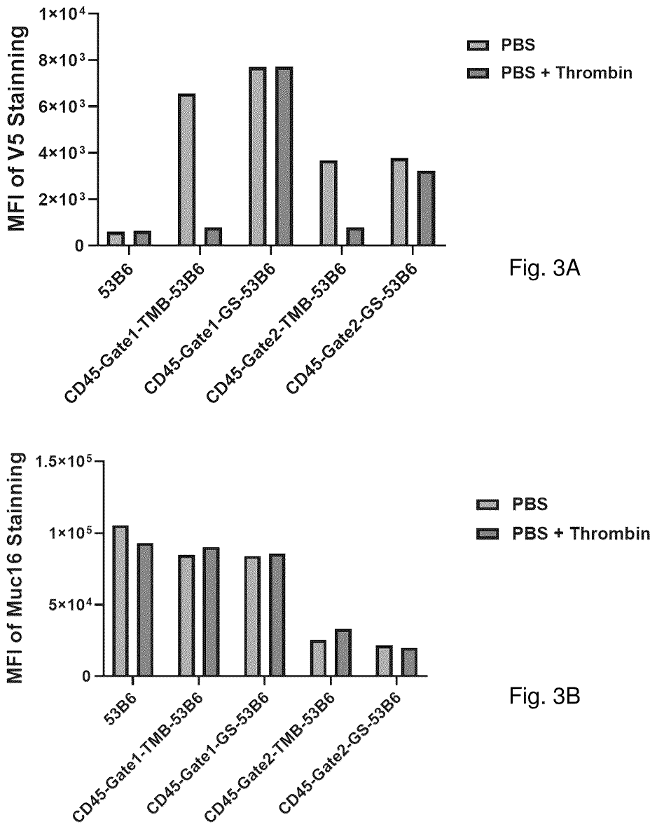

To evaluate whether the CD45-gate can be released by the corresponding protease, the detection of V5 tag that is imbedded in the cleavable or non-cleavable linkers was assessed. The protease cleavage was carried out by incubating the CAR T cells in PBS with 2 microgram/ml of thrombin at 37° for 30 mins. The successful cleavage of the protease linker was verified by the removal of V5 tag after cleavage. As the data in FIG. 3A show, the removal of V5 tag was only observed in the clone of CD45-Gate1-TMB-53B6 and CD45-Gate2-TMB-53B6 after cleavage, indicating that the CD45-gate can be released from CAR by the specific cleavage by the protease.

Next, whether the CD45-gate directly blocks the ligand binding was evaluated by assessing the binding of soluble MUC16 ligand to each CAR clone, before or after protease cleavage. It was observed that 53B6, CD45-Gate1-TMB-53B6, CD45-Gate1-GS-53B6 show very similar ligand-binding intensity, regardless of protease cleavage (see data in FIG. 3B). CD45-Gate2-TMB-53B6 and CD45-Gate2-GS-53B6 exhibited decreased ligand-binding intensity, likely due to inferior expression of the modified CAR molecule (see data in FIG. 3B). Nonetheless, the binding was not affected by protease cleavage and the release of CD45-gate. Overall, these data demonstrate that CD45 gate does not block binding to soluble target.

### CD45-Gate CAR T Cell Cytotoxicity

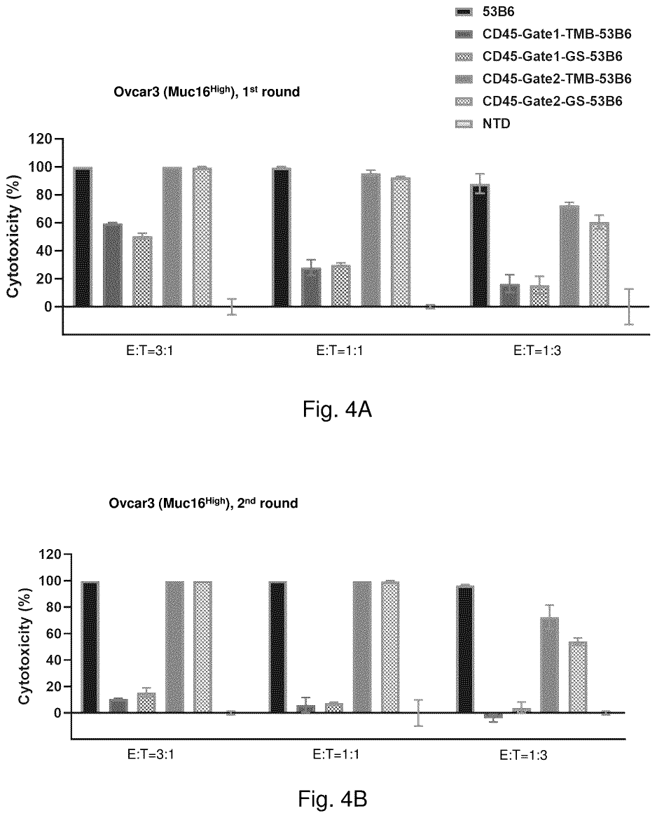

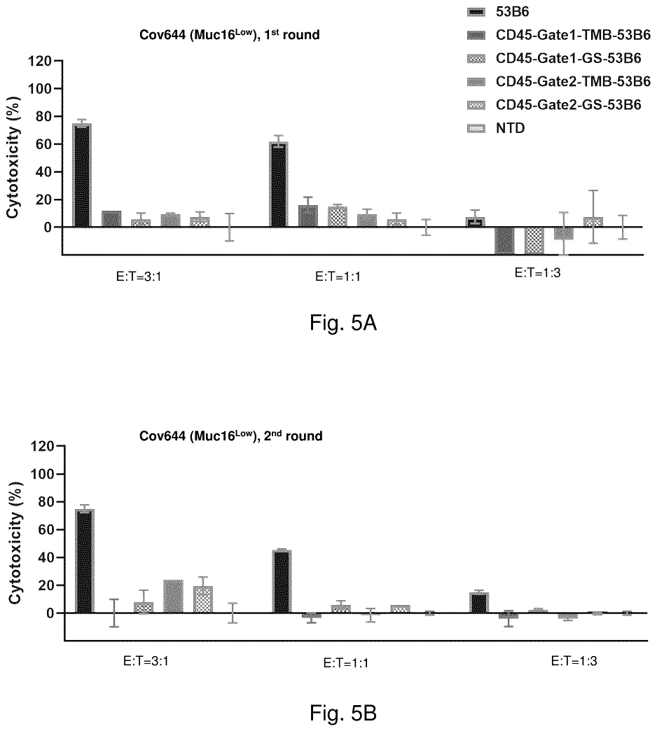

To understand whether the CD45-gate can regulate the CAR function, two target cell lines, Ovcar3 (MUC16High) and Cov644 (MUC16Low), were used to assess the cytotoxicity of each clone. Both target cells are incorporated with luciferase gene for facile quantification of viable cells. In brief, CAR T cells were normalized to the same CAR+ percentage and incubated with 10,000 target cells at E:T (Effector: Target) ratio of 3:1, 1:1, or 1:3 in 200 μl of RPMI medium with 10% of FBS. After the 48-hour co-culture, 100 μl of the supernatant containing CAR T cells was transferred to new wells with 10,000 fresh target cells, while the remaining samples were used to quantify the viable target cells by luciferase assay. After another 48-hour co-culture, the second round of cytotoxicity assessment was carried out in the same way. As FIGS. 4A-4B show, CD45-Gate1 moderately inhibited the CAR function to kill the Ovcar3 (MUC16High) target cells in the first round of cytotoxicity assay and the inhibition became more pronounced in the second round. However, the CD45-Gate2 showed very limited inhibition of the CAR activity; a reduction in cytotoxic activity was observed only at low E:T ratio (FIGS. 4A and 4B). In contrast, both CD45-Gate1 and CD45-Gate2 substantially inhibited the CAR cytotoxicity against the Cov644 (MUC16Low) target cells (FIGS. 5A-5B). These data together illustrate that the CD45-gate can effectively control CAR function and the level or degree of inhibition can be affected by and vary with the antigen density.

### Regulation of CD45-Gate CAR T Cell Cytotoxicity

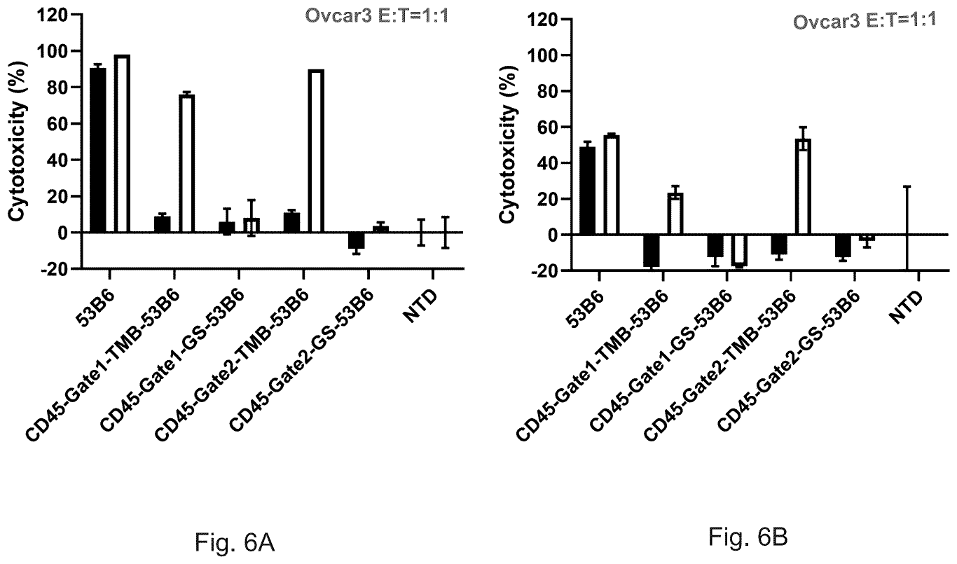

Next, whether the protease cleavage and the removal of CD45-gate from CAR can restore its cytotoxicity was evaluated. To test this, the cytotoxicity assay was carried out with the MUC16High Ovcar3 target cells similarly as above, except that serum-free medium was used in the assay to allow thrombin activity. The cytotoxicity of each clone was evaluated against the target cells at E:T of 1:1, with or without thrombin protease (final concentration of 2 μg/ml). In this condition, the regular 53B6 CAR T cells still can efficiently kill the target cells, albeit that the activity was weaker than their previous performance in the serum-rich medium. There is no change in 53B6 CAR activity when thrombin is introduced into the culture (FIG. 6A). Importantly, data from the clone of CD45-Gate1-TMB-53B6 and CD45-Gate2-TMB-53B6 demonstrates that the CD45-gate effectively inhibited the CAR activity and the cytotoxicity was mostly restored when the protease was present. In contrast, the CD45-gate in the clone of CD45-Gate1-GS-53B6 or CD45-Gate2-GS-53B6 could not be efficiently released and thus the CAR activity remained inhibited despite the presence of the protease.

### Example 2. Expression and Activity of Anti-MUC16 CAR

### CAR T Production

T cells were isolated from human peripheral blood mononuclear cells (PBMCs) obtained by density gradient centrifugation (Ficoll Paque, GE Healthcare, Pittsburgh, PA) using a Pan T cell isolation kit (Miltenyi Biotec, Auburn, CA) and cryopreserved.

To make lentivirus encoding 53B6 CAR, HEK-293T cells were plated at 0.45 million cells per mL in 2 mL of DMEM (Gibco) supplemented with 10% FBS (Hyclone or JR Scientific) per well of a 6-well plate on Day 0. On Day 1, the lentivirus was prepared by mixing together lentiviral packaging vectors 1.5 μg psPAX2, 0.5 μg pMD2G, and 1 μg of the appropriate transfer CAR vector in 250 μL Opti-MEM (Gibco) per well of the 6-well plate (“DNA mix”). 10 μL Lipofectamine 2000 (Invitrogen) in 250 μL Opti-MEM was incubated at room temperature for 5 minutes and then added to the DNA mix. The mixture was incubated at room temperature for 20 minutes and the total volume of 500 μL was slowly added to the sides of the wells containing HEK-293T. Purified T cells were activated in T cell transduction media (X-Vivo-15 medium [Lonza] with 10% FBS) supplemented with 20 ng/ml of human IL-2 (Miltenyi Biotec) and human T cell TransAct reagent (Miltenyi Biotec), as recommended by the manufacturer. On Day 2, the media from each well of the 6-well plate was replaced with 2 mL per well of T cell transduction media. On Day 3, T cells were resuspended at 0.33 million cells per mL in 1 mL of T cell transduction media. The lentiviral supernatant from HEK293T cells was harvested and passed through 0.45 micron filter (EMD Millipore) to remove cell debris, and then added to 0.5 million activated T cells along with 20 ng/ml human IL-2. Alternatively, to enhance the transduction efficiency, lentiviruses can be prepared with scaled up ratio as previous protocol and concentrated with Lenti-X™ Concentrator (Takara), following the manufacturer's protocol. Activated T cells were transduced at approximately MOI of 5 to generate CAR T cells.

On Day 5, 4.5 mL of T cell expansion media (X-Vivo-15 with 5% human AB serum [Gemini Bio]) was added to each well of a Grex-24 (Wilson Wolf) plate. IL-2 (20 ng/ml) was supplemented every 2-3 days. On Day 9 and Day 13, transduction efficiency was determined by detecting the percentage of T cells that recognize recombinant MUC16 protein (in-house) using flow cytometry. On Day 14, 53B6 CAR-T cells were cryopreserved.

### Flow Cytometry Analysis (FIGS. 7A-7B)

To determine the percentage of T cells that were successfully transduced with 53B6 CAR, T cells were first incubated with 3 μg/ml biotinylated recombinant MUC16 protein (in house) in FACS buffer (PBS+0.5% BSA with 2 mM EDTA) for 30 minutes at 4° C. The cells were then washed with FACS buffer, stained with streptavidin-PE labelled protein (Thermo Fisher, Cat #21627) at 1:250 ratio and analyzed using flow cytometry. As example, FIG. 7A shows that 53B6 CAR T cells were successfully generated from multiple productions using different T cell donors. The flow cytometry plot of 53B6 CAR T cells was from one of the productions. Empty vector (EV) control T cells were included in the studies.

Alternatively, another recombinant MUC16 conjugated with Alexa Fluor® 647 were used to directly stain CAR T cells at approximately final concentration of 3 μg/ml to evaluate the binding of 53B6 CAR in FIGS. 2A and 3B.

Phenotypes (FIG. 7B) of the CAR T cells were also determined on Day 13 according to CD62L (Biolegend) and CD45RO (Biolegend) expression within the CAR+ T cell population as follows: stem cell memory (Tscm; CD45RO−/CD62L+), central memory (Tcm; CD45RO+/CD62L+), effector memory (Tem; CD45RO+/CD62L−), effector cells (Teff; CD45RO−/CD62L−). Lineage markers using antibodies toward CD3 (BD Biosciences), CD4 (Biolegend) and CD8 (Biolegend) were also included in the stain.

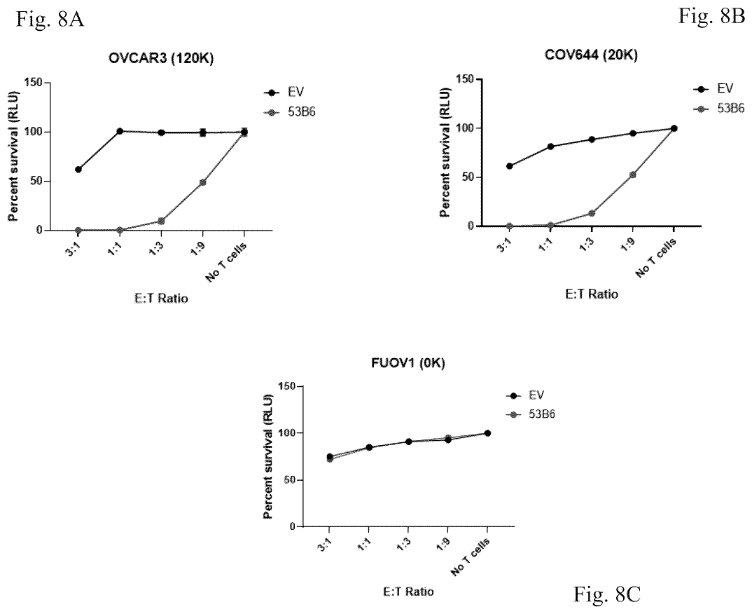

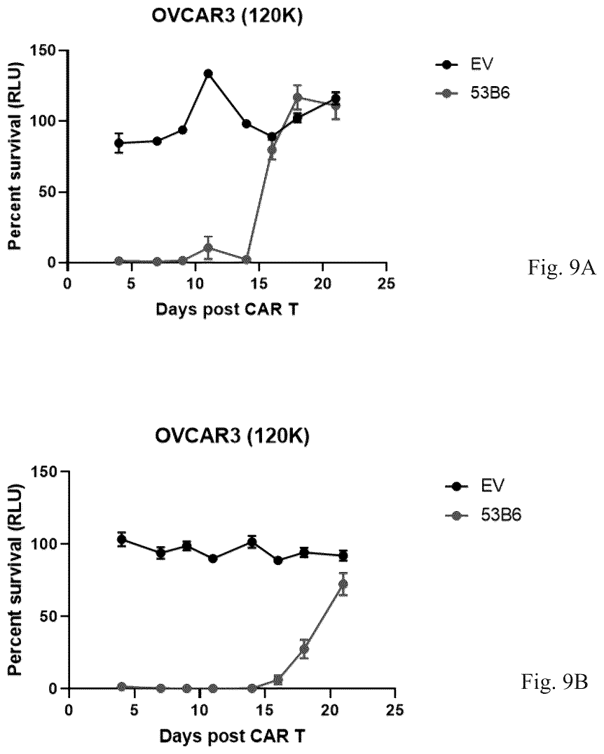

### Cytotoxicity Assay (FIGS. 8A-8C and FIGS. 9A-9B)

### Short Term Killing Assay

This example describes experiments used to determine the specificity and in vitro activity of 53B6 CAR.

OVCAR3, COV644 and FUOV1 ovarian cell lines were purchased from ATCC or Sigma, and surface expression of MUC16 protein was determined using a quantitative analysis kit (Agilent Cat. #K007811-8), according to manufacturer's recommendation. To determine the activity of 53B6 CAR T cells, 1×10⁴ luciferase-expressing ovarian target cells were co-cultured in RPMI 1640 medium (Gibco) supplemented with 10% FBS with 53B6 CAR+ T cells at defined Effector: Target (E:T) ratio in 96-well assay plates (Corning). Empty vector (EV) control T cells were also included in the study. Cell viability was measured after 72 hours using One-Glo reagent (Promega). Each condition was assayed in replicate wells. Percent target cell survival after being exposed to T cells was determined by comparing to target cells alone. Average percentage of live cells and standard deviation are shown (FIGS. 8A-8C).

### Long Term Serial Killing Assay

A serial killing assay involves repeated exposure of CAR T cells to their target causing the CAR T cells to undergo proliferation and in certain cases, differentiation and exhaustion.

To determine the persistent killing of 53B6 CAR T cells, 1×10⁴ luciferase-expressing OVCAR3 target cells were co-cultured in RPMI 1640 medium (Gibco) supplemented with 10% FBS with 53B6 CAR+ T cells at Effector: Target ratio of 1:1 in a serial killing assay using 96-well assay plates. Every 2-3 days thereafter, 100 μl medium from each well were transferred to freshly plated target cells (1×10⁴ cells per well) and percent target cell survival of the previously plated target cells after being exposed to CAR T cells was determined by comparing to target cells alone using One-Glo reagent. Each condition was assayed in 3 to 6 replicates. Empty vector (EV) control T cells were also included in the study. Average percentage of live cells and standard deviation are shown. Data presented are the activity of 53B6 CAR T cells from two different donors (FIGS. 9A-9B).

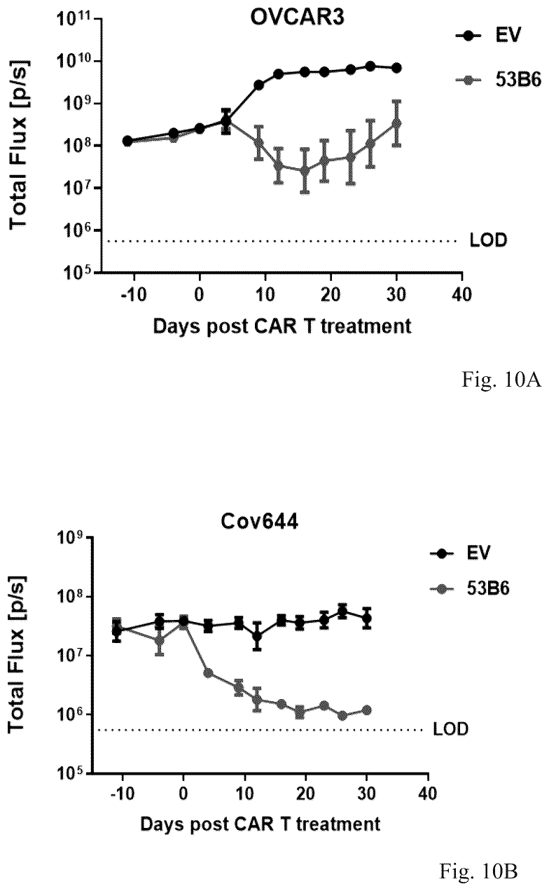

### In Vivo Activity (FIGS. 10A-10B)

To test the anti-tumor activity of 53B6 CAR T cells, two different orthotopic ovarian tumor models were used. Luciferase-labeled OVCAR3 and COV644 ovarian cells were cultured in complete growth medium (RPMI 1640 supplemented with 10% FBS). Cells were collected and diluted to 15×10⁶ viable cells/ml in PBS, and cell suspension was kept on ice until inoculation. NSG mice (Jackson Laboratory), aged 7-8 weeks old, were injected intra-peritoneally (IP) with 200 μl (3×10⁶ target cells per mouse) of OVCAR3 or COV644. Tumor burden was monitored using the IVIS Spectrum (Perkin Elmer), where the animals were injected intra-peritoneally with 200 μl of luciferin (15 mg/ml; Pierce) and bioluminescent intensity was measured. Mice were randomized into groups of 5 based on total body bioluminescence about two weeks post tumor implantation. 53B6 CAR T cells were then thawed and resuspended in PBS. OVCAR3-tumor bearing mice received 1×10⁶ 53B6 CAR+ T cells per animal, whereas COV644-tumor bearing mice received 3×10⁶ 53B6 CAR+ T cells per animal. Empty-vector control T cells were also included in the study. All animals infused with T cells were dosed via ip injection in a volume of 200 μl/mouse. The in vivo activity of the 53B6 CAR T cells against the tumor was monitored by bioluminescence, and the results demonstrated anti-tumor activity in both models (FIGS. 10A-10B).

### Example 3. Test of CD45-Gate CAR T Cell Activity

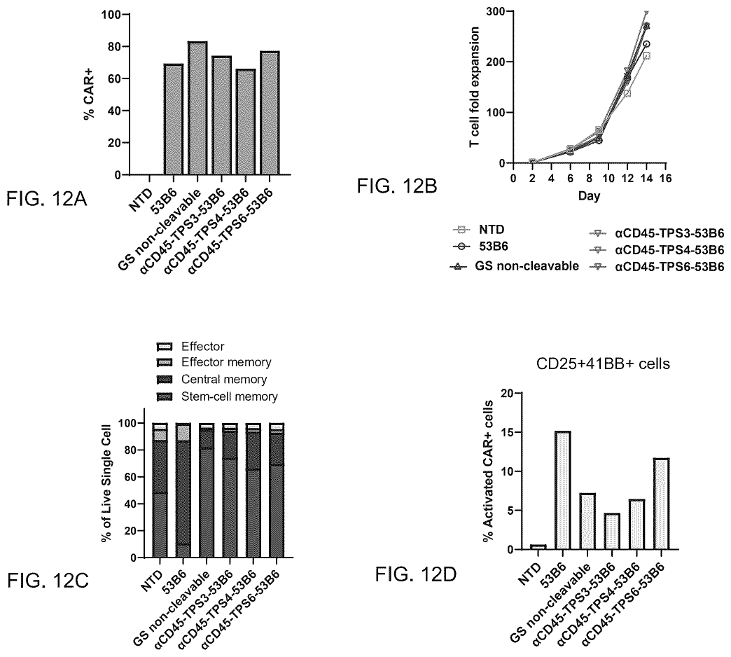

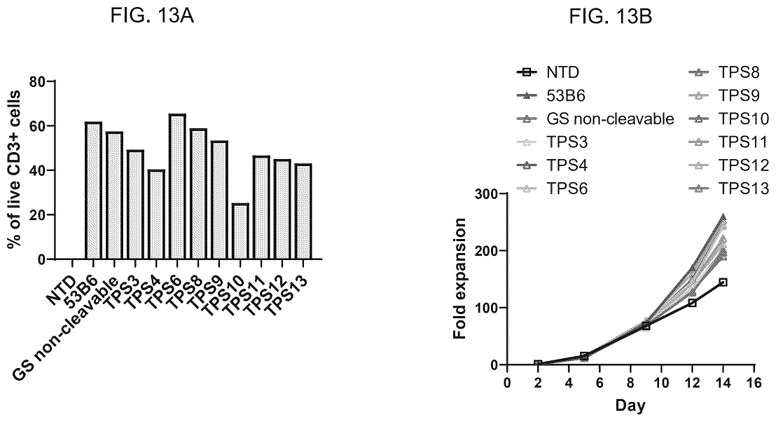

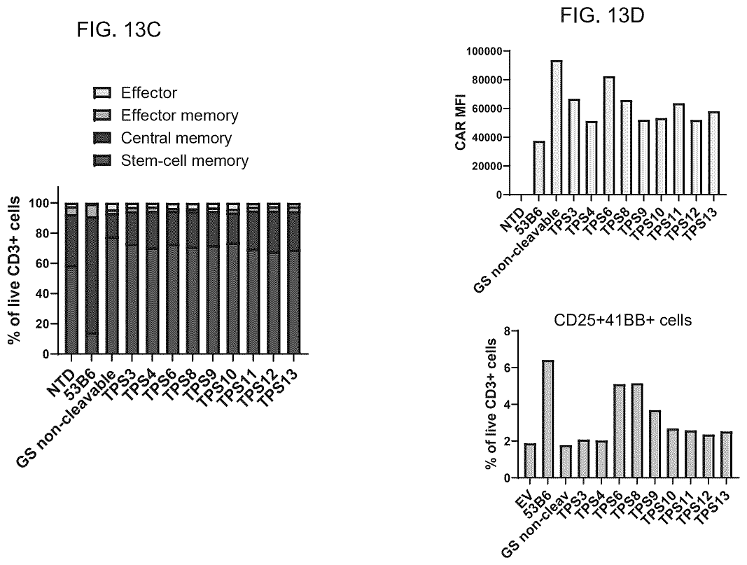

### 3.1. Production of New CD45-Gate CAR T (Effector) Cells (FIGS. 12A-12D and FIGS. 13A-13D)

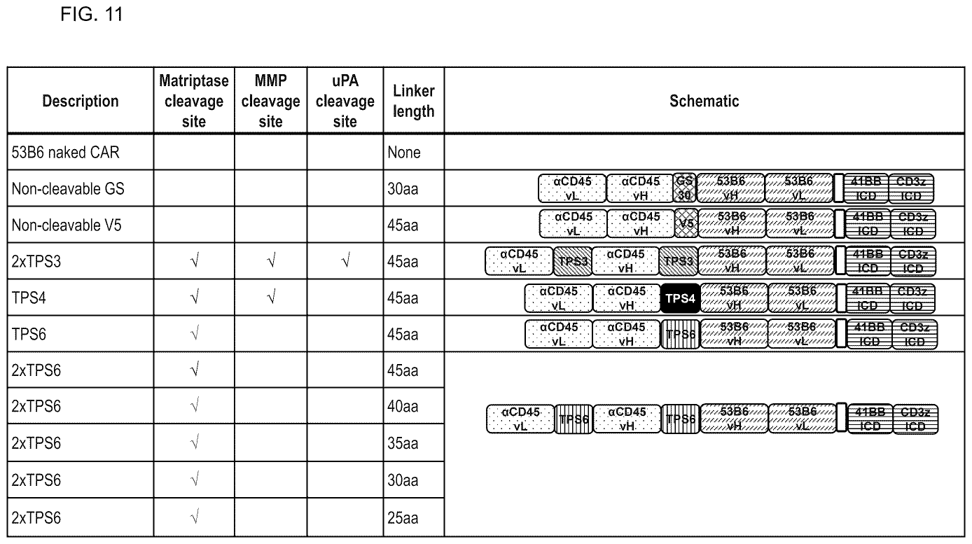

CAR T cells expressing a CD45-gate MUC16 CAR comprising a TPS linker selected from a panel of TPS linkers (similar to the CD45-gate CARs diagrammed in FIG. 11), were successfully generated and characterized using flow cytometry according to methods described in Example 2 and below. In addition to cells expressing CARs comprising one of the cleavable TPS linkers, cells expressing a CAR comprising a non-cleavable GS linker were prepared as a negative control expected not to exhibit CAR activity because the CD45-gate cannot be released by proteases. Cells expressing a non-gated MUC16 CAR (53B6) were included as a second, positive control expected to be active in the presence or absence of proteases.

Each CAR+ T cell population was detected with AlexaFluor 647-conjugated recombinant MUC16 protein (SEA1-4) using flow cytometry (FIG. 12A). The overall expansion of T cells remains similar among all the clones and is comparable to non-transduced control (NTD), indicating no obvious fratricide (FIG. 12B). Autoactivation and memory phenotypes of the CAR T cells were also determined using flow cytometry by analyzing the expression of CD25/41BB and CD45RO/CD62L on Day 9 and Day 14, respectively. Overall, CD45-gate CAR T cells contain a higher percentage of stem-cell memory subset (CD45RO−/CD62L+, FIG. 12C) and exhibit less tonic signaling (CD25+/41BB+, FIG. 12D) than naked CAR T cells, suggesting a potential benefit of CD45-gate CARs in controlling T cell exhaustion. At the end of the production (Day 14-16), all T cells were cryopreserved in 90% FBS/10% DMSO using rate-controlled freezing chambers and stored in liquid nitrogen.

A second set of CAR T cell populations, each expressing a CD45-gate MUC16 CAR comprising a TPS linker selected from a second panel of TPS linkers, were generated successfully and characterized according to methods described in Example 2 and this section (results shown in FIGS. 13A-13D). The success in generating CD45-gate CAR T cells was surprising because T cells express endogenous CD45, however no noticeable fratricide was observed that would have hindered the production of the CD45-gate CAR T cells.

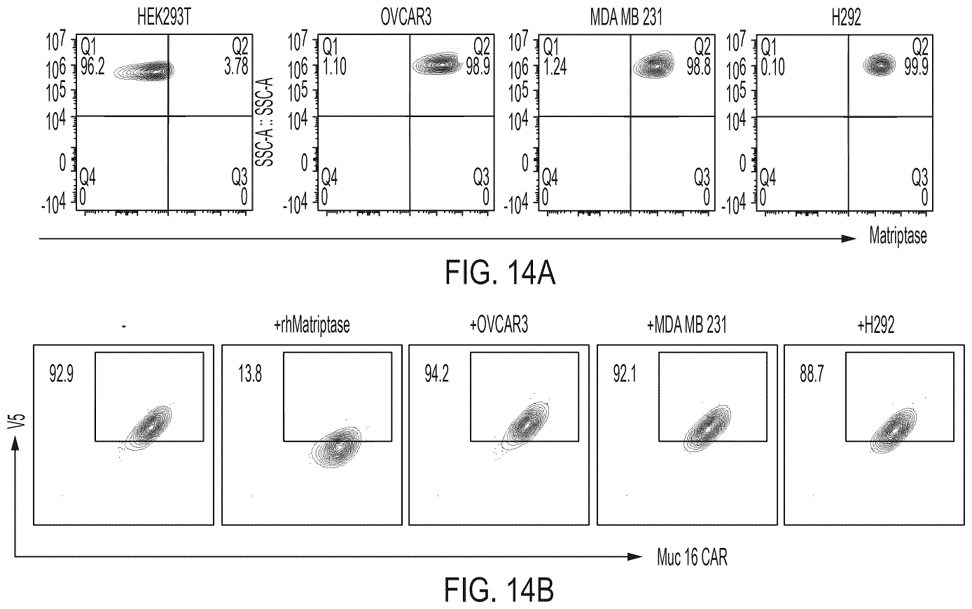

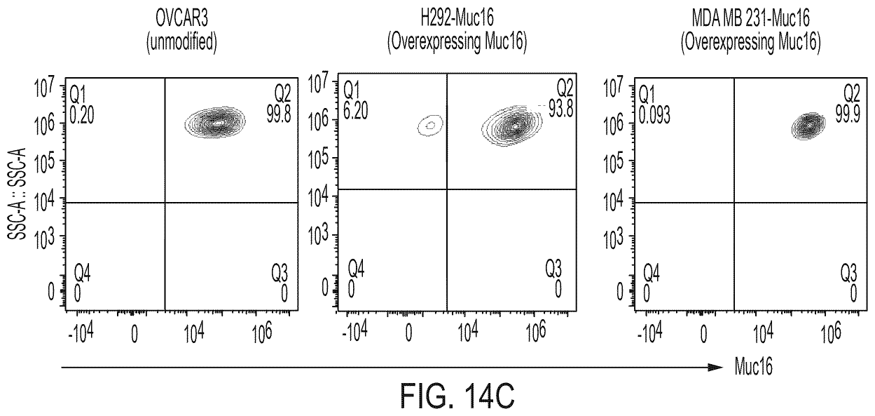

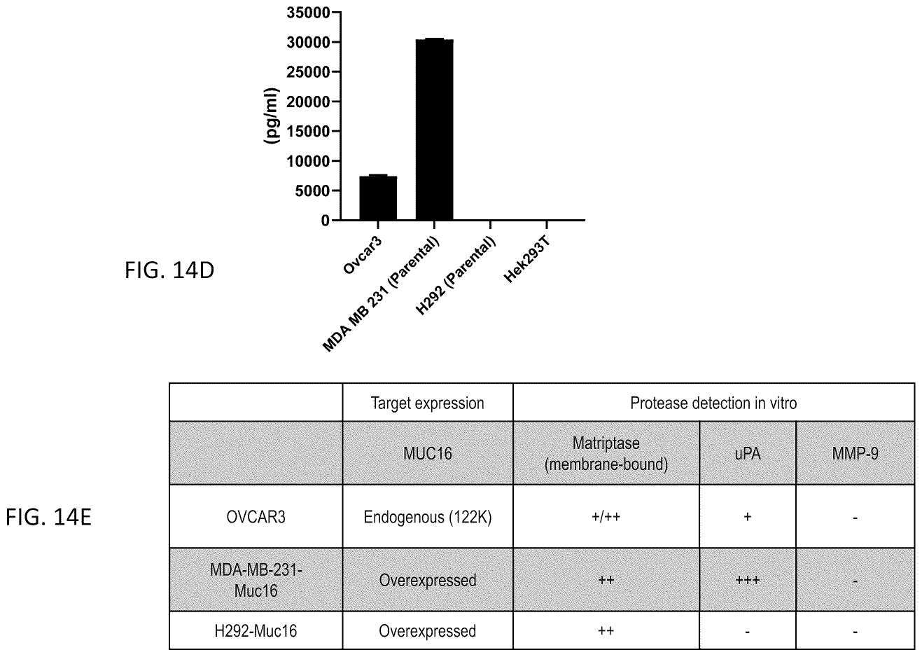

### 3.2. Generation and Analysis of Reagent Cell Lines (FIGS. 14A-14E)

Target cell lines were developed in house based on three cell lines that are positive for (i.e. express active) tumor-associated proteases, including an ovarian cancer cell line OVCAR3, a breast cancer cell line MDA-MB-231, and a lung carcinoma cell line NCI-H292. The surface expression of matriptase (3 μg/mL; R&D, BAF3946) was confirmed using flow cytometry (FIG. 14A) and the secretion of urokinase (uPA) was detected in conditioned media by an ELISA kit (R&D, DUPA00) (FIG. 14D). In addition, the activity of surface expressed matriptase was assessed through the detection of a V5 tag that is attached to CAR via a matriptase cleavable linker TPS6, as previously described in Example 1 (FIG. 14B). Compared to the recombinant matriptase which effectively mediated linker cleavage and removal of V5 tag, the endogenous matriptase showed limited activity, which is, without wishing to be bound by specific mechanisms, believed to be due to the lack of activation of matriptase in the in vitro assay settings. For example, FIG. 14B shows that in this experiment in vitro matriptase is not active, consistent with the CD45-Gate CAR with TPS6 also not being active.

To study the target-mediated CAR activation, MDA-MB-231 and H292 cell lines engineered to overexpress human MUC16 SEA1-4 and OVCAR3 cell line expressing endogenous MUC16 were used as target cells. The expression of the target protein MUC16 was validated by flow cytometry (FIG. 14C). All three cell lines were then engineered to co-express a firefly luciferase and a nuclear GFP using a 2A peptide to enable sensitive measurement of tumor killing using luminescence reading or Incucyte imaging.

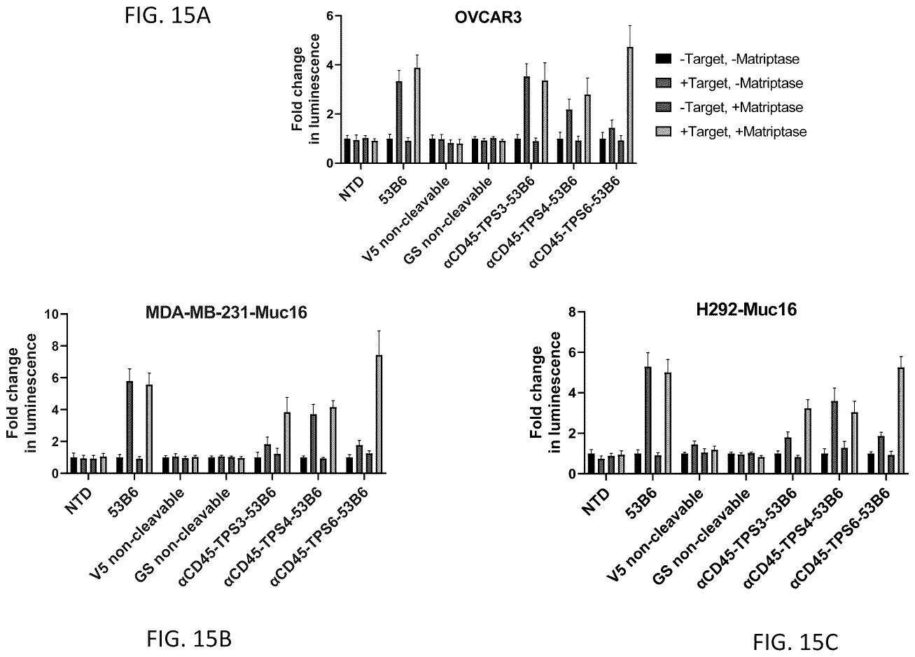

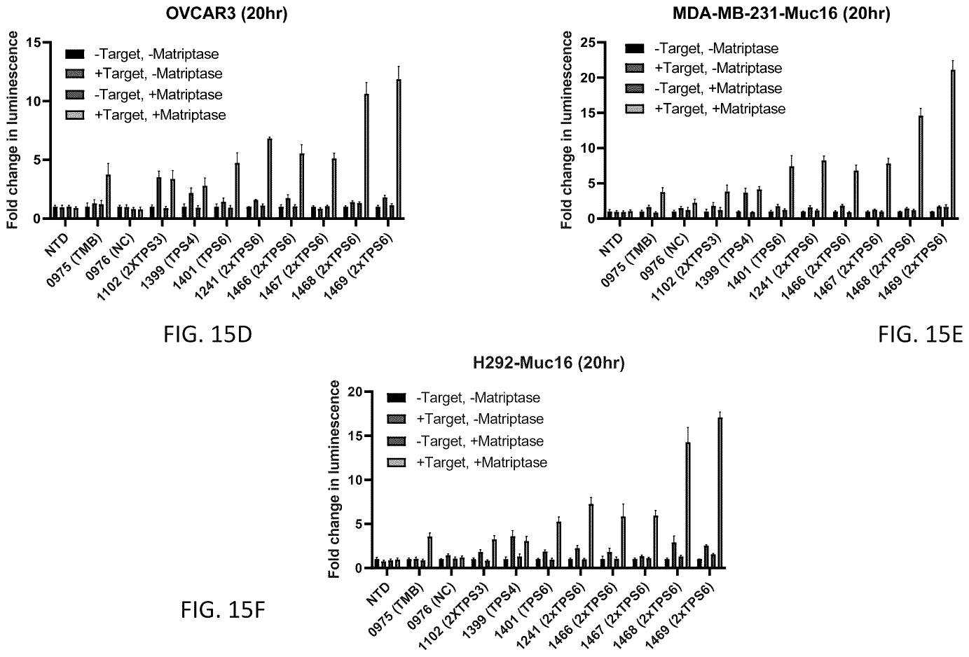

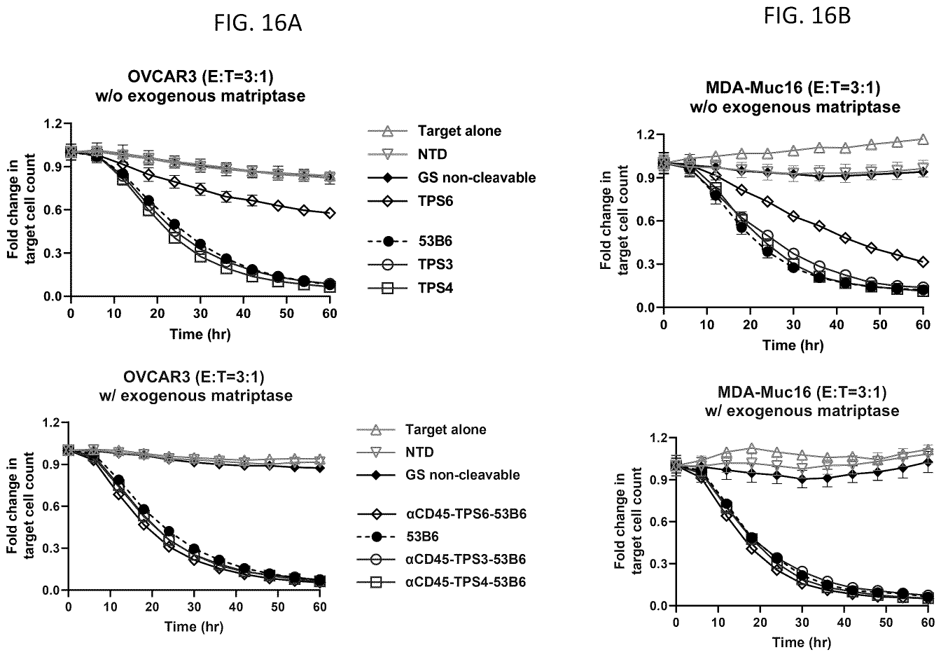

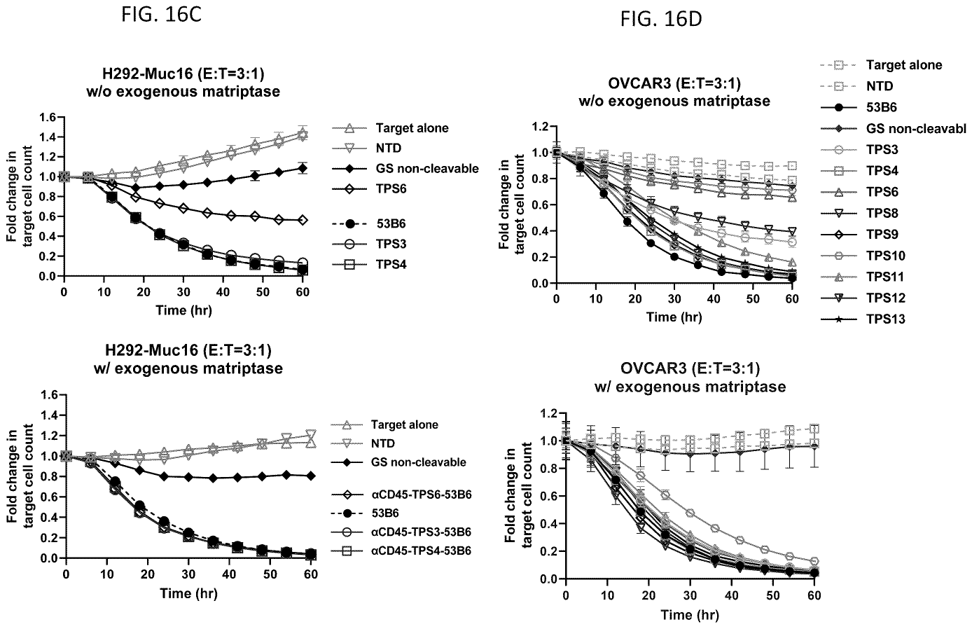

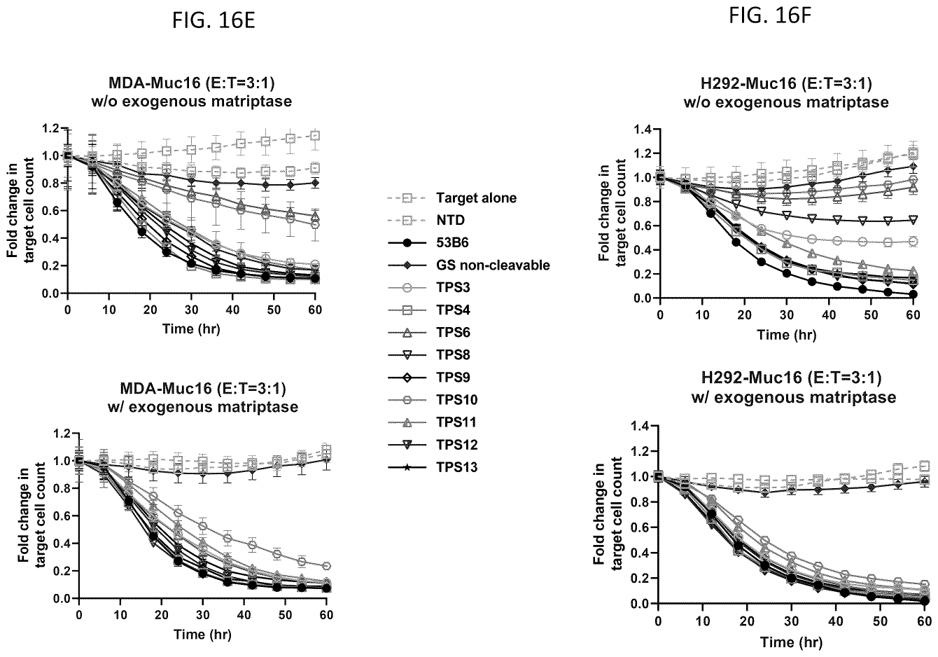

### 3.3 In Vitro Analysis of CD45-Gate CAR T Cell Activity, Protease Dependence and Sensitivity (FIGS. 15A-15F and FIGS. 16A-16F)

A Jurkat-NFAT reporter cell line was used to evaluate the activity of CD45-gate CARs. The Jurkat-NFAT reporter cell line contains a nuclear factor of activated T-cell (NFAT) promoter upstream of a luciferase gene, which allows the quantitative measurement of signaling activation upon stimulation. To generate CAR reporter cells, Jurkat-NFAT cells were transduced with lentivirus encoding CD45-gate CARs and expression was analyzed after 6 days using recombinant MUC16 protein (SEA1-4). Transduced cells were then cocultured with target cells (1×10⁴/well) at a 1:1 E:T ratio in 96-well white flat-bottom plates. Wells containing only Jurkat cells also were included as negative controls. All cocultures were performed in CTS OpTmizer T cell expansion SFM (Gibco) with or without human recombinant matriptase (0.2 μg/mL, R&D, 3946-SEB-010). After overnight incubation at 37° C., the reporter gene activation was detected by luminescence and normalized to the untreated control signal when no target cells were added.

As shown in FIGS. 15A-15F, exposure to MUC16 positive target cells led to the activation of 53B6 CAR while no activation was detected with a CD45-gate CAR with non-cleavable linker. However, the activation can be restored via the cleavable linkers upon exposure to endogenous (“−Matriptase” in FIGS. 15A-15C) or exogenous proteases (“+Matriptase” in FIGS. 15A-15C). This shows that the CD45-gate suppresses CAR activity and the suppression can be protease-sensitive when the linker comprises one or more protease sites. The data comprise clones 1241 (2×TPS6), 1466 (2×TPS6), 1467 (2×TPS6), 1468 (2×TPS6), and 1469 (2×TPS6), each of which utilizes linkers with various lengths (45, 35, 30, 25, 20-mers) and yet they enable highly similar functionality. The following constructs were used in FIGS. 15D-15F:
0975 (TMB) refers to SEQ ID NO: 16 (N-HA-4131scFv-V5-2G4STMB-53B6, comprising the linker GSTMB (45 aa) having the amino acid sequence of SEQ ID NO: 8
0976 (NC) refers to SEQ ID NO: 17 (non-cleavable)
1102 (2×TPS3) refers to SEQ ID NO: 24 (N-HA-4131-V_{L}-RS-VH-TPS1-53B6, comprising the linker GSTPS1 (45 aa), having the amino acid sequence of SEQ ID NO: 9, and comprising the linker TPS cleavage, having the amino acid sequence of SEQ ID NO: 53 flanked on both ends by GS sequences
1399 (TPS4) refers to SEQ ID NO: 137, a construct containing the elements V5-GS20-4131scFv-TPS4-53B6, comprising the linker TPS4 45 aa (SEQ ID NO: 107) and a GS sequence
1401 (TPS6) refers to SEQ ID NO: 138, a construct containing the elements V5-GS20-4131scFv-TPS6-53B6, comprising the linker TPS6 45 aa having the amino acid sequence of SEQ ID NO: 113
1241 (2×TPS6) refers to SEQ ID NO: 144, a construct containing the elements N-HA-4131scFv-2×TPS6-53B6, comprising the linker TPS6 35aa having the amino acid sequence of SEQ ID NO: 112 and a second linker comprising the cleavage site MTSP (3) (SEQ ID NO: 95) flanked on both ends by a GS sequence
1466 (2×TPS6) refers to SEQ ID NO: 153, a construct containing the elements V5-GS20-4131scFv-2×TPS6-35-53B6, comprising 2 copies of the linker TPS6 35 aa, each copy having the amino acid sequence of SEQ ID NO: 112
1467 (2×TPS6) refers to SEQ ID NO: 154, a construct containing the elements V5-GS20-4131scFv-2×TPS6-30-53B6, comprising the linker TPS6 35 aa (SEQ ID NO: 112) and the linker TPS6 30 aa (SEQ ID NO: 111)
1468 (2×TPS6) refers to SEQ ID NO: 155, a construct containing the elements V5-GS20-4131scFv-2×TPS6-25-53B6, comprising the linker TPS6 35 aa (SEQ ID NO:112) and the linker TPS6 25 aa (SEQ ID NO: 110)
1469 (2×TPS6) refers to SEQ ID NO: 156, a construct containing the elements V5-GS20-4131scFv-2×TPS6-20-53B6, comprising the linker TPS6 35aa (SEQ ID NO: 112) and the linker TPS6 20aa (SEQ ID NO: 109)

### 3.4. Cytotoxicity of CD45-Gate CAR T Cells

The short-term cytotoxicity of CD45-gate CAR T cells were characterized using Incucyte. For kinetic analysis of tumor cell killing, target cells MDA-MB-231-MUC16, H292-MUC16 and OVCAR3 cells engineered to express nuclear GFP were plated at a concentration of 1×10⁴ cells per well in 96-well black flat-bottom plates. The following day, T cells were thawed and added at an effector: target (E:T) ratio of 3:1. All cocultures were performed in CTS OpTmizer T cell expansion SFM (Serum-Free Medium; Gibco) with or without human recombinant matriptase (0.2 μg/mL, R&D, 3946-SEB-010). The number of viable target cells was monitored by fluorescent imaging every 6 hours over 60 hours using the IncuCyte Live Cell Analysis System (Essen BioScience). Live-cell numbers were quantified by IncuCyte S3 software (Essen BioScience) and normalized to the initial counts at time 0 (time zero). Average fold change in target cell count and standard deviation are shown in FIGS. 16A-16F. CD45-gate CARs with non-cleavable GS linker effectively inhibited CAR function. In the presence of endogenous proteases (“w/o [without] exogenous matriptase”) (top panels of FIGS. 16A-16F), CD45-gate CARs with various TPS linkers showed a spectrum of activity, with some of the linkers (e.g., TPS4) exhibiting activity comparable to that of non-gated 53B6 CAR. In the presence of exogenous matriptase (“w/[with] exogenous matriptase”) (bottom panels of FIGS. 16A-16F), all CD45-gate CARs with TPS linkers showed cytotoxic activities. FIGS. 16A-16C show results using linkers from the first set of linkers (TPS3, TPS4, TPS6) plus controls; FIGS. 16D-16F show results using linkers from the first and second sets of linkers (TPS3, TPS4, TPS6, TPS8-TPS13) plus controls.

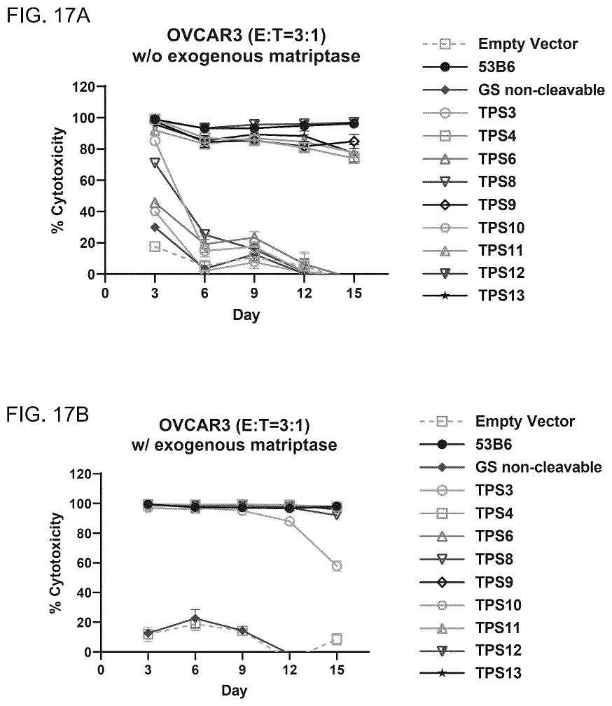

### 3.5 Long-Term Killing Assay (FIGS. 17A-17B)

To determine the persistent killing of CD45-gate CAR T cells upon repeated exposure to target, luciferase-expressing OVCAR3 cells were plated in CTS OpTmizer T cell expansion SFM (Gibco) at a concentration of 1×10⁴ cells per well in 96-well white flat-bottom plates. The following day, T cells were thawed and added at a E:T ratio of 3:1. All cocultures were performed with or without human recombinant matriptase (0.2 μg/mL, R&D, 3946-SEB-010). Every 2-3 days, half of the supernatant containing CAR T cells from each well was transferred to freshly plated target cells (1×10⁴/well) and target cell viability was measured by luminescence using One-Glo assay (Promega) as previously described. Data presented are the cytotoxicity of CD45-gate CAR T cells, showing that those with cleavable linkers maintained long-term cytotoxicity in the presence of endogenous (FIG. 17A) or exogenous (FIG. 17B) proteases.

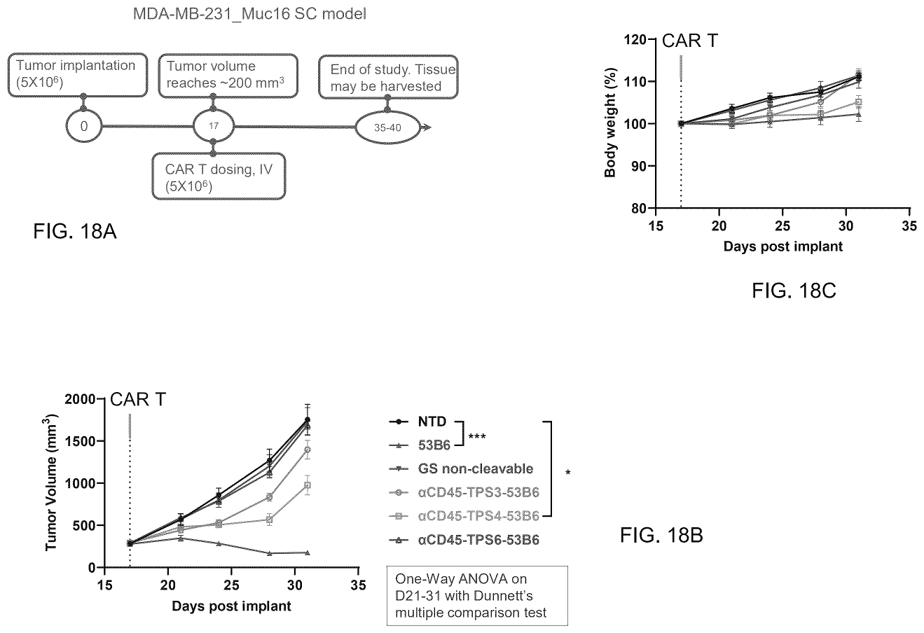

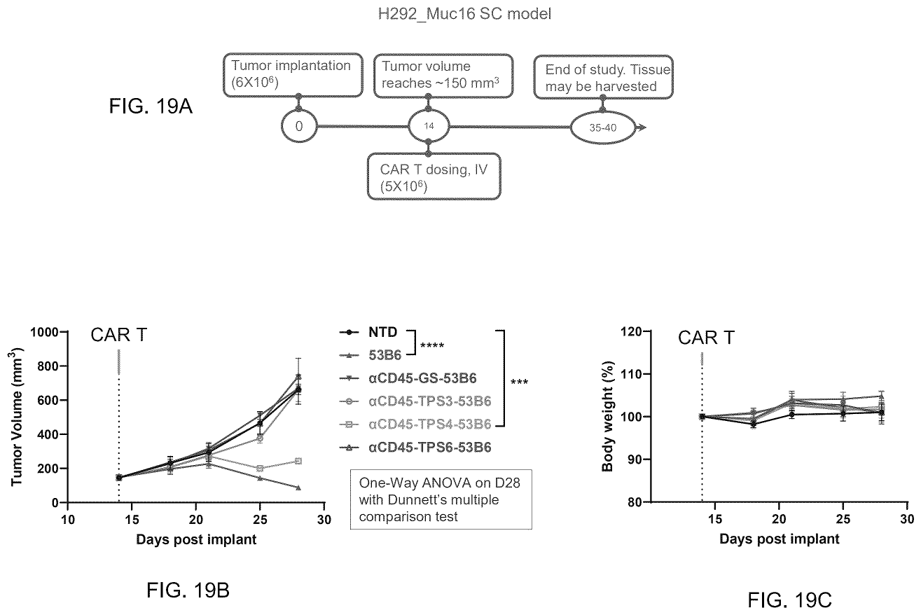

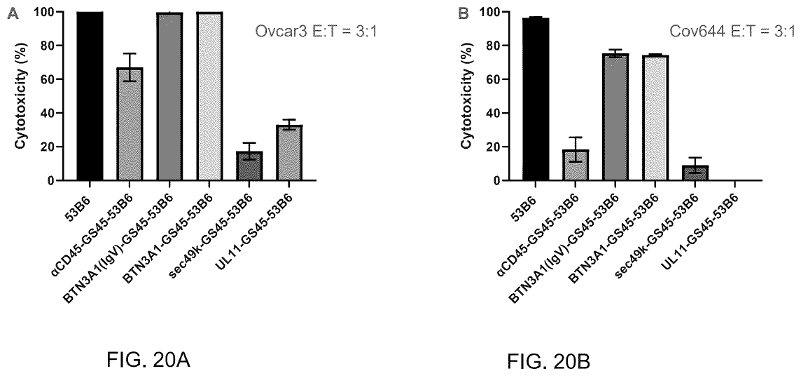

### In Vivo Assay (FIGS. 18A-18C and FIGS. 19A-19C)

In vivo anti-tumor efficacy of CD45-gate CAR T cells was evaluated in a breast cancer xenograft model (FIG. 18A shows schematic outline of experiment). 7-8 weeks old NSG mice (Jackson Laboratory) were injected subcutaneously (SC) with 200 μl of MDA-MB-231-MUC16 cells (5×10⁶ cells mixed with Matrigel at 1:1 ratio). Mice were randomized into 6 groups (n=7-8) when average tumor size reached 290 mm³ on day 17. Each animal received 5 million CAR+ cells via tail vein injection. Total T cell numbers were kept constant across all groups by normalizing with non-transduced T cells. Tumors were measured twice a week and tumor volume was calculated as length (mm)×width (mm)×height (mm)/2. Statistical analysis of observed differences was performed by one-way ANOVA followed by Dunnett's test for multiple comparisons.

As shown in FIG. 18B, anti-tumor CAR activity cannot be activated in a CD45-gate CAR that comprises only a non-cleavable GS linker in the MDA-MB-231 model. In contrast, anti-tumor CAR activity can be activated in CD45-gate CARs with cleavable linkers (e.g., TPS4). Tumor growth was reduced in mice treated with the CD45-gate CAR αCD45-TPS4-53B6.

Treatment with CD45-gate CAR T cells had no effect on the body weight of the mice (FIG. 18C).

In vivo anti-tumor efficacy of CD45-gate CAR T cells was also evaluated in a non-small cell lung cancer xenograft model (FIG. 19A shows schematic outline of experiment). NSG mice (Jackson Laboratory) were injected subcutaneously (SC) with 200 μl of NCI-H292-MUC16 cells (6×10⁶ cells mixed with Matrigel at 1:1 ratio). Mice were randomized into 6 groups (n=6-7) when average tumor size reached 150 mm³ on day 14. CAR T cells were then thawed and infused immediately (5×10⁶ CAR+ cells per animal) via tail vein injection. Total T cell numbers were kept constant across all groups by normalizing with non-transduced T cells. Tumors were measured twice a week and tumor volume was calculated as length (mm)×width (mm)×height (mm)/2. Statistical analysis of observed differences was performed by one-way ANOVA followed by Dunnett's test for multiple comparisons. Without wishing to be limited to specific mechanisms, the results likely reflect the various levels of protease activity at the tumor site in this in vivo tumor model.

As shown in FIG. 19B, a CD45-gate CAR with a non-cleavable GS linker lacked any CAR anti-tumor activity in the H292 model. In contrast, a CD45-gate CAR with a cleavable linker (TPS4) showed anti-tumor activity.

Treatment with CD45-gate CAR T cells had no effect on the body weight of the mice (FIG. 19C), indicating that the CD45-gate CAR T cells did not lead to general toxicity in vivo.

### REFERENCES

1. Ledbetter J A, Tonks N K, Fischer E H, Clark E A. CD45 regulates signal transduction and lymphocyte activation by specific association with receptor molecules on T or B cells. Proc Natl Acad Sci USA. 1988. doi: 10.1073/pnas.85.22.8628
2. McNeill L, Salmond R J, Cooper J C, et al. The Differential Regulation of Lck Kinase Phosphorylation Sites by CD45 Is Critical for T Cell Receptor Signaling Responses. Immunity. 2007. doi: 10.1016/j.immuni.2007.07.015
3. Felberg J, Johnson P. Characterization of Recombinant CD45 Cytoplasmic Domain Proteins. J Biol Chem. 1998. doi: 10.1074/jbc.273.28.17839
4. Davis S J, van der Merwe P A. The kinetic-segregation model: TCR triggering and beyond. Nat Immunol. 2006. doi: 10.1038/ni1369
5. Chang V T, Fernandes R A, Ganzinger K A, et al. Initiation of T cell signaling by CD45 segregation at “close contacts.” Nat Immunol. 2016. doi: 10.1038/ni.3392
6. Razvag Y, Neve-Oz Y, Sajman J, Reches M, Sherman E. Nanoscale kinetic segregation of TCR and CD45 in engaged microvilli facilitates early T cell activation. Nat Commun. 2018. doi: 10.1038/s41467-018-03127-w 7 Thiel N, Zischke J, Elbasani E, Kay-Fedorov P, Messerle M. Viral interference with functions of the cellular receptor tyrosine phosphatase CD45. Viruses. 2015. doi: 10.3390/v7031540
8. Payne K K, Mine J A, Biswas S, et al. BTN3A1 governs antitumor responses by coordinating ab and gd T cells. Science (80-). 2020. doi: 10.1126/science.aay2767
9. Bakalar M H, Joffe A M, Schmid E M, Son S, Podolski M, Fletcher D A. Size-Dependent Segregation Controls Macrophage Phagocytosis of Antibody-Opsonized Targets. Cell. 2018. doi: 10.1016/j.cell.2018.05.059
10. Weidle U H, Tiefenthaler G, Georges G. Proteases as activators for cytotoxic prodrugs in antitumor therapy. Cancer Genomics and Proteomics. 2014.
11. Gialeli C, Theocharis A D, Karamanos N K. Roles of matrix metalloproteinases in cancer progression and their pharmacological targeting. FEBS J. 2011. doi: 10.1111/j.1742-4658.2010.07919.x
12. Geiger M, Stubenrauch K G, Sam J, et al. Protease-activation using anti-idiotypic masks enables tumor specificity of a folate receptor 1-T cell bispecific antibody. Nat Commun. 2020. doi: 10.1038/s41467-020-16838-w
13. Desnoyers L R, Vasiljeva O, Richardson J H, et al. Tumor-specific activation of an EGFR-targeting probody enhances therapeutic index. Sci Transl Med. 2013. doi: 10.1126/scitranslmed.3006682
14. Sommer C, Cheng H Y, Nguyen D, et al. Allogeneic FLT3 CAR T Cells with an Off-Switch Exhibit Potent Activity against AML and Can Be Depleted to Expedite Bone Marrow Recovery. Mol Ther. 2020. doi: 10.1016/j.ymthe.2020.06.022
15. Sommer C, Boldajipour B, Kuo T C, et al. Preclinical Evaluation of Allogeneic CAR T Cells Targeting BCMA for the Treatment of Multiple Myeloma. Mol Ther. 2019. doi: 10.1016/j.ymthe.2019.04.001

1-52. (canceled)

53. A method of reducing on-target off-tumor toxicity of CAR T cells comprising administering to a patient the engineered immune cell, expressing a protease-activating CD45-gate chimeric antigen receptor (CD45-gate CAR) comprising an extracellular domain, a transmembrane domain, and an intracellular domain, wherein the extracellular domain comprises: a CD45 recruiting domain comprising an anti-CD45 antibody, or an antigen binding fragment thereof, an antigen binding domain, and a linker comprising one or more protease cleavage sites that is cleavable by at least one protease, wherein the on-target, off-tumor toxicity is lower than the on-target, off-tumor toxicity of a control cell administered under the same conditions, wherein the control cell comprises a CAR that lacks at least one of a CD45 recruiting domain and a linker that comprises a protease cleavage site.

54-84. (canceled)

85. The method of claim 53, wherein the intracellular domain comprises at least one signaling domain that is reversibly inactivated by CD45.

86. The method of claim 53, wherein the linker is between the CD45 recruiting domain and the antigen binding domain.

87. The method of claim 53, wherein the CD45 recruiting domain comprises one or more linkers.

88. The method of claim 53, wherein the linker connects the carboxy terminus of the CD45 recruiting domain to the amino terminus of the antigen binding domain, and further wherein the intracellular domain comprises at least one signaling domain that is reversibly inactivated by CD45.

89. The method of claim 53, wherein the CD45 recruiting domain comprises an anti-CD45 antibody or an antigen-binding fragment thereof.

90. The method of claim 53, wherein the CD45 recruiting domain comprises the amino acid sequence of SEQ ID NO: 3, SEQ ID NO: 4, SEQ ID NO: 5, or SEQ ID NO: 6 without the signal sequence of SEQ ID NO:1.

91. The method of claim 53, wherein the antigen binding domain specifically binds BCMA, MUC16 EGFR, EGFRVIII, MUC1, Flt-3, WT-1, CD20, CD23, CD30, CD38, CD70, CD33, CD133, MHC-WT1, TSPAN10, MHC-PRAME, MHC-NY-ESO1, HER2, Carbonic anhydrase IX, LIV1, ADAM10, CHRNA2, LeY, NKG2D, CS1, CD44v6, ROR1, CD19, Claudin-18.2, PSCA, DLL3, Mud 7, Ly6G6D, PSMA, MSLN, or RNF43.

92. The method of claim 53, wherein the linker comprising at least one protease cleavage site has a length of 15-100 amino acids.

93. The method of claim 53, wherein the linker comprises one or more protease cleavage sites that are cleavable by at least one protease which is present in a tumor microenvironment.

94. The method of claim 53, wherein the linker comprises at least one protease cleavage site that is cleavable by a protease selected from the group consisting of: matrix metalloproteinase (MMP), matriptase (MT-SP1), trypsin, plasmin, prostate-specific antigen (PSA), urokinase plasminogen activator (uPA), urokinase plasminogen activator receptor (uPAR), legumain, a disintegrin and metalloproteinase (ADAM), a transmembrane Serine Protease (TMPRSS), Granzyme B, activated protein C, Caspase, Cathepsin, Chymase, Elastase, Guanidinobenzoatase, HtrAl, Human Neutrophil Elastase, Lactoferrin, Marapsin, NS3/4A, PACE4, tissue plasminogen activator (tPA), thrombin, DESC1, DPP-4, FAP, Hepsin, Matriptase-2, secretase, kallikrein-related peptidase (KLK), and tryptase.

95. The method of claim 53, wherein the linker comprises one or more protease cleavage sites comprising the amino acid sequence selected from the group consisting of SEQ ID NO: 32, 91-98, 103-105 or SEQ ID NO: 106.

96. The method of claim 53, wherein the linker comprises one or more amino acid sequences selected from the group consisting of SEQ ID NO: 8-10, 53, 89, 90, 99-102, 107-120, 172-176 or 177

97. The method of claim 53, wherein the intracellular domain comprises the cytoplasmic signaling domain of one or more of CD3 zeta, CD28, and CD2.

98. The method of claim 53, wherein the intracellular domain comprises at least one costimulatory domain selected from the group consisting of signaling region of CD28, OX-40, 4-1BB/CD137, CD2, CD7, CD27, CD30, CD40, inducible T cell costimulator (ICOS), lymphocyte function-associated antigen-1 (LFA-1), CD3 gamma, CD3 delta, CD3 epsilon, CD247, CD276 (B7-H3), LIGHT (TNFSF14), NKG2C, Ig alpha (CD79a), DAP-10, Fc gamma receptor, MHC class I molecule, Signaling Lymphocytic Activation Molecules (SLAM proteins), BTLA, a Toll ligand receptor, ICAM-1, CDS, GITR, BAFFR, HVEM (LIGHTR), KIRDS2, NKp80 (KLRF1), NKp44, NKp30, NKp46, CD19, CD4, CD8alpha, CD8beta, IL-2R beta, IL-2R gamma, IL-7R alpha, ITGA4, VLA1, ITGA6, ITGAD, ITGAEITGAL, IT GAM, ITGAX, ITGB1, ITGB7, NKG2D, TNFR2, TRANCE/RANKL, DNAMI (CD226), CD84, CD96 (Tactile), CEACAM1, CRT AM, Ly9 (CD229), CD 160 (BY55), PSGL1, CD100 (SEMA4D), CD69, SELPLG (CD162), LTBR, LAT, GADS, SLP-76, PAG/Cbp, CD 19a, a ligand that specifically binds with CD83, or any combination thereof.

99. The method of claim 53, wherein the protease-activating CD45-gate CAR comprises the amino acid sequence of any one of SEQ ID NOs: 11, 16-31, 56, 75, 76, 77, 78, 79, and 121-170 or a variant thereof, wherein the variant does not comprise the amino acid sequence of the HA tag of SEQ ID NO: 2, does not comprise the amino acid sequence of the V5 peptide motif of SEQ ID NO: 55, or does not comprise the signal sequence of SEQ ID NO: 1.
